(19) United States
(12) Patent Application Publication
(10) Pub. No.: US 2026/0250656 A1
(43) Pub. Date: Aug. 27, 2026

(54) SPLICER: A Highly Efficient Base Editing Toolbox That Enables In Vivo Therapeutic Exon Skipping

(71) Applicant: The Brd. of Trustees of the Univ. of Illinois, Urbana, IL (US)

(72) Inventors: Pablo Perez-Pinera, Urbana, IL (US); Jackson Scott Winter, Urbana, IL (US); Wendy S. Woods, Champaign, IL (US); Michael Gapinske, Urbana, IL (US); Angelo Miskalis, Urbana, IL (US); Shraddha Shirguppe, Urbana, IL (US)

(73) Assignee: The Brd. of Trustees of the Univ. of Illinois, Urbana, IL (US)

(21) Appl. No.: 19/387,082

(22) Filed: Nov. 12, 2025

Related U.S. Application Data
(60) Provisional application No. 63/719,304, filed on Nov. 12, 2024.

(51) Int. Cl. C12N 9/78; A61P 25/28; C12N 9/22; C12N 15/11; C12N 15/86; C12N 15/90

(52) U.S. Cl. CPC C12N 9/78; A61P 25/28; C12N 9/226; C12N 15/11; C12N 15/86; C12N 15/907; C12Y 305/04001; C12Y 305/04004; C12N 2310/20; C12N 2320/33; C12N 2750/14143

(57) ABSTRACT

Provided herein are methods and compositions for modifying RNA splicing of a pre-mRNA molecule to produce a modified mRNA molecule. The methods can comprise delivering to a cell one or more vectors or proteins comprising: (i) a nucleic acid molecule encoding one or more PAM-less or nearly PAM-less Cas nickases and one or more cytosine or adenosine deaminases or one or more PAM-less or nearly PAM-less Cas proteins and one or more cytosine or adenosine deaminase proteins; and a nucleic acid molecule encoding one, two, or more single guide RNA (sgRNA) molecules targeting a splice acceptor site and/or a splice donor site of a DNA molecule encoding the pre-mRNA. One or more exons or one or more portions of an exon are excluded during RNA splicing, resulting in the production of the modified mRNA molecule.

### PRIORITY

This application claims the benefit of U.S. Ser. No. 63/719,304, filed on Nov. 12, 2024, which is incorporated by reference herein in its entirety.

### GOVERNMENT SUPPORT

This invention was made with government support under U01 NS122102 awarded by the National Institutes of Health. The government has certain rights in the invention.

### SEQUENCE LISTING

The specification incorporates by reference a Sequence Listing. The Sequence Listing.xml file, identified as 771191 is 500,755 bytes in size and was created on Dec. 9, 2025.

### BACKGROUND

Exon skipping technologies enable exclusion of targeted exons from mature mRNA transcripts, which has broad applications in molecular biology, medicine, and biotechnology. Existing exon skipping techniques include antisense oligonucleotides, targetable nucleases, and base editors, which, while effective for specific applications at some target exons, remain hindered by shortcomings, including transient effects for oligonucleotides, genotoxicity for nucleases and inconsistent exon skipping for base editors. Methods are needed in the art to improve exon skipping techniques.

### SUMMARY

An aspect provides a method of modifying RNA splicing of a pre-mRNA molecule to produce a modified mRNA molecule comprising delivering to a cell one or more vectors or proteins. The method can comprise delivering:
(i) (a) a nucleic acid molecule encoding one or more PAM-less or nearly PAM-less Cas nickases and one or more cytosine or adenosine deaminases or (b) one or more PAM-less or nearly PAM-less Cas proteins and one or more cytosine or adenosine deaminase proteins; and
ii) a nucleic acid molecule encoding one or more single guide RNA (sgRNA) molecules targeting both a splice acceptor site and a splice donor site of a DNA molecule encoding the pre-mRNA; wherein one or more exons or one or more portions of an exon are excluded during RNA splicing, resulting in the production of the modified mRNA molecule.

The nucleic acid molecule encoding one or more PAM-less or nearly PAM-less Cas nickases and the nucleic acid molecule encoding the one or more cytosine or adenosine deaminases can be linked on one vector such that a fusion protein of the one or more PAM-less or nearly PAM-less Cas nickases and the one or more cytosine or adenosine deaminases can be expressed or wherein the or one or more PAM-less or nearly PAM-less Cas proteins and one or more cytosine or adenosine deaminase proteins are present in a fusion protein. The PAM sequence recognized by the one or more PAM-less or nearly PAM-less Cas nickases can be NN, NNN, NNNN, NNNNN, NNNNNN, NG, NRG, NNN, NRNH, NRN, or NDN. The one or more one or more PAM-less or nearly PAM-less Cas nickases can be SpCas9-NG, xCas9-NG, NAG-Cas9, SpCas9-NRNH, SpG Cas9, or SpRY Cas9, SaCas9-KKH, SpCas9-VQR, SpCas9-VRER, or xCas9. The one or more cytosine or adenosine deaminases can be CBE4max, CBE evoFERNY, CBE SsAPOBEC3B (R54Q), CBE RrA3F (F130L), TadCBE, CBEPpAPOBEC1 (H122A), ABE8e, ABE8.20m, ABE9, ABE8.20m, hA3A-BE4, hA3G-BE4, evoCDA-BE4, A3G-BE4.4, A3G-BE5.13, A3G-BE5.14, ABE6.3, ABE7.10, ABE8e, ABE9, or ABE8.20m, or ABE8.20. An amount of cryptic splicing can be reduced as compared to a cell not receiving the one or more vectors or proteins. Intron retention can be reduced as compared to a control. Full exon skipping can be increased as compared to a control. The one or more vectors can be AAV vectors. The one or more vectors, e.g., AAV vectors, or proteins can be delivered systemically, intravenously, intrathecally, intracerebrally, intraventricularly, intranasally, intratracheally, intra-aurally, intra-ocularly, intracerebral, or peri-ocularly, orally, rectally, transmucosally, inhalationally, transdermally, parenterally, subcutaneously, intradermally, intramuscularly, intracisternally, intranervally, intrapleurally, by lumbar puncture, intralymphatically, intracisternally, or intranerve. The modified mRNA molecule can be an amyloid precursor protein (APP) modified mRNA molecule. The one or more exons or one or more portions of an exon that are removed can be from exon 17 of an amyloid precursor protein (APP) gene. The modified mRNA molecule can be a huntingtin (HT7) modified mRNA molecule. The one or more exons or one or more portions of an exon that are removed can be from exon 12 or 13 of a huntingtin (HTT) gene.

In an aspect, the one or more vectors can comprise:
(i) a first vector comprising an inverted terminal repeat (ITR) sequence, a promoter, a nucleic acid molecule encoding one or more cytosine or adenosine deaminases, and a nucleic acid molecule encoding an N-terminal portion of a PAM-less or nearly PAM-less Cas nickase, an N-terminal fragment of a dimerization protein, and an ITR sequence; and
(ii) a second vector comprising an ITR sequence, a promoter, a C-terminal fragment of a dimerization protein, a nucleic acid molecule encoding a C-terminal portion of a PAM-less or nearly PAM-less Cas nickase, a nucleic acid molecule encoding a uracil glycosylase inhibitor, and an ITR sequence,
wherein the first vector, the second vector, or both the first vector and second vector further comprise a nucleic acid molecule encoding one or more sgRNA molecules targeting a splice acceptor site and/or a splice donor site of a DNA molecule encoding the pre-mRNA. The one or more cytosine or adenosine deaminases can be CBE4max, CBE evoFERNY, CBE SsAPOBEC3B (R54Q), CBE RrA3F (F130L), TadCBE, CBEPpAPOBEC1 (H122A), ABE8e, ABE8.20m, ABE9, ABE8.20m, hA3A-BE4, hA3G-BE4, evoCDA-BE4, A3G-BE4.4, A3G-BE5.13, A3G-BE5.14, ABE6.3, ABE7.10, ABE8e, ABE9, or ABE8.20m, or ABE8.20. The first vector, the second vector, or both the first vector and second vector further can comprise one or more nuclear localization signal (NLS) sequences. The nucleic acid molecule encoding one or more sgRNA molecules targeting a splice acceptor site and/or a splice donor site of a DNA molecule encoding the pre-mRNA can be operably linked to a promoter. The N-terminal fragment of a dimerization protein can be an N-terminal fragment of an intein, and the C-terminal fragment of a dimerization protein can be a C-terminal fragment of an intein. The C-terminal fragment of a dimerization protein and the N-terminal fragment of a dimerization protein can be derived from PhoRadA, RmaDnaB^{Δ286}, SspDnaB^{Δ275} SspDnaB^{M86Δ275}, SspDnaX, TvoVMA, NpuDnaE, NpuDnaB^{Δ283}, SspGyrB, AceL-TerL, PchPRP8, PfuRIR1-1, Psp-GDBPol-1, MtuRecA^{Δ228}, PfuRIR1-2, SceVMA^{Δ206}, RmaDnaB^{Δ271}, MtuRecA^{Δ285}, SspDnaB^{Δ274}, gp41-8, SceVMA^{Δ227}, IMPDH-1, NrdJ-1, MtuRecA^{Δ297}, gp41-1, AovDnaE, AspDnaE, AvaDnaE, Cra(C5505)DnaE, Csp(CCY0110)DnaE, Csp(PCC8801)DnaE, CwaDnaE, Maer(NIES843)DnaE, Mcht(PCC7420)DnaE, MtuRec^{Δ300}, NspDnaE, O/iDnaE, Se/(PC7942)DnaE, SspDnaE, Ssp(PCC7002)DnaE, TerDnaE-3, TelDnaE, TvuDnaE, NeqPol, TerThyX^{Δ132}, or combinations thereof. An amino acid sequence of the N-terminal fragment of an intein can comprise SEQ ID NO:377. An amino acid sequence of the C-terminal fragment of an intein can comprise SEQ ID NO:389. The first vector can further comprise a linker between the one or more cytosine or adenosine deaminases and the nucleic acid molecule encoding an N-terminal portion of a PAM-less or nearly PAM-less Cas nickase. The second vector can further comprise a linker between the nucleic acid molecule encoding a C-terminal portion of a PAM-less or nearly PAM-less Cas nickase and the nucleic acid molecule encoding an uracil glycosylase inhibitor. The first vector and/or second vector can further comprise one or more polynucleotides encoding a protein tag. The linker can encode a polypeptide molecule comprising SEQ ID NO:370, 371, 372, 373, or 374. The N-terminal fragment of the Cas nickase and the C-terminal fragment of the Cas nickase can form a full-length Cas nickase when combined. The PAM-less or nearly PAM-less Cas nickase can be split into a N-terminal fragment and a C-terminal fragment at a split point.

In an aspect: (a) the split point is localized at any amino acid between position 564 and 584, and the N-terminal fragment of PAM-less or nearly PAM-less Cas nickase comprises nucleotides from position 1 of the PAM-less or nearly PAM-less Cas nickase to the split point and the C-terminal fragment of a PAM-less or nearly PAM-less Cas nickase comprises nucleotides from the split point to position 1371 of the PAM-less or nearly PAM-less Cas nickase;
(b) the split point is localized at any amino acid between position 249 and 269, and the N-terminal fragment of the PAM-less or nearly PAM-less Cas nickase comprises nucleotides from position 1 of the PAM-less or nearly PAM-less Cas nickase to the split point and the C-terminal fragment of the PAM-less or nearly PAM-less Cas nickase comprises nucleotides from the split point to position 1371 of the PAM-less or nearly PAM-less Cas nickase; or
(c) the split point is localized at any amino acid between position 265 and 285, and the N-terminal fragment of the PAM-less or nearly PAM-less Cas nickase comprises nucleotides from position 1 of the PAM-less or nearly PAM-less Cas nickase to the split point and the C-terminal fragment of the PAM-less or nearly PAM-less Cas nickase comprises nucleotides from the split point to position 1371 of the PAM-less or nearly PAM-less Cas nickase.

In an aspect:
(a) the split point is localized at any amino acid between position 703 and 723, and the N-terminal fragment of the PAM-less or nearly PAM-less Cas nickase comprises nucleotides from position 1 of the PAM-less or nearly PAM-less Cas nickase to the split point and the C-terminal fragment of the PAM-less or nearly PAM-less Cas nickase comprises nucleotides from the split point to position 1371 of the PAM-less or nearly PAM-less Cas nickase;
(b) the split point is localized at any amino acid between position 935 and 965, and the N-terminal fragment of the PAM-less or nearly PAM-less Cas nickase comprises nucleotides from position 1 of the PAM-less or nearly PAM-less Cas nickase to the split point and the C-terminal fragment of the PAM-less or nearly PAM-less Cas nickase comprises nucleotides from the split point to position 1371 of the PAM-less or nearly PAM-less Cas nickase;
(c) the split point is localized at any amino acid between position 1044 and 1064 and, the N-terminal fragment of the PAM-less or nearly PAM-less Cas nickase comprises nucleotides from position 1 of the PAM-less or nearly PAM-less Cas nickase to the split point and the C-terminal fragment of the PAM-less or nearly PAM-less Cas nickase comprises nucleotides from the split point to position 1371 of the PAM-less or nearly PAM-less Cas nickase; or
(d) the split point is localized at any amino acid between position 1105 and 1125, and the N-terminal fragment of the PAM-less or nearly PAM-less Cas nickase comprises nucleotides from position 1 of the PAM-less or nearly PAM-less Cas nickase to the split point and the C-terminal fragment of the PAM-less or nearly PAM-less Cas nickase comprises nucleotides from the split point to position 1371 of the PAM-less or nearly PAM-less Cas nickase.

An aspect provides a method of treating Alzheimer's disease comprising delivering to a patient in need thereof one or more vectors or proteins comprising:
(i) a nucleic acid molecule encoding one or more PAM-less or nearly PAM-less Cas nickases and one or more cytosine or adenosine deaminases or one or more PAM-less or nearly PAM-less Cas proteins and one or more cytosine or adenosine deaminase proteins; and
(ii) a nucleic acid molecule encoding one or more single guide RNA (sgRNA) molecules targeting a splice acceptor site, a splice donor site, or both a splice acceptor site and a splice donor site of a target amyloid precursor protein gene (APP), wherein one or more exons or one or more portions of an exon of the target amyloid precursor protein gene (APP) are excluded during RNA splicing.

The one or more cytosine or adenosine deaminases can be CBE4max, CBE evoFERNY, CBE SsAPOBEC3B (R54Q), CBE RrA3F (F130L), TadCBE, CBEPpAPOBEC1 (H122A), ABE8e, ABE8.20m, ABE9, ABE8.20m, hA3A-BE4, hA3G-BE4, evoCDA-BE4, A3G-BE4.4, A3G-BE5.13, A3G-BE5.14, ABE6.3, ABE7.10, ABE8e, ABE9, or ABE8.20m, or ABE8.20. The one or more one or more PAM-less or nearly PAM-less Cas nickases can be SpCas9-NG, xCas9-NG, NAG-Cas9, SpCas9-NRNH, SpG Cas9, or SpRY Cas9, SaCas9-KKH, SpCas9-VQR, SpCas9-VRER, or xCas9. The amount of expression of Aβ42 peptides can be reduced as compared to a patient that does not receive the one or more vectors or proteins. The one or more exons or one or more portions of an exon that are removed can be exon 17 of an amyloid precursor protein gene (APP). The sgRNA targeting the splice donor site targets an intronic cryptic GT splice donor site of exon 17 of an APP gene. The level of mature RNA cannot be reduced as compared to a patient that does not receive the one or more vectors or proteins. The one or more vectors, e.g., Adeno-associated virus (AAV) vectors, or proteins can be delivered systemically, intravenously, intrathecally, intracerebrally, intraventricularly, intranasally, intratracheally, intra-aurally, intra-ocularly, intracerebral, or peri-ocularly, orally, rectally, transmucosally, inhalationally, transdermally, parenterally, subcutaneously, intradermally, intramuscularly, intracisternally, intranervally, intrapleurally, by lumbar puncture, intralymphatically, intracisternally, or intranerve.

An aspect provides a method of treating Huntington's disease comprising delivering one or more vectors or proteins to a patient in need thereof:
(i) a nucleic acid molecule encoding one or more PAM-less or nearly PAM-less Cas nickases and one or more cytosine or adenosine deaminases or one or more PAM-less or nearly PAM-less Cas proteins and one or more cytosine or adenosine deaminase proteins; and
(ii) a nucleic acid molecule encoding one or more single guide RNA (sgRNA) molecules targeting a splice acceptor site, a splice donor site, or both a splice acceptor site and splice donor site of a target huntingtin gene (HTT);
   wherein one or more exons or one or more portions of an exon of the huntingtin gene (HTT) are excluded during RNA splicing.

A caspase-6 cleavage site can be disrupted when one or more exons or one or more portions of an exon of the huntingtin gene (HTT) are excluded during RNA splicing.

The one or more cytosine or adenosine deaminases can be CBE4max, CBE evoFERNY, CBE SsAPOBEC3B (R54Q), CBE RrA3F (F130L), TadCBE, CBEPpAPOBEC1 (H122A), ABE8e, ABE8.20m, ABE9, ABE8.20m, hA3A-BE4, hA3G-BE4, evoCDA-BE4, A3G-BE4.4, A3G-BE5.13, A3G-BE5.14, ABE6.3, ABE7.10, ABE8e, ABE9, or ABE8.20m, or ABE8.20. The one or more one or more PAM-less or nearly PAM-less Cas nickases can be SpCas9-NG, xCas9-NG, NAG-Cas9, SpCas9-NRNH, SpG Cas9, or SpRY Cas9, SaCas9-KKH, SpCas9-VQR, SpCas9-VRER, or xCas9. The one or more exons or one or more portions of an exon that are removed from a mature RNA can comprise exon 12 or exon 13 of a huntingtin (HTT) gene. The sgRNA targeting the splice donor site can target an intronic cryptic GT splice acceptor site of exon 13 of a HTT gene. The amount of N-terminal HTT protein can be reduced by 20% or more in the patient as compared to a patient that does not receive the one or more vectors or proteins. The amount of intraneuronal inclusions of mHTT protein can be reduced in the patient as compared to a patient that does not receive the one or more vectors or proteins. The amount of cells within the striatum that are positive for reactive astrocyte marker glial fibrillary acidic protein (GFAP) can be reduced as compared to a control. The one or more vectors, e.g., Adeno-associated virus (AAV) vectors, or proteins can be delivered systemically, intravenously, intrathecally, intracerebrally, intraventricularly, intranasally, intratracheally, intra-aurally, intra-ocularly, intracerebral, or peri-ocularly, orally, rectally, transmucosally, inhalationally, transdermally, parenterally, subcutaneously, intradermally, intramuscularly, intracisternally, intranervally, intrapleurally, by lumbar puncture, intralymphatically, intracisternally, or intranerve.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-I Spry Cas9 in Combination with Different Deaminases Enables Efficient Disruption of Targeted Splice Acceptors. (1A) Schematic representation of the approach to disrupting splicing elements with near-PAMless ABEs (blue) or CBEs (green) by tiling eight sgRNAs per exon (Created in BioRender. Miskalis, A. (2024) BioRender.com/x92p416). Comparison of SpCas9 and SpRY Cas9 editing efficiency at the splice acceptors (SAs) of target exons using ABEs (1B, 1C) or CBEs (1D, 1E). Summary of editing efficiency at individual targets accomplished with SpCas9 or SpRY Cas9 ABEs (1C) or CBEs (1E). Comparison of DNA editing efficiency at multiple SAs using SpRY Cas9 fused with one of four different adenosine deaminases (1F, 1G) or one of six different cytosine deaminases (1H, 1I). Summary of editing efficiency at individual targets accomplished with different SpRY ABEs (1G) or different SpRY CBEs (11). For (1F-1I), DNA editing rates were normalized to the protein expression of each deaminase from FIG. 6 by dividing each DNA editing rate by the normalized expression of each base editor. Values represent means and error bars indicate SD. For (1B, 1D, 1F, and 1H), n=2; n=10 for (C); n=9 for (E, G); n=8 for (1). All replicates are biological replicates; ns, not significant; *p<0.05, **p<0.01; ***p<0.001; two-tailed, unpaired t-test except for (1G, 1I), which were analyzed via one-way ANOVA with Tukey's post hoc.

FIG. 2A-D. Simultaneous Editing of Splice Sites with SPLICER Enhances Exon Skipping. (2A) Illustration of SPLICER strategy for simultaneously targeting SAs and SDs with ABEs or CBEs followed by screening to identify effective BE systems and characterization next-generation sequencing (Created in BioRender. Miskalis, A. (2024) BioRender.com/x43x713). (2B-2D) Editing of AG dinucleotides in the SA (top panel) and GT dinucleotides in the SD (medium panel) and exon skipping (bottom panel) with ABEs (highlighted blue) or CBEs (highlighted green). Improvements in exon skipping were synergistic when targeting LMNA exon 11, EGFR exon 23 and JAG1 exon 12 (2B). Improvements in exon skipping were additive when targeting HSF1 exon 11 and RELA exon 7 (2C). Editing of SA and SD of AHCY exon 9 with ABEs did not improve exon skipping rates compared with editing of SA or SD alone (2D). All measurements of full-length exon skipping were performed by NGS except for LMNA exon 11, which were measured via Sanger Sequencing. Values represent means and error bars indicate SD. All replicates are biological replicates originating from three independent transfections of each sgRNA set; n=3 for all experiments; ns, not significant; *p<0.05; **p<0.01; ****p<0.0001; one-way ANOVA, Tukey's post hoc comparing SA/SD to SA and SD except for the comparison showing the DNA editing rates for JAG1 exon 12, which was performed via a two-tailed, unpaired t test.

FIG. 3 panels A-C. SPLICER Improves Exon Skipping Through Reduction of Both Cryptic Splicing and Intron Retention. (A) Sashimi plot describing exon splicing following ABE editing of LMNA exon 11 with SPLICER, in which a cryptic SD site is recognized in the middle of the exon when editing the SD (left), leading to no exon skipping and partial retention of exon 11 (right). This aberrant splicing event is reduced 3-fold by ABE editing of both SA and SD sites. HHGQVGGPI is SEQ ID NO:390; CACCACGGCCAGGTGGGCGGACCCATC is SEQ ID NO:391; HHGQSPQ is SEQ ID NO:392; CACCACGGCCAGAGCCCCCAG is SEQ ID NO:393. (B) Sashimi plot representing splicing of HSF1 exon 11 before and after ABE editing of splicing elements demonstrating a cryptic SA recognized in HSF1 exon 11 (left). This cryptic splicing leads to a frameshift mutation in a protein, which results in a premature termination codon in exon 11 (right). Cryptic splicing is reduced 10-fold when editing both sites (left). LDLFSPSVTV is SEQ ID NO:394; CTGGACCTGTTCAGCCCCTCGGTGACCGTG is SEQ ID NO:394; LDPLGDRA is SEQ ID NO: 395; LDPLGDRA is SEQ ID NO:396; CACCACCCCCTCGGTGTGGACCGTTGA is SEQ ID NO:397. (C) Sashimi plot describing splicing of BAP1 exon 2 following editing with CBEs (left). Cryptic splicing and intron retention occur with SA and SD editing alone, leaving an in-frame, but mutated protein (right). Both events are minimized with SPLICER (left). All measurements of full and cryptic exon skipping were performed by NGS except for LMNA exon 11, in which full exon skipping was measured by RT-PCR densitometry. For sashimi plots, values represent the mean. All replicates are biological replicates originating from three independent transfections of each sgRNA set; n=3 for all experiments; *p<0.05; **p<0.01; ***p<0.001; ****p<0.0001; one-way ANOVA, Tukey's post hoc comparing cryptic splicing events in SA or SD to the rate of that cryptic splicing event in simultaneous SA/SD editing. DPGLEDF is SEQ ID NO:398; GACCCAGGCCTCGAAGATTTCGGTGTC is SEQ ID NO:399; DPDFGVKGVQ is SEQ ID NO:400; CACCCAGATTTCGGTGTCAAGGGGGTGCAA is SEQ ID NO:401; VEDFGV is SEQ ID NO:402; GTGGAAGATTTCGATAAGGCAGGTGTC is SEQ ID NO:403; VEDFARAGV is SEQ ID NO:404; GTGCAAGATTTCGCTAAGGCAGGTGTC is SEQ ID NO:405.

FIG. 4A-L SPLICER Skips APP Exon 17 In Vitro And Reduces Production of Aβ42. (4A) Schematic representation of APP and sequence of APP exon 17 and introns 16 and 17. TTCTTTTTCTTAATTTGTTTTCAAGGTGTTCTTT GCAGATGTGTTCATCATGGTGTGGTGGAGGTAGGTAA is SEQ ID NO:406;AAGAAAAAGATTAAACAAAAGTTCCACAAGAACGTCTACACAAGTAGTACCAC ACCACCTCCATCCATT is SEQ ID NO:407. (4B) Genomic DNA editing rates at the SA of APP exon 17 with ABE8e and CBE4max fused with SpCas9 or SpRY Cas9 in HEK293T cells. (4C) SA DNA editing rates with SpRY Cas9 ABE8e when editing the SA alone or in combination with a panel of sgRNAs targeting the SD. (4D) SD DNA editing rates with sgRNAs targeting the SD alone or in combination with a sgRNA targeting the SA. Editing at the canonical and cryptic SDs is highlighted. GTGGAGGTAGGTAAA is SEQ ID NO:408. (4E) APP exon 17 exon skipping rates with an sgRNA targeting the SA in combination with one of 3 different sgRNAs targeting the SD measured by NGS. (4F) qPCR quantification of total APP mRNA. (4G, 4H) Quantification of APP exon 17 exon splicing (top) and schematic representation of the corresponding splicing event (bottom) following targeting of the SA with a sgRNA and SD with two different sgRNAs. VFFAEDVI is SEQ ID NO:409; AGGTGTTCTTTGCAGAAGATATA is SEQ ID NO:410; KMWV is SEQ ID NO:411; AGAAGATGTGGGTTTAG is SEQ ID NO:412; VEVDA is SEQ ID NO:414; GTGGAGGTAGGTAGGTTGACGCC is SEQ ID NO:415; VEVG is SEQ ID NO:416; GTGGAGGTAGGTTGA is SEQ ID NO:417. (4I) DNA editing at the SA and (J) SD of APP exon 17 in BE(2)-M17 neuroblastoma cells following enrichment with puromycin. AGGTAGGTAA is SEQ ID NO:413. (4K) Exon skipping rates for APP exon 17 in puromycin selected BE(2)-M17 neuroblastoma cells. (4L) Aβ42 levels in BE(2)-M17 cells following skipping of APP exon 17 in comparison with control cells using ELISA. All measurements of full-length and cryptic exon skipping were performed by NGS while all DNA editing rates were measured via Sanger sequencing. For all bar graphs, values represent means and error bars indicate SD. For heatmaps, values represent means. For sashimi plots, values represent means. In experiments performed in HEK293 cells, each replicate is a biological replicate originating from an independent transfection of each sgRNA. In experiments with BE(2)-M17 cells, a single transfection was performed with each sgRNA set to generate a puromycin selected cell line. Following cell line generation, each sgRNA set was maintained amongst three separate plates with each replicate deriving from one plate; n=3 for all experiments; ns, not significant; *p<0.05; **p<0.01; ***p<0.001; one-way ANOVA, Tukey's post hoc comparing SA/SD to SA and SD except for (4B, 4L), which were analyzed via a two-tailed, unpaired t-test.

FIG. 5 panels A-E. SPLICER Enables Efficient DNA Editing and Full Exon Skipping in a Humanized Mouse Model of Alzheimer's Disease. (A) Experimental workflow of in vivo editing following AAVrh10 injection of BEs targeting the SA and SD of APP exon 17 in the hippocampus of R1.40 mice (Created in BioRender. Miskalis, A. (2024) BioRender.com/f96o812). GTGGAGGTAGGTAAA is SEQ ID NO:408. (B) In vivo genomic DNA editing of APP exon 17 SA and (C) SD in bulk hippocampus tissue measured using NGS. (D) In vivo exon skipping of APP exon 17 in bulk hippocampus tissue following treatment with BEs targeting the SA and SD. (E) Sashimi plot of splicing events in control mice and mice injected with BEs targeting the SA and SD of APP exon 17. For all bar graphs, values represent means and error bars indicate SD. For heatmaps, values represent means. For sashimi plots, values represent means. Each replicate value is from the hippocampus of an individual mouse. All measurements of exon skipping were performed by NGS and all DNA editing rates were measured by NGS. n=3 for all groups; *p<0.05; **p<0.01; two-tailed, unpaired t-test.

FIG. 6 panels A-C. SPLICER Enhances Exon Skipping. (A) Quantification of exon skipping by NGS in samples transfected with a non-targeted plasmid control samples transfected controls at targeted exons; n=3. (B) DNA editing and RNA exon skipping following targeting of BAP1 exon 2 (top panel) and EGFR exon 23 (bottom panel) with CBEs. (C) DNA editing and RNA exon skipping following targeting of RELA exon 7, LMNA exon 11, HSF1 exon 11 with ABEs. Exon skipping rates were measured by RT-PCR gel densitometry.

FIG. 7. Reduction of Cryptic Splicing for HSF1 Exon 11 with SPLICER. Sashimi plot showing a cryptic splice donor event following target the SA or SD individually or simultaneously (top) leading to a frameshifted protein starting at exon 12. *, p<0.05; ****, p<0.0001; One-Way ANOVA, Tukey's Post Hoc comparing SA/SD to SA and SD. SLASIQELLS is SEQ ID NO:418; AGCCTGGCCAGTATCCAAGAGCTCCTGTCT is SEQ ID NO:419; SLAYPRAPVS is SEQ ID NO:420; AGCCTGGCGTATCCAAGAGCTCCTGTCTCC is SEQ ID NO:421.

FIG. 8A-F. Analysis of Global Changes in Patterns of mRNA Expression and Off-target Editing Following Induction of Exon Skipping with SPLICER. (8A) Schematic illustrating the pathogenic events leading to formation of plaques in Alzheimer's disease through secretase-mediated cleavage of the APP protein at exon 17. (8B-8D) counts-per-million (cpm) heatmap from RNA-seq data showing no change of the expression levels for genes within the (8B) base excision repair pathway (KEGG pathway hsa03430), (8C) mismatch repair pathway (KEGG pathway hsa03410), and (8D) Alzheimer's disease pathway (KEGG pathway hsa05010) in cells treated with BEs in comparison with control cells. (8E) APP exon 17 exon skipping rates in puromycin-selected BE(2)-M17 cells following treatment with BEs targeting the SA, the SD or both. (8F) Analysis of off-target effects of sgRNAs targeting the SA (top panel) or SD (bottom panel) of APP exon 17 at 5 computationally predicted sites using NGS. n=3; ns, no significance; ****, p<0.0001; One-Way ANOVA, Tukey's Post Hoc comparing SA/SD to SA and SD.

FIG. 9A-C. Transduction Efficiency and DNA Editing in the Hippocampus Following Nuclei Enrichment via FACS. (9A) AAV transduction efficiency in the hippocampus isolated from mice treated with EGFP-KASH BE targeted to the ROSA26 locus or APP exon 17 following FACS. (9B, 9C) DNA editing rates at the SA and SD in FACS-sorted nuclei. n=3; **, p<0.01; ***, p,0.001; ****, p<0.0001; One-Way ANOVA, Tukey's Post Hoc. GTGGAGGTAGGTAAA is SEQ ID NO:408.

FIG. 10 panels (a)-(g).CRISPR base editing of HTT exon 13 splice acceptor (SA) sites. (a) (Top) Schematic representation of the pathogenesis of Huntington's disease and (bottom) the location of the caspase-6 cleavage site in the HTT protein. (b) Overview of the strategy for targeting the SA for HTT exon 13 by CRISPR base editing. AGGTGTTAGACGGT is SEQ ID NO:422; VLDG is SEQ ID NO:393; GTGTTAGACGGT is SEQ ID NO:424. (c) Editing rates in HEK293T cells for the base editor variants targeting the exon 13 SA in HTT, as measured by next-generation sequencing (NGS; n=3). (d) Schematic of the split-intein architecture used for AAV delivery. Abbreviations: ITR, inverted terminal repeat; CAG, cytomegalovirus early enhancer/chicken β-actin promoter; Rm, Rhodothermus marinus, NLS, nuclear localization signal; UGI, uracil glycosylase inhibitor; U6, human U6 promoter; 3xHA, three repeats of the human influenza hemagglutinin (HA) epitope; V5, epitope of the V protein of the paramyxovirus of the simian virus 5 family. (e) Heat map showing the editing frequencies for the split-intein version of CBE-HTT-4 in HEK293T cells, as determined by NGS (n=3). The target base is shown in red. CCCTTGAACCGTTTAGGTGT is SEQ ID NO:423. (f) Relative quantification of exon skipping rates for the full-length and split-intein version of CBE4-HTT-4 in HEK293T cells, as determined by NGS (n=3). (g) Sashimi plot depicting the splicing outcomes following treatment with CBE-HTT-4. Two major splicing patterns are shown: partial skipping of exon 13 and full skipping of exon 13. A GFP-encoding plasmid was used as a control for all experiments. All data points are biologically independent samples. Values are means and error bars indicate S.D. *P<0.05, ***P<0.001, ****P<0.0001. Data for (c), (e) and (f) compared using a one-tailed unpaired t-test. Data for (g) compared using a one-way ANOVA with Tukey's post-hoc test.

FIG. 11A-1. CRISPR base editing of HTT exon 13 influences splicing and protein expression. (11A) Overview of the workflow for creating isogenic HEK293T cell lines. (11B) (Top) Representative Sanger sequencing traces of a wild-type (WT) and (bottom) CBE-HTT-4-modified cell line with the target edit at the exon 13 SA in HTT. TTTAGGTGTTAGACGGT is SEQ ID NO:425; TTTAAATGTTAGACGGT is SEQ ID NO:426. (11C) Sashimi plot depicting the relative predicted splicing outcomes in WT cells and CBE-HTT-4-modified cells. (11D) Representation of alternative splicing before and after utilization of a cryptic SA in HTT exon 13. Outcomes include partial skipping of exon 13, complete skipping of exon 13, and retention of the intron upstream of exon 13. (11E) Relative HTT mRNA in WT and CBE-HTT-4-modified cells (n=3). QVSEIVLDGQITGISMALQ is SEQ ID NO:427; CAGGTCTCTGAAATTGTGTTAGACGGTCAGATTACAGGTATTTCCATGGCCCTTCAA is SEQ ID NO:428; QVSEIVLDGQITGISMALQ is SEQ ID NO:4219; CAGGTCTCTGAAATTGTGTTAGACGGTCAGATTACAGGTATTTCCATGGCCCTTCAA is SEQ ID NO:430; QVSEIITGISMALQ is SEQ ID NO:431; CAGGTCTCTGAAATTATTACAGGTATTTCCATGGCCCTTCAA is SEQ ID NO:432; QVSEIPF is SEQ ID NO:433; CAGGTCTCTGAAATTCCCTTCTGA is SEQ ID NO:434; CAGGTCTCTGAAATTAACAGCCAGGTGTTAGACGGTCAGATTACAGGTATTTCCATGG CCCTTCAA is SEQ ID NO:435; QVSEIPPLNRLSVRR is SEQ ID NO:436; CAGGTCTCTGAAATTCCCTTGAACCGTTTAGTGGTTAGACGGTGA is SEQ ID NO:437. (11F) Western blot of HTT protein from WT and CBE-HTT-4-modified cells following incubation with purified caspase-6 enzyme. (11G) Quantification of relative full-length HTT protein, C-terminal and N-terminal HTT protein fragments in the WT or CBE-HTT-4-modified cells. All values are normalized to GAPDH (n=3). Values are means and error bars indicate S.D. **P<0.01, ***P<0.001, ****P<0.0001. (11H) and (11I) show The CBE-modified clones and native cells had equivalent amounts of caspase-6. Data for (11C)compared using a one-way ANOVA with Tukey's post-hoc test. Data for (11E) and (11G) and compared using a one-tailed, unpaired t-test.

FIG. 12A-L Base editing of the HTT exon 13 SA in YAC128 mice. (12A) Schematic of the study in YAC128 mice. (12B) DNA editing frequencies in the striatum of 12-month-old YAC128 mice injected with 1×10⁹ vector genomes (VGs) of AAV9-CAG-CBE-HTT-4 (n=10) or with AAV9-CAG-CBE-mRosa26 (n=7). The target base is shown in red. CCCTTGAACCGTTTAGGTGT is SEQ ID NO:438. (12C) (Left) Sashimi plot depicting the splicing events in the striatum of 12-month-old YAC128 mice injected with 1×10⁹ VGs of AAV9-CAG-CBE-HTT-4 (n=10) relative to those injected with AAV9-CAG-CBE-mRosa26 (n=6). (Right) Summary of HTT exon 13 skipping rates, as determined by NGS. (12D) (Left) Western blot for human HTT protein products from the striatum of 12-month-old YAC128 mice injected with 1×10⁹ VGs of AAV9-CAG-CBE-HTT-4 (n=3) or AAV9-CAG-CBE-mRosa26 (n=3). (Right) Quantification of relative protein expression normalized to GAPDH (n=3). (12E, 12F) Normalized (12E) striatum and (12F) cortex volumes of 12-month-old YAC128 mice injected with 1×10⁹ VGs of AAV9-CAG-CBE-HTT-4 (n=4) or AAV9-CAG-CBE-mRosa26 (n=4). All data normalized to 12-month-old wild-type FVB/NJ mice (n=6). (12B) Representative immunofluorescence staining of mHTT inclusions in the striatum of 12-month-old YAC128 mice injected with 1×10⁹ VGs of AAV9-CAG-CBE-HTT-4 (n=6) or AAV9-CAG-CBE-mRosa26 (n=6). Scale bar, 52 μm. (12H, 12I, 12J, 12K, 12L) Quantification of (12H) mHTT inclusion density per total number of cells in the striatum, (12I) the percentage of cells with >1 mHTT inclusion per total number of cells, (12J) mHTT inclusion density per unit area, (12K) the percentage of cells with >1 mHTT inclusion per area unit, and (12L) the number of mHTT inclusions per cell. All data normalized to YAC182 mice injected with AAV9-CAG-CBE-mRosa26. Values are means and error bars indicate S.D. *P<0.05, **P<0.01, ***P<0.001, ****P<0.0001. Data for (12B), (12D, 12E), and (12H, 12I, 12J, 12K, 12L) compared using a one-tailed unpaired t-test. Data for (c) compared using a one-way ANOVA with Tukey's post-hoc analysis.

FIG. 13A-I Disruption of the HTT exon 13 SA using near-PAMless CRISPR base editors. (13A) Schematic of the approach to disrupt HTT exon 13 SA using near-PAMless base editor variants. CACAGCCCCCTTGAACCGTTTAGGTGTTAGACGGTACCGACAACCAGTAT is SEQ ID NO:439; GTGTAGGGGGAACTTGGCAAATCCACAATCTGCCATGGCTGTTGGTCATA is SEQ ID NO:440; VLDGTDNQY is SEQ ID NO:441. (13B, 13C) Editing rates in HEK293T cells at the exon 13 SA, as determined by NGS (n=3). (13D) Violin plot depicting the editing efficiencies of SpRY-CBE-HTT-6 and SpRY-ABE-HTT-3 (n=3). (13E) Schematic of the split-intein architecture used for AAV delivery. (13F) Heat map depicting the editing frequencies for the split-intein version of SpRY-ABE-HTT-3 in HEK293T cells, as determined by NGS (n=2). The target base is shown in red. TTAGGTGTTAGACGGTACCG is SEQ ID NO:304. (13G) Quantification of exon skipping rates induced by the full-length and split-intein version of SpRY-ABE-HTT-3 in HEK293T cells, as determined by NGS. (13H) Quantification of the percentage of transcript variants with exon 13 partially skipped (grey) or exon 13 fully skipped (orange), as measured by NGS (n=2). (13I) Sashimi plot depicting the splicing events for SpRY-ABE-HTT-3: partial skipping of exon 13 or full skipping of exon 13 relative to cells transfected with a GFP-encoding control plasmid. All data points are biologically independent samples. Values are means and error bars indicate S.D. **P<0.01, ***P<0.001, ****P<0.0001; Data for (13B, 13C, 13D) and (13F, 13G, 13H) compared using a one-tailed unpaired t-test. Data for (131) compared using a one-way ANOVA with Tukey's post-hoc analysis.

FIG. 14A-O. Disrupting HTT exon 13 SA using a near-PAMless base editor improves HD-related deficits in YAC128 mice. (14A) Schematic of the experiments conducted in YAC128 mice. (14B) DNA editing frequencies in the striatum of 12-month-old YAC128 mice injected with 1×10¹¹ VGs of AAV9-CAG-SpRY-ABE-HTT-3 (n=5), with a statistical comparison to YAC128 mice injected with AAV9-CAG-CBE-mRosa26 (n=7). The target base is shown in red. TTAGGTGTTAGACGGTACCG is SEQ ID NO:304. (14C) (Left) Sashimi plot depicting the splicing outcomes in the striatum of 12-month-old YAC128 mice injected with 1×10¹¹ VGs of AAV9-CAG-SpRY-ABE-HTT-3 (n=5) relative to AAV9-CAG-SpRY-ABE-mRosa26 (n=7). (Right) Summary of HTT exon 13 skipping rates, as determined by NGS. (14D) (Left) Western blot for human HTT protein products in the striatum of 12-month-old YAC128 mice injected with 1×10¹¹ VGs of AAV9-CAG-SpRY-ABE-HTT-3 (n=3) or AAV9-CAG-SpRY-ABE-mRosa26 (n=3). (Right) Quantification of relative protein. Values are normalized to GAPDH. (14E, 14F) Normalized (14E) striatum and (14F) cortex volumes of 12-month-old YAC128 mice injected with 1×10¹¹ VGs of AAV9-CAG-SpRY-ABE-HTT-3 (n=4) or AAV9-CAG-SpRY-ABE-mRosa26 (n=7). Data are normalized to age-matched wild-type FVB/NJ mice (n=5). (14G) Representative immunofluorescence staining of mHTT inclusions in the striatum of 12-month-old YAC128 mice injected with 1×10¹¹ VGs of AAV9-CAG-SpRY-ABE-HTT-3 (2 sections analyzed per biological replicate; n=5) or AAV9-CAG-SpRY-ABE-mRosa26 (2 sections analyzed per biological replicate; n=6). Scale bar, 52 μm. (14H, 14I, 14J, 14K) Quantification of (14H) the percentage of cells with mHTT inclusions, (14I) the percentage of cells with >15 mHTT inclusions, (14J) the percentage of cells with mHTT inclusions per unit area, and (14K) the percentage of cells with >15 mHTT inclusions per unit area. All data are normalized to YAC128 mice injected AAV9-CAG-SpRY-ABE-mRosa26. (14L) Clasping, (14M) grips strength, (14N) rotarod, and (14O) elevated plus maze of 12-month old YAC128 mice injected with 1×10¹¹ VGs of AAV9-mCAG-SpRY-ABE-HTT-3 compared to uninjected mice. Values are means and error bars indicate S.D. *P<0.05, **P<0.01, *** P<0.001, ****P<0.0001. Data for (14B), (14D, 14E), and (14H, 14I, 14J, 14K, 14L, 14M, 14N, 14O) compared using a one-tailed unpaired t-test. Data for (14C) compared using a one-way ANOVA with Tukey's post-hoc analysis.

FIG. 15A-C. CRISPR base editing of potential sites in HTT exon 12 and exon 13. (15A) Schematic representation of HTT and summary of target sites in HTT exon 12 and exon 13. (15B) Genomic DNA editing rates in HEK293T cells with base editors targeting the splice acceptor of exon 12, 2 different splice enhancers within exon 12, 3 sequences encoding target sites for caspase-3 and a sequence encoding the target site for caspase-6 (n=2). Guides that target the SA of exon 13 are not included in this analysis. (15C) Relative exon skipping rates following treatment with the base editors describe above measured by gel densitometry of RT-PCR products (n=2). Values represent means and error bars represent S.D. Exact sequences of guide RNAs, PAMs, and base editors are disclosed in Table 5. TTCAGCCTCAGTGA is SEQ ID NO:443; CGGTCACAGCAC is SEQ ID NO:444; GACTCAGTGGAT is SEQ ID NO:445; GATGAGGAGGAT is SEQ ID NO:446; GACCTGAATGAT is SEQ ID NO:447; TCAGCTGTTACC is SEQ ID NO:448; TTAGGTGTTAGACGGT is SEQ ID NO:449; RSQH is SEQ ID NO:450; DVSD is SEQ ID NO:451; DEED is SEQ ID NO:391; DLND is SEQ ID NO:392; SAVT is SEQ ID NO:454; VLDG is SEQ ID NO:393; TCACAGC is SEQ ID NO:395;

FIG. 16 panels (a)-(b). Split CBE-HTT-4 outperforms its full-length counterpart when targeting the SA of HTT exon 13 in vitro. (a) Base editing rates in genomic DNA in HEK293T cells at the SA of exon 13 following treatment with full-length or split CBE-HTT-4 quantified by NGS (n=3). (b) Gel image of RT-PCR products detecting HTT exon 13 skipping following treatment with full-length or split CBE-HTT-4 (n=3). Plasmid encoding GFP was used as a control in all experiments. Values represent means and error bars indicate S.D.; **, P<0.01; Data compared using one-tailed unpaired t-test.

FIG. 17A-B. Computational prediction of potential splice acceptor sites in HTT exon 13. (17A) Splice AI network predicts potential cryptic splice acceptor sites in HTT exon 13 in a sequence-specific context. Panel (a) shows the raw splice site scores at potential ‘AG’ sites in the wild-type HTT exon 13 sequence; panel (b) displays the predicted increase or decrease in score at splice acceptor sites when the consensus splice acceptor site “AG” is modified (AG>AA); panel 3 displays the predicted increase or decrease in scores at splice acceptor sites when consensus SA-editing is coupled by a bystander editing at +1-bp from the canonical SA site (AGG>AAA). Size and direction of bars represents the gain or loss in scores with respect to the raw splice site score of the wild type transcript in panel 1.

TCACAGCCCCCTTGAACCGTTTAGGTCTTACACGGTACCGACAACCAGT ATTTGGGCCTGCAGATTGGACAGCCCCAGGATGAAGATGAGG is SEQ ID NO:452. (17B) Strength of consensus splice acceptor and potential cryptic splice acceptor sites within HTT exon 13 as predicted by different algorithms including Splice Rover, Human Splicing Finder (HSF), and MaxEntScan.

FIG. 18. CBE-HTT-4 did not edit computationally predicted off-target sites. Chromosomal locations and gene IDs of the target sequence for CBE-HTT-4 and eight potential off-target sites identified using the specificity score described by Hsu et al. DNA targeting specificity of RNA-guided Cas9 nucleases. Nature Biotechnology 2013, 31:827-832) is shown on the left. Tables describing the nucleotide frequencies for C>T editing, obtained by deep sequencing, for each off-target site is shown on the right. Frequencies are presented as the average percentage of edited reads (n=3). ACACCTAAACGGTTCAAGGG is SEQ ID NO:267; ACAGCTATTGGGTTCAAGGG is SEQ ID NO:454; ACAGTCAAAGGGTTCAAGGG is SEQ ID NO:455; ACATTTAAATGGTTCAAGGG is SEQ ID NO:456; ACATTTGAACCGTTCAAGGG is SEQ ID NO:457; AGACACAAACTGTTCAAGGG is SEQ ID NO:458; ACAGGTTAACGTTTCAAGGG is SEQ ID NO:459; ACCCTGAAACGTTTCAAGGG is SEQ ID NO:460; AATCCTAAAAGGTTGAAGGG is SEQ ID NO:461.

FIG. 19 panels (a)-(c). AAV9-transduction of striatum and cortex regions of YAC128 HD mice. (a) Schematic workflow for in vivo AAV transduction. (b-c) Representative immunofluorescence staining of dorsal sagittal section of the brain of mice injected with 1×10⁹ VGs of AAV9-CAG-CBE-HTT-4, per hemisphere, (b) striatum (STR; scale bar, 50 μm), (c) cortex (CTX; scale bar, 50 μm) staining for DAPI and V5-epitope tag.

FIG. 20 panels (a)-(f). Targeting HTT exon 13 SA with CBE-HTT-4 did not induce neuroinflammation in the striatum or cortex in YAC128 mice. Representative immunofluorescence staining and corresponding quantification for (a-c) GFAP and (d-f) Iba1 in sections from the striatum and cortex of YAC128 mice injected with AAV9-CAG-CBE-HTT-4 or AAV9-CAG-CBE-mRosa26. Scale bar, 22.7 μm. Error bars indicate S.D. (n=3). *, P<0.05. Data for (b-c), (e-f), compared using one-tailed unpaired t-test.

FIG. 21 panels (a)-(f). Editing profiles induced by SpRY variants targeting the SA of HTT exon 13. (a) DNA editing and (b) exon skipping rates by canonical and non-canonical SpRY (Missing A61R and Li111R) variants. (c-f) Genomic DNA editing rates measuring bystander modifications using (c,e) SpRY-CBE4max and (d,f) SpRY-ABE8e. (n=3). Values represent means and error bars indicate SD.

FIG. 22. SpRY-ABE-HTT-3 did not edit computationally predicted off-target sites. Chromosomal locations and gene IDs of the target sequences for SpRY-ABE-HTT-3 and five potential off-target sites identified using Cas-OFF finder (Bae S, Park J, Kim J-S: Cas-OFFinder: a fast and versatile algorithm that searches for potential off-target sites of Cas9 RNA-guided endonucleases. Bioinformatics 2014, 30:1473-1475) are shown to the left. Tables describing the nucleotide frequencies for A>G editing, obtained by deep sequencing, for each off-target site is shown on the right. Frequencies are presented as the average percentage of edited reads (n=3). TTAGGTGTTAGACGGTACCG is SEQ ID NO:304; TTACAGGTGTTAGACGGTAG is SEQ ID NO:463; TTACTGGTGTTAGATGGTAC is SEQ ID NO:464; TTAGGTGTTACACGCTACCC is SEQ ID NO:465; TTAGGGTTAGACGGTCACC is SEQ ID NO:466; TTAGGTGTAAGAGGTCCACC is SEQ ID NO:467.

FIG. 23. AAV9 dose escalation study. Genomic DNA editing rates of HTT exon 13 SA in YAC128 mice injected in the striatum with CAG-SpRY-ABE-HTT-3 packaged into AAV9. The doses used were 1×10⁹VGs, 1×10¹⁰ VGs, and 1×10¹¹ VGs. Measurements were performed at 2 months and 5 months of age. Values represent means and error bars indicate S.D. Data compared using one-tailed unpaired t-test.

FIG. 24 panels (a)-(c). Targeting HTT exon 13 SA site by AAV9-SpRY-ABE-HTT-3 affected neuronal count in the striatum and cortex regions of YAC128 HD mice. (a) Representative immunofluorescence NeuN staining in sections from the striatum and cortex following injection of YAC128 mice with AAV9-CAG-SpRY-ABE-HTT-3 or AAV9-CAG-SpRY-ABE-mRosa26. Scale bar, 36.1 μm. (b-c) Quantification of NeuN-positive cells in the (b) striatum and (c) cortex in YAC128 mice with AAV9-CAG-SpRY-ABE-HTT-3 or AAV9-CAG-SpRY-ABE-mRosa26. Error bars indicate S.D. (n=3). *, P<0.05; **, P<0.01. Data compared using one-tailed unpaired t-test.

FIG. 25A-B qRT-PCR standard curves. (25A) Standard curve for qRT-PCR primers that bind to human GAPDH. (25B) Standard curve for qRT-PCR primers that bind to human HTT.

FIG. 26A-B Splicing events in long-read HTT transcripts (a,b) Quantification and schematics of splicing events by long-read sequencing of HTT mRNA of clonal HEK293T cells at junctions for (26A) exon 13 and (26B) exon 4. Values represent means and error bars indicate SD (n=3). *, P<0.05; Data compared using one-tailed unpaired t-test.

FIG. 27A-B. Antibody validation on clonal HTT full and partial KO cells. Western blot for human HTT protein products from HTT KO, HTT partial KO and WT HEK293s using (27A) rabbit anti-HTT, clone EPR5526 and (27B) mouse anti-HTT antibody, clone 5HU-1 H6. (n=3) KO cells generated using APOBEC3A base editor targeting first glutamine in Exon 1 to change CAG>TAG.

FIG. 28 panels (a)-(c) SpRY-ABE-HTT-3 did not edit computationally predicted off-target sites. (a) Chromosomal locations and gene IDs of the target sequence for SpRY-ABE-HTT-3 and 23 potential off-target sites identified using a computational algorithm, Cas-OFFinder and COSMID; Frequencies are presented as the average percentage of edited reads from HEK293T cells three days after transfection with plasmids encoding SpRY-ABE-HTT-3 (n=3). (b) Differential gene expression analysis following targeting of SA in exon 13 in comparison with mRosa26 cells (adjusted p<0.05). (c) Total mRNA A>G mutations in transfected HEK293T cells (n=3).

FIG. 29. AAV dose-dependent response in editing of HTT exon 13 SA site. DNA editing of HTT exon 13 SA following the delivery of 1×10⁹ (n=2),1×10¹⁰ (n=2), and 1×10¹¹ (n=2) VGs of AAV9 encoding CAG-SpRY-ABE-HTT-3. Analysis was conducted at 2 months and 5 months of age.

FIG. 30 panels (a)-(f). Analysis of AAV9 transduction efficiency. (a) Schematic for workflow for AAV9-EGFP-KASH Transduction and Enrichment. (b) Genomic DNA editing rates in EGFP-KASH sorted cells of 1 month old YAC128HD mice injected with AAV9-SpRY-ABE-HTT-3 (n=4). Tissues collected 4 weeks after injection. Values represent means and error bars indicate SD. (c) Transduction efficiency in both striatum and cortex (d-e) Percent of EGFP+ transduced cells that also were NeuN+ or GFAP+ in the (d) striatum and (e) cortex. (n=2) (f) Representative immunofluorescence staining with EGFP and NeuN or GFAP marker in sections from the striatum and cortex of YAC128 HD mice injected with AAV9-SpRY-ABE-HTT-3 (scale bar, 50 μm).

FIG. 31 panels (a)-(b). Lack of editing outside of striatum and cortex in the CNS and peripheral organs in ABE injected YAC128 HD mice. (a,b) DNA editing frequencies in (a) the central nervous system and (b) peripheral organs of 12-month-old YAC128 mice injected with 1×10¹¹ VGs of AAV9-SpRY-ABE-HTT-3 (n=6).

FIG. 32 panels (a)-(b). Additional functional assessments of 12-month-old YAC128 mice (a) Open field, and (b) body weight normalized to WT mice assessments for 12-month-old YAC128 mice injected with 1×10¹¹ VGs of AAV9-mCAG-SpRY-ABE-HTT-3 compared to uninjected mice. Values are means and error bars indicate S.D. Data for (a) compared using a one-tailed unpaired t-test. Data for (b) compared using a one-way ANOVA with Tukey's post-hoc analysis.

FIG. 33. Representative MRI of YAC128 mice. Coronal MRI showing the striatum in wild-type (WT) healthy mice and YAC128 mice treated with SpRY-ABE-HTT-3 and mRosa26. Approximated striatal tissue outlined in white.

FIG. 34 panels (a)-(k). Targeting HTT exon 13 SA site by AAV9-SpRY-ABE-HTT-3 affected neuronal count in the striatum and cortex regions of YAC128 HD mice. (a) Representative immunofluorescence staining using anti-NeuN antibodies (scale bar, 36.1 μm), in sections from the striatum and cortex, respectively, of YAC128 HD mice injected with AAV9-CAG-SpRY-ABE-HTT-3 and AAV9-CAG-SpRY-ABE-mRosa26. (b-c) Quantification of NeuN-positive cells in the (b) striatum and (c) cortex regions, respectively. (d) Quantification of mRNA expression in striatum for NeuN. (e-h) Quantification of tubulin stained tissue sections for (e) primary projections, (f) neurite length, average soma count per (g) unit area and (h) cell. (i) Representative immunofluorescence staining for tubulin in sections from the striatum of YAC128 HD mice injected with AAV9-CAG-SpRY-ABE-HTT-3 and uninjected mice (scale bar, 40 μm). (j-k) Quantification of mRNA expression in striatum for (j) GFAP and (k) IBA1. Error bars indicate SD (n=3). *, P<0.05, **, P<0.01. Data for (b-h, j, k) compared using one-tailed unpaired t-test.

FIG. 35A-E. Standard curves for qRT-PCR. (35A) Standard curve for qRT-PCR primers that bind to the housekeeping gene, human GAPDH sequence. (35B) Standard curve for qRT-PCR primers that bind to the target gene, human HTT sequence. (35C) Standard curve for qRT-PCR primers that bind to the target gene, mouse NeuN sequence. (35D) Standard curve for qRT-PCR primers that bind to the target gene, mouse GFAP sequence. (35E) Standard curve for qRT-PCR primers that bind to the target gene, mouse IBA1 sequence.

### DETAILED DESCRIPTION

Provided herein is a toolbox of next-generation base editors comprised of near-PAMless Cas9 nickase variants fused to adenosine or cytosine deaminases for the editing of splice acceptor (SA) and splice donor (SD) sequences. Synchronized SA and SD editing with SPLICER can improve exon skipping, reduce aberrant outcomes, including cryptic splicing and intron retention, and enable skipping of exons refractory to single splice-site editing. To demonstrate the therapeutic potential of SPLICER, APP exon 17 was targeted, which encodes the amino acid residues that are cleaved to form the Ap plaques in Alzheimer's disease. SPLICER reduced the formation of Aβ42 peptides in vitro and enabled efficient exon skipping in a mouse model of Alzheimer's disease. Overall, the compositions and methods making up SPLICER are a widely applicable and efficient toolbox for exon skipping with broad therapeutic applications.

### INTRODUCTION

Programmable genome-editing nucleases have fundamentally transformed biotechnology and medicine by providing a facile means to introduce targeted modifications in the genome of living cells. Nonetheless, their reliance on DNA double-strand breaks (DSBs) has been linked to undesirable effects, including chromosomal deletions, chromotripsis, activation of the p53-mediated damage response pathway, and mRNA misregulation, all of which can hinder their implementation. For these reasons, emergent gene editing technologies capable of introducing precise and predictable mutations without DSBs are becoming increasingly adopted for a range of applications.

One such technology are base editors (BEs), which utilize a fusion of a Cas9 nickase and a cytosine deaminase to introduce C>T (cytosine base editors (CBEs)) or an adenosine deaminase (adenosine base editors (ABEs)) to introduce A>G mutations at genomic loci with complementarity to a single-guide RNA (sgRNA). BEs can be used as described herein for the programmed modulation of RNA splicing, which can be used to modify the transcriptional landscape of a cell for basic biology and therapeutic applications. Excluding exons from mature mRNA transcripts can eliminate mutated sequences and recover reading frames lost to chromosomal deletions.

Multiple challenges exist that prevent the broad implementation of SpCas9-comprised BEs for exon skipping. First, the native SpCas9 protein can only target splice sites with NGG protospacer adjacent (PAM) motifs nearby the target locus, a restriction that can limit the number of exons that can be effectively engaged. Second, the adenosine and cytosine deaminase domains most commonly used in first-generation BEs possess context-dependent preferences that can prevent their effective editing of a range of splice sites. Third, even when SpCas9 BE technologies are able to edit splice sites, there exist in many exons cryptic splice sites that can be recognized by the spliceosome, which can lead to partial skipping. Furthermore, in addition to cryptic splicing sequences, the targeting of SA or splice donor (SD) sites can lead to the partial or full retention of introns in mature transcripts, which can result in the incorporation of novel sequences into open reading frames or create early termination codons that could undesirably repress expression.

To overcome these limitations, a toolbox of near-PAMless BEs comprised of enhanced deaminase domains that enable the simultaneous targeting of SD and SA sites, which improves exon skipping, reduces aberrant splicing outcomes, including cryptic splicing and intron retention, and enables the skipping of exons previously found to be resilient to skipping. Furthermore, to highlight these compositions and methods, we demonstrate that SPLICER can be used to induce skipping of exon 17 in the gene encoding the amyloid precursor protein (APP), a transmembrane receptor that contributes to the formation of the amyloid deposits in the brain of patients afflicted by Alzheimer's disease. SPLICER reduced the formation of Aβ42 peptides in vitro and mediated efficient exon skipping in a mouse model of Alzheimer's disease.

### Polynucleotides and Polypeptides

Polynucleotides refer to nucleic acid molecules comprising deoxyribonucleic acid (DNA) or ribonucleic acid (RNA). Nucleic acid molecules include but are not limited to genomic DNA, cDNA, mRNA, iRNA, miRNA, tRNA, ncRNA, rRNA, and recombinantly produced and chemically synthesized molecules such as aptamers, plasmids, anti-sense DNA strands, shRNA, ribozymes, nucleic acids conjugated, oligonucleotides or combinations thereof. Polynucleotides can be present as a single-stranded or double-stranded and linear or covalently circularly closed molecule.

Polynucleotides can be obtained from nucleic acid molecules present in, for example, a mammalian cell. Polynucleotides can also be synthesized in the laboratory, for example, using an automatic synthesizer. An amplification method such as PCR can be used to amplify polynucleotides from either genomic DNA or cDNA encoding the polypeptides.

Polynucleotides can be isolated. An isolated polynucleotide can be a naturally-occurring polynucleotide that is not immediately contiguous with one or both of the 5′ and 3′ flanking genomic sequences that it is naturally associated with. An isolated polynucleotide can be, for example, a recombinant DNA molecule of any length, provided that the nucleic acid molecules naturally found immediately flanking the recombinant DNA molecule in a naturally-occurring genome is removed or absent. Isolated polynucleotides also include non-naturally occurring nucleic acid molecules. Polynucleotides can encode full-length polypeptides, polypeptide fragments, and variant or fusion polypeptides. “Isolated polynucleotides” can be (i) amplified in vitro, for example via polymerase chain reaction (PCR), (ii) produced recombinantly by cloning, (iii) purified, for example, by cleavage and separation by gel electrophoresis, (iv) synthesized, for example, by chemical synthesis, or (v) extracted from a sample.

A polynucleotide can comprise, for example, a gene, open reading frame, non-coding region, or regulatory element. A gene is any polynucleotide molecule that encodes a polypeptide, protein, or fragment thereof, optionally including one or more regulatory elements preceding (5′ non-coding sequences) and following (3′ non-coding sequences) the coding sequence. In one aspect, a gene does not include regulatory elements preceding and following the coding sequence. A native or wild-type gene or cell refers to a gene or cell as found in nature, optionally with its own regulatory elements preceding and following the coding sequence. A chimeric or recombinant gene refers to any gene that is not a native or wild-type gene, optionally comprising regulatory elements preceding and following the coding sequence, wherein the coding sequences and/or the regulatory elements, in whole or in part, are not found together in nature. Thus, a chimeric gene or recombinant gene comprise regulatory elements and coding sequences that are derived from different sources, or regulatory elements and coding sequences that are derived from the same source, but arranged differently than is found in nature. A gene can encompass full-length gene sequences (e.g., as found in nature and/or a gene sequence encoding a full-length polypeptide or protein) and can also encompass partial gene sequences (e.g., a fragment of the gene sequence found in nature and/or a gene sequence encoding a protein or fragment of a polypeptide or protein). A gene can include modified gene sequences (e.g., modified as compared to the sequence found in nature). Thus, a gene is not limited to the natural or full-length gene sequence found in nature.

Polynucleotides can be purified free of other components, such as proteins, lipids and other polynucleotides. For example, the polynucleotide can be 50%, 75%, 90%, 95%, 96%, 97%, 98%, 99%, or 100% purified. A polynucleotide existing among hundreds to millions of other polynucleotide molecules within, for example, cDNA or genomic libraries, or gel slices containing a genomic DNA restriction digest are not to be considered a purified polynucleotide. Polynucleotides can encode the polypeptides described herein (e.g., a Cas9 nickase, or a fragment thereof, a N-terminal fragment of an intein, or a C-terminal fragment of an intein).

Degenerate polynucleotide sequences encoding polypeptides described herein, as well as homologous nucleotide sequences are contemplated herein. A homologous nucleotide sequence can be at least about 30, 40, 50, 60, 70, 80, or about 85, 90, 91, 92, 93, 94, 95, 96, 97, 98, or 99% identical to polynucleotides described herein and the complements thereof are also polynucleotides. Degenerate nucleotide sequences are polynucleotides that encode a polypeptide described herein or fragments thereof, but differ in nucleic acid sequence from the wild-type polynucleotide sequence, due to the degeneracy of the genetic code. Complementary DNA (cDNA) molecules, species homologs, and variants of polynucleotides that encode biologically functional polypeptides also are polynucleotides.

Polynucleotides can comprise coding sequences for naturally occurring polypeptides or can encode altered sequences that do not occur in nature.

Unless otherwise indicated, the term polynucleotide or gene includes reference to the specified sequence as well as the complementary sequence thereof.

The expression products of genes or polynucleotides are often proteins, or polypeptides, but in non-protein coding genes such as rRNA genes or tRNA genes, the product is a functional RNA. The process of gene expression is used by all known life forms, i.e., eukaryotes (including multicellular organisms), prokaryotes (bacteria and archaea), and viruses, to generate the macromolecular machinery for life. Several steps in the gene expression process can be modulated, including the transcription, up-regulation, RNA splicing, translation, and post-translational modification of a protein.

A polypeptide is a polymer of two or more amino acids covalently linked by amide bonds. A polypeptide can be post-translationally modified. A purified polypeptide is a polypeptide preparation that is substantially free of cellular material, other types of polypeptides, chemical precursors, chemicals used in synthesis of the polypeptide, or combinations thereof. A polypeptide preparation that is substantially free of cellular material, culture medium, chemical precursors, chemicals used in synthesis of the polypeptide, etc., has less than about 30%, 20%, 10%, 5%, 1% or more of other polypeptides, culture medium, chemical precursors, and/or other chemicals used in synthesis. Therefore, a purified polypeptide is about 70%, 80%, 90%, 95%, 99% or more pure. A purified polypeptide does not include unpurified or semi-purified cell extracts or mixtures of polypeptides that are less than 70% pure.

The term “polypeptides” can refer to one or more of one type of polypeptide (a set of polypeptides). “Polypeptides” can also refer to mixtures of two or more different types of polypeptides (a mixture of polypeptides). The terms “polypeptides” or “polypeptide” can each also mean “one or more polypeptides.”

As used herein, the term “polypeptide of interest” or “polypeptides of interest”, “protein of interest”, “proteins of interest” includes any or a plurality of any of the Cas proteins or fragments thereof, deaminases, N-terminal fragments of dimerization protein, C-terminal fragments of dimerization protein, linkers, protein tags, or other polypeptides (including fragment polypeptides) described herein.

A mutated protein or polypeptide comprises at least one deleted, inserted, and/or substituted amino acid, which can be accomplished via mutagenesis of polynucleotides encoding these amino acids. Mutagenesis includes well-known methods in the art, and includes, for example, site-directed mutagenesis by means of PCR or via oligonucleotide-mediated mutagenesis as described in Sambrook et al., Molecular Cloning-A Laboratory Manual, 2nd ed., Vol. 1-3 (1989).

As used herein, the term “sufficiently similar” means a first amino acid sequence that contains a sufficient or minimum number of identical or equivalent amino acid residues relative to a second amino acid sequence such that the first and second amino acid sequences have a common structural domain and/or common functional activity. For example, amino acid sequences that comprise a common structural domain that is at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100%, identical are defined herein as sufficiently similar Variants will be sufficiently similar to the amino acid sequence of the polypeptides described herein. Such variants generally retain the functional activity of the polypeptides described herein. Variants include peptides that differ in amino acid sequence from the native and wild-type peptide, respectively, by way of one or more amino acid deletion(s), addition(s), and/or substitution(s). These may be naturally occurring variants as well as artificially designed ones.

As used herein, the term “percent (%) sequence identity” or “percent (%) identity,” also including “homology,” is defined as the percentage of amino acid residues or nucleotides in a candidate sequence that are identical with the amino acid residues or nucleotides in the reference sequences after aligning the sequences and introducing gaps, if necessary, to achieve the maximum percent sequence identity, and not considering any conservative substitutions as part of the sequence identity. Optimal alignment of the sequences for comparison may be produced, besides manually, by means of the local homology algorithm of Smith and Waterman, 1981, Ads App. Math. 2, 482, by means of the local homology algorithm of Neddleman and Wunsch, 1970, J. Mol. Biol. 48, 443, by means of the similarity search method of Pearson and Lipman, 1988, Proc. Natl. Acad. Sci. USA 85, 2444, or by means of computer programs which use these algorithms (GAP, BESTFIT, FASTA, BLAST P, BLAST N and TFASTA in Wisconsin Genetics Software Package, Genetics Computer Group, 575 Science Drive, Madison, Wis.).

Polypeptides and polynucleotides that are sufficiently similar to polypeptides and polynucleotides described herein (e.g., Cas proteins, deaminases, dimerization protein, linkers, protein tags, other proteins, or polypeptide fragments thereof) can be used herein. Polypeptides and polynucleotides that are about 85, 90, 91, 92, 93, 94 95, 96, 97, 98, 99 99.5% or more homologous or identical to polypeptides and polynucleotides described herein (e.g., Cas proteins, deaminases, dimerization proteins, linkers, protein tags, or polypeptide fragments thereof) can also be used herein.

### Cas Nickases

In an aspect, a nucleic acid molecule encoding a Cas nickase is provided. A Cas nickase protein can be, e.g., a Cas9 nickase, a dead Cas protein, or a dead Cas9. Cas9 nickases comprise only one catalytically active domain (either the HNH domain or the RuvC domain). Cas9 nickases retain DNA binding based on gRNA specificity, but are capable of cutting only one strand of DNA resulting in a single-strand break (e.g. a “nick”).

A Cas nickase can be a Cas endonuclease Dead (also known as dead Cas or dCas), a mutant form of the protein whose endonuclease activity is removed through point mutations in its endonuclease domains. Any Cas enzyme can be modified to generate a Cas nickase protein.

High fidelity Cas nickase enzymes have improved specificity and can reduce the frequency of off-target events can also be used in the compositions and methods described herein.

A Cas9 nickase can be a Cas9 protein having an amino acid substitution at position 10 or at position 840 or at position 863. Any amino acid substitution that removes the aspartic acid at position 10 (D10), as well as any amino acid substitution that removes the histidine at position 840 (H840) can be used to alter the catalytic activity of the enzyme. For example, the introduction of a H840A substitution in a Cas9 nuclease, through which the 840 amino acid histidine is replaced by an alanine, inactivates one of the nuclease domains. With only one functioning domain, the catalytically impaired Cas9 (H840A Cas9) can only introduce a single strand nick.

Various alterations of Cas9 can lead to the generation of a Cas9 nickase, non-limiting examples of Cas9 nickases include D10A Cas9, D10N Cas9, H840N Cas9, H840Y Cas9, H840A Cas9, or N863A Cas9. Equivalent modifications can be applied to Cas9 variants, as well as to any alternative Cas protein.

In an aspect, a Cas nickase is PAM-less or nearly PAM-less Cas nickase. While the PAM sequence itself is necessary for cleavage, it is not included in a single guide RNA sequence. Binding of an RNA-guided DNA endonuclease to its target sequence can thus happen in the absence of a protospacer adjacent motif (PAM).

A PAM-less or nearly PAM-less Cas nickase is a Cas nickase enzyme that has been engineered to have a reduced or relaxed requirement for a protospacer-adjacent motif (PAM) as compared to a Cas having a NGG PAM. That is, a 2 base PAM having an N in at least one position, a 3 base PAM having an N in at least two positions, or a 4 base PAM having an N in at least two positions. For example, a PAM sequence recognized by the one or more PAM-less or nearly PAM-less Cas nickases can be NN, NNN, NNNN, NNNNN, NNNNNN, NG, NRG, NNN, NRNH, NRN, or NDN. An “N” means that any nucleotide can be present at that position.

A PAM-less or nearly PAM-less Cas nickases can be, for example, SpCas9-NG, xCas9-NG, NAG-Cas9, SpCas9-NRNH, SpG Cas9, SpRY Cas9, SaCas9-KKH, SpCas9-VQR, SpCas9-VRER, and xCas9.

Guide RNA (gRNA) Molecules

Guide RNA (gRNA) is a nucleic acid molecule that promotes the specific association or targeting of a Cas molecule to a target sequence in a cell. gRNAs can be unimolecular (comprising a single RNA molecule (a sgRNA), or can comprise 2 or more separate RNA molecules, such as a crRNA and a tracrRNA, which are usually associated with one another via, e.g., duplexing.

Type II CRISPR systems comprise a CRISPR RNA (crRNA) that includes a 5′ region that is complementary to a foreign sequence, and a trans-activating crRNA (tracrRNA) that includes a 5′ region that is complementary to, and forms a duplex with, a 3′ region of the crRNA. A crRNA and tracrRNA can be joined into a single guide RNA by, for example a four nucleotide (e.g. GAAA) “tetraloop” or “linker” sequence bridging complementary regions of the crRNA (at its 3′ end) and the tracrRNA (at its 5′ end).

Guide RNAs, whether single or comprising more than one component, include a targeting domain that is fully or partially complementary to a target domain within a target sequence, such as a DNA sequence in the genome of a cell where editing is desired. Targeting domains are typically 10-30 nucleotides in length, and in certain aspects are 16-24 nucleotides in length (for instance, 16, 17, 18, 19, 20, 21, 22, 23 or 24 nucleotides in length), and are at or near the 5′ terminus of in the case of a Cas9 gRNA.

In addition to a targeting domain a, a gRNA can include a plurality of domains that can influence the formation or activity of gRNA/Cas complexes. For example, a duplexed structure formed by first and secondary complementarity domains of a gRNA can interact with the recognition (REC) lobe of Cas9 and can mediate the formation of Cas9/gRNA complexes. The first and/or second complementarity domains can contain one or more poly-A tracts, which can be recognized as a termination signal. The sequence of the first and second complementarity domains can optionally be modified to eliminate these tracts and promote the complete in vitro transcription of gRNAs, for instance through the use of A-G swaps or A-U swaps.

Along with the first and second complementarity domains, Cas gRNAs can include two or more additional duplexed regions that are involved in nuclease activity. A first stem-loop one near the 3′ portion of the second complementarity domain can be referred to as the “proximal domain,” “stem loop 1,” or the “nexus.” One or more additional stem loop structures are generally present near the 3′ end of the gRNA, with the number varying by species: S. pyogenes gRNAs typically include two 3′ stem loops for a total of four stem loop structures including the first and secondary complementary domains of a gRNA, while S. aureus and other species have only one (for a total of three stem loop structures).

Although structural differences can exist between gRNAs from different prokaryotic species, the principles by which gRNAs operate are generally consistent. Because of this consistency of operation, gRNAs can be defined, in broad terms, by their targeting domain sequences, and those skilled in the art will understand that a given targeting domain sequence can be incorporated in any suitable gRNA, including a unimolecular or chimeric gRNA, or a gRNA that includes one or more chemical modifications and/or sequential modifications (substitutions, additional nucleotides, truncations, etc.).

A gRNA can encompass any suitable gRNA that can be used with any Cas nickase, and not only those gRNAs that are compatible with a particular species of Cas or Cas9. An gRNA can include a gRNA for use with any CRISPR system.

Methods for selection and validation of target sequences as well as off-target analyses have been described previously. gRNA design can involve the use of a software tool to optimize the choice of potential target sequences corresponding to a target sequence, e.g., to minimize total off-target activity across the genome. These software tools include, for example, GenCRISPR gRNA Design Tool, GuideMaker, CRISPR Guide RNA Design Tool by Benchling, CRISPR Design Tool by Synthego, and SnapGene.

### Base Editors

Provided herein are nucleic acid molecules encoding base editors. Base editors are fusion proteins that comprise a Cas nickase protein fused to a deaminase such as an adenosine deaminase or a cytosine deaminase. Base editors can be targeted to a specific nucleic acid molecule using a gRNA such as a single guide RNA (sgRNA). Base editing uses a deaminase enzyme to modify a single base without altering the bases around it. Cytosine base editors (CBEs) comprise a cytidine deaminase that converts cytidine to uridine within a small editing window near a protospacer adjacent motif (PAM) site. Uridine is then converted to thymidine through base excision repair, creating a cytosine (C) to thymine (T) change (i.e., a guanosine to adenine change on the opposite strand). Adenosine base editors (ABEs) comprise an adenine deaminase that creates an adenine (A) to guanosine (G) edit. A CBE can be used with a uracil DNA glycosylase inhibitor (UGI) to block base excision repair. In some aspects, a UGI domain can be included as part of the base editor fusion protein. In other aspects, a UGI domain is provided to the cell as a separate component.

Third and fourth generation base editors have been developed with improved efficiency. For example, the third generation CBE base editor BE3 (i.e., base editor 3) uses a Cas9 nickase to nick the unmodified DNA strand so that it appears to be newly synthesized to the cell, forcing the cell to repair the DNA using the deaminated strand as a template. BE3 is a functional cytosine base editor (CBE) that comprises Rat APOBEC1 (rA1).

Fourth generation base editor systems (i.e., base editor 4 (BE4)) employ two copies of base excision repair inhibitor UGI. BE4 is a CBE construct that was developed using BE3 as the starter construct. BE4 has an extended rAPOBEC1-Cas9n linker, a Cas9n-UGI linker, and a second copy of UGI appended to the C terminus of the construct. The linker between Cas9 nickase and the rat APOBEC1 cytosine deaminase domain is increased from 16 amino acids to 32 amino acids, the linker between the Cas9 nickase and the uracil glycosylase inhibitor is increased from 4 amino acids to 9 amino acids, and a second uracil glycosylase inhibitor is appended to the C-terminus of the new cytosine base editor using another 9 amino acid linkers.

ABEs were developed using directed evolution of the bacterial dsRNA adenosine deaminase enzyme ecTadA. 14 amino acid changes in TadA were made, which was then fused to an additional wtTadA and nCas9 D10A to generate ABE7.10. Phage-assisted evolution of the wtTaDA domain was performed to provide a more processive and 590-fold more active form of ABE, named ABE8e. ABE8e allows for efficient target adenosine base conversion to guanine at positions 4-8 of the protospacer target site.

In some aspects, a BE3 or BE4 cytosine base editor is used in the methods of the present invention. In other aspects, a CBE such as CBE4max, hA3A-BE4, hA3G-BE4, evoFERNY-BE4, or evoCDA-BE4, CBE SsAPOBEC3B (R54Q), CBE RrA3F (F130L), TadCBE, CBEPpAPOBEC1 (H122A), A3G-BE4.4, A3G-BE5.13, and A3G-BE5.14 is used. In other aspects, an ABE base editor, such as ABE6.3, ABE7.10, ABE8e, ABE9, or ABE8.20m, or ABE8.20 is used. In some aspects, the base editor enzymes are mutated or modified to confer a desired functionality such as reduced guide-independent off-target editing, reduced guide-dependent off-target editing, an altered editing window, an altered editing context preference, an altered target site specificity, or more precise target editing.

### Promoters

A promoter is a polynucleotide that is capable of controlling the expression of a coding sequence or gene. Promoters are generally located 5′ of the sequence that they regulate. Promoters can be derived in their entirety from a native gene, or be composed of different elements derived from promoters found in nature, and/or comprise synthetic nucleotide segments. Those skilled in the art will readily ascertain that different promoters can regulate expression of a coding sequence or gene in response to a particular stimulus, e.g., in a cell- or tissue-specific manner, in response to different environmental or physiological conditions, or in response to specific compounds. Promoters are typically classified into two classes: inducible and constitutive. A constitutive promoter refers to a promoter that allows for continual transcription of the coding sequence or gene under its control.

An inducible promoter refers to a promoter that initiates increased levels of transcription of the coding sequence or gene under its control in response to a stimulus or an exogenous environmental condition. If inducible, there are inducer polynucleotides present therein that mediate regulation of expression so that the associated polynucleotide is transcribed only when an inducer molecule is present. A directly inducible promoter refers to a regulatory region, wherein the regulatory region is operably linked to a gene encoding a protein or polypeptide, where, in the presence of an inducer of the regulatory region, the protein or polypeptide is expressed. An indirectly inducible promoter refers to a regulatory system comprising two or more regulatory regions, for example, a first regulatory region that is operably linked to a first gene encoding a first protein, polypeptide, or factor, e.g., a transcriptional regulator, which is capable of regulating a second regulatory region that is operably linked to a second gene, the second regulatory region may be activated or repressed, thereby activating or repressing expression of the second gene. Both a directly inducible promoter and an indirectly inducible promoter are encompassed by inducible promoter.

A promoter can be any polynucleotide that shows transcriptional activity in the chosen host organism. A promoter can be naturally-occurring, can be composed of portions of various naturally-occurring promoters, or may be partially or totally synthetic. Guidance for the design of promoters is derived from studies of promoter structure, such as that of Harley and Reynolds, Nucleic Acids Res., 15, 2343-61 (1987). In addition, the location of the promoter relative to the transcription start can be optimized. Many suitable promoters for use in mammalian cells and other cells available, as are polynucleotides that enhance expression of an associated expressible polynucleotide. Non-limiting examples of promoters that can be used to in the present compositions can include cytomegalovirus (CMV) promoter and the Rous sarcoma virus promoter, that allows for unregulated expression in mammalian cells.

### Nuclear Localization Signals

A nuclear localization signal or sequence (NLS) is an amino acid sequence that ‘tags’ a protein for import into the nucleus by nuclear transport. Typically, this signal comprises one or more short sequences of positively charged lysines or arginines exposed on the protein surface. Different nuclear localized proteins can share the same NLS. There are two types of NLSs, the classical and the non-classical NLS.

Classical NLSs can be classified as either monopartite or bipartite, depending on the presence of a short spacer sequence separating the two basic amino acid clusters (present in bipartite NLSs).

An example of a monopartite NLS includes PKKKRKV (SEQ ID NO:368) from the SV40 Large T-antigen; while the NLS of nucleoplasmin, KR[PAATKKAGQA]KKKK (SEQ ID NO:369) is an example of bipartite signal. Both signals can recognized by importin α. Importin α contains a bipartite NLS itself, which is specifically recognized by importin p, considered as the actual import mediator.

Many other non-classical NLS are also known, such as the acidic M9 domain of hnRNP A1, the sequence KIPIK (SEQ ID NO:394) in yeast transcription repressor Mata2, and the complex signals of U snRNPs. Most of these NLSs appear to be recognized directly by specific receptors of the importin β family without the intervention of an importin a-like protein.

Any NLS can be used in the compositions described herein, including inducible NLSs such as light-inducible NLSs for example.

Polynucleotide molecules described herein can include one or more NLS. For example, a polynucleotide can comprise 1, 2, 3, 4, or more NLSs of any type. For example, a polynucleotide can comprise 1, 2, 3, 4, or more NLSs, wherein the 1, 2, 3, 4, or more NLSs are classical NLSs; a polynucleotide can comprise 1, 2, 3, 4, or more NLSs, wherein the 1, 2, 3, 4, or more NLSs are non-classical NLSs; or a polynucleotide can comprise 1, 2, 3, 4, or more NLSs, some of 1, 2, 3, 4, or more of the NLSs are classical NLSs and the remaining of the 1, 2, 3, 4, or more of the NLSs are non-classical NLSs. The NLS sequence can occur anywhere in the molecule. For example, a NLS sequence can be incorporated at the 5′ end of a polynucleotide molecule, at the 3′ end of the molecule, or both at the 5′ and at the 3′ end of the molecule.

### Linkers

Linkers are polynucleotide sequences than can encode a polypeptide joining the Cas nickase and deaminase in a prime editor. Linkers can influence the activity of a prime editor.

In an aspect polynucleotide molecule encoding a Cas nickase can further comprise a linker, so that the linker can join the deaminase and the Cas nickase. The linker can encode a polypeptide molecule comprising SEQ ID NO:370, 371, 372, 373, or 374.

linker
SEQ ID NO: 370
SGGSSGGSSGSETPGTSESATPESSGGSSGGSST
linker
SEQ ID NO: 371
SGGSSGGSSGSETPGTSESATPESSGGSSGGSSTLEPGEKPYKCPECGKS
FSQSGALTRHQRTHTRDKKYSIGLDIGTNSVGWAVITDEYKVPLE
linker
SEQ ID NO: 372
ASGGGGSGGGGSGGGGSGGGGSGGGGSLE
linker
SEQ ID NO: 373
SGGSSGGSSGSETPGTSESATPESSGGSSGGSST
linker
SEQ ID NO: 374
ASASSGGSSGGSSGSETPGTSESATPESSGGSSGGSGGGGSGGGGSGG
GGSGGGGSGGGGSGTLE

### Protein Tags

Proteins tags are small polypeptide sequences that can be used for protein detection. Proteins tags do not modify activity of the prime editors described herein, but can be used for isolation or detection of the prime editors.

Non limiting examples of protein tags include V5, His, or FLAG.

In an aspect, the first polynucleotide molecule can comprise one or more polynucleotides encoding a protein tag and the second polynucleotide molecule can comprise one or more polynucleotides encoding a protein tag.

Polynucleotide molecules described herein can include one or more protein tags. For example, a polynucleotide can comprise 1, 2, 3, 4, or more protein tags of any type. The protein tag sequence can occur anywhere in the molecule. For example, a protein tag sequence can be incorporated at the 5′ end of a polynucleotide molecule, at the 3′ end of the molecule, or both at the 5′ and at the 3′ end of the molecule.

### RNA Splicing

RNA splicing is a process by which introns (noncoding regions of genes) are cut out of a primary pre-messenger RNA transcript, and the exons (coding regions of genes) are joined together to make mature messenger RNA. Mature messenger RNA carries the instructions a cell needs for making a specific protein.

Introns are removed from primary transcripts (i.e., pre-mRNAs) by cleavage at conserved splice site sequences. Splice sites are located at the 5′ and 3′ ends of introns. The RNA sequence that is removed often begins with the dinucleotide GU (sometimes this can be AU) at its 5′ end, and ends with AG (sometimes this can be AC) at its 3′ end. A branch point is located anywhere from 18 to 40 nucleotides upstream from the 3′ end of an intron. The branch point contains an adenine, but it is otherwise loosely conserved. A typical sequence is YNYYRAY, where Y indicates a pyrimidine, N denotes any nucleotide, R denotes any purine, and A denotes adenine.

Splicing occurs in several steps and is catalyzed by small nuclear ribonucleoproteins (snRNPs). Initially, a pre-mRNA is cleaved at the 5′ end of the intron following the attachment of a snRNP called U1 to its complementary sequence within the intron. The cut end then attaches to the conserved branch point region downstream through pairing of guanine and adenine nucleotides from the 5′ end and the branch point, respectively, to form a looped structure. The bonding of the guanine and adenine bases takes place via transesterification, in which a hydroxyl (OH) group on a carbon atom of the adenine “attacks” the bond of the guanine nucleotide at the splice site. The guanine residue is thus cleaved from the RNA strand and forms a new bond with the adenine.

Next, snRNPs U2 and U4/U6 contribute to positioning of the 5′ end and the branch point in proximity. With the participation of U5, the 3′ end of the intron is brought into proximity, cut, and joined to the 5′ end. This step occurs by transesterification; in this case, an OH group at the 3′ end of the exon attacks the phosphodiester bond at the 3′ splice site. The adjoining exons are covalently bound, and the resulting lariat is released with U2, U5, and U6 bound to it.

Introns are removed from pre-mRNA molecules by splicing to form mature mRNA molecules made up of exons. The compositions and methods described herein can be used to alter the splicing of pre-mRNA molecules so that exon skipping or partial exon skipping can be used to restore or alter a reading frame of a gene. That is, all or part of an exon (e.g., a part of an exon may be about 5, 10, 50, 100, 200, 300, 400, 500, 750, 1,000 or more nucleotides in length) does not appear in the mature mRNA molecule.

In some aspects, an amount of cryptic splicing is reduced as compared to a cell not subjected to the methods described herein. Cryptic splicing occurs when splice junctions form between known splice sites and new locations within introns. Cryptic splicing can be caused by a mutation in a gene, usually at an authentic splice site, that activates cryptic splice sites. In an aspect, cryptic splicing is reduced by about 10, 20, 30, 40, 50, 60, 70% or more.

In some aspects, intron retention is reduced as compared to a control (e.g., a control cell) not subjected to the methods described herein. Intron retention is where an intron remains in a mature mRNA instead of being spliced out. In an aspect, intron retention is reduced by about 10, 20, 30, 40, 50, 60, 70% or more.

In some aspects, full exon skipping is increased as compared to a control (e.g., a cell not subjected to the methods described herein). Full exon skipping is where an entire exon is skipped and does not appear in the mature mRNA. In an aspect, full exon skipping is increased by about 10, 20, 30, 40, 50, 60, 70% or more.

In an aspect, exons having exon splice enhancer (ESE) sequences can be targeted using one or more gRNAs targeting splice donor and splice acceptor sites. In an aspect, splice donor and splice acceptor sites can be targeted where a target splice donor site and/or target splice acceptor sites are within 10, 50, 100, 500, 750, or 1,000 nucleotides of one or more ESE sequences.

### Vectors

Polynucleotides can be delivered to cells (e.g., a plurality of different cells or cell types including target cells or cell types and/or non-target cell types) in a vector (e.g., an expression vector). A vector is a nucleic acid molecule comprising an intact replicon such that the vector can be replicated when placed within a cell, for example by a process of transfection, infection, or transformation. Once inside a cell, a vector can replicate as an extrachromosomal (episomal) element or can be integrated into a host cell chromosome. Vectors can include nucleic acids derived from retroviruses, adenoviruses, herpesvirus, baculoviruses, modified baculoviruses, papovaviruses, or otherwise modified naturally-occurring viruses. Exemplary non-viral vectors for delivering nucleic acid molecules include naked DNA; DNA complexed with cationic lipids, alone or in combination with cationic polymers; anionic and cationic liposomes; DNA-protein complexes and particles comprising DNA condensed with cationic polymers such as heterogeneous polylysine, defined-length oligopeptides, and polyethyleneimine, contained in liposomes; and the use of ternary complexes comprising a virus and polylysine-DNA.

Vectors that contain both a promoter and a cloning site into which a polynucleotide can be operably linked can be used in the compositions and methods described herein. These vectors are capable of transcribing RNA in vitro or in vivo. To optimize expression and/or in vitro transcription, it can be necessary to remove, add, or alter 5′ and/or 3′ untranslated portions of cloned transgenes to eliminate extra, potential inappropriate alternative translation initiation codons or other sequences that can interfere with or reduce expression, either at the level of transcription or translation. Consensus ribosome binding sites can be inserted immediately 5′ of the start codon to enhance expression.

One or more vectors can comprise one or more nucleic acid molecules including for example, promoters, gRNA molecules, NLSs, linkers, and/or nucleic acid molecules encoding Cas nickases, dimerization proteins, protein tags, deaminases, etc.

A viral vector is a recombinantly produced virus or viral particle that contains a polynucleotide to be delivered into a host cell, either in vivo, ex vivo or in vitro. Examples of viral vectors include retroviral vectors, AAV vectors, lentiviral vectors, adenovirus vectors, alphavirus vectors and the like.

Nucleic acid molecules described herein can delivered to a cell via a viral vector (e.g., retroviral, adenoviral, AAV, helper-dependent adenoviral systems, hybrid adenoviral systems, herpes simplex, pox virus, lentivirus, and Epstein-Barr virus), and non-viral systems, such as physical systems (naked DNA, DNA bombardment, electroporation, hydrodynamic, ultrasound, and magnetofection), and chemical system (cationic lipids, different cationic polymers, and lipid polymers).

The cloning capacity of vectors or viral expression vectors can vary. For example, AAV vectors typically have a packaging capacity of 4.8 kb, lentiviruses typically have a capacity of 8 kb, adenoviruses typically have a capacity of 7.5 kb and alphaviruses typically have a capacity of 7.5 kb. Some viruses can have larger packaging capacities, for example herpesvirus can have a capacity of >30 kb and vaccinia a capacity of 25 kb. Advantages of using AAV for gene therapy include low pathogenicity, very low frequency of integration into the host genome, and the ability to infect dividing and non-dividing cells.

Viral gene therapy vectors or gene delivery vectors can have the ability to be reproducible and stably propagated and purified to high titers; to mediate targeted delivery (e.g., to deliver the transgene specifically to a tissue or organ of interest without widespread vector dissemination elsewhere or off-target delivery); and to mediate gene delivery and/or transgene expression without inducing harmful side effects or off-target effects.

An adeno-associated virus (AAV) is a member of the class of viruses associated with this name and belonging to the genus Dependoparvovirus, family Parvoviridae. Adeno-associated virus is a single-stranded DNA virus that grows in cells in which certain functions are provided by a co-infecting helper virus. General information and reviews of AAV can be found in, for example, Carter, 1989, Handbook of Parvoviruses, Vol. 1, pp. 169-228, and Berns, 1990, Virology, pp. 1743-1764, Raven Press, (New York). It is fully expected that the same principles described in these reviews will be applicable to additional AAV serotypes characterized in the future because the various serotypes are quite closely related, both structurally and functionally, even at the genetic level. See, for example, Blacklowe, 1988, pp. 165-174 of Parvoviruses and Human Disease, J. R. Pattison, ed.; and Rose, Comprehensive Virology 3: 1-61 (1974). For example, all AAV serotypes apparently exhibit very similar replication properties mediated by homologous rep genes; and all have three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control. Multiple serotypes of this virus are known to be suitable for gene delivery; all known serotypes can infect cells from various tissue types. Non-limiting exemplary serotypes useful in the methods disclosed herein include any of the serotypes, e.g., AAV2, AAV8, AAV9, or variant serotypes, e.g., AAV-DJ and AAV PHP.B. The AAV particle comprises, consists essentially of, or consists of three major viral proteins: VPI, VP2 and VP3. In some aspects, the AAV refers to the serotype AAV1, AAV2, AAV4, AAV5, AAV6, AAV7, AAV8, AAV9, AAV10, AAV11, AAV12, AAV13, AAVPHP.B, AAVrh74 or AAVrh.10.

AAVs and recombinant AAV include, but are not limited to all serotypes (e.g., AAV1, AAV2, AAV3, AAV4, AAV5, AAV6, AAV7, AAV8, AAV9, AAV10, AAV11, AAV12, AAV13, AAVPHP.B, AAVrh74 and AAVrh.10), self-complementary AAV (scAAV) and AAV hybrids containing the genome of one serotype and the capsid of another serotype (e.g., AAV2/5, AAV-DJ and AAV-DJ8).

In some aspects, a vector comprises 1, 2, 3, 4, or more AAV ITR (inverted terminal repeat) sequences. An AAV ITR can comprise any AAV ITR sequence known in the art. In some aspects, an AAV ITR sequence can be an AAV1 ITR sequence, an AAV2 ITR sequence, an AAV4 ITR sequence, an AAV5 ITR sequence, an AAV6 ITR sequence, an AAV7 ITR sequence, an AAV8 ITR sequence, an AAV9 ITR sequence, an AAV10 ITR sequence, an AAV11 ITR sequence, an AAV12 ITR sequence, an AAV13 ITR sequence, an AAVrh74 ITR sequence, or an AAVrh.10 ITR sequence.

SEQ ID NO:375 and 376 are examples of AAV ITR sequences.

SEQ ID NO:375

AGGAACCCCT AGTGATGGAG TTGGCCACTC CCTCTCTGCG CGCTCGCTCG
CTCACTGAGG CCGGGCGACC AAAGGTCGCC CGACGCCCGG GCTTTGCCCG
GGCGGCCTCA GTGAGCGAGC GAGCGCGCAG CTGGCGTAAT AGCGAAGAGG
CCCGCACCGA TCGCCCTTC
SEQ ID NO: 376
CTGCGCGCTC GCTCGCTCAC TGAGGCCGCC CGGGCAAAGC CCGGGCGTCG
GGCGACCTTT GGTCGCCCGG CCTCAGTGAG CGAGCGAGCG CGCAGAGAGG
GAGTGG

An AAV viral vector or pharmaceutical composition can be for administration to the subject at a dose ranging from about 101 to about 10¹⁸viral vector particles. An AAV viral vector or pharmaceutical composition can be for administration to the subject at a dose ranging from about 10¹³ to about 10¹⁶ viral vector particles.

An AAV viral vector, other vector, or pharmaceutical composition can be for administration to the subject systemically, intravenously, intrathecally, intracerebrally, intraventricularly, intranasally, intratracheally, intra-aurally, intra-ocularly, intracerebral, or peri-ocularly, orally, rectally, transmucosally, inhalationally, transdermally, parenterally, subcutaneously, intradermally, intramuscularly, intracisternally, intranervally, intrapleurally, by lumbar puncture, intralymphatically, intracisternally, or intranerve.

### Split Base Editor/Nickase Constructs

In some aspects, a vector may not be large enough to package nucleic acid molecules encoding all necessary components to achieve a desired effect. In these cases dimerization proteins can be used to assemble all necessary proteins to a cell or patient.

Dimerization proteins are intron-like proteins that can splice proteins. Dimerization proteins can spontaneously excise themselves from two host proteins or protein domains and splice its flanking N- and C-terminal domains to become a mature protein. Protein splicing is a post-translational process that includes peptide bond cleavage and backbone conjugation activities, which requires no cofactor or ATP hydrolysis.

Non limiting examples of dimerization proteins include inteins, inducible dimers, or other non-inteins. In one aspect, the dimerization protein is an intein. Similar split system using FKBP/FRB, SNAPtag/HaloTag, light-inducible dimers, any dimerizing protein pairs, or any other protein that can be used to enforce transient or permanent dimerization can be in place of inteins.

Inteins can be divided into three major regions, an amino (or N) terminal splicing domain (INn), a carboxy (or C) terminal splicing domain (INc), and an optional endonuclease region. The N- and C-terminal splicing domains comprise conserved amino acid motifs shared by all known inteins, and including a cysteine (or serine or threonine) residue following the scissile peptide bonds at the N-terminal and C-terminal splice junctions, as well as a highly conserved asparagine at the C-terminus of the intein. These amino acid residues appear to directly participate in the cleavage of the two flanking peptide bonds and linkage of the external protein sequences.

In an aspect, an N-terminal fragment of an intein comprises an N-terminal splicing domain (INn). In an aspect, a C-terminal fragment of an intein comprises a C-terminal splicing domain (INc).

Inteins can excise themselves out of the host protein while reconnecting the remaining N and C exteins (i.e., the protein previously bound to the inteins) via a new peptide bond, therefore, inteins can be used to generate fusion protein. For example, if a cell expresses a first polypeptide encoding a Cas nickase (such as a PAM-less or nearly PAM-less nickase), and an N-terminal fragment of an intein, and a second polypeptide encoding a C-terminal fragment of an intein; upon translation of both proteins, the N-terminal fragment of an intein and the C-terminal fragment of an intein can perform an autocatalytic reaction to generate new bonds between the two intein fragments, excise themselves, and generate a new peptidic bond.

Many inteins are known in the art, and they can be derived from various organisms. For example, a C-terminal fragment of an intein and a N-terminal fragment of an intein can be derived from PhoRadA, RmaDnaB^{Δ286}, SspDnaB^{Δ275}, SspDnaB^{M86Δ275}, SspDnaX, TvoVMA, NpuDnaE, NpuDnaB^{Δ283}, SspGyrB, AceL-TerL, PchPRP8, PfuRIR1-1, Psp-GDBPol-1, MtuRecA^{Δ228}, PfuRIR1-2, SceVMA^{Δ206}, RmaDnaB^{Δ271}, MtuRecA^{Δ285} SspDnaB^{Δ274}, gp41-8, SceVMA^{Δ227}, IMPDH-1, NrdJ-1, MtuRecA^{Δ297}, gp41-1, AovDnaE, AspDnaE, AvaDnaE, Cra(C5505)DnaE, Csp(CCY0110)DnaE, Csp(PCC8801)DnaE, CwaDnaE, Maer(NIES843)DnaE, Mcht(PCC7420)DnaE, MtuRec^{Δ300}, NspDnaE, OliDnaE, Sel(PC7942)DnaE, SspDnaE, Ssp(PCC7002)DnaE, TerDnaE-3, TelDnaE, TvuDnaE, NeqPol, or TerThyX^{Δ132}.

An aspect provides a system comprising: a first vector encoding an N-terminal fragment of a Cas nickase, and an N-terminal fragment of a dimerization protein; and a second vector encoding a C-terminal fragment of a dimerization protein and a C-terminal fragment of Cas nickase protein. The N-terminal fragment of the Cas nickase protein and the C-terminal fragment of the Cas nickase protein can form a full-length Cas nickase protein when combined. See, e.g., US Pat. Publ. 20240026381, which is incorporated herein in its entirety.

In an aspect, an N-terminal fragment of a dimerization protein can be the N-terminal fragment of an intein; and a C-terminal fragment of a dimerization protein can be the C-terminal fragment of an intein.

In an aspect, a first vector can comprise one or more nuclear localization signals and the second vector can comprise one or more nuclear localization signals.

In an aspect, a first vector can comprise two NLS sequences, an N-terminal fragment of Cas nickase, and an N-terminal fragment of an intein. The Cas nickase can be a Cas9 nickase, a dead Cas protein, a dead Cas9, or an active Cas protein. For example, a first vector can comprise a first NLS sequence, a second NLS sequence, a N-terminal fragment of a Cas nickase, and a N-terminal fragment of an intein. In an aspect, a N-terminal fragment of an intein can comprise SEQ ID NO:377:

CLSYETEILTVEYGLLPIGKIVEKRIECTVYSVDNNGNIYTQPVAQWHD
RGEQEVFEYCLEDGSLIRATKDHKFMTVDGQMLPIDEIFERELDLMRVD
NLPN

In another aspect, a second vector can comprise two NLS sequences, a C-terminal fragment of Cas nickase, and a C-terminal fragment of an intein. For example, a second vector can comprise a first NLS sequence, a C-terminal fragment of an intein, a C-terminal fragment of a Cas9 nickase, and a second NLS sequence. In an aspect, a C-terminal fragment of an intein can comprise SEQ ID NO:378: IKIATRKYLGKQNVYDIGVERDHNFALKNGFIASN.

In an aspect, Cas nickase can be split into a N-terminal fragment and a C-terminal fragment.

In an aspect, the Cas9 nickase can be split into a N-terminal fragment and a C-terminal fragment at a split point.

In an aspect, the split point can be localized at any amino acid between position 564 and 584 (e.g. 564, 565, 566, 567, 568, 569, 570, 571, 572, 573, 574, 575, 576, 577, 578, 579, 580, 581, 582, 583, 584), and the N-terminal fragment of Cas9 nickase can comprise nucleotides from position 1 of a Cas9 nickase to the split point and the C-terminal fragment of Cas9 nickase can comprise nucleotides from the split point to position 1371 of a Cas9 nickase. In an aspect, a first polynucleotide molecule can comprise nucleotides 1-574 of a Cas9 nickase and an N-terminal fragment of an intein. A second polynucleotide molecule can comprise a C-terminal fragment of an intein and nucleotides 575-1371 of a Cas9 nickase. For example, a first polynucleotide molecule can encode SEQ ID NO:379, and a second polynucleotide molecule can encode SEQ ID NO:380.

SEQ ID NO: 379
MGPKKKRKVGGSSTLNIEDEYRLHETSKEPDVSLGSTWLSDFPQAWAETG
GMGLAVRQAPLIIPLKATSTPVSIKQYPMSQEARLGIKPHIQRLLDQGILVPCQSPWN
TPLLPVKKPGTNDYRPVQDLREVNKRVEDIHPTVPNPYNLLSGLPPSHQWYTVLDL
KDAFFCLRLHPTSQPLFAFEWRDPEMGISGQLTWTRLPQGFKNSPTLFNEALHRDL
ADFRIQHPDLILLQYVDDLLLAATSELDCQQGTRALLQTLGNLGYRASAKKAQICQK
QVKYLGYLLKEGQRWLTEARKETVMGQPTPKTPRQLREFLGKAGFCRLFIPGFAE
MAAPLYPLTKPGTLFNWGPDQQKAYQEIKQALLTAPALGLPDLTKPFELFVDEKQG
YAKGVLTQKLGPWRRPVAYLSKKLDPVAAGWPPCLRMVAAIAVLTKDAGKLTMGQ
PLVILAPHAVEALVKQPPDRWLSNARMTHYQALLLDTDRVQFGPVVALNPATLLPL
PEEGLQHNCLDILAEAHGTRPDLTDQPLPDADHTWYTDGSSLLQEGQRKAGAAVT
TETEVIWAKALPAGTSAQRAELIALTQALKMAEGKKLNVYTDSRYAFATAHIHGEIYR
RRGWLTSEGKEIKNKDEILALLKALFLPKRLSIIHCPGHQKGHSAEARGNRMADQAA
RKAAITETPDTSTLLIENSSPSGGSKRTADGSEFEPKKKRKVSGGSSGGSSGSETP
GTSESATPESSGGSSGGSSTLEPGEKPYKCPECGKSFSQSGALTRHQRTHTRDK
KYSIGLDIGTNSVGWAVITDEYKVPSKKFKVLGNTDRHSIKKNLIGALLFDSGETAEA
TRLKRTARRRYTRRKNRICYLQEIFSNEMAKVDDSFFHRLEESFLVEEDKKHERHPI
FGNIVDEVAYHEKYPTIYHLRKKLVDSTDKADLRLIYLALAHMIKFRGHFLIEGDLNPD
NSDVDKLFIQLVQTYNQLFEENPINASGVDAKAILSARLSKSRRLENLIAQLPGEKKN
GLFGNLIALSLGLTPNFKSNFDLAEDAKLQLSKDTYDDDLDNLLAQIGDQYADLFLA
AKNLSDAILLSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRKQRTFDN
GSIPHQIHLGELHAILRRQEDFYPFLKDNREKIEKILTFRIPYYVGPLARGNSRFAWM
TRKSEETITPWNFEEVVDKGASAQSFIERMTNFDKNLPNEKVLPKHSLLYEYFTVYN
ELTKVKYVTEGMRKPAFLSGEQKKAIVDLLFKTNRKVTVKQLKEDYFKKIECLSYET
EILTVEYGLLPIGKIVEKRIECTVYSVDNNGNIYTQPVAQWHDRGEQEVFEYCLEDG
SLIRATKDHKFMTVDGQMLPIDEIFERELDLMRVDNLPN
SEQ ID NO: 380
MKRTADGSEFESPKKKRKVIKIATRKYLGKQNVYDIGVERDHNFALKNGFIA
SNCFDSVEISGVEDRFNASLGTYHDLLKIIKDKDFLDNEENEDILEDIVLTLTLFEDRE
MIEERLKTYAHLFDDKVMKQLKRRRYTGWGRLSRKLINGIRDKQSGKTILDFLKSDG
FANRNFMQLIHDDSLTFKEDIQKAQVSGQGDSLHEHIANLAGSPAIKKGILQTVKVV
DELVKVMGRHKPENIVIEMARENQTTQKGQKNSRERMKRIEEGIKELGSQILKEHP
VENTQLQNEKLYLYYLQNGRDMYVDQELDINRLSDYDVDAIVPQSFLKDDSIDNKVL
TRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDF
QFYKVREINNYHHAHDAYLNAVVGTALIKKYPKLESEFVYGDYKVYDVRKMIAKSEQ
EIGKATAKYFFYSNIMNFFKTEITLANGEIRKRPLIETNGETGEIVWDKGRDFATVRK
VLSMPQVNIVKKTEVQTGGFSKESILPKRNSDKLIARKKDWDPKKYGGFDSPTVAY
SVLVVAKVEKGKSKKLKSVKELLGITIMERSSFEKNPIDFLEAKGYKEVKKDLIIKLPK
YSLFELENGRKRMLASAGELQKGNELALPSKYVNFLYLASHYEKLKGSPEDNEQK
QLFVEQHKHYLDEIIEQISEFSKRVILADANLDKVLSAYNKHRDKPIREQAENIIHLFTL
TNLGAPAAFKYFDTTIDRKRYTSTKEVLDATLIHQSITGLYETRIDLSQLGGDSGGST
NLSDIIEKETGKQLVIQESILMLPEEVEEVIGNKPESDILVHTAYDESTDENVMLLTSD
APEYKPWALVIQDSNGENKIKMLSYPYDVPDYAYPYDVPDYAYPYDVPDYASGGS
PKKKRKV

In another aspect, the split point can be localized at any amino acid between position 249 and 269 (e.g., 249, 250, 251, 252, 253, 254, 255, 256, 257, 258, 259, 260, 261, 262, 263, 264, 265, 266, 267, 268, 269), and the N-terminal fragment of Cas9 nickase can comprise nucleotides from position 1 of a Cas9 nickase to the split point and the C-terminal fragment of Cas9 nickase can comprise nucleotides from the split point to position 1371 of a Cas9 nickase. In an aspect, a first polynucleotide molecule can comprise an N-terminal fragment of an intein, and nucleotides 1-259 of a Cas9 nickase. A second polynucleotide molecule comprising nucleotides 260-1371 of a Cas9 nickase and a C-terminal fragment of an intein.

In an aspect, the split point can be localized at any amino acid between position 265 and 285 (e.g., 265, 266, 267, 268, 269, 270, 271, 272, 273, 274, 275, 276, 277, 278, 279, 280, 281, 282, 283, 284, 285), and the N-terminal fragment of Cas9 nickase can comprise nucleotides from position 1 of a Cas9 nickase to the split point and the C-terminal fragment of Cas9 nickase can comprise nucleotides from the split point to position 1371 of a Cas9 nickase. In an aspect, a first vector can comprise nucleotides 1-275 of a Cas9 nickase and an N-terminal fragment of an intein. A second vector can comprise nucleotides 276-1371 of a Cas9 nickase and a C-terminal fragment of an intein.

Another aspect provides a system comprising: a first vector encoding a N-terminal fragment of a Cas nickase protein, and a N-terminal fragment of a dimerization protein; and a second vector encoding a C-terminal fragment of a dimerization protein, and a C-terminal fragment of Cas nickase protein.

In an aspect, a N-terminal fragment of a dimerization protein can be the N-terminal fragment of an intein; and a C-terminal fragment of a dimerization protein can be the C-terminal fragment of an intein.

In an aspect, a first vector can comprise one or more nuclear localization signals and the second vector can comprise one or more nuclear localization signals.

In an aspect, a first vector can comprise two NLS sequences, an N-terminal fragment of Cas nickase, and an N-terminal fragment of an intein transcriptase. The Cas nickase can be a Cas9 nickase, a dead Cas protein, a dead Cas9, or an active Cas protein. For example, a first vector can comprise a first NLS sequence, a N-terminal fragment of a Cas9 nickase, a second NLS sequence, and a N-terminal fragment of an intein. In an aspect, a N-terminal fragment of an intein can comprise SEQ ID NO:377.

In another aspect, a second vector can comprise three NLS sequences, and a C-terminal fragment of an intein a C-terminal fragment of Cas nickase. For example, a second polynucleotide molecule can comprise a first NLS sequence, a C-terminal fragment of an intein, a second NLS sequence, a C-terminal fragment of a Cas9 nickase, and a third NLS sequence. In an aspect, a C-terminal fragment of an intein can comprise SEQ ID NO:378.

In an aspect, the Cas nickase can be split into a N-terminal fragment and a C-terminal fragment at a split point.

In an aspect, the split point can be localized at any amino acid between position 703 and 723 (e.g., 712), and the N-terminal fragment of Cas9 nickase can comprise nucleotides from position 1-713 of the Cas9 nickase to the split point and the C-terminal fragment of Cas9 nickase can comprise nucleotides from the split point to position 714-1371 of the Cas9 nickase. In an aspect, a first vector comprising an N-terminal fragment of an intein, and nucleotides 1-713 of a Cas9 nickase. A second vector can comprise nucleotides 714-1371 of a Cas9 nickase and a C-terminal fragment of an intein. For example, a first vector can encode SEQ ID NO:381, and a second vector can encode SEQ ID NO:382.

N-terminus fragment of Split 713
SEQ ID NO: 381
MKRTADGSEFESPKKKRKVDKKYSIGLDIGTNSVGWAVITDEYKVPSKKFKV
LGNTDRHSIKKNLIGALLFDSGETAEATRLKRTARRRYTRRKNRICYLQEIFSNEMA
KVDDSFFHRLEESFLVEEDKKHERHPIFGNIVDEVAYHEKYPTIYHLRKKLVDSTDK
ADLRLIYLALAHMIKFRGHFLIEGDLNPDNSDVDKLFIQLVQTYNQLFEENPINASGV
DAKAILSARLSKSRRLENLIAQLPGEKKNGLFGNLIALSLGLTPNFKSNFDLAEDAKL
QLSKDTYDDDLDNLLAQIGDQYADLFLAAKNLSDAILLSDILRVNTEITKAPLSASMIK
RYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNR
EKIEKILTFRIPYYVGPLARGNSRFAWMTRKSEETITPWNFEEVVDKGASAQSFIER
MTNFDKNLPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKIIKDKDFLDN
EENEDILEDIVLTLTLFEDREMIEERLKTYAHLFDDKVMKQLKRRRYTGWGRLSRKLI
NGIRDKQSGKTILDFLKSDGFANRNFMQLIHDDSLTFKEDIQKAQVCLAGDTLITLAD
GRRVPIRELVSQQNFSVWALNPQTYRLERARVSRAFCTGIKPVYRLTTRLGRSIRA
TANHRFLTPQGWKRVDELQPGDYLALPRRIPTAS
C-terminus fragment of Split 713
SEQ ID NO: 382
MAAACPELRQLAQSDVYWDPIVSIEPDGVEEVFDLTVPGPHNFVANDIIAHN
SGQGDSLHEHIANLAGSPAIKKGILQTVKVVDELVKVMGRHKPENIVIEMARENQTT
QKGQKNSRERMKRIEEGIKELGSQILKEHPVENTQLQNEKLYLYYLQNGRDMYVD
QELDINRLSDYDVDAIVPQSFLKDDSIDNKVLTRSDKNRGKSDNVPSEEVVKKMKN
YWRQLLNAKLITQRKFDNLTKAERGGLSELDKAGFIKRQLVETRQITKHVAQILDSR
MNTKYDENDKLIREVKVITLKSKLVSDFRKDFQFYKVREINNYHHAHDAYLNAVVGT
ALIKKYPKLESEFVYGDYKVYDVRKMIAKSEQEIGKATAKYFFYSNIMNFFKTEITLA
NGEIRKRPLIETNGETGEIVWDKGRDFATVRKVLSMPQVNIVKKTEVQTGGFSKESI
LPKRNSDKLIARKKDWDPKKYGGFDSPTVAYSVLVVAKVEKGKSKKLKSVKELLGIT
IMERSSFEKNPIDFLEAKGYKEVKKDLIIKLPKYSLFELENGRKRMLASAGELQKGNE
LALPSKYVNFLYLASHYEKLKGSPEDNEQKQLFVEQHKHYLDEIIEQISEFSKRVILA
DANLDKVLSAYNKHRDKPIREQAENIIHLFTLTNLGAPAAFKYFDTTIDRKRYTSTKE
VLDATLIHQSITGLYETRIDLSQLGGDSGGSSGGSSGSETPGTSESATPESSGGSS
GGSSTLNIEDEYRLHETSKEPDVSLGSTWLSDFPQAWAETGGMGLAVRQAPLIIPL
KATSTPVSIKQYPMSQEARLGIKPHIQRLLDQGILVPCQSPWNTPLLPVKKPGTNDY
RPVQDLREVNKRVEDIHPTVPNPYNLLSGLPPSHQWYTVLDLKDAFFCLRLHPTSQ
PLFAFEWRDPEMGISGQLTWTRLPQGFKNSPTLFNEALHRDLADFRIQHPDLILLQY
VDDLLLAATSELDCQQGTRALLQTLGNLGYRASAKKAQICQKQVKYLGYLLKEGQR
WLTEARKETVMGQPTPKTPRQLREFLGKAGFCRLFIPGFAEMAAPLYPLTKPGTLF
NWGPDQQKAYQEIKQALLTAPALGLPDLTKPFELFVDEKQGYAKGVLTQKLGPWR
RPVAYLSKKLDPVAAGWPPCLRMVAAIAVLTKDAGKLTMGQPLVILAPHAVEALVK
QPPDRWLSNARMTHYQALLLDTDRVQFGPVVALNPATLLPLPEEGLQHNCLDILAE
AHGTRPDLTDQPLPDADHTWYTDGSSLLQEGQRKAGAAVTTETEVIWAKALPAGT
SAQRAELIALTQALKMAEGKKLNVYTDSRYAFATAHIHGEIYRRRGWLTSEGKEIKN
KDEILALLKALFLPKRLSIIHCPGHQKGHSAEARGNRMADQAARKAAITETPDTSTLL
IENSSPSGGSKRTADGSEFEPKKKRKV

In an aspect, the split point can be localized at any amino acid between position 935 and 965 (e.g., 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965), and the N-terminal fragment of Cas9 nickase can comprise nucleotides from position 1-945 of the Cas9 nickase to the split point and the C-terminal fragment of Cas9 nickase can comprise nucleotides from the split point to position 946-1371 of the Cas9 nickase. Cas9 nickase can be split into a N-terminal fragment and a C-terminal fragment at amino acid 945. In an aspect, a first vector can comprise nucleotides 1-945 of a Cas9 nickase and an N-terminal fragment of an intein. A second vector can comprise a C-terminal fragment of intein nucleotides 946-1371 of a Cas9 nickase. For example, a first vector can encode SEQ ID NO:383, and a second vector can encode SEQ ID NO:384.

N-terminus fragment of Split 945
SEQ ID NO: 383
MKRTADGSEFESPKKKRKVDKKYSIGLDIGTNSVGWAVITDEYKVPSKKFKV
LGNTDRHSIKKNLIGALLFDSGETAEATRLKRTARRRYTRRKNRICYLQEIFSNEMA
KVDDSFFHRLEESFLVEEDKKHERHPIFGNIVDEVAYHEKYPTIYHLRKKLVDSTDK
ADLRLIYLALAHMIKFRGHFLIEGDLNPDNSDVDKLFIQLVQTYNQLFEENPINASGV
DAKAILSARLSKSRRLENLIAQLPGEKKNGLFGNLIALSLGLTPNFKSNFDLAEDAKL
QLSKDTYDDDLDNLLAQIGDQYADLFLAAKNLSDAILLSDILRVNTEITKAPLSASMIK
RYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNR
EKIEKILTFRIPYYVGPLARGNSRFAWMTRKSEETITPWNFEEVVDKGASAQSFIER
MTNFDKNLPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKIIKDKDFLDN
EENEDILEDIVLTLTLFEDREMIEERLKTYAHLFDDKVMKQLKRRRYTGWGRLSRKLI
NGIRDKQSGKTILDFLKSDGFANRNFMQLIHDDSLTFKEDIQKAQVSGQGDSLHEHI
ANLAGSPAIKKGILQTVKVVDELVKVMGRHKPENIVIEMARENQTTQKGQKNSRER
MKRIEEGIKELGSQILKEHPVENTQLQNEKLYLYYLQNGRDMYVDQELDINRLSDYD
VDAIVPQSFLKDDSIDNKVLTRSDKNRGKSDNVPSEEVVKKMKNYWRQLLNAKLIT
QRKFDNLTKAERGGLSELDKAGFIKRQLVETRQITKHVAQILDSRMNCLAGDTLITL
ADGRRVPIRELVSQQNFSVWALNPQTYRLERARVSRAFCTGIKPVYRLTTRLGRSI
RATANHRFLTPQGWKRVDELQPGDYLALPRRIPTAS
C-terminus fragment of Split 945
SEQ ID NO: 384
MAAACPELRQLAQSDVYWDPIVSIEPDGVEEVFDLTVPGPHNFVANDIIAHN
TKYDENDKLIREVKVITLKSKLVSDFRKDFQFYKVREINNYHHAHDAYLNAVVGTALI
KKYPKLESEFVYGDYKVYDVRKMIAKSEQEIGKATAKYFFYSNIMNFFKTEITLANGE
IRKRPLIETNGETGEIVWDKGRDFATVRKVLSMPQVNIVKKTEVQTGGFSKESILPK
RNSDKLIARKKDWDPKKYGGFDSPTVAYSVLVVAKVEKGKSKKLKSVKELLGITIME
RSSFEKNPIDFLEAKGYKEVKKDLIIKLPKYSLFELENGRKRMLASAGELQKGNELAL
PSKYVNFLYLASHYEKLKGSPEDNEQKQLFVEQHKHYLDEIIEQISEFSKRVILADAN
LDKVLSAYNKHRDKPIREQAENIIHLFTLTNLGAPAAFKYFDTTIDRKRYTSTKEVLD
ATLIHQSITGLYETRIDLSQLGGDPIAGSKASPKKKRKVGRAGGSSGSETPGTSESA
TPESSGGSSGGSSTLNIEDEYRLHETSKEPDVSLGSTWLSDFPQAWAETGGMGLA
VRQAPLIIPLKATSTPVSIKQYPMSQEARLGIKPHIQRLLDQGILVPCQSPWNTPLLP
VKKPGTNDYRPVQDLREVNKRVEDIHPTVPNPYNLLSGLPPSHQWYTVLDLKDAFF
CLRLHPTSQPLFAFEWRDPEMGISGQLTWTRLPQGFKNSPTLFNEALHRDLADFRI
QHPDLILLQYVDDLLLAATSELDCQQGTRALLQTLGNLGYRASAKKAQICQKQVKYL
GYLLKEGQRWLTEARKETVMGQPTPKTPRQLREFLGKAGFCRLFIPGFAEMAAPL
YPLTKPGTLFNWGPDQQKAYQEIKQALLTAPALGLPDLTKPFELFVDEKQGYAKGV
LTQKLGPWRRPVAYLSKKLDPVAAGWPPCLRMVAAIAVLTKDAGKLTMGQPLVILA
PHAVEALVKQPPDRWLSNARMTHYQALLLDTDRVQFGPVVALNPATLLPLPEEGL
QHNCLDILAEAHGTRPDLTDQPLPDADHTWYTDGSSLLQEGQRKAGAAVTTETEVI
WAKALPAGTSAQRAELIALTQALKMAEGKKLNVYTDSRYAFATAHIHGEIYRRRGW
LTSEGKEIKNKDEILALLKALFLPKRLSIIHCPGHQKGHSAEARGNRMADQAARKAAI
TETPDTSTLLIENSSPSGGSKRTADGSEFEPKKKRKV

In another aspect, the split point can be localized at any amino acid between position 1044 and 1064 (e.g., 1044, 1045, 1046, 1047, 1048, 1049, 1050, 1051, 1052, 1053, 1054, 1055, 1056, 1057, 1058, 1059, 1060, 1061, 1062, 1063, 1064) and, the N-terminal fragment of Cas9 nickase can comprise nucleotides from position 1-1054 of the Cas9 nickase to the split point and the C-terminal fragment of Cas9 nickase can comprise nucleotides from the split point to position 1055-1371 of the Cas9 nickase. Cas9 nickase can be split into a N-terminal fragment and a C-terminal fragment at amino acid 1054. In an aspect, a vector can comprise nucleotides 1-1054 of a Cas9 nickase and an N-terminal fragment of an intein. A vector can comprise a C-terminal fragment of an intein nucleotides 1055-1371 of a Cas9 nickase. For example, a first vector can encode SEQ ID NO:385, and a second vector can encode SEQ ID NO:386.

N-terminus fragment of Split 1054
SEQ ID NO: 385
MDYKDHDGDYKDHDIDYKDDDDKMAPKKKRKVGRGMDKKYSIGLAIGTNS
VGWAVITDEYKVPSKKFKVLGNTDRHSIKKNLIGALLFDSGETAEATRLKRTARRRY
TRRKNRICYLQEIFSNEMAKVDDSFFHRLEESFLVEEDKKHERHPIFGNIVDEVAYH
EKYPTIYHLRKKLVDSTDKADLRLIYLALAHMIKFRGHFLIEGDLNPDNSDVDKLFIQL
VQTYNQLFEENPINASGVDAKAILSARLSKSRRLENLIAQLPGEKKNGLFGNLIALSL
GLTPNFKSNFDLAEDAKLQLSKDTYDDDLDNLLAQIGDQYADLFLAAKNLSDAILLS
DILRVNTEITKAPLSASMIKRYDEHHQDLTLLKALVRQQLPEKYKEIFFDQSKNGYAG
YIDGGASQEEFYKFIKPILEKMDGTEELLVKLNREDLLRKQRTFDNGSIPHQIHLGEL
HAILRRQEDFYPFLKDNREKIEKILTFRIPYYVGPLARGNSRFAWMTRKSEETITPW
NFEEVVDKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISGVEDRFNA
SLGTYHDLLKIIKDKDFLDNEENEDILEDIVLTLTLFEDREMIEERLKTYAHLFDDKVM
KQLKRRRYTGWGRLSRKLINGIRDKQSGKTILDFLKSDGFANRNFMQLIHDDSLTFK
EDIQKAQVSGQGDSLHEHIANLAGSPAIKKGILQTVKVVDELVKVMGRHKPENIVIE
MARENQTTQKGQKNSRERMKRIEEGIKELGSQILKEHPVENTQLQNEKLYLYYLQN
GRDMYVDQELDINRLSDYDVDAIVPQSFLKDDSIDNKVLTRSDKNRGKSDNVPSEE
VVKKMKNYWRQLLNAKLITQRKFDNLTKAERGGLSELDKAGFIKRQLVETRQITKHV
AQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFF
KTEITLANGCLAGDTLITLADGRRVPIRELVSQQNFSVWALNPQTYRLERARVSRAF
CTGIKPVYRLTTRLGRSIRATANHRFLTPQGWKRVDELQPGDYLALPRRIPTAS
C-terminus fragment of Split 1054
SEQ ID NO: 386
MKRTADGSEFESPRKKRKVDKKYSIGLDIGTNSVGWAVITDEYKVPSKKFKV
LGNTDRHSIKKNLIGALLFDSGETAEATRLKRTARRRYTRRKNRICYLQEIFSNEMA
KVDDSFFHRLEESFLVEEDKKHERHPIFGNIVDEVAYHEKYPTIYHLRKKLVDSTDK
ADLRLIYLALAHMIKFRGHFLIEGDLNPDNSDVDKLFIQLVQTYNQLFEENPINASGV
DAKAILSARLSKSRRLENLIAQLPGEKKNGLFGNLIALSLGLTPNFKSNFDLAEDAKL
QLSKDTYDDDLDNLLAQIGDQYADLFLAAKNLSDAILLSDILRVNTEITKAPLSASMIK
RYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNR
EKIEKILTFRIPYYVGPLARGNSRFAWMTRKSEETITPWNFEEVVDKGASAQSFIER
MTNFDKNLPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKIIKDKDFLDN
EENEDILEDIVLTLTLFEDREMIEERLKTYAHLFDDKVMKQLKRRRYTGWGRLSRKLI
NGIRDKQSGKTILDFLKSDGFANRNFMQLIHDDSLTFKEDIQKAQVSGQGDSLHEHI
ANLAGSPAIKKGILQTVKVVDELVKVMGRHKPENIVIEMARENQTTQKGQKNSRER
MKRIEEGIKELGSQILKEHPVENTQLQNEKLYLYYLQNGRDMYVDQELDINRLSDYD
VDAIVPQSFLKDDSIDNKVLTRSDKNRGKSDNVPSEEVVKKMKNYWRQLLNAKLIT
QRKFDNLTKAERGGLSELDKAGFIKRQLVETRQITKHVAQILDSRMNTKYDENDKLI
REVKVITLKSKLVSDFRKDFQFYKVREINNYHHAHDAYLNAVVGTALIKKYPKLESEF
VYGDYKVYDVRKMIAKSEQEIGKATAKYFFYSNIMNFFKTEITLANGSGGSSGGSS
GSETPGTSESATPESSGGSSGGSSCLSYETEILTVEYGLLPIGKIVEKRIECTVYSVD
NNGNIYTQPVAQWHDRGEQEVFEYCLEDGSLIRATKDHKFMTVDGQMLPIDEIFER
ELDLMRVDNLPNSGGSPKKKRKVPKKKRKV

In an aspect, the split point can localized at any amino acid between position 1105 and 1125 (e.g., 1105, 1106, 1107, 1108, 1109, 1110, 1111, 1112, 1113, 1114, 1115, 1116, 1117, 1118, 1119, 1120, 1121, 1122, 1123, 1124, 1125), and the N-terminal fragment of Cas9 nickase can comprise nucleotides from position 1-1115 of the Cas9 nickase to the split point and the C-terminal fragment of Cas9 nickase can comprise nucleotides from the split point to position 1116-1371 of the Cas9 nickase.Cas9 nickase can be split into a N-terminal fragment and a C-terminal fragment at amino acid 1115. In an aspect, a first vector can comprise nucleotides 1-1115 of a Cas9 nickase and an N-terminal fragment of an intein. A second vector can comprise a C-terminal fragment of an intein nucleotides 1116-1371 of a Cas9 nickase. For example, a first vector can encode SEQ ID NO:387, and a second vector can encode SEQ ID NO:388.

N-terminus fragment of Split 1115
SEQ ID NO: 387
MKRTADGSEFESPRKKRKVDKKYSIGLDIGTNSVGWAVITDEYKVPSKKFKV
LGNTDRHSIKKNLIGALLFDSGETAEATRLKRTARRRYTRRKNRICYLQEIFSNEMA
KVDDSFFHRLEESFLVEEDKKHERHPIFGNIVDEVAYHEKYPTIYHLRKKLVDSTDK
ADLRLIYLALAHMIKFRGHFLIEGDLNPDNSDVDKLFIQLVQTYNQLFEENPINASGV
DAKAILSARLSKSRRLENLIAQLPGEKKNGLFGNLIALSLGLTPNFKSNFDLAEDAKL
QLSKDTYDDDLDNLLAQIGDQYADLFLAAKNLSDAILLSDILRVNTEITKAPLSASMIK
RYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNR
EKIEKILTFRIPYYVGPLARGNSRFAWMTRKSEETITPWNFEEVVDKGASAQSFIER
MTNFDKNLPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKIIKDKDFLDN
EENEDILEDIVLTLTLFEDREMIEERLKTYAHLFDDKVMKQLKRRRYTGWGRLSRKLI
NGIRDKQSGKTILDFLKSDGFANRNFMQLIHDDSLTFKEDIQKAQVSGQGDSLHEHI
ANLAGSPAIKKGILQTVKVVDELVKVMGRHKPENIVIEMARENQTTQKGQKNSRER
MKRIEEGIKELGSQILKEHPVENTQLQNEKLYLYYLQNGRDMYVDQELDINRLSDYD
VDAIVPQSFLKDDSIDNKVLTRSDKNRGKSDNVPSEEVVKKMKNYWRQLLNAKLIT
QRKFDNLTKAERGGLSELDKAGFIKRQLVETRQITKHVAQILDSRMNTKYDENDKLI
REVKVITLKSKLVSDFRKDFQFYKVREINNYHHAHDAYLNAVVGTALIKKYPKLESEF
VYGDYKVYDVRKMIAKSEQEIGKATAKYFFYSNIMNFFKTEITLANGEIRKRPLIETN
GETGEIVWDKGRDFATVRKVLSMPQVNIVKKTEVQTGGFSKESILPKRNSGGGGS
GGGGSGGGGSCLAGDTLITLADGRRVPIRELVSQQNFSVWALNPQTYRLERARVS
RAFCTGIKPVYRLTTRLGRSIRATANHRFLTPQGWKRVDELQPGDYLALPRRIPTAS
C-terminus fragment of Split 1115
SEQ ID NO: 388
MAAACPELRQLAQSDVYWDPIVSIEPDGVEEVFDLTVPGPHNFVANDIIAHN
GGGGSGGGGSGGGGSDKLIARKKDWDPKKYGGFDSPTVAYSVLVVAKVEKGKSK
KLKSVKELLGITIMERSSFEKNPIDFLEAKGYKEVKKDLIIKLPKYSLFELENGRKRML
ASAGELQKGNELALPSKYVNFLYLASHYEKLKGSPEDNEQKQLFVEQHKHYLDEIIE
QISEFSKRVILADANLDKVLSAYNKHRDKPIREQAENIIHLFTLTNLGAPAAFKYFDTT
IDRKRYTSTKEVLDATLIHQSITGLYETRIDLSQLGGDSGGSSGGSSGSETPGTSES
ATPESSGGSSGGSSTLNIEDEYRLHETSKEPDVSLGSTWLSDFPQAWAETGGMGL
AVRQAPLIIPLKATSTPVSIKQYPMSQEARLGIKPHIQRLLDQGILVPCQSPWNTPLL
PVKKPGTNDYRPVQDLREVNKRVEDIHPTVPNPYNLLSGLPPSHQWYTVLDLKDAF
FCLRLHPTSQPLFAFEWRDPEMGISGQLTWTRLPQGFKNSPTLFNEALHRDLADF
RIQHPDLILLQYVDDLLLAATSELDCQQGTRALLQTLGNLGYRASAKKAQICQKQVK
YLGYLLKEGQRWLTEARKETVMGQPTPKTPRQLREFLGKAGFCRLFIPGFAEMAA
PLYPL

Therefore, a Cas nickase, such as a Cas9 nickase can be split into a N-terminal fragment and a C-terminal fragment at many different positions.

The amino acid positions described herein to split Cas9 nickase (i.e., amino acid positions 259, 275, 574, 713, 945, 1054, and 1115) located within a short β-strand; therefore, splitting Cas9 at any of the amino acid within the β-strand is expected to be as efficient as splitting Cas9 at the exact amino acid. β-strand, or β-pleated sheet are common motifs of regular secondary structure in proteins. Beta sheets consist of beta strands (also β-strand) connected laterally by at least two or three backbone hydrogen bonds, forming a generally twisted, pleated sheet. A β-strand is a stretch of polypeptide chain typically 3 to 10 amino acids long with backbone in an extended conformation. Accordingly, a split of a Cas nickase, such as Cas9, can be located 1, 2, 3, 4, 5, 6, 7, 8, 9, or 10 amino acid upstream or downstream of the split position. Additionally, target sites for splitting the Cas nickase protein can be used. For example, any surface exposed loops, any other site that does not interfere with Cas nickase activity, or any site where reconstituted split Cas nickase retains full or partial activity of full-length Cas nickase (such as position 111 or 112) can be used as a split point.

For example, a Cas nickase, such as a Cas9 nickase, can be split at position 259, or at position 258, 257, 256, 255, 254, 253, 252, 251, 250, or 249 (i.e., up to 10 amino acid upstream of position 259), or at position 260, 262, 262, 263, 264, 265, 266, 267, 268, or 269 (up to 10 amino acid downstream of position 259).

A Cas9 nickase can be split at position 275, or at position 274, 273, 272, 271, 270, 269, 268, 267, 266, or 265 (i.e., up to 10 amino acids upstream of position 275), or at position 276, 277, 278, 279, 280, 281, 282, 283, 284, or 285 (up to 10 amino acids downstream of position 275).

A Cas9 nickase can be split at position 574, or at position 573, 572, 571, 570, 569, 568, 567, 566, 565, or 564 (i.e., up to 10 amino acids upstream of position 574), or at position 575, 576, 577, 578, 579, 580, 581, 582, 583, or 584 (up to 10 amino acid downstream of position 574).

A Cas9 nickase can be split at position 713, or at position 712, 711, 710, 709, 708, 707, 706, 705, 704, or 703 (i.e., up to 10 amino acids upstream of position 713), or at position 714, 715, 716, 717, 718, 719, 720, 721, 722, or 723 (up to 10 amino acids downstream of position 713).

A Cas9 nickase can be split at position 945, or at position 944, 943, 942, 941, 940, 939, 938, 937, 936, or 935 (i.e., up to 10 amino acids upstream of position 945), or at position 946, 947, 948, 949, 950, 951, 952, 953, 954, or 955 (up to 10 amino acids downstream of position 945).

A Cas9 nickase can be split at position 1054, or at position 1053, 1052, 1051, 1050, 1049, 1048, 1047, 1046, 1045, or 1044 (i.e., up to 10 amino acids upstream of position 1054), or at position 1055, 1056, 1057, 1058, 1059, 1060, 1061, 1062, 1063, or 1064 (up to 10 amino acids downstream of position 1054).

A Cas9 nickase can be split at position 1115, or at position 1114, 1113, 1112, 1111, 1110, 1109, 1108, 1107, 1106, or 1105 (i.e., up to 10 amino acids upstream of position 1115), or at position 1116, 1117, 1118, 1119, 1120, 1121, 1122, 1123, 1124, or 1125 (up to 10 amino acid downstream of position 1115).

In an aspect, one or more vectors to be delivered to a cell or patient can comprise: (i) a first vector comprising an inverted terminal repeat (ITR) sequence, a promoter, a nucleic acid molecule encoding one or more cytosine or adenosine deaminases, and a nucleic acid molecule encoding an N-terminal portion of a PAM-less or nearly PAM-less Cas nickase, an N-terminal fragment of a dimerization protein, and an ITR sequence; and (ii) a second vector comprising an ITR sequence, a promoter, a C-terminal fragment of a dimerization protein, a nucleic acid molecule encoding a C-terminal portion of a PAM-less or nearly PAM-less Cas nickase, a nucleic acid molecule encoding a uracil glycosylase inhibitor, and an ITR sequence. The first vector, the second vector, or both the first vector and second vector can further comprise a nucleic acid molecule encoding one or more sgRNA molecules targeting a splice acceptor site and/or a splice donor site of a DNA molecule encoding a targeted pre-mRNA molecule. The system can be used to modify RNA splicing of the targeted pre-mRNA molecule to produce a modified mRNA molecule. One or more portions of an exon can be excluded during RNA splicing, resulting in the production of the modified mRNA molecule.

A uracil glycosylase inhibitor (UGI) inhibits the uracil-excision repair system. A cytidine deaminase converts cytosine to uracil, which is then converted to thymine through DNA replication or repair. In some aspects, a UGI prevents base excision repair which changes the U back to a C. In an aspect, contacting a cell and/or polynucleotide with a UGI and a deaminase prevents base excision repair which changes the U back to a C. An exemplary UGI can be GenBank YP_009283008.1.

The one or more cytosine or adenosine deaminases can be CBE4max, CBE evoFERNY, CBE SsAPOBEC3B (R54Q), CBE RrA3F (F130L), TadCBE, CBEPpAPOBEC1 (H122A), ABE8e, ABE8.20m, ABE9, ABE8.20m, hA3A-BE4, hA3G-BE4, evoCDA-BE4, A3G-BE4.4, A3G-BE5.13, A3G-BE5.14, ABE6.3, ABE7.10, ABE8e, ABE9, or ABE8.20m, or ABE8.20. The first vector, the second vector, or both the first vector and second vector can further comprise one or more nuclear localization signal (NLS) sequences. The nucleic acid molecule encoding one or more sgRNA molecules targeting a splice acceptor site and/or a splice donor site of a DNA molecule encoding the pre-mRNA can be operably linked to a promoter. The N-terminal fragment of the dimerization protein can be an N-terminal fragment of an intein, and the C-terminal fragment of the dimerization protein can be a C-terminal fragment of an intein.

The C-terminal fragment of a dimerization protein and the N-terminal fragment of a dimerization protein can be derived from PhoRadA, RmaDnaB^{Δ286}, SspDnaB^{Δ275}, SspDnaB^{M86Δ275}, SspDnaX, TvoVMA, NpuDnaE, NpuDnaB^{Δ283}, SspGyrB, AceL-TerL, PchPRP8, PfuRIR1-1, Psp-GDBPol-1, MtuRecA^{Δ228}, PfuRIR1-2, SceVMA^{Δ206} RmaDnaB^{Δ271}, MtuRecA^{Δ285}, SspDnaB^{Δ274}, gp41-8, SceVMA^{Δ227}, IMPDH-1, NrdJ-1, MtuRecA^{Δ297}, gp41-1, AovDnaE, AspDnaE, AvaDnaE, Cra(C5505)DnaE, Csp(CCYO110)DnaE, Csp(PCC8801)DnaE, CwaDnaE, Maer(NIES843)DnaE, Mcht(PCC7420)DnaE, MtuRecA^{Δ300}, NspDnaE, OliDnaE, Sel(PC7942)DnaE, SspDnaE, Ssp(PCC7002)DnaE, TerDnaE-3, TelDnaE, TvuDnaE, NeqPol, TerThyX^{Δ132}, or combinations thereof.

An amino acid sequence of the N-terminal fragment of an intein can comprise SEQ ID NO:377. (An amino acid sequence of the C-terminal fragment of an intein can comprise SEQ ID NO:389.

SEQ ID NO: 389
IKIATRKYLGKQNVYDIGVERDHNFALKNGFIASNGRAGGSSGSETPGT
SESATPESSGGSSGGSS

The first vector can further comprise a linker between the one or more cytosine or adenosine deaminases and the nucleic acid molecule encoding an N-terminal portion of a PAM-less or nearly PAM-less Cas nickase. The second vector can further comprise a linker between the nucleic acid molecule encoding a C-terminal portion of a PAM-less or nearly PAM-less Cas nickase and the nucleic acid molecule encoding an uracil glycosylase inhibitor.

The first vector and/or second vector can further comprise one or more polynucleotides encoding a protein tag. The linker can encode a polypeptide molecule comprising SEQ ID NO:370, 371, 372, 373, or 374.

The N-terminal fragment of the Cas nickase and the C-terminal fragment of the Cas nickase can form a full-length Cas nickase when combined. The PAM-less or nearly PAM-less Cas nickase can be split into a N-terminal fragment and a C-terminal fragment at a split point. A split point can be localized at any amino acid between position 564 and 584, and the N-terminal fragment of PAM-less or nearly PAM-less Cas nickase comprises nucleotides from position 1 of the PAM-less or nearly PAM-less Cas nickase to the split point and the C-terminal fragment of a PAM-less or nearly PAM-less Cas nickase comprises nucleotides from the split point to position 1371 of the PAM-less or nearly PAM-less Cas nickase. A split point can be localized at any amino acid between position 249 and 269, and the N-terminal fragment of the PAM-less or nearly PAM-less Cas nickase comprises nucleotides from position 1 of the PAM-less or nearly PAM-less Cas nickase to the split point and the C-terminal fragment of the PAM-less or nearly PAM-less Cas nickase comprises nucleotides from the split point to position 1371 of the PAM-less or nearly PAM-less Cas nickase. A split point can be localized at any amino acid between position 265 and 285, and the N-terminal fragment of the PAM-less or nearly PAM-less Cas nickase comprises nucleotides from position 1 of the PAM-less or nearly PAM-less Cas nickase to the split point and the C-terminal fragment of the PAM-less or nearly PAM-less Cas nickase comprises nucleotides from the split point to position 1371 of the PAM-less or nearly PAM-less Cas nickase. A split point can be localized at any amino acid between position 703 and 723, and the N-terminal fragment of the PAM-less or nearly PAM-less Cas nickase comprises nucleotides from position 1 of the PAM-less or nearly PAM-less Cas nickase to the split point and the C-terminal fragment of the PAM-less or nearly PAM-less Cas nickase comprises nucleotides from the split point to position 1371 of the PAM-less or nearly PAM-less Cas nickase. A split point can be localized at any amino acid between position 935 and 965, and the N-terminal fragment of the PAM-less or nearly PAM-less Cas nickase comprises nucleotides from position 1 of the PAM-less or nearly PAM-less Cas nickase to the split point and the C-terminal fragment of the PAM-less or nearly PAM-less Cas nickase comprises nucleotides from the split point to position 1371 of the PAM-less or nearly PAM-less Cas nickase. A split point can be localized at any amino acid between position 1044 and 1064 and, the N-terminal fragment of the PAM-less or nearly PAM-less Cas nickase comprises nucleotides from position 1 of the PAM-less or nearly PAM-less Cas nickase to the split point and the C-terminal fragment of the PAM-less or nearly PAM-less Cas nickase comprises nucleotides from the split point to position 1371 of the PAM-less or nearly PAM-less Cas nickase. A split point can be localized at any amino acid between position 1105 and 1125, and the N-terminal fragment of the PAM-less or nearly PAM-less Cas nickase comprises nucleotides from position 1 of the PAM-less or nearly PAM-less Cas nickase to the split point and the C-terminal fragment of the PAM-less or nearly PAM-less Cas nickase comprises nucleotides from the split point to position 1371 of the PAM-less or nearly PAM-less Cas nickase.

### Methods of Modifying RNA Splicing of a Target RNA Molecule

Provided herein are methods of modifying RNA splicing of a pre-mRNA molecule to produce a modified mRNA molecule. The methods comprise delivering to a cell or patient one or more vectors, e.g., AAV vectors. The vectors can comprise a nucleic acid molecule encoding one or more PAM-less or nearly PAM-less Cas nickases and one or more cytosine or adenosine deaminases; and a nucleic acid molecule encoding one or more guide RNAs (e.g., sgRNAs) (e.g., 1, 3, 3, 4, or more) molecules targeting both a splice acceptor site and a splice donor site of a DNA molecule encoding the pre-mRNA. One or more exons or one or more portions of an exon can be excluded during RNA splicing, resulting in the production of the modified mRNA molecule.

In an aspect one or more PAM-less or nearly PAM-less Cas proteins and one or more cytosine or adenosine deaminase proteins are delivered as proteins instead of using nucleic acid molecules encoding these proteins. The one or more PAM-less or nearly PAM-less Cas proteins and one or more cytosine or adenosine deaminase proteins can be present as a fusion protein.

The nucleic acid molecule encoding one or more PAM-less or nearly PAM-less Cas nickases and the nucleic acid molecule encoding the one or more cytosine or adenosine deaminases can be linked on one vector such that a fusion protein of the one or more PAM-less or nearly PAM-less Cas nickases and the one or more cytosine or adenosine deaminases can be expressed. The PAM sequence recognized by the one or more PAM-less or nearly PAM-less Cas nickases can be, for example, NN, NNN, NNNN, NNNNN, NNNNNN, NG, NRG, NNN, NRNH, NRN, or NDN.

The amount of amount of cryptic splicing can be reduced by 20, 30, 40, 50, 60, 70, 80, 90% or more as compared to a cell or a patient not receiving the one or more vectors or proteins. Intron retention can be reduced by 20, 30, 40, 50, 60, 70, 80, 90% or more as compared to a control cell or control patient. Full exon skipping can be increased by 20, 30, 40, 50, 60, 70, 80, 90% or more as compared to a control (e.g., a cell or patient not subjected to the treatment).

In an aspect, one or more single guide RNA (sgRNA) molecules target a splice acceptor site, a splice donor site, or both sites of a gene, such as an amyloid precursor protein (APP) gene. In an aspect, the one or more exons or one or more portions of an exon that are removed are from exon 17 of an amyloid precursor protein (APP) gene.

In some aspects, one or more single guide RNA (sgRNA) molecules target a splice acceptor site, a splice donor site, or both sites of a gene, such as a huntingtin (HTT) gene. In an aspect, the one or more exons or one or more portions of an exon that are removed are from exon 12 or 13 of a huntingtin (HTT) gene.

### Treating Alzheimer's Disease

Provided herein are methods of treating Alzheimer's disease comprising delivering to a patient in need thereof one or more vectors or proteins. The vectors comprise a nucleic acid molecule encoding one or more PAM-less or nearly PAM-less Cas nickases and one or more cytosine or adenosine deaminases and a nucleic acid molecule encoding one or more guide RNAs (e.g., sgRNA) molecules targeting a splice acceptor site, a splice donor site, or both a splice acceptor site and a splice donor site of a target amyloid precursor protein gene (APP). In an aspect, a gRNA is shown in SEQ ID NO:70-81. In an aspect, a gRNA is shown in SEQ ID NO:50, 51, or 52. In an aspect one or more PAM-less or nearly PAM-less Cas proteins and one or more cytosine or adenosine deaminase proteins are delivered as proteins instead of using nucleic acid molecules encoding these proteins. The one or more PAM-less or nearly PAM-less Cas proteins and one or more cytosine or adenosine deaminase proteins can be present as a fusion protein.

One or more exons or one or more portions of an exon of the target amyloid precursor protein gene (APP) are excluded during RNA splicing. The amount of expression of Aβ42 peptides can be reduced by 20, 30, 40, 50, 60, 70, 80, 90% or more as compared to a patient that does not receive the one or more vectors or proteins. The amount of expression of Aβ42 peptides can be measured using, for example, an ELISA.

The one or more exons or one or more portions of an exon that are removed can be from exon 17 of an amyloid precursor protein gene (APP). In an aspect, one or more sgRNAs targeting the splice donor site can target an intronic cryptic GT splice donor site of exon 17 of an APP gene. Cryptic splicing can be reduced by 20, 30, 40, 50, 60, 70, 80, 90% or more as compared to a cell that does not receive the one or more vectors or proteins.

In an aspect, the level of mature RNA (i.e., target mature RNA) is not reduced as compared to a patient that does not receive the one or more vectors or proteins.

In an aspect, the one or more vectors (e.g., AAV) or proteins are delivered via systemically, intravenously, intrathecally, intracerebrally, intraventricularly, intranasally, intratracheally, intra-aurally, intra-ocularly, intracerebral, or peri-ocularly, orally, rectally, transmucosally, inhalationally, transdermally, parenterally, subcutaneously, intradermally, intramuscularly, intracisternally, intranervally, intrapleurally, by lumbar puncture, intralymphatically, intracisternally, or intranerve.

### Treating Huntington's Disease

Huntington's disease (HD) is an inherited and ultimately fatal neurodegenerative disorder caused by an expanded polyglutamine-encoding CAG repeat within exon 1 of the huntingtin (HTT) gene, which produces a mutant protein that destroys striatal and cortical neurons. Importantly, a critical event in the pathogenesis of HD is the proteolytic cleavage of the mutant HTT protein by caspase-6, which generates fragments of the N-terminal domain of the protein that form highly toxic aggregates. Given the role that proteolysis of the mutant HTT protein plays in HD, strategies for preventing this process hold potential for treating the disorder. By screening 141 CRISPR base editor variants targeting splice elements in the HTT gene, platforms capable of producing HTT protein isoforms resistant to caspase-6-mediated proteolysis via editing of the splice acceptor sequence for exon 13 were identified. When delivered to the striatum of a rodent HD model, these base editors induced efficient exon skipping and decreased the formation of the N-terminal fragments, which in turn reduced HTT protein aggregation and attenuated striatal and cortical atrophy.

Though the exact mechanism by which the mutant HTT (mHTT) protein destroys neurons remains unknown, the N-terminal domain of the mutant protein is believed to play an important role in HD. For instance, fragments of the N-terminal domain are found in nuclear inclusions in the brains of HD patients while its overexpression is sufficient to induce a severe phenotype that recapitulates aspects of the disorder.

In addition to aberrant alternative splicing, an event that can produce HTT1a, an HTT transcript variant that encodes the HTT exon 1 protein, proteolysis of the mHTT protein at Asp 586 also contributes to the formation of these N-terminal fragments. This proteolytic event releases the N-terminal HTT1-586 fragment, which is subsequently processed to even smaller fragments that carry risk for aggregation. Importantly, genetically ablating the proteolytic cleavage site in mHTT at D586, which is hypothesized to be recognized by caspase-6, can prevent the formation of the N-terminal HTT1-586 fragment, limiting its subsequent proteolysis and providing protection from neuronal dysfunction and neurodegeneration. As such, strategies for inhibiting the formation of the N-terminal domain of the mHTT protein hold potential as therapies for HD.

The methods provided herein can genetically ablate the proteolytic cleavage site for caspase-6 in the mHTT gene and prevent the formation of the N-terminal HTT1-586 fragment to limit its subsequent proteolysis. This can provide protection from neuronal dysfunction and neurodegeneration.

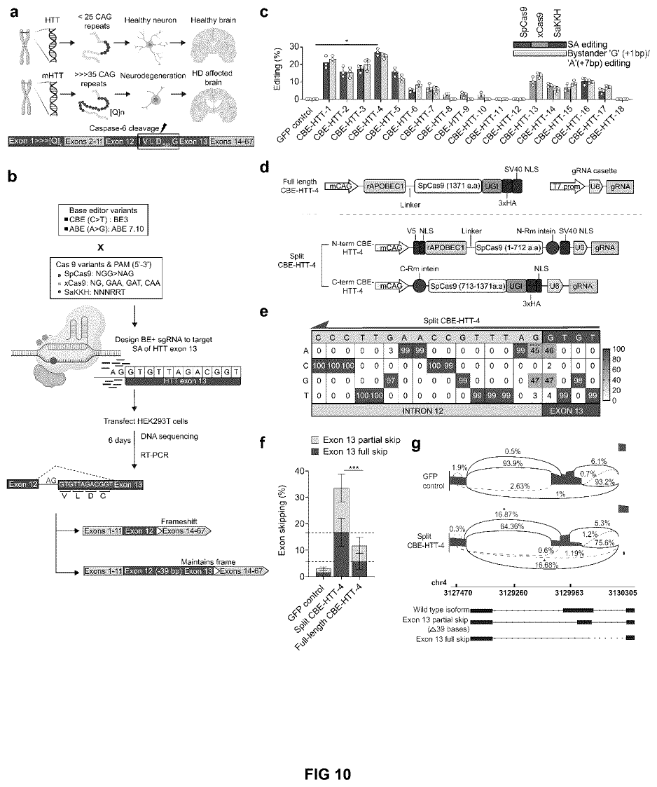

In an aspect, the formation N-terminal fragments of the mHTT is modulated via exon skipping. As a consensus proteolytic cleavage site for caspase-6 is fortuitously encoded in the sequence spanning exons 12 (amino acid: Ile) and 13 (amino acids: Val-Leu-Asp-Gly) of the HTT gene, skipping either exon can be used to produce HTT protein isoforms lacking this site (FIG. 10a). This in turn can prevent the proteolysis and aggregation of the mHTT protein without fully depleting the reservoir of full-length (FL) HTT protein.

Herein CRISPR base editing technologies are used to facilitate targeted exon skipping via editing of highly conserved “AG” dinucleotide motifs in splice acceptor (SA) sequences. Notably, this editing can prevent the recognition of a SA by the spliceosome and promote the skipping of the targeted exon. Skipping exon 12 or 13 of the HTT gene can facilitate the production of HTT protein isoforms more resistant to proteolysis and aggregation.

141 base editors targeting multiple splicing elements in the HTT gene were screened, which enabled the identification of CRISPR base editing platforms that by disruption of the SA for exon 13 generate HTT protein isoforms resistant to proteolysis by caspase-6. When delivered, e.g., intra-striatally, these base editors induced skipping of HTT exon 13, an outcome that disrupted the proteolysis of the mHTT protein in vivo, leading to a substantial decrease in the accumulation of mHTT aggregates and prevention of atrophy in both the striatum and cortex.

Provided herein are methods of treating Huntington's disease comprising delivering one or more vectors or proteins to a patient in need thereof. The one or more vectors can comprise a nucleic acid molecule encoding one or more PAM-less or nearly PAM-less Cas nickases and one or more cytosine or adenosine deaminases and a nucleic acid molecule encoding one or more guide RNAs (e.g., sgRNA) molecules targeting a splice acceptor site, a splice donor site, or both a splice acceptor site and splice donor site of a target huntingtin gene (HTT). In an aspect, a gRNA is shown in SEQ ID NO:179-321. In an aspect, a gRNA is shown in SEQ ID NO:201-219, 224, 229, 234, 247, 257, OR 263. In an aspect one or more PAM-less or nearly PAM-less Cas proteins and one or more cytosine or adenosine deaminase proteins are delivered as proteins instead of using nucleic acid molecules encoding these proteins. The one or more PAM-less or nearly PAM-less Cas proteins and one or more cytosine or adenosine deaminase proteins can be present as a fusion protein.

One or more exons or one or more portions of an exon of the huntingtin gene (HTT) can be excluded during RNA splicing. In an aspect, a caspase-6 cleavage site can be disrupted when one or more exons or one or more portions of an exon of the huntingtin gene (HTT) are excluded during RNA splicing. The one or more exons or one or more portions of an exon that are removed from a mature RNA can comprise exon 12 or exon 13 of a huntingtin (HTT) gene.

In an aspect, one more sgRNAs targeting the splice donor site targets an intronic cryptic GT splice acceptor site of exon 13 of a HTT gene.

In an aspect, the amount of N-terminal HTT protein is reduced by 5, 10, 20, 30, 40, 50% or more in the patient as compared to a patient that does not receive the one or more vectors or proteins.

In an aspect, the amount of intraneuronal inclusions of mHTT protein is reduced by 5, 10, 20, 30, 40, 50% or more in the patient as compared to a patient that does not receive the one or more vectors or proteins.

In an aspect, the amount of cells within the striatum that are positive for reactive astrocyte marker glial fibrillary acidic protein (GFAP) is reduced by 5, 10, 20, 30, 40, 50% or more as compared to a control.

In an aspect, the one or more vectors (e.g., AAV) or proteins are delivered via systemically, intravenously, intrathecally, intracerebrally, intraventricularly, intranasally, intratracheally, intra-aurally, intra-ocularly, intracerebral, or peri-ocularly, orally, rectally, transmucosally, inhalationally, transdermally, parenterally, subcutaneously, intradermally, intramuscularly, intracisternally, intranervally, intrapleurally, by lumbar puncture, intralymphatically, intracisternally, or intranerve.

All patents, patent applications, and other scientific or technical writings referred to anywhere herein are incorporated by reference herein in their entirety. The aspects illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by aspects, optional features, modification and variation of the concepts herein disclosed may be resorted to by those skilled in the art, and that such modifications and variations are considered to be within the scope of this invention as defined by the description and the appended claims.

Any single term, single element, single phrase, group of terms, group of phrases, or group of elements described herein can be each be specifically excluded from the claims.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods

In addition, where features or aspects of the invention are described in terms of Markush groups or other grouping of alternatives, those skilled in the art will recognize that the invention is also thereby described in terms of any individual member or subgroup of members of the Markush group or other group.

The following are provided for exemplification purposes only and are not intended to limit the scope of the invention described in broad terms above.

### Examples

### Example 1: Methods

### Plasmids and Cloning

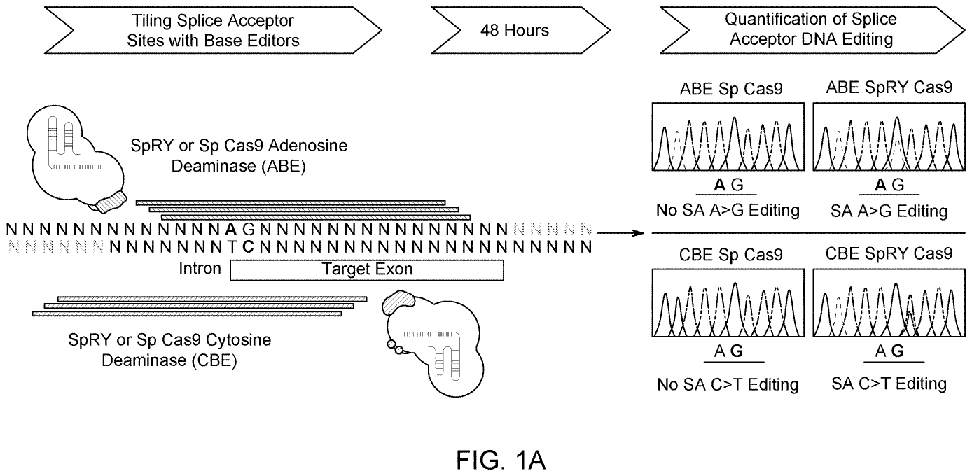

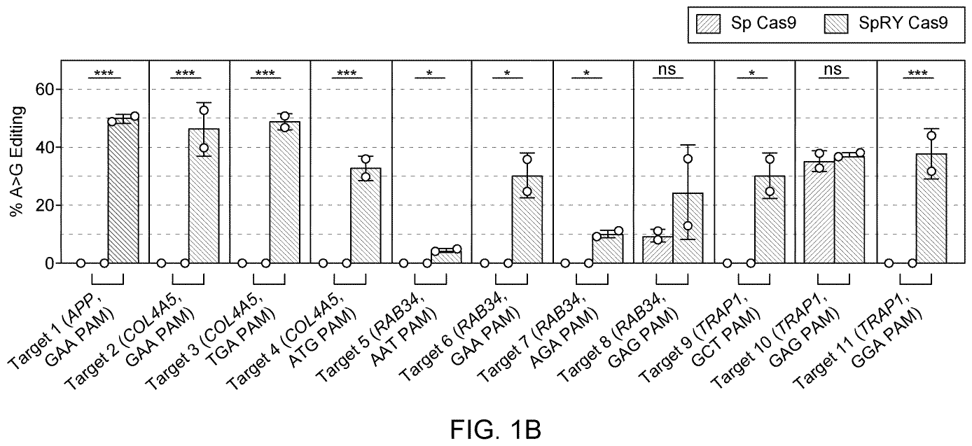

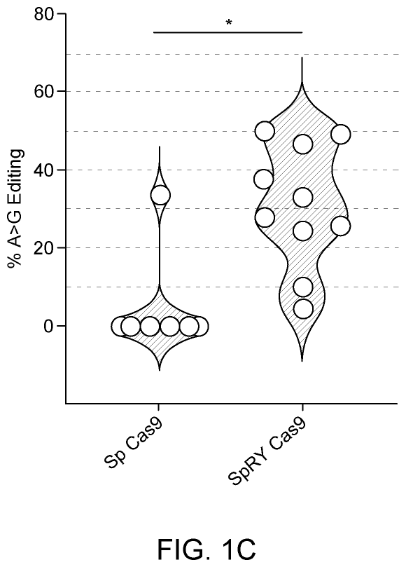

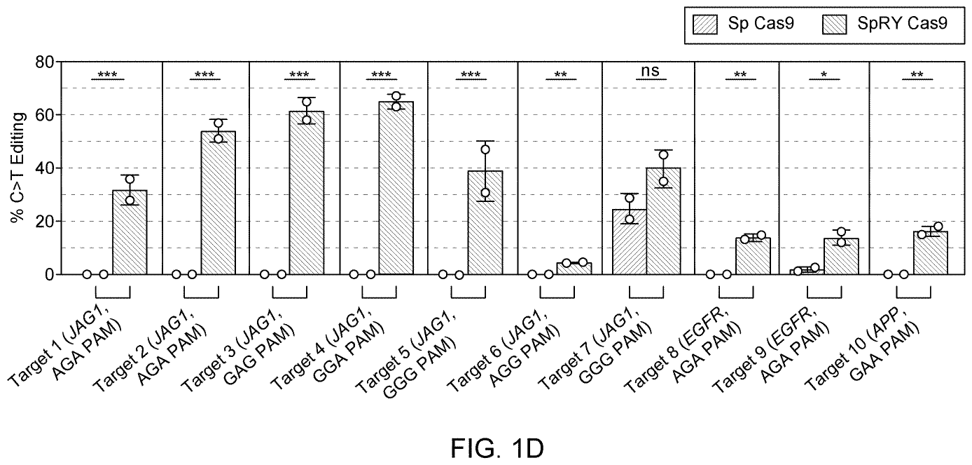

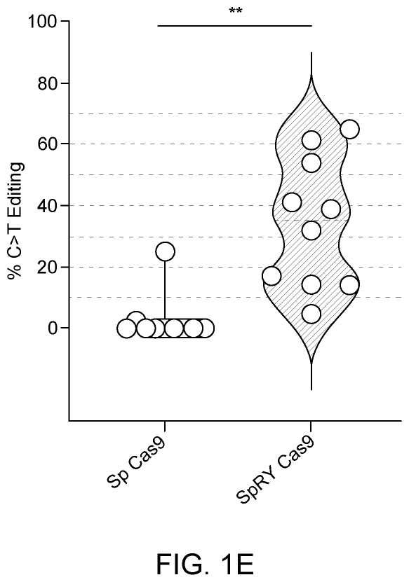

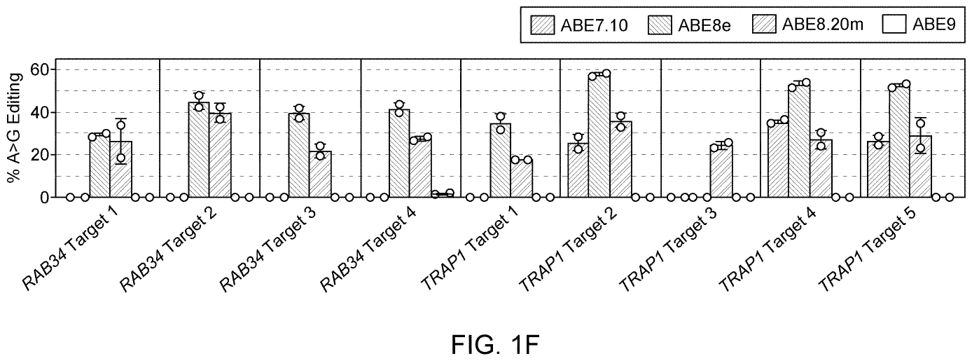

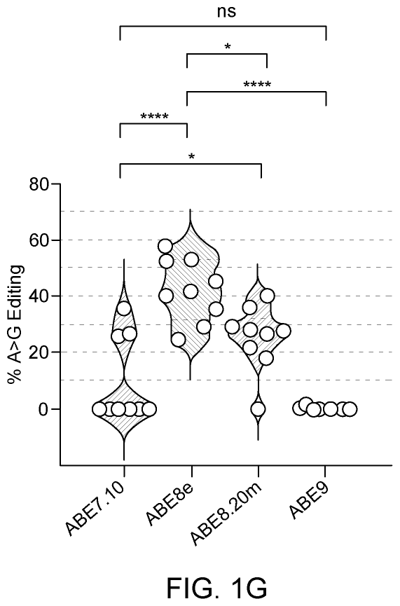

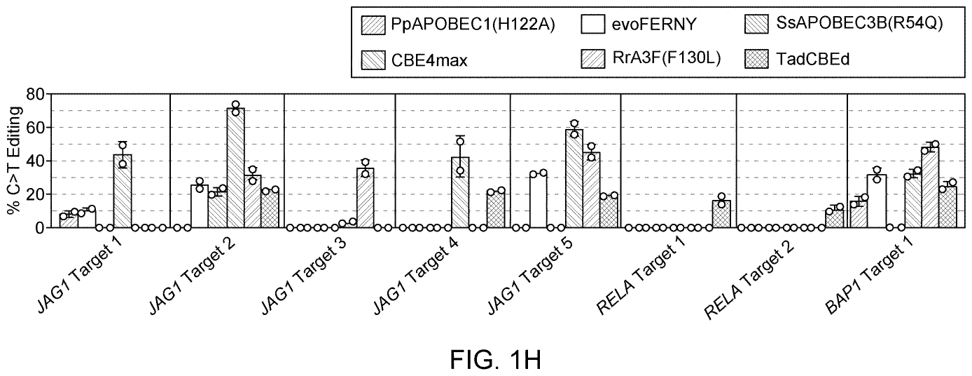

The plasmid encoding the U6-sgRNA expression cassette was obtained from Addgene (#47108). The full-length SpRY CBE4max construct was purchased from Addgene (Plasmid #139999). The full-length SpRY ABE8e plasmid was generated through Gibson Assembly of gBlock Gene Fragments (Integrated DNA Technologies) containing the adenosine deaminase in Addgene Plasmid #138489 and the SpRY CBE4max backbone. To build the ABE and CBE constructs consisting of different deaminases described in FIG. 1, first SpRY mutations were cloned via Gibson Assembly of gBlock Gene Fragments (Integrated DNA Technologies) into BE-expressing plasmids described in our previous work. Then, different deaminases were cloned into the plasmids via Gibson Assembly of gBlock Gene Fragments. For experiments in BE(2)-M17 cells, which utilized a plasmid encoding SpRYABE8e-T2A-Puro, SpRY mutations, ABE8e deaminase, and the puromycin resistance gene were cloned via Gibson Assembly of gBlock Gene Fragments into the lentiviral expression plasmid pLenti-EFS-FNLS-P2A-BlastR. Plasmids encoding the split AAV SpRY ABE8e constructs were cloned via Gibson Assembly of gBlock Gene Fragments into our previously described AAV split Cas9 BE3 plasmids to insert SpRY mutations as well as to replace APOBEC1 with ABE8e. All amino acid sequences from these plasmid constructs are provided in Table 1.

TABLE 1
Amino Acid Sequence of Cas9 Base Editors

Full Length SpRY ABE8e (SEQ ID NO: 1)
MKRTADGSEFESPKKKRKVSEVEFSHEYWMRHALTLAKRARDEREVPVGAVLVLNNRVI
GEGWNRAIGLHDPTAHAEIMALRQGGLVMQNYRLIDATLYVTFEPCVMCAGAMIHSRIGR
WVFGVRNSKRGAAGSLMNVLNYPGMNHRVEITEGILADECAALLCDFYRMPRQVFNAQK
KAQSSINSGGSSGGSSGSETPGTSESATPESSGGSSGGSDKKYSIGLAIGTNSVGWAVIT
DEYKVPSKKFKVLGNTDRHSIKKNLIGALLFDSGETAEATRLKRTARRRYTRRKNRICYLQ
EIFSNEMAKVDDSFFHRLEESFLVEEDKKHERHPIFGNIVDEVAYHEKYPTIYHLRKKLVDS
TDKADLRLIYLALAHMIKFRGHFLIEGDLNPDNSDVDKLFIQLVQTYNQLFEENPINASGVDA
KAILSARLSKSRRLENLIAQLPGEKKNGLFGNLIALSLGLTPNFKSNFDLAEDAKLQLSKDTY
DDDLDNLLAQIGDQYADLFLAAKNLSDAILLSDILRVNTEITKAPLSASMIKRYDEHHQDLTL
LKALVRQQLPEKYKEIFFDQSKNGYAGYIDGGASQEEFYKFIKPILEKMDGTEELLVKLNRE
DLLRKQRTFDNGSIPHQIHLGELHAILRRQEDFYPFLKDNREKIEKILTFRIPYYVGPLARGN
SRFAWMTRKSEETITPWNFEEVVDKGASAQSFIERMTNFDKNLPNEKVLPKHSLLYEYFT
VYNELTKVKYVTEGMRKPAFLSGEQKKAIVDLLFKTNRKVTVKQLKEDYFKKIECFDSVEIS
GVEDRFNASLGTYHDLLKIIKDKDFLDNEENEDILEDIVLTLTLFEDREMIEERLKTYAHLFD
DKVMKQLKRRRYTGWGRLSRKLINGIRDKQSGKTILDFLKSDGFANRNFMQLIHDDSLTFK
EDIQKAQVSGQGDSLHEHIANLAGSPAIKKGILQTVKVVDELVKVMGRHKPENIVIEMAREN
QTTQKGQKNSRERMKRIEEGIKELGSQILKEHPVENTQLQNEKLYLYYLQNGRDMYVDQE
LDINRLSDYDVDHIVPQSFLKDDSIDNKVLTRSDKNRGKSDNVPSEEVVKKMKNYWRQLL
NAKLITQRKFDNLTKAERGGLSELDKAGFIKRQLVETRQITKHVAQILDSRMNTKYDENDKL
IREVKVITLKSKLVSDFRKDFQFYKVREINNYHHAHDAYLNAVVGTALIKKYPKLESEFVYG
DYKVYDVRKMIAKSEQEIGKATAKYFFYSNIMNFFKTEITLANGEIRKRPLIETNGETGEIVW
DKGRDFATVRKVLSMPQVNIVKKTEVQTGGFSKESILPKRNSDKLIARKKDWDPKKYGGF
LWPTVAYSVLVVAKVEKGKSKKLKSVKELLGITIMERSSFEKNPIDFLEAKGYKEVKKDLIIK
LPKYSLFELENGRKRMLASAKQLQKGNELALPSKYVNFLYLASHYEKLKGSPEDNEQKQL
FVEQHKHYLDEIIEQISEFSKRVILADANLDKVLSAYNKHRDKPIREQAENIIHLFTLTRLGAP
RAFKYFDTTIDPKQYRSTKEVLDATLIHQSITGLYETRIDLSQLGGDSGGSKRTADGSEFEP
KKKRKV*
Full Length SpRY CB4max (SEQ ID NO: 2)
MKRTADGSEFESPKKKRKVSSETGPVAVDPTLRRRIEPHEFEVFFDPRELRKETCLLYEIN
WGGRHSIWRHTSQNTNKHVEVNFIEKFTTERYFCPNTRCSITWFLSWSPCGECSRAITEF
LSRYPHVTLFIYIARLYHHADPRNRQGLRDLISSGVTIQIMTEQESGYCWRNFVNYSPSNE
AHWPRYPHLWVRLYVLELYCIILGLPPCLNILRRKQPQLTFFTIALQSCHYQRLPPHILWAT
GLKSGGSSGGSSGSETPGTSESATPESSGGSSGGSDKKYSIGLAIGTNSVGWAVITDEYK
VPSKKFKVLGNTDRHSIKKNLIGALLFDSGETAERTRLKRTARRRYTRRKNRICYLQEIFSN
EMAKVDDSFFHRLEESFLVEEDKKHERHPIFGNIVDEVAYHEKYPTIYHLRKKLVDSTDKA
DLRLIYLALAHMIKFRGHFLIEGDLNPDNSDVDKLFIQLVQTYNQLFEENPINASGVDAKAIL
SARLSKSRRLENLIAQLPGEKKNGLFGNLIALSLGLTPNFKSNFDLAEDAKLQLSKDTYDDD
LDNLLAQIGDQYADLFLAAKNLSDAILLSDILRVNTEITKAPLSASMIKRYDEHHQDLTLLKAL
VRQQLPEKYKEIFFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPYYVGPLARGNSRFA
WMTRKSEETITPWNFEEVVDKGASAQSFIERMTNFDKNLPNEKVLPKHSLLYEYFTVYNE
LTKVKYVTEGMRKPAFLSGEQKKAIVDLLFKTNRKVTVKQLKEDYFKKIECFDSVEISGVED
RFNASLGTYHDLLKIIKDKDFLDNEENEDILEDIVLTLTLFEDREMIEERLKTYAHLFDDKVM
KQLKRRRYTGWGRLSRKLINGIRDKQSGKTILDFLKSDGFANRNFMQLIHDDSLTFKEDIQ
KAQVSGQGDSLHEHIANLAGSPAIKKGILQTVKVVDELVKVMGRHKPENIVIEMARENQTT
QKGQKNSRERMKRIEEGIKELGSQILKEHPVENTQLQNEKLYLYYLQNGRDMYVDQELDI
NRLSDYDVDHIVPQSFLKDDSIDNKVLTRSDKNRGKSDNVPSEEVVKKMKNYWRQLLNAK
LITQRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYGDYK
VYDVRKMIAKSEQEIGKATAKYFFYSNIMNFFKTEITLANGEIRKRPLIETNGETGEIVWDKG
RDFATVRKVLSMPQVNIVKKTEVQTGGFSKESIRPKRNSDKLIARKKDWDPKKYGGFLWP
TVAYSVLVVAKVEKGKSKKLKSVKELLGITIMERSSFEKNPIDFLEAKGYKEVKKDLIIKLPKY
SLFELENGRKRMLASAKQLQKGNELALPSKYVNFLYLASHYEKLKGSPEDNEQKQLFVEQ
HKHYLDEIIEQISEFSKRVILADANLDKVLSAYNKHRDKPIREQAENIIHLFTLTRLGAPRAFK
YFDTTIDPKQYRSTKEVLDATLIHQSITGLYETRIDLSQLGGDSGGSGGSGGSTNLSDIIEKE
TGKQLVIQESILMLPEEVEEVIGNKPESDILVHTAYDESTDENVMLLTSDAPEYKPWALVIQ
DSNGENKIKMLSGGSGGSGGSTNLSDIIEKETGKQLVIQESILMLPEEVEEVIGNKPESDILV
HTAYDESTDENVMLLTSDAPEYKPWALVIQDSNGENKIKMLSGGSKRTADGSEFEPKKKR
KVGGGGSGATNFSLLKQAGDVEENPGPMVSKGEELFTGVVPILVELDGDVNGHKFSVSG
EGEGDATYGKLTLKFICTTGKLPVPWPTLVTTLTYGVQCFSRYPDHMKQHDFFKSAMPEG
YVQERTIFFKDDGNYKTRAEVKFEGDTLVNRIELKGIDFKEDGNILGHKLEYNYNSHNVYIM
ADKQKNGIKVNFKIRHNIEDGSVQLADHYQQNTPIGDGPVLLPDNHYLSTQSALSKDPNEK
RDHMVLLEFVTAAGITLGMDELYK*
N-SpRY ABE8e (SEQ ID NO: 3)
MKRTADGSEFESPKKKRKVSEVEFSHEYWMRHALTLAKRARDEREVPVGAVLVLNNRVI
GEGWNRAIGLHDPTAHAEIMALRQGGLVMQNYRLIDATLYVTFEPCVMCAGAMIHSRIGR
WVFGVRNSKRGAAGSLMNVLNYPGMNHRVEITEGILADECAALLCDFYRMPRQVFNAQK
KAQSSINSGGSSGGSSGSETPGTSESATPESSGGSSGGSDKKYSIGLAIGTNSVGWAVIT
DEYKVPSKKFKVLGNTDRHSIKKNLIGALLFDSGETAEATRLKRTARRRYTRRKNRICYLQ
EIFSNEMAKVDDSFFHRLEESFLVEEDKKHERHPIFGNIVDEVAYHEKYPTIYHLRKKLVDS
TDKADLRLIYLALAHMIKFRGHFLIEGDLNPDNSDVDKLFIQLVQTYNQLFEENPINASGVDA
KAILSARLSKSRRLENLIAQLPGEKKNGLFGNLIALSLGLTPNFKSNFDLAEDAKLQLSKDTY
DDDLDNLLAQIGDQYADLFLAAKNLSDAILLSDILRVNTEITKAPLSASMIKRYDEHHQDLTL
LKALVRQQLPEKYKEIFFDQSKNGYAGYIDGGASQEEFYKFIKPILEKMDGTEELLVKLNRE
DLLRKQRTFDNGSIPHQIHLGELHAILRRQEDFYPFLKDNREKIEKILTFRIPYYVGPLARGN
SRFAWMTRKSEETITPWNFEEVVDKGASAQSFIERMTNFDKNLPNEKVLPKHSLLYEYFT
VYNELTKVKYVTEGMRKPAFLSGEQKKAIVDLLFKTNRKVTVKQLKEDYFKKIECFDSVEIS
GVEDRFNASLGTYHDLLKIIKDKDFLDNEENEDILEDIVLTLTLFEDREMIEERLKTYAHLFD
DKVMKQLKRRRYTGWGRLSRKLINGIRDKQSGKTILDFLKSDGFANRNFMQLIHDDSLTFK
EDIQKAQVCLAGDTLITLADGRRVPIRELVSQQNFSVWALNPQTYRLERARVSRAFCTGIK
PVYRLTTRLGRSIRATANHRFLTPQGWKRVDELQPGDYLALPRRIPTAS*
C-SpRY ABE (SEQ ID NO: 4)
MAAACPELRQLAQSDVYWDPIVSIEPDGVEEVFDLTVPGPHNFVANDIIAHNSGQGDSLH
EHIANLAGSPAIKKGILQTVKVVDELVKVMGRHKPENIVIEMARENQTTQKGQKNSRERMK
RIEEGIKELGSQILKEHPVENTQLQNEKLYLYYLQNGRDMYVDQELDINRLSDYDVDHIVPQ
SFLKDDSIDNKVLTRSDKNRGKSDNVPSEEVVKKMKNYWRQLLNAKLITQRKFDNLTKAE
RGGLSELDKAGFIKRQLVETRQITKHVAQILDSRMNTKYDENDKLIREVKVITLKSKLVSDF
RKDFQFYKVREINNYHHAHDAYLNAVVGTALIKKYPKLESEFVYGDYKVYDVRKMIAKSEQ
EIGKATAKYFFYSNIMNFFKTEITLANGEIRKRPLIETNGETGEIVWDKGRDFATVRKVLSMP
QVNIVKKTEVQTGGFSKESILPKRNSDKLIARKKDWDPKKYGGFLWPTVAYSVLVVAKVEK
GKSKKLKSVKELLGITIMERSSFEKNPIDFLEAKGYKEVKKDLIIKLPKYSLFELENGRKRML
ASAKQLQKGNELALPSKYVNFLYLASHYEKLKGSPEDNEQKQLFVEQHKHYLDEIIEQISEF
SKRVILADANLDKVLSAYNKHRDKPIREQAENIIHLFTLTRLGAPRAFKYFDTTIDPKQYRST
KEVLDATLIHQSITGLYETRIDLSQLGGDSGGSGGSGGSTNLSDIIEKETGKQLVIQESILML
PEEVEEVIGNKPESDILVHTAYDESTDENVMLLTSDAPEYKPWALVIQDSNGENKIKMLSG
GSKRTADGSEFEPKKKRKV*
N-WT ABE8e (SEQ ID NO: 5)
MKRTADGSEFESPKKKRKVSEVEFSHEYWMRHALTLAKRARDEREVPVGAVLVLNNRVI
GEGWNRAIGLHDPTAHAEIMALRQGGLVMQNYRLIDATLYVTFEPCVMCAGAMIHSRIGR
WVFGVRNSKRGAAGSLMNVLNYPGMNHRVEITEGILADECAALLCDFYRMPRQVFNAQK
KAQSSINSGGSSGGSSGSETPGTSESATPESSGGSSGGSDKKYSIGLAIGTNSVGWAVIT
DEYKVPSKKFKVLGNTDRHSIKKNLIGALLFDSGETAEATRLKRTARRRYTRRKNRICYLQ
EIFSNEMAKVDDSFFHRLEESFLVEEDKKHERHPIFGNIVDEVAYHEKYPTIYHLRKKLVDS
TDKADLRLIYLALAHMIKFRGHFLIEGDLNPDNSDVDKLFIQLVQTYNQLFEENPINASGVDA
KAILSARLSKSRRLENLIAQLPGEKKNGLFGNLIALSLGLTPNFKSNFDLAEDAKLQLSKDTY
DDDLDNLLAQIGDQYADLFLAAKNLSDAILLSDILRVNTEITKAPLSASMIKRYDEHHQDLTL
LKALVRQQLPEKYKEIFFDQSKNGYAGYIDGGASQEEFYKFIKPILEKMDGTEELLVKLNRE
DLLRKQRTFDNGSIPHQIHLGELHAILRRQEDFYPFLKDNREKIEKILTFRIPYYVGPLARGN
SRFAWMTRKSEETITPWNFEEVVDKGASAQSFIERMTNFDKNLPNEKVLPKHSLLYEYFT
VYNELTKVKYVTEGMRKPAFLSGEQKKAIVDLLFKTNRKVTVKQLKEDYFKKIECFDSVEIS
GVEDRFNASLGTYHDLLKIIKDKDFLDNEENEDILEDIVLTLTLFEDREMIEERLKTYAHLFD
DKVMKQLKRRRYTGWGRLSRKLINGIRDKQSGKTILDFLKSDGFANRNFMQLIHDDSLTFK
EDIQKAQVCLAGDTLITLADGRRVPIRELVSQQNFSVWALNPQTYRLERARVSRAFCTGIK
PVYRLTTRLGRSIRATANHRFLTPQGWKRVDELQPGDYLALPRRIPTAS*
C-WT ABE (SEQ ID NO: 6)
MAAACPELRQLAQSDVYWDPIVSIEPDGVEEVFDLTVPGPHNFVANDIIAHNSGQGDSLH
EHIANLAGSPAIKKGILQTVKVVDELVKVMGRHKPENIVIEMARENQTTQKGQKNSRERMK
RIEEGIKELGSQILKEHPVENTQLQNEKLYLYYLQNGRDMYVDQELDINRLSDYDVDHIVPQ
SFLKDDSIDNKVLTRSDKNRGKSDNVPSEEVVKKMKNYWRQLLNAKLITQRKFDNLTKAE
RGGLSELDKAGFIKRQLVETRQITKHVAQILDSRMNTKYDENDKLIREVKVITLKSKLVSDF
RKDFQFYKVREINNYHHAHDAYLNAVVGTALIKKYPKLESEFVYGDYKVYDVRKMIAKSEQ
EIGKATAKYFFYSNIMNFFKTEITLANGEIRKRPLIETNGETGEIVWDKGRDFATVRKVLSMP
QVNIVKKTEVQTGGFSKESILPKRNSDKLIARKKDWDPKKYGGFDSPTVAYSVLVVAKVEK
GKSKKLKSVKELLGITIMERSSFEKNPIDFLEAKGYKEVKKDLIIKLPKYSLFELENGRKRML
ASAGELQKGNELALPSKYVNFLYLASHYEKLKGSPEDNEQKQLFVEQHKHYLDEIIEQISEF
SKRVILADANLDKVLSAYNKHRDKPIREQAENIIHLFTLTNLGAPAAFKYFDTTIDRKRYTST
KEVLDATLIHQSITGLYETRIDLSQLGGDSGGSGGSGGSTNLSDIIEKETGKQLVIQESILML
PEEVEEVIGNKPESDILVHTAYDESTDENVMLLTSDAPEYKPWALVIQDSNGENKIKMLSG
GSKRTADGSEFEPKKKRKV*
N-SpRY ABE7.10 (SEQ ID NO: 7)
MKRTADGSEFESPKKKRKVSEVEFSHEYWMRHALTLAKRAWDEREVPVGAVLVHNNRVI
GEGWNRPIGRHDPTAHAEIMALRQGGLVMQNYRLIDATLYVTLEPCVMCAGAMIHSRIGR
WVFGARDAKTGAAGSLMDVLHHPGMNHRVEITEGILADECAALLSDFFRMRRQEIKAQKK
AQSSTDSGGSSGGSSGSETPGTSESATPESSGGSSGGSSEVEFSHEYWMRHALTLAKR
ARDEREVPVGAVLVLNNRVIGEGWNRAIGLHDPTAHAEIMALRQGGLVMQNYRLIDATLY
VTFEPCVMCAGAMIHSRIGRVVFGVRNAKTGAAGSLMDVLHYPGMNHRVEITEGILADEC
AALLCYFFRMPRQVFNAQKKAQSSTDASGGSSGGSSGSETPGTSESATPESSGGSSGG
SDKKYSIGLAIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHPIFGNIV
DEVAYHEKYPTIYHLRKKLVDSTDKADLRLIYLALAHMIKFRGHFLIEGDLNPDNSDVDKLFI
QLVQTYNQLFEENPINASGVDAKAILSARLSKSRRLENLIAQLPGEKKNGLFGNLIALSLGLT
PNFKSNFDLAEDAKLQLSKDTYDDDLDNLLAQIGDQYADLFLAAKNLSDAILLSDILRVNTEI
TKAPLSASMIKRYDEHHQDLTLLKALVRQQLPEKYKEIFFDQSKNGYAGYIDGGASQEEFY
KFIKPILEKMDGTEELLVKLNREDLLRKQRTFDNGSIPHQIHLGELHAILRRQEDFYPFLKDN
REKIEKILTFRIPYYVGPLARGNSRFAWMTRKSEETITPWNFEEVVDKGASAQSFIERMTN
FDKNLPNEKVLPKHSLLYEYFTVYNELTKVKYVTEGMRKPAFLSGEQKKAIVDLLFKTNRK
VTVKQLKEDYFKKIECFDSVEISGVEDRFNASLGTYHDLLKIIKDKDFLDNEENEDILEDIVLT
LTLFEDREMIEERLKTYAHLFDDKVMKQLKRRRYTGWGRLSRKLINGIRDKQSGKTILDFL
KSDGFANRNFMQLIHDDSLTFKEDIQKAQVCLAGDTLITLADGRRVPIRELVSQQNFSVWA
LNPQTYRLERARVSRAFCTGIKPVYRLTTRLGRSIRATANHRFLTPQGWKRVDELQPGDY
LALPRRIPTAS*
N-SpRY ABE8.20m (SEQ ID NO: 8)
MKRTADGSEFESPKKKRKVSEVEFSHEYWMRHALTLAKRARDEREVPVGAVLVLNNRVI
GEGWNRAIGLHDPTAHAEIMALRQGGLVMQNYRLYDATLYSTFEPCVMCAGAMIHSRIGR
WVFGVRNAKTGAAGSLMDVLHHPGMNHRVEITEGILADECAALLCRFFRMPRRVFNAQKK
AQSSTDSGGSSGGSSGSETPGTSESATPESSGGSSGGSDKKYSIGLAIGTNSVGWAVITD
EYKVPSKKFKVLGNTDRHSIKKNLIGALLFDSGETAEATRLKRTARRRYTRRKNRICYLQEI
FSNEMAKVDDSFFHRLEESFLVEEDKKHERHPIFGNIVDEVAYHEKYPTIYHLRKKLVDST
DKADLRLIYLALAHMIKFRGHFLIEGDLNPDNSDVDKLFIQLVQTYNQLFEENPINASGVDA
KAILSARLSKSRRLENLIAQLPGEKKNGLFGNLIALSLGLTPNFKSNFDLAEDAKLQLSKDTY
DDDLDNLLAQIGDQYADLFLAAKNLSDAILLSDILRVNTEITKAPLSASMIKRYDEHHQDLTL
LKALVRQQLPEKYKEIFFDQSKNGYAGYIDGGASQEEFYKFIKPILEKMDGTEELLVKLNRE
DLLRKQRTFDNGSIPHQIHLGELHAILRRQEDFYPFLKDNREKIEKILTFRIPYYVGPLARGN
SRFAWMTRKSEETITPWNFEEVVDKGASAQSFIERMTNFDKNLPNEKVLPKHSLLYEYFT
VYNELTKVKYVTEGMRKPAFLSGEQKKAIVDLLFKTNRKVTVKQLKEDYFKKIECFDSVEIS
GVEDRFNASLGTYHDLLKIIKDKDFLDNEENEDILEDIVLTLTLFEDREMIEERLKTYAHLFD
DKVMKQLKRRRYTGWGRLSRKLINGIRDKQSGKTILDFLKSDGFANRNFMQLIHDDSLTFK
EDIQKAQVSGQGDSLHEHIANLAGSPAIKKGILQTVKVVDELVKVMGRHKPENIVIEMAREN
QTTQKGQKNSRERMKRIEEGIKELGSQILKEHPVENTQLQNEKLYLYYLQNGRDMYVDQE
LDINRLSDYDVDHIVPQSFLKDDSIDNKVLTRSDKNRGKSDNVPSEEVVKKMKNYWRQLL
NAKLITQRKFDNLTKAERGGLSELDKAGFIKRQLVETRQITKHVAQILDSRMNTKYDENDKL
IREVKVITLKSKLVSDFRKDFQFYKVREINNYHHAHDAYLNAVVGTALIKKYPKLESEFVYG
DYKVYDVRKMIAKSEQEIGKATAKYFFYSNIMNFFKTEITLANGEIRKRPLIETNGETGEIVW
DKGRDFATVRKVLSMPQVNIVKKTEVQTGGFSKESILPKRNSDKLIARKKDWDPKKYGGF
LWPTVAYSVLVVAKVEKGKSKKLKSVKELLGITIMERSSFEKNPIDFLEAKGYKEVKKDLIIK
LPKYSLFELENGRKRMLASAKQLQKGNELALPSKYVNFLYLASHYEKLKGSPEDNEQKQL
FVEQHKHYLDEIIEQISEFSKRVILADANLDKVLSAYNKHRDKPIREQAENIIHLFTLTRLGAP
RAFKYFDTTIDPKQYRSTKEVLDATLIHQSITGLYETRIDLSQLGGDSGGSKRTADGSEFEP
KKKRKV*
N-SpRY ABE9 (SEQ ID NO: 9)
MKRTADGSEFESPKKKRKVSEVEFSHEYWMRHALTLAKRARDEREVPVGAVLVLNNRVI
GEGWNRAIGLHDPTAHAEIMALRQGGLVMQNYRLIDATLYVTFEPCVMCAGAMIHSRIGR
WVFGVRQSKRGAAGSLMNVLNYPGMNHRVEITEGILADECAALTCDFYRMPRQVFNAQK
KAQSSINSGGSSGGSSGSETPGTSESATPESSGGSSGGSDKKYSIGLAIGTNSVGWAVIT
DEYKVPSKKFKVLGNTDRHSIKKNLIGALLFDSGETAEATRLKRTARRRYTRRKNRICYLQ
EIFSNEMAKVDDSFFHRLEESFLVEEDKKHERHPIFGNIVDEVAYHEKYPTIYHLRKKLVDS
TDKADLRLIYLALAHMIKFRGHFLIEGDLNPDNSDVDKLFIQLVQTYNQLFEENPINASGVDA
KAILSARLSKSRRLENLIAQLPGEKKNGLFGNLIALSLGLTPNFKSNFDLAEDAKLQLSKDTY
DDDLDNLLAQIGDQYADLFLAAKNLSDAILLSDILRVNTEITKAPLSASMIKRYDEHHQDLTL
LKALVRQQLPEKYKEIFFDQSKNGYAGYIDGGASQEEFYKFIKPILEKMDGTEELLVKLNRE
DLLRKQRTFDNGSIPHQIHLGELHAILRRQEDFYPFLKDNREKIEKILTFRIPYYVGPLARGN
SRFAWMTRKSEETITPWNFEEVVDKGASAQSFIERMTNFDKNLPNEKVLPKHSLLYEYFT
VYNELTKVKYVTEGMRKPAFLSGEQKKAIVDLLFKTNRKVTVKQLKEDYFKKIECFDSVEIS
GVEDRFNASLGTYHDLLKIIKDKDFLDNEENEDILEDIVLTLTLFEDREMIEERLKTYAHLFD
DKVMKQLKRRRYTGWGRLSRKLINGIRDKQSGKTILDFLKSDGFANRNFMQLIHDDSLTFK
EDIQKAQVCLAGDTLITLADGRRVPIRELVSQQNFSVWALNPQTYRLERARVSRAFCTGIK
PVYRLTTRLGRSIRATANHRFLTPQGWKRVDELQPGDYLALPRRIPTAS*
N-SpRY CBE4max (SEQ ID NO: 10)
MGKPIPNPLLGLDSTKRTADGSEFESPKKKRKVSSETGPVAVDPTLRRRIEPHEFEVFFDP
RELRKETCLLYEINWGGRHSIWRHTSQNTNKHVEVNFIEKFTTERYFCPNTRCSITWFLSW
SPCGECSRAITEFLSRYPHVTLFIYIARLYHHADPRNRQGLRDLISSGVTIQIMTEQESGYC
WRNFVNYSPSNEAHWPRYPHLWVRLYVLELYCIILGLPPCLNILRRKQPQLTFFTIALQSC
HYQRLPPHILWATGLKSGGSSGGSSGSETPGTSESATPESSGGSSGGSDKKYSIGLAIGT
NSVGWAVITDEYKVPSKKFKVLGNTDRHSIKKNLIGALLFDSGETAEATRLKRTARRRYTR
RKNRICYLQEIFSNEMAKVDDSFFHRLEESFLVEEDKKHERHPIFGNIVDEVAYHEKYPTIY
HLRKKLVDSTDKADLRLIYLALAHMIKFRGHFLIEGDLNPDNSDVDKLFIQLVQTYNQLFEE
NPINASGVDAKAILSARLSKSRRLENLIAQLPGEKKNGLFGNLIALSLGLTPNFKSNFDLAED
AKLQLSKDTYDDDLDNLLAQIGDQYADLFLAAKNLSDAILLSDILRVNTEITKAPLSASMIKR
YDEHHQDLTLLKALVRQQLPEKYKEIFFDQSKNGYAGYIDGGASQEEFYKFIKPILEKMDG
TEELLVKLNREDLLRKQRTFDNGSIPHQIHLGELHAILRRQEDFYPFLKDNREKIEKILTFRIP
YYVGPLARGNSRFAWMTRKSEETITPWNFEEVVDKGASAQSFIERMTNFDKNLPNEKVLP
KHSLLYEYFTVYNELTKVKYVTEGMRKPAFLSGEQKKAIVDLLFKTNRKVTVKQLKEDYFK
KIECFDSVEISGVEDRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLKSDGFANRNFM
QLIHDDSLTFKEDIQKAQVCLAGDTLITLADGRRVPIRELVSQQNFSVWALNPQTYRLERA
RVSRAFCTGIKPVYRLTTRLGRSIRATANHRFLTPQGWKRVDELQPGDYLALPRRIPTAS*
C-SpRY CBE (SEQ ID NO: 11)
MAAACPELRQLAQSDVYWDPIVSIEPDGVEEVFDLTVPGPHNFVANDIIAHNSGQGDSLH
EHIANLAGSPAIKKGILQTVKVVDELVKVMGRHKPENIVIEMARENQTTQKGQKNSRERMK
RIEEGIKELGSQILKEHPVENTQLQNEKLYLYYLQNGRDMYVDQELDINRLSDYDVDHIVPQ
SFLKDDSIDNKVLTRSDKNRGKSDNVPSEEVVKKMKNYWRQLLNAKLITQRKFDNLTKAE
RGGLSELDKAGFIKRQLVETRQITKHVAQILDSRMNTKYDENDKLIREVKVITLKSKLVSDF
RKDFQFYKVREINNYHHAHDAYLNAVVGTALIKKYPKLESEFVYGDYKVYDVRKMIAKSEQ
EIGKATAKYFFYSNIMNFFKTEITLANGEIRKRPLIETNGETGEIVWDKGRDFATVRKVLSMP
QVNIVKKTEVQTGGFSKESIRPKRNSDKLIARKKDWDPKKYGGFLWPTVAYSVLVVAKVE
KGKSKKLKSVKELLGITIMERSSFEKNPIDFLEAKGYKEVKKDLIIKLPKYSLFELENGRKRM
LASAKQLQKGNELALPSKYVNFLYLASHYEKLKGSPEDNEQKQLFVEQHKHYLDEIIEQISE
FSKRVILADANLDKVLSAYNKHRDKPIREQAENIIHLFTLTRLGAPRAFKYFDTTIDPKQYRS
TKEVLDATLIHQSITGLYETRIDLSQLGGDSGGSGGSGGSTNLSDIIEKETGKQLVIQESILM
LPEEVEEVIGNKPESDILVHTAYDESTDENVMLLTSDAPEYKPWALVIQDSNGENKIKMLS
GGSGGSGGSTNLSDIIEKETGKQLVIQESILMLPEEVEEVIGNKPESDILVHTAYDESTDEN
VMLLTSDAPEYKPWALVIQDSNGENKIKMLYPYDVPDYAYPYDVPDYAYPYDVPDYASGG
SPKKKRKV*
N-WT CBE4max (SEQ ID NO: 12)
MGKPIPNPLLGLDSTKRTADGSEFESPKKKRKVSSETGPVAVDPTLRRRIEPHEFEVFFDP
RELRKETCLLYEINWGGRHSIWRHTSQNTNKHVEVNFIEKFTTERYFCPNTRCSITWFLSW
SPCGECSRAITEFLSRYPHVTLFIYIARLYHHADPRNRQGLRDLISSGVTIQIMTEQESGYC
WRNFVNYSPSNEAHWPRYPHLWVRLYVLELYCIILGLPPCLNILRRKQPQLTFFTIALQSC
HYQRLPPHILWATGLKSGGSSGGSSGSETPGTSESATPESSGGSSGGSDKKYSIGLAIGT
NSVGWAVITDEYKVPSKKFKVLGNTDRHSIKKNLIGALLFDSGETAEATRLKRTARRRYTR
RKNRICYLQEIFSNEMAKVDDSFFHRLEESFLVEEDKKHERHPIFGNIVDEVAYHEKYPTIY
HLRKKLVDSTDKADLRLIYLALAHMIKFRGHFLIEGDLNPDNSDVDKLFIQLVQTYNQLFEE
NPINASGVDAKAILSARLSKSRRLENLIAQLPGEKKNGLFGNLIALSLGLTPNFKSNFDLAED
AKLQLSKDTYDDDLDNLLAQIGDQYADLFLAAKNLSDAILLSDILRVNTEITKAPLSASMIKR
YDEHHQDLTLLKALVRQQLPEKYKEIFFDQSKNGYAGYIDGGASQEEFYKFIKPILEKMDG
TEELLVKLNREDLLRKQRTFDNGSIPHQIHLGELHAILRRQEDFYPFLKDNREKIEKILTFRIP
YYVGPLARGNSRFAWMTRKSEETITPWNFEEVVDKGASAQSFIERMTNFDKNLPNEKVLP
KHSLLYEYFTVYNELTKVKYVTEGMRKPAFLSGEQKKAIVDLLFKTNRKVTVKQLKEDYFK
KIECFDSVEISGVEDRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLKSDGFANRNFM
QLIHDDSLTFKEDIQKAQVCLAGDTLITLADGRRVPIRELVSQQNFSVWALNPQTYRLERA
RVSRAFCTGIKPVYRLTTRLGRSIRATANHRFLTPQGWKRVDELQPGDYLALPRRIPTAS*
C-WT CBE4max (SEQ ID NO: 13)
MAAACPELRQLAQSDVYWDPIVSIEPDGVEEVFDLTVPGPHNFVANDIIAHNSGQGDSLH
EHIANLAGSPAIKKGILQTVKVVDELVKVMGRHKPENIVIEMARENQTTQKGQKNSRERMK
RIEEGIKELGSQILKEHPVENTQLQNEKLYLYYLQNGRDMYVDQELDINRLSDYDVDHIVPQ
SFLKDDSIDNKVLTRSDKNRGKSDNVPSEEVVKKMKNYWRQLLNAKLITQRKFDNLTKAE
RGGLSELDKAGFIKRQLVETRQITKHVAQILDSRMNTKYDENDKLIREVKVITLKSKLVSDF
RKDFQFYKVREINNYHHAHDAYLNAVVGTALIKKYPKLESEFVYGDYKVYDVRKMIAKSEQ
EIGKATAKYFFYSNIMNFFKTEITLANGEIRKRPLIETNGETGEIVWDKGRDFATVRKVLSMP
QVNIVKKTEVQTGGFSKESILPKRNSDKLIARKKDWDPKKYGGFDSPTVAYSVLVVAKVEK
GKSKKLKSVKELLGITIMERSSFEKNPIDFLEAKGYKEVKKDLIIKLPKYSLFELENGRKRML
ASAGELQKGNELALPSKYVNFLYLASHYEKLKGSPEDNEQKQLFVEQHKHYLDEIIEQISEF
SKRVILADANLDKVLSAYNKHRDKPIREQAENIIHLFTLTNLGAPAAFKYFDTTIDRKRYTST
KEVLDATLIHQSITGLYETRIDLSQLGGDSGGSTNLSDIIEKETGKQLVIQESILMLPEEVEEV
IGNKPESDILVHTAYDESTDENVMLLTSDAPEYKPWALVIQDSNGENKIKMLSYPYDVPDY
AYPYDVPDYAYPYDVPDYASGGSPKKKRKV*
N-SpRY PpAPOBEC1(H122A) (SEQ ID NO: 14)
MGKPIPNPLLGLDSTKRTADGSEFESPKKKRKVTSEKGPSTGDPTLRRRIESWEFDVFYD
PRELRKETCLLYEIKWGMSRKIWRSSGKNTTNHVEVNFIKKFTSERRFHSSISCSITWFLS
WSPCWECSQAIREFLSQHPGVTLVIYVARLFWAMDQRNRQGLRDLVNSGVTIQIMRASEY
YHCWRNFVNYPPGDEAHWPQYPPLWMMLYALELHCIILSLPPCLKISRRWQNHLAFFRLH
LQNCHYQTIPPHILLATGLIHPSVTWRLKSGGSSGGSSGSETPGTSESATPESSGGSSGG
SDKKYSIGLAIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHPIFGNIV
DEVAYHEKYPTIYHLRKKLVDSTDKADLRLIYLALAHMIKFRGHFLIEGDLNPDNSDVDKLFI
QLVQTYNQLFEENPINASGVDAKAILSARLSKSRRLENLIAQLPGEKKNGLFGNLIALSLGLT
PNFKSNFDLAEDAKLQLSKDTYDDDLDNLLAQIGDQYADLFLAAKNLSDAILLSDILRVNTEI
TKAPLSASMIKRYDEHHQDLTLLKALVRQQLPEKYKEIFFDQSKNGYAGYIDGGASQEEFY
KFIKPILEKMDGTEELLVKLNREDLLRKQRTFDNGSIPHQIHLGELHAILRRQEDFYPFLKDN
REKIEKILTFRIPYYVGPLARGNSRFAWMTRKSEETITPWNFEEVVDKGASAQSFIERMTN
FDKNLPNEKVLPKHSLLYEYFTVYNELTKVKYVTEGMRKPAFLSGEQKKAIVDLLFKTNRK
VTVKQLKEDYFKKIECFDSVEISGVEDRFNASLGTYHDLLKIIKDKDFLDNEENEDILEDIVLT
LTLFEDREMIEERLKTYAHLFDDKVMKQLKRRRYTGWGRLSRKLINGIRDKQSGKTILDFL
KSDGFANRNFMQLIHDDSLTFKEDIQKAQVCLAGDTLITLADGRRVPIRELVSQQNFSVWA
LNPQTYRLERARVSRAFCTGIKPVYRLTTRLGRSIRATANHRFLTPQGWKRVDELQPGDY
LALPRRIPTAS*
N-SpRY evoFERNY (SEQ ID NO: 15)
MGKPIPNPLLGLDSTKRTADGSEFESPKKKRKVSFERNYDPRELRKETYLLYEIKWGKSG
KLWRHWCQNNRTQHAEVYFLENIFNARRFNPSTHCSITWYLSWSPCAECSQKIVDFLKEH
PNVNLEIYVARLYYPENERNRQGLRDLVNSGVTIRIMDLPDYNYCWKTFVSDQGGDEDY
WPGHFAPWIKQYSLKLSGGSSGGSSGSETPGTSESATPESSGGSSGGSDKKYSIGLAIGT
NSVGWAVITDEYKVPSKKFKVLGNTDRHSIKKNLIGALLFDSGETAEATRLKRTARRRYTR
RKNRICYLQEIFSNEMAKVDDSFFHRLEESFLVEEDKKHERHPIFGNIVDEVAYHEKYPTIY
HLRKKLVDSTDKADLRLIYLALAHMIKFRGHFLIEGDLNPDNSDVDKLFIQLVQTYNQLFEE
NPINASGVDAKAILSARLSKSRRLENLIAQLPGEKKNGLFGNLIALSLGLTPNFKSNFDLAED
AKLQLSKDTYDDDLDNLLAQIGDQYADLFLAAKNLSDAILLSDILRVNTEITKAPLSASMIKR
YDEHHQDLTLLKALVRQQLPEKYKEIFFDQSKNGYAGYIDGGASQEEFYKFIKPILEKMDG
TEELLVKLNREDLLRKQRTFDNGSIPHQIHLGELHAILRRQEDFYPFLKDNREKIEKILTFRIP
YYVGPLARGNSRFAWMTRKSEETITPWNFEEVVDKGASAQSFIERMTNFDKNLPNEKVLP
KHSLLYEYFTVYNELTKVKYVTEGMRKPAFLSGEQKKAIVDLLFKTNRKVTVKQLKEDYFK
KIECFDSVEISGVEDRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLKSDGFANRNFM
QLIHDDSLTFKEDIQKAQVCLAGDTLITLADGRRVPIRELVSQQNFSVWALNPQTYRLERA
RVSRAFCTGIKPVYRLTTRLGRSIRATANHRFLTPQGWKRVDELQPGDYLALPRRIPTAS*
N-SpRY SsAPOBEC3B(R54Q) (SEQ ID NO: 16)
MGKPIPNPLLGLDSTKRTADGSEFESPKKKRKVDPQRLRQWPGPGPASRGGYGQRPRIR
NPEEWFHELSPRTFSFHFRNLRFASGQNRSYICCQVEGKNCFFQGIFQNQVPPDPPCHA
ELCFLSWFQSWGLSPDEHYYVTWFISWSPCCECAAKVAQFLEENRNVSLSLSAARLYYF
WKSESREGLRRLSDLGAQVGIMSFQDFQHCWNNFVHNLGMPFQPWKKLHKNYQRLVTE
LKQILREEPATYGSPQAQGKVRIGSTAAGLRHSHSHTRSEAHLRPNHSSRQHRILNPPRE
ARARTCVLVDASWICYRSGGSSGGSSGSETPGTSESATPESSGGSSGGSDKKYSIGLAIG
TNSVGWAVITDEYKVPSKKFKVLGNTDRHSIKKNLIGALLFDSGETAEATRLKRTARRRYT
RRKNRICYLQEIFSNEMAKVDDSFFHRLEESFLVEEDKKHERHPIFGNIVDEVAYHEKYPTI
YHLRKKLVDSTDKADLRLIYLALAHMIKFRGHFLIEGDLNPDNSDVDKLFIQLVQTYNQLFE
ENPINASGVDAKAILSARLSKSRRLENLIAQLPGEKKNGLFGNLIALSLGLTPNFKSNFDLAE
DAKLQLSKDTYDDDLDNLLAQIGDQYADLFLAAKNLSDAILLSDILRVNTEITKAPLSASMIK
RYDEHHQDLTLLKALVRQQLPEKYKEIFFDQSKNGYAGYIDGGASQEEFYKFIKPILEKMD
GTEELLVKLNREDLLRKQRTFDNGSIPHQIHLGELHAILRRQEDFYPFLKDNREKIEKILTFRI
PYYVGPLARGNSRFAWMTRKSEETITPWNFEEVVDKGASAQSFIERMTNFDKNLPNEKVL
PKHSLLYEYFTVYNELTKVKYVTEGMRKPAFLSGEQKKAIVDLLFKTNRKVTVKQLKEDYF
KKIECFDSVEISGVEDRFNASLGTYHDLLKIIKDKDFLDNEENEDILEDIVLTLTLFEDREMIE
ERLKTYAHLFDDKVMKQLKRRRYTGWGRLSRKLINGIRDKQSGKTILDFLKSDGFANRNF
MQLIHDDSLTFKEDIQKAQVCLAGDTLITLADGRRVPIRELVSQQNFSVWALNPQTYRLER
ARVSRAFCTGIKPVYRLTTRLGRSIRATANHRFLTPQGWKRVDELQPGDYLALPRRIPTAS
*
N-SpRY RrA3F(F130L) (SEQ ID NO: 17)
MGKPIPNPLLGLDSTKRTADGSEFESPKKKRKVKPQIRDHRPNPMEAMYPHIFYFHFENLE
KAYGRNETWLCFTVEIIKQYLPVPWKKGVFRNQVDPETHCHAEKCFLSWFCNNTLSPKKN
YQVTWYTSWSPCPECAGEVAEFLAEHSNVKLTIYTARLYYLWDTDYQEGLRSLSEEGASV
EIMDYEDFQYCWENFVYDDGEPFKRWKGLKYNFQSLTRRLREILQSGGSSGGSSGSETP
GTSESATPESSGGSSGGSDKKYSIGLAIGTNSVGWAVITDEYKVPSKKFKVLGNTDRHSIK
KNLIGALLFDSGETAEATRLKRTARRRYTRRKNRICYLQEIFSNEMAKVDDSFFHRLEESFL
VEEDKKHERHPIFGNIVDEVAYHEKYPTIYHLRKKLVDSTDKADLRLIYLALAHMIKFRGHFL
IEGDLNPDNSDVDKLFIQLVQTYNQLFEENPINASGVDAKAILSARLSKSRRLENLIAQLPGE
KKNGLFGNLIALSLGLTPNFKSNFDLAEDAKLQLSKDTYDDDLDNLLAQIGDQYADLFLAAK
NLSDAILLSDILRVNTEITKAPLSASMIKRYDEHHQDLTLLKALVRQQLPEKYKEIFFDQSKN
GYAGYIDGGASQEEFYKFIKPILEKMDGTEELLVKLNREDLLRKQRTFDNGSIPHQIHLGEL
HAILRRQEDFYPFLKDNREKIEKILTFRIPYYVGPLARGNSRFAWMTRKSEETITPWNFEEV
VDKGASAQSFIERMTNFDKNLPNEKVLPKHSLLYEYFTVYNELTKVKYVTEGMRKPAFLSG
EQKKAIVDLLFKTNRKVTVKQLKEDYFKKIECFDSVEISGVEDRFNASLGTYHDLLKIIKDKD
FLDNEENEDILEDIVLTLTLFEDREMIEERLKTYAHLFDDKVMKQLKRRRYTGWGRLSRKLI
NGIRDKQSGKTILDFLKSDGFANRNFMQLIHDDSLTFKEDIQKAQVCLAGDTLITLADGRRV
PIRELVSQQNFSVWALNPQTYRLERARVSRAFCTGIKPVYRLTTRLGRSIRATANHRFLTP
QGWKRVDELQPGDYLALPRRIPTAS*
N-SpRY TadCBEd (SEQ ID NO: 18)
MGKPIPNPLLGLDSTKRTADGSEFESPKKKRKVSEVEFSHEYWMRHALTLAKRARDERKA
PVGAVLVLNNRVIGEGWNRAIGLHDPTAHAEIIALRQGGLVMQNYRLIDATLYVTFEPCVM
CAGAMINSRIGRVVFGVRNSKRGAAGSLMNVLNYPGMNHRVEITEGILADECAALLCDFY
RMPRQVFNAQKKAQSSINSGGSSGGSSGSETPGTSESATPESSGGSSGGSDKKYSIGLA
IGTNSVGWAVITDEYKVPSKKFKVLGNTDRHSIKKNLIGALLFDSGETAEATRLKRTARRRY
TRRKNRICYLQEIFSNEMAKVDDSFFHRLEESFLVEEDKKHERHPIFGNIVDEVAYHEKYPT
IYHLRKKLVDSTDKADLRLIYLALAHMIKFRGHFLIEGDLNPDNSDVDKLFIQLVQTYNQLFE
ENPINASGVDAKAILSARLSKSRRLENLIAQLPGEKKNGLFGNLIALSLGLTPNFKSNFDLAE
DAKLQLSKDTYDDDLDNLLAQIGDQYADLFLAAKNLSDAILLSDILRVNTEITKAPLSASMIK
RYDEHHQDLTLLKALVRQQLPEKYKEIFFDQSKNGYAGYIDGGASQEEFYKFIKPILEKMD
GTEELLVKLNREDLLRKQRTFDNGSIPHQIHLGELHAILRRQEDFYPFLKDNREKIEKILTFRI
PYYVGPLARGNSRFAWMTRKSEETITPWNFEEVVDKGASAQSFIERMTNFDKNLPNEKVL
PKHSLLYEYFTVYNELTKVKYVTEGMRKPAFLSGEQKKAIVDLLFKTNRKVTVKQLKEDYF
KKIECFDSVEISGVEDRFNASLGTYHDLLKIIKDKDFLDNEENEDILEDIVLTLTLFEDREMIE
ERLKTYAHLFDDKVMKQLKRRRYTGWGRLSRKLINGIRDKQSGKTILDFLKSDGFANRNF
MQLIHDDSLTFKEDIQKAQVCLAGDTLITLADGRRVPIRELVSQQNFSVWALNPQTYRLER
ARVSRAFCTGIKPVYRLTTRLGRSIRATANHRFLTPQGWKRVDELQPGDYLALPRRIPTAS
*
N-SpRY ABE8e (in vivo studies) (SEQ ID NO: 19)
MGKPIPNPLLGLDSTKRTADGSEFESPKKKRKVSEVEFSHEYWMRHALTLAKRARDEREV
PVGAVLVLNNRVIGEGWNRAIGLHDPTAHAEIMALRQGGLVMQNYRLIDATLYVTFEPCV
MCAGAMIHSRIGRVVFGVRNSKRGAAGSLMNVLNYPGMNHRVEITEGILADECAALLCDF
YRMPRQVFNAQKKAQSSINSGGSSGGSSGSETPGTSESATPESSGGSSGGSDKKYSIGL
AIGTNSVGWAVITDEYKVPSKKFKVLGNTDRHSIKKNLIGALLFDSGETAEATRLKRTARRR
YTRRKNRICYLQEIFSNEMAKVDDSFFHRLEESFLVEEDKKHERHPIFGNIVDEVAYHEKYP
TIYHLRKKLVDSTDKADLRLIYLALAHMIKFRGHFLIEGDLNPDNSDVDKLFIQLVQTYNQLF
EENPINASGVDAKAILSARLSKSRRLENLIAQLPGEKKNGLFGNLIALSLGLTPNFKSNFDLA
EDAKLQLSKDTYDDDLDNLLAQIGDQYADLFLAAKNLSDAILLSDILRVNTEITKAPLSASMI
KRYDEHHQDLTLLKALVRQQLPEKYKEIFFDQSKNGYAGYIDGGASQEEFYKFIKPILEKM
DGTEELLVKLNREDLLRKQRTFDNGSIPHQIHLGELHAILRRQEDFYPFLKDNREKIEKILTF
RIPYYVGPLARGNSRFAWMTRKSEETITPWNFEEVVDKGASAQSFIERMTNFDKNLPNEK
VLPKHSLLYEYFTVYNELTKVKYVTEGMRKPAFLSGEQKKAIVDLLFKTNRKVTVKQLKED
YFKKIECFDSVEISGVEDRFNASLGTYHDLLKIIKDKDFLDNEENEDILEDIVLTLTLFEDREM
IEERLKTYAHLFDDKVMKQLKRRRYTGWGRLSRKLINGIRDKQSGKTILDFLKSDGFANRN
FMQLIHDDSLTFKEDIQKAQVCLAGDTLITLADGRRVPIRELVSQQNFSVWALNPQTYRLE
RARVSRAFCTGIKPVYRLTTRLGRSIRATANHRFLTPQGWKRVDELQPGDYLALPRRIPTA
S*
C-SpRY ABE8e (in vivo studies) (SEQ ID NO: 20)
MAAACPELRQLAQSDVYWDPIVSIEPDGVEEVFDLTVPGPHNFVANDIIAHNSGQGDSLH
EHIANLAGSPAIKKGILQTVKVVDELVKVMGRHKPENIVIEMARENQTTQKGQKNSRERMK
RIEEGIKELGSQILKEHPVENTQLQNEKLYLYYLQNGRDMYVDQELDINRLSDYDVDHIVPQ
SFLKDDSIDNKVLTRSDKNRGKSDNVPSEEVVKKMKNYWRQLLNAKLITQRKFDNLTKAE
RGGLSELDKAGFIKRQLVETRQITKHVAQILDSRMNTKYDENDKLIREVKVITLKSKLVSDF
RKDFQFYKVREINNYHHAHDAYLNAVVGTALIKKYPKLESEFVYGDYKVYDVRKMIAKSEQ
EIGKATAKYFFYSNIMNFFKTEITLANGEIRKRPLIETNGETGEIVWDKGRDFATVRKVLSMP
QVNIVKKTEVQTGGFSKESILPKRNSDKLIARKKDWDPKKYGGFLWPTVAYSVLVVAKVEK
GKSKKLKSVKELLGITIMERSSFEKNPIDFLEAKGYKEVKKDLIIKLPKYSLFELENGRKRML
ASAKQLQKGNELALPSKYVNFLYLASHYEKLKGSPEDNEQKQLFVEQHKHYLDEIIEQISEF
SKRVILADANLDKVLSAYNKHRDKPIREQAENIIHLFTLTRLGAPRAFKYFDTTIDPKQYRST
KEVLDATLIHQSITGLYETRIDLSQLGGDSGGSGGSGGSTNLSDIIEKETGKQLVIQESILML
PEEVEEVIGNKPESDILVHTAYDESTDENVMLLTSDAPEYKPWALVIQDSNGENKIKMLSG
GSKRTADGSEFEPKKKRKV*
EGFP-KASH (SEQ ID NO: 21)
MVSKGEELFTGVVPILVELDGDVNGHKFSVSGEGEGDATYGKLTLKFICTTGKLPVPWPTL
VTTLTYGVQCFSRYPDHMKQHDFFKSAMPEGYVQERTIFFKDDGNYKTRAEVKFEGDTL
VNRIELKGIDFKEDGNILGHKLEYNYNSHNVYIMADKQKNGIKVNFKIRHNIEDGSVQLADH
YQQNTPIGDGPVLLPDNHYLSTQSALSKDPNEKRDHMVLLEFVTAAGITLGMDELYKSGL
RSREEEEETDSRMPHLDSPGSSQPRRSFLSRVIRAALPLQLLLLLLLLLACLLPASEDDYSC
TQANNFARSFYPMLRYTNGPPPT*

All oligonucleotides used in this work were obtained from integrated DNA Technologies. The oligonucleotides for sgRNA generation were hybridized, phosphorylated and cloned following the U6 sgRNA vector using BbsI, Bsa1, of BsmBI sites with sequences are provided in Table 2.

TABLE 2
Oligonucleotide Sequences Used for Base Editing of Splice Sites

| Base | | | | | |
|---|---|---|---|---|---|
| Editor | Gene | Exon | Sequence | PAM | Figure |
| ABE | TRAP1 | 4 | GTAGGTGTTTATACGGGAGC | TGA | 1F-G |
| | | | SEQ ID NO: 22 | | |
| ABE | TRAP1 | 4 | TGTAGGTGTTTATACGGGAG | CTG | 1F-G |
| | | | SEQ ID NO: 23 | | |
| ABE | TRAP1 | 4 | CTGTAGGTGTTTATACGGGA | GCT | 1B-C, 1F-G |
| | | | SEQ ID NO: 24 | | |
| ABE | TRAP1 | 4 | CCTGTAGGTGTTTATACGGG | AGC | 1F-G |
| | | | SEQ ID NO: 25 | | |
| ABE | TRAP1 | 4 | TCCTGTAGGTGTTTATACGG | GAG | 1B-C, 1F-G |
| | | | SEQ ID NO: 26 | | |
| ABE | TRAP1 | 4 | TTCCTGTAGGTGTTTATACG | GGA | 1B-C, 1F-G |
| | | | SEQ ID NO: 27 | | |
| ABE | RAB34 | 9 | GTCAGGTGAGAATGTCCGAG | AAT | 1B-C, 1F-G |
| | | | SEQ ID NO: 28 | | |
| ABE | RAB34 | 9 | TGTCAGGTGAGAATGTCCGA | GAA | 1B-C, 1F-G |
| | | | SEQ ID NO: 29 | | |
| ABE | RAB34 | 9 | GTGTCAGGTGAGAATGTCCG | AGA | 1B-C, 1F-G |
| | | | SEQ ID NO: 30 | | |
| ABE | RAB34 | 9 | TGTGTCAGGTGAGAATGTCC | GAG | 1B-C, 1F-G |
| | | | SEQ ID NO: 31 | | |
| ABE | Col4A5 | 21 | ACTCAGGTGATGAGATATGT | GAA | 1B-C |
| | | | SEQ ID NO: 32 | | |
| ABE | Col4A5 | 21 | TACTCAGGTGATGAGATATG | TGA | 1B-C |
| | | | SEQ ID NO: 33 | | |
| ABE | Col4A5 | 21 | TCTTACTCAGGTGATGAGAT | ATG | 1B-C |
| | | | SEQ ID NO: 34 | | |
| ABE | APP | 17 | TTTTCAAGGTGTTCTTTGCA | GAA | 1B-C, 4B-C, E, G-H, |
| | | | SEQ ID NO: 35 | | K, 5B, 8F, 9B |
| ABE | RELA | 7 | ACTGCCTCAGGTGCCCCCAA | CAC | 6B |
| | | | SEQ ID NO: 36 | | |
| ABE | RELA | 7 | TCACTGCCTCAGGTGCCCCC | AAC | 2C, |
| | | | SEQ ID NO: 37 | | |
| ABE | RELA | 7 | TCACTGCCTCAGGTGCCCCC | AAC | 20, 6B |
| | | | SEQ ID NO: 38 | | |
| ABE | RELA | 7 | TATACCTTTCTGCACCTTGT | CAC | 6B |
| | | | SEQ ID NO: 39 | | |
| ABE | JAG1 | 12 | TTCTAGGATTTGGTTAATGG | TTA | 2B |
| | | | SEQ ID NO: 40 | | |
| ABE | JAG1 | 12 | TCACCTGACAGAGGTTTCCA | GAG | 2B |
| | | | SEQ ID NO: 41 | | |
| ABE | HSF1 | 6 | CGCAGCTGTTCAGCCCCTCG | GTG | 2C |
| | | | SEQ ID NO: 42 | | |
| ABE | HSF1 | 6 | CCGCAGCTGTTCAGCCCCTC | GGT | S1B |
| | | | SEQ ID NO: 43 | | |
| ABE | HSF1 | 6 | TACGCACACTGGCCAGGCTG | CTG | 20, 6B |
| | | | SEQ ID NO: 44 | | |
| ABE | HSF1 | 6 | CTACGCACACTGGCCAGGCT | GCT | 6B |
| | | | SEQ ID NO: 45 | | |
| ABE | LMNA | 11 | TCCCAGGGCTCCCACTGCAG | CAG | 6B |
| | | | SEQ ID NO: 46 | | |
| ABE | LMNA | 11 | TTCCCAGGGCTCCCACTGCA | GCA | 2B |
| | | | SEQ ID NO: 47 | | |
| ABE | LMNA | 11 | GACAACTCACCTGGGTTCGG | GGG | 2B, 6B |
| | | | SEQ ID NO: 48 | | |
| ABE | LMNA | 11 | AGACAACTCACCTGGGTTCG | GGG | 6B |
| | | | SEQ ID NO: 49 | | |
| ABE, SD 1 | APP | 17 | AAGTTTACCTACCTCCACCA | CAC | 4D, L, 5C, 8F, 9C |
| | | | SEQ ID NO: 50 | | |
| ABE, SD 2 | APP | 17 | ACCTACCTCCACCACACCAT | GAT | 4D, L, 5C |
| | | | SEQ ID NO: 51 | | |
| ABE, SD 3 | APP | 17 | AGTTTACCTACCTCCACCAC | ACC | 4D, L, 5C |
| | | | SEQ ID NO: 52 | | |
| CBE | JAG1 | 12 | CTAGAAGAGGAGAAGGGGAG | AGA | 1B-C |
| | | | SEQ ID NO: 53 | | |
| CBE | JAG1 | 12 | TCCTAGAAGAGGAGAAGGGG | AGA | 1D-E, H-I, 2B |
| | | | SEQ ID NO: 54 | | |
| CBE | JAG1 | 12 | ATCCTAGAAGAGGAGAAGGG | GAG | 1D-E |
| | | | SEQ ID NO: 55 | | |
| CBE | JAG1 | 12 | AATCCTAGAAGAGGAGAAGG | GGA | 1D-E, H-I |
| | | | SEQ ID NO: 56 | | |
| CBE | JAG1 | 12 | AAATCCTAGAAGAGGAGAAG | GGG | 1D-E, H-I |
| | | | SEQ ID NO: 57 | | |
| CBE | JAG1 | 12 | ACCATTAACCAAATCCTAGAAG | AGG | 1D-E, H-I |
| | | | SEQ ID NO: 58 | | |
| CBE | JAG1 | 12 | CAAATCCTAGAAGAGGAGAA | GGG | 1D-E, H-I |
| | | | SEQ ID NO: 59 | | |
| CBE | RELA | 7 | CACCTGAGGCAGTGAAAACA | AGG | 1H-I |
| | | | SEQ ID NO: 60 | | |
| CBE | RELA | 7 | GCACCTGAGGCAGTGAAAAC | AAG | 1H-I |
| | | | SEQ ID NO: 61 | | |
| CBE | BAP1 | 2 | GAGGCCTGGGTGGGGCGACA | AGA | 1H-1, 2D, 6B |
| | | | SEQ ID NO: 62 | | |
| CBE | BAP1 | 2 | CTTACCGAAATCTTCCACGA | GCA | 2D |
| | | | SEQ ID NO: 63 | | |
| CBE | BAP1 | 2 | CTCTTACCGAAATCTTCCAC | GAG | 6B |
| | | | SEQ ID NO: 64 | | |
| CBE | AHCY | 9 | CGGTCCACCTACACGCAGGC | AGG | 2D |
| | | | SEQ ID NO: 65 | | |
| CBE | AHCY | 9 | CCCACCTTCTTGGGCAGGAA | ATG | 2D |
| | | | SEQ ID NO: 66 | | |
| CBE | EGFR | 23 | ACCCCTGAGAGGATGAAGCA | AGA | 2B, 6B |
| | | | SEQ ID NO: 67 | | |
| CBE | EGFR | 23 | CACACTTGACCATGATCATG | TAG | 2B |
| | | | SEQ ID NO: 68 | | |
| CBE | EGFR | 23 | ACTCACACTTGACCATGATC | ATG | 6B |
| | | | SEQ ID NO: 69 | | |
| CBE | JAG1 | 12 | TTGCCACCACTCACCTGACA | GAG | 2B |
| | | | SEQ ID NO: 70 | | |
| CBE | APP | 17 | ACACCTTGAAAACAAATTAA | GAA | 4B |
| | | | SEQ ID NO: 71 | | |
| ABE | APP | SA OT 1 | TTTTAAAGGTGTTCTTTGCG | GAG | 8F |
| | | | SEQ ID NO: 72 | | |
| ABE | APP | SA OT 2 | TTTTGAAGGTGTTCTTTACA | GAG | 8F |
| | | | SEQ ID NO: 73 | | |
| ABE | APP | SA OT 3 | TTTACCAGGTGTTCTTTGCT | GAA | 8F |
| | | | SEQ ID NO: 74 | | |
| ABE | APP | SA OT 4 | TTATCAAGGTGTTCTATACA | GAA | 8F |
| | | | SEQ ID NO: 75 | | |
| ABE | APP | SA OT 5 | CTTTCAAGTTGTTCTGTGCA | GAG | 8F |
| | | | SEQ ID NO: 76 | | |
| ABE | APP | SD OT 1 | AAGTTTCCCTACCGCCACCA | TGG | 8F |
| | | | SEQ ID NO: 77 | | |
| ABE | APP | SD OT 2 | AAGGTTACCTTCCTCCACCA | TTC | 8F |
| | | | SEQ ID NO: 78 | | |
| ABE | APP | SD OT 3 | AAGTTTCCCTACATCCACCA | TTG | 8F |
| | | | SEQ ID NO: 79 | | |
| ABE | APP | SD OT 4 | AAGTCTACGTACCTCCACCA | TCT | 8F |
| | | | SEQ ID NO: 80 | | |
| ABE | APP | SD OT 5 | TAGTTTACCTCCCTCCACCC | TCT | 8F |
| | | | SEQ ID NO: 81 | | |

### Cell Culture and Transfections

The cell line HEK293T was obtained from the American Type Culture Collection (ATCC) and was maintained in DMEM supplemented with 10% fetal bovine serum and 1% penicillin/streptomycin at 37° C. with 5% C02. BE(2)-M17 cells was obtained from the ATCC and maintained in 1:1 EMEM:Ham's F1 2K supplemented with 10% fetal bovine serum and 1% penicillin/streptomycin at 37° C. with 5% C02.

HEK293T cells were transfected in 24-well plates with Lipofectamine 2000 (Invitrogen) via reverse transfection following the manufacturer's instructions. The amount of DNA used for lipofection was 1 μg per transfection. In experiments with split base editors, 500 ng of each split were used. For simultaneous targeting experiments in HEK293 Ts, 500 ng of plasmid encoding full-length BEs were transfected with 500 ng of plasmid with the sgRNA expression cassette when one sgRNA was expressed. Whenever two separate sgRNAs were transfected, 333.33 ng of plasmid encoding BEs was used with 333.33 ng of each sgRNA cassette. Transfection efficiency was routinely higher than 90% for HEK293T cells as determined by fluorescent microscopy following delivery of 1 μg of a GFP expression plasmid.

### AA V Vector Production

AAV was produced according to the protocol described previously.⁹¹ HEK293T cells were seeded onto 15-cm plates to be 80% confluent after 16 hours and maintained in DMEM supplemented with 10% (v/v) FBS and 1% (v/v) penicillin/streptomycin. After 16 hours, cells were transfected with 65 pg total of AAV vector plasmid (pAAV-CAG-N-ABE8e-SpRY-APPex17sgRNA, or pAAV-CAG-C-ABE8e-SpRY-APPex17sgRNA, or pAAV-CAG-GFP-KASH), pAAVrh10, and pHelper in a 1:1:1 mol ratio using PEI-max (pH=8). XmaI digestion was used to confirm the integrity of the pAAV plasmids before transfection. Cell media was replaced 4 hours after transfection. Cells were harvested 72 hours after transfection by manual dissociation using a cell scraper and centrifuged at 1500×g for 5 min at room temperature. The supernatant was collected into a fresh tube and mixed with 40% polyethylene glycol 8000 (Thermo Fisher) solution in a ratio of 4:1 by volume and stored overnight at 4° C. Cell pellets were then resuspended in 2 mL of lysis buffer (50 mM Tris-HCl and 150 mM NaCl, pH 8.0) per plate. The next day, the supernatant solution was centrifuged at 3,000 RPM for 30 minutes at 4° C. The pellets of the supernatant were resuspended in the same lysis buffer as described above and mixed with their respective cell lysates. These cell suspensions were then frozen in liquid Nitrogen and thawed at 37° C., for three freeze-thaw cycles to extract AAV vectors. Supernatants were then treated with 0.5% Triton X-100 (Thermo Fisher) and 50 units/mL Benzonase (Merck) shaking at 37° C. for 1 hour. We then centrifuged the lysate at 10,000×g for 15 minutes at room temperature. The resulting supernatant was overlaid onto an iodixanol density gradient using 15%, 25%, 40% and 60% Opti-Prep solution (Sigma-Aldrich) and the virus was isolated by ultracentrifugation at 58,400 RPM at 18° C. This step was repeated to perform a second gradient purification using iodixanol fractions of 30%, 40% and 60%. Following extraction, AAV was filter-dialyzed with 1×PBS containing 0.001% Tween-20 using an Amicon Ultra 100 kDa MWCO column (Merck). The titer of the purified AAV was determined, post-treatment with DNase I (Millipore Sigma), by quantitative real-time PCR using primers that amplify the bGH PolyA sequence (Table 3) and the SsoFast Evagreen mix (Bio-Rad). The virus was stored at −80° C.

TABLE 3
Primer Sequences for DNA/RNA amplification and Sequencing

| Target | Exon | Purpose | Sequence |
|---|---|---|---|
| TRAP1 | 4 | gDNA FW | AACGATCACTTGAGCCTGGG SEQ ID NO: 82 |
| TRAP1 | 4 | gDNA RV | TGAGGGAGGTACCTGGATGG SEQ ID NO: 83 |
| TRAP1 | 4 | gDNA sequencing | GCAACATAGCGAGACCCTGT SEQ ID NO: 84 |
| RAB34 | 9 | gDNA FW | GGCTCTGCTGTGACACTCTT SEQ ID NO: 85 |
| RAB34 | 9 | gDNA RV | AGTAGGGGGCATCCAGTCTT SEQ ID NO: 86 |
| RAB34 | 9 | gDNA sequencing | AGCTCTGGAGGAATGAGGGT SEQ ID NO: 87 |
| EGFR | 23 | gDNA FW | TTGGGTCCTTACAGCAATCC SEQ ID NO: 88 |
| EGFR | 23 | gDNA RV | GATGCAAAGGCCTCAGCTGTTT SEQ ID NO: 89 |
| EGFR | 23 | gDNA sequencing | GAGGTAGACTGAGGCTTCCAGC SEQ ID NO: 90 |
| EGFR | 23 | RT-PCR FW | GCTCAACTGGTGTGTGCAGATC SEQ ID NO: 91 |
| EGFR | 23 | RT-PCR RV | TCACGGAACTTTGGGCGACTAT SEQ ID NO: 92 |
| EGFR | 23 | NGS cDNA FW | TCGTCGGCAGCGTCAGATGTGTATAAGAGACAG |
| | | | GCTCAACTGGTGTGTGCAGATC |
| | | | SEQ ID NO: 93 |
| EGFR | 23 | NGS cDNA RV | GTCTCGTGGGCTCGGAGATGTGTATAAGAGACAG |
| | | | TCACGGAACTTTGGGCGACTAT |
| | | | SEQ ID NO: 94 |
| JAG1 | 12 | gDNA FW | GCTGAAGCCCTGTGTTTGTG SEQ ID NO: 95 |
| JAG1 | 12 | gDNA RV | GCTGTAGGGACTGCCAATGT SEQ ID NO: 96 |
| JAG1 | 12 | gDNA sequencing | TCGTCAGTATCTCCTTCCACC SEQ ID NO: 97 |
| JAG1 | 12 | RT-PCR FW | CGGATTTAAGTGTGTGTGCCCC SEQ ID NO: 98 |
| JAG1 | 12 | RT-PCR RV | TTCTGGCAGGGATTAGGCTCAC SEQ ID NO: 99 |
| JAG1 | 12 | NGS cDNA FW | TCGTCGGCAGCGTCAGATGTGTATAAGAGACAGCGGATTTAAGTGTGTGTGC |
| | | | CCC SEQ ID NO: 100 |
| JAG1 | 12 | NGS cDNA RV | GTCTCGTGGGCTCGGAGATGTGTATAAGAGACAGTTCTGGCAGGGATTAGGC |
| | | | TCAC SEQ ID NO: 101 |
| BAP1 | 2 | gDNA FW | TGAATAAGGGCTGGCTGGAG SEQ ID NO: 102 |
| BAP1 | 2 | gDNA RV | ATTGTGTGACCGGGGTCTTC SEQ ID NO: 103 |
| BAP1 | 2 | gDNA sequencing | GTAGGGTTCCTGGCACTGTC SEQ ID NO: 104 |
| BAP1 | 2 | RT-PCR FW | GTCTGTGTGTGGGACTGAGG SEQ ID NO: 105 |
| BAP1 | 2 | RT-PCR RV | GTTGGGTATCAGCTGGTGGG SEQ ID NO: 106 |
| BAP1 | 2 | NGS cDNA FW | TCGTCGGCAGCGTCAGATGTGTATAAGAGACAG |
| | | | GTCTGTGTGTGGGACTGAGG SEQ ID NO: 107 |
| BAP1 | 2 | NGS cDNA RV | GTCTCGTGGGCTCGGAGATGTGTATAAGAGACAG |
| | | | GTTGGGTATCAGCTGGTGGG SEQ ID NO: 108 |
| AHCY | 9 | gDNA FW | AGAGACGGGCTTTCACTGTG SEQ ID NO: 109 |
| AHCY | 9 | gDNA RV | TTAGACACGTGACCCTTGGC SEQ ID NO: 110 |
| AHCY | 9 | gDNA sequencing | CGGCCAGCTCAACATTTCTT SEQ ID NO: 111 |
| AHCY | 9 | RT-PCR FW | CATCTTTGTCACCACCACAGGC SEQ ID NO: 112 |
| AHCY | 9 | RT-PCR RV | AGGTACTGGGCTTGCTTCTCAG SEQ ID NO: 113 |
| AHCY | 9 | NGS cDNA FW | TCGTCGGCAGCGTCAGATGTGTATAAGAGACAG |
| | | | GTCAAGTGGCTCAACGAGAACG SEQ ID NO: 114 |
| AHCY | 9 | NGS cDNA RV | GTCTCGTGGGCTCGGAGATGTGTATAAGAGACAG |
| | | | TCCAAGACCACTGAGCTCATGG SEQ ID NO: 115 |
| RELA | 7 | gDNA FW | CAGATTGGCACCCACTGGACTA SEQ ID NO: 116 |
| RELA | 7 | gDNA RV | CCTGGTCCCGTGAAATACACCT SEQ ID NO: 117 |
| RELA | 7 | gDNA sequencing | GGGTCAGTGTGTCTAACCCTCC SEQ ID NO: 118 |
| RELA | 7 | RT-PCR FW | CCCACGAGCTTGTAGGAAAGGA SEQ ID NO: 119 |
| RELA | 7 | RT-PCR RV | CCTGGTCCCGTGAAATACACCT SEQ ID NO: 120 |
| RELA | 7 | NGS cDNA FW | TCGTCGGCAGCGTCAGATGTGTATAAGAGACAG |
| | | | CCCACGAGCTTGTAGGAAAGGA SEQ ID NO: 121 |
| RELA | 7 | NGS cDNA RV | GTCTCGTGGGCTCGGAGATGTGTATAAGAGACAG |
| | | | CCTGGTCCCGTGAAATACACCT SEQ ID NO: 122 |
| LMNA | 11 | gDNA FW | GTCCCCAAGTCTTCCTGAGC SEQ ID NO: 123 |
| LMNA | 11 | gDNA RV | TCCTACCCCTCGATGACCAG SEQ ID NO: 124 |
| LMNA | 11 | gDNA sequencing | TGTCTTCCCTCTCCTCCTCC SEQ ID NO: 125 |
| LMNA | 11 | gDNA sequencing | ACCCATCTCCTCTGGCTCTT SEQ ID NO: 126 |
| LMNA | 11 | RT-PCR FW | GACGACGAGGATGAGGATGGAG SEQ ID NO: 127 |
| LMNA | 11 | RT-PCR RV | CACCCCTTTCCCTTGGCTTCTA SEQ ID NO: 128 |
| LMNA | 11 | NGS cDNA FW | TCGTCGGCAGCGTCAGATGTGTATAAGAGACAG |
| | | | ACGGCTCTCATCAACTCCAC SEQ ID NO: 129 |
| LMNA | 11 | NGS cDNA RV | GTCTCGTGGGCTCGGAGATGTGTATAAGAGACAG |
| | | | TGAGGAGGACGCAGGAAG SEQ ID NO: 130 |
| HSF1 | 11 | gDNA FW | CTGTTCTGACTTCCCTCCCTCC SEQ ID NO: 131 |
| HSF1 | 11 | gDNA RV | TGGGACTTGGCTCACCTGAATC SEQ ID NO: 132 |
| HSF1 | 11 | gDNA sequencing | TGGGACTTGGCTCACCTGAATC SEQ ID NO: 133 |
| HSF1 | 11 | RT-PCR FW | TGCCTGGACAAGAATGAGCTCA SEQ ID NO: 134 |
| HSF1 | 11 | RT-PCR RV | CTCTAGGAGACAGTGGGGTCCT SEQ ID NO: 135 |
| HSF1 | 11 | NGS cDNA FW | TCGTCGGCAGCGTCAGATGTGTATAAGAGACAG |
| | | | TGCCTGGACAAGAATGAGCTCA SEQ ID NO: 136 |
| HSF1 | 11 | NGS cDNA RV | GTCTCGTGGGCTCGGAGATGTGTATAAGAGACAG |
| | | | CTCTAGGAGACAGTGGGGTCCT SEQ ID NO: 137 |
| APP | 17 | gDNA FW | CCAACCAGTTGGGCAGAGAATA SEQ ID NO: 138 |
| APP | 17 | gDNA RV | ACCTGGTTGTATTGAGAGGCCA SEQ ID NO: 139 |
| APP | 17 | gDNA sequencing | AGTTCTTAGCAAAAAGCTAAGCCTAA SEQ ID NO: 140 |
| APP | 17 | RT-PCR FW | GAAGTTGAGCCTGTTGATGCCC SEQ ID NO: 141 |
| APP | 17 | RT-PCR RV | CGTTCTGCTGCATCTTGGACAG SEQ ID NO: 142 |
| APP | 17 | NGS cDNA FW | TCGTCGGCAGCGTCAGATGTGTATAAGAGACAGGAAGTTGAGCCTGTTGATG |
| | | | CCC SEQ ID NO: 143 |
| APP | 17 | NGS cDNA RV | GTCTCGTGGGCTCGGAGATGTGTATAAGAGACAGCGTTCTGCTGCATCTTGGA |
| | | | CAG SEQ ID NO: 144 |
| APP | 17 | NGS gDNA FW | TCGTCGGCAGCGTCAGATGTGTATAAGAGACAG ATCCAAATGTCCCCTGCAT |
| | | (mice experiments) | SEQ ID NO: 145 |
| APP | 17 | NGS gDNA RV (mice | GTCTCGTGGGCTCGGAGATGTGTATAAGAGACAG |
| | | experiments) | CATGGAAGCACACTGATTCG SEQ ID NO: 146 |
| APP | 17 | NGS cDNA FW (mice | TCGTCGGCAGCGTCAGATGTGTATAAGAGACAG |
| | | experiments) | ACACAGAAAACGAAGTTGAGC SEQ ID NO: 147 |
| APP | 17 | NGS cDNA RV (mice | GTCTCGTGGGCTCGGAGATGTGTATAAGAGACAG |
| | | experiments) | GCGATAATGAGTAAATCATAAA SEQ ID NO: 148 |
| APP | SA OT 1: hg38 | NGS gDNA FW | TCGTCGGCAGCGTCAGATGTGTATAAGAGACAG |
| | chr8: | | TTTGACCAGATCCCTCTCTCA SEQ ID NO: 149 |
| | 95309336- | | |
| | 95309936 | | |
| APP | SA OT 1: hg38 | NGS gDNA RV | GTCTCGTGGGCTCGGAGATGTGTATAAGAGACAG |
| | chr8: | | GGGTCTGGTCCCTACCCTTA SEQ ID NO: 150 |
| | 95309336- | | |
| | 95309936 | | |
| APP | SA OT 2: hg38 | NGS gDNA FW | TCGTCGGCAGCGTCAGATGTGTATAAGAGACAG |
| | chr1: | | CAACCTTCCAACCAAGCAAT SEQ ID NO: 151 |
| | 58551430- | | |
| | 58551930 | | |
| APP | SA OT 2: hg38 | NGS gDNA RV | GTCTCGTGGGCTCGGAGATGTGTATAAGAGACAG |
| | chr1: | | CAGTGTGACCCTTCAAGCAA SEQ ID NO: 152 |
| | 58551430- | | |
| | 58551930 | | |
| APP | SA OT 3: hg38 | NGS gDNA FW | TCGTCGGCAGCGTCAGATGTGTATAAGAGACAG |
| | chr7: | | ACAGCCAAACCAAAAATTGC SEQ ID NO: 153 |
| | 81700000- | | |
| | 81700496 | | |
| APP | SA OT 3: hg38 | NGS gDNA RV | GTCTCGTGGGCTCGGAGATGTGTATAAGAGACAG |
| | chr7: | | TGGGTGGAATTGACAATGAA SEQ ID NO: 154 |
| | 81700000- | | |
| | 81700497 | | |
| APP | SA OT 4: hg38 | NGS gDNA FW | TCGTCGGCAGCGTCAGATGTGTATAAGAGACAG |
| | chr8: | | GTGCATGGGATGTTCATCAG SEQ ID NO: 155 |
| | 107193000- | | |
| | 107193476 | | |
| APP | SA OT 4: hg38 | NGS gDNA RV | GTCTCGTGGGCTCGGAGATGTGTATAAGAGACAG |
| | chr8: | | CTCAGCCAGACTGCCAACTT SEQ ID NO: 156 |
| | 107193000- | | |
| | 107193477 | | |
| APP | SA OT 5: hg38 | NGS gDNA FW | TCGTCGGCAGCGTCAGATGTGTATAAGAGACAG |
| | chr4: | | TGCAAGGCCCAAGTAATAGC SEQ ID NO: 157 |
| | 94419456- | | |
| | 94419956 | | |
| APP | SA OT 5: hg38 | NGS gDNA RV | GTCTCGTGGGCTCGGAGATGTGTATAAGAGACAG |
| | chr4: | | CATTTTCAGCACCAGAACCA SEQ ID NO: 158 |
| | 94419456- | | |
| | 94419957 | | |
| APP | SD OT 1: hg38 | NGS gDNA FW | TCGTCGGCAGCGTCAGATGTGTATAAGAGACAG |
| | chr2: | | CCTGTTTCTTAGCCGTCCTG |
| | 238138000- | | SEQ ID NO: 159 |
| | 238138478 | | |
| APP | SD OT 1: hg38 | NGS gDNA RV | GTCTCGTGGGCTCGGAGATGTGTATAAGAGACAG |
| | chr2: | | CACGTGGGAAAACGTGATTA SEQ ID NO: 160 |
| | 238138000- | | |
| | 238138479 | | |
| APP | SD OT 2: hg38 | NGS gDNA FW | TCGTCGGCAGCGTCAGATGTGTATAAGAGACAG |
| | chr13: | | CCCCAAGATATGGGATGTTG SEQ ID NO: 161 |
| | 80179531- | | |
| | 80179999 | | |
| APP | SD OT 2: hg38 | NGS gDNA RV | GTCTCGTGGGCTCGGAGATGTGTATAAGAGACAG |
| | chr13: | | TTAGCAAGCCCCTTTTGTTC SEQ ID NO: 162 |
| | 80179531- | | |
| | 80179999 | | |
| APP | SD OT 3: hg38 | NGS gDNA FW | TCGTCGGCAGCGTCAGATGTGTATAAGAGACAG |
| | chr16: | | ATGGACTTGGTCCCAAACAG SEQ ID NO: 163 |
| | 35480069- | | |
| | 35480569 | | |
| APP | SD OT 3: hg38 | NGS gDNA RV | GTCTCGTGGGCTCGGAGATGTGTATAAGAGACAG |
| | chr16: | | AGCTCCACCATATCGTGTCC SEQ ID NO: 164 |
| | 35480069- | | |
| | 35480569 | | |
| APP | SD OT 4: hg38 | NGS gDNA FW | TCGTCGGCAGCGTCAGATGTGTATAAGAGACAG |
| | chr9: | | CGGCTGGCCATACACTTTTA SEQ ID NO: 165 |
| | 88814241- | | |
| | 88814741 | | |
| APP | SD OT 4: hg38 | NGS gDNA RV | GTCTCGTGGGCTCGGAGATGTGTATAAGAGACAG |
| | chr9: | | TGCCTGAGATGTGAGTTTGC SEQ ID NO: 166 |
| | 88814241- | | |
| | 88814742 | | |
| APP | SD OT 5: hg38 | NGS gDNA FW | TCGTCGGCAGCGTCAGATGTGTATAAGAGACAG |
| | chr10: | | CTGGGACATGAAGCTCGTCT SEQ ID NO: 167 |
| | 16760535- | | |
| | 16760999 | | |
| APP | SD OT 5: hg38 | NGS gDNA RV | GTCTCGTGGGCTCGGAGATGTGTATAAGAGACAG |
| | chr10: | | GCACTCCAGCCTAGGTGACA SEQ ID NO: 168 |
| | 16760535- | | |
| | 16761000 | | |
| APP | | qPCR | GAGGTACCCACTGATGGTAATG SEQ ID NO: 169 |
| APP | | qPCR | CTGAATCCCACTTCCCATTCT SEQ ID NO: 170 |
| GAPDH | | qPCR | CAATGACCCCTTCATTGACC SEQ ID NO: 171 |
| GAPDH | | qPCR | TTGATTTTGGAGGGATCTCG SEQ ID NO: 172 |

### RT-PCR

RNA was harvested from cell pellets using the RNeasy Plus Mini Kit (Qiagen) according to manufacturer's instructions. cDNA synthesis was performed using the qScript cDNA Synthesis Kit (Quanta Biosciences) from 1 μg of RNA with cycling conditions performed as directed by the supplier. PCR was performed using KAPA2G Robust PCR kits from Kapa Biosystems. The 25 μL reactions used 25 ng of cDNA, Buffer A (5 μL), Enhancer (5 μL), dNTPs (0.5 μL), 10 μM forward primer (1.25 μL), 10 μM reverse primer (1.25 μL), KAPA2G Robust DNA Polymerase (0.5 U) and water (up to 25 μL). Cycling parameters were used a recommended by the manufacturer. The PCR products were visualized in 2% agarose gels stained with ethidium bromide and images were captured using a ChemiDoc-It2 (UVP). The DNA sequences of the primers for each target are provided in Table 3.

### Densitometry Analysis

Skipping efficiencies for screening of simultaneous sgRNA candidates were determined by densitometry analysis of the PCR products obtained from RT-PCR and analyzed by agarose gel electrophoresis using ImageJ software. After subtracting background noise, band intensity was compared using the following formula: % exon skipping=(Skipped Band Intensity)/(Non Skipped Band Intensity+Skipped Band Intensity) where band intensity is the sum of each pixel grayscale value within the selected area of the band.

### Analysis of DNA Editing Rates

Genomic DNA was isolated using a DNeasy Blood and Tissue Kit (Qiagen) and PCR amplification was performed with KAPA2G Robust PCR kits (KAPA Biosystems) as described above, using 20-100 ng of template DNA and primers listed in Table 3.

Sanger sequencing of the PCR amplicons was performed by the Roy J. Carver Biotechnology Center at the University of Illinois at Urbana-Champaign. Base editing efficiencies were estimated by analyzing sequencing traces using EditR with the primers listed in Table 3.

### Next-Generation Sequencing for RNA Exon Skipping and DNA Amplicons

After cDNA synthesis (qScript, QuantaBio), amplicons were generated using KAPA HiFi HotStart (Roche), according to manufacturer's instructions with primers containing overhangs compatible with Nextera XT indexing (IDT). Following validation of the quality of PCR products by gel electrophoresis, the PCR products were isolated using an AMPure XP PCR purification beads (Beckman Coulter). Indexed amplicons were then generated with a Nextera XT DNA Library Prep Kit (Illumina) and pooled. Libraries were sequenced with a MiSeq Nano Flow Cell for 251 cycles from each end of the fragment using a MiSeq Reagent Kit v2 (500-cycles). FASTQ files were created and demultiplexed using bcl2fastq v2.17.1.14 Conversion Software (Illumina). Deep sequencing was performed by the W.M. Keck Center for Comparative and Functional Genomics at the University of Illinois, Urbana, IL.

Exon skipping rates were quantified using the STAR RNA-Seq aligner on Galaxy. Forward and reverse reads were combined and aligned to the human reference genome (GRCh38) using STAR. 2-pass mapping was used for splice junction analysis with a MAPQ value of 60 for .bam files. Splice junctions formed were determined from STAR SJ.out.tab files where the percentage of a junction event was defined as the number of reads for the target junction divided by the total number of events at the specified junction. Sashimi plots for splice junctions were generated from the Integrative Genomics Viewer (IGV's) sashimi plot tool using the .bam and .bai files outputted from RNA-STAR. These sashimi plots were illustrated herein by tracing the images generated from IGV in Adobe Illustrator.

For DNA amplicons, samples were isolated with the DNeasy Blood and Tissue Kit and the RNeasy Plus Mini Kit per manufacturer's specifications. Amplicons were generated by PCR with KAPA HiFi Hotstart using primers containing overhangs compatible with Nextera XT indexing primers listed in Table 3. Amplification, indexing, pooling, and sequencing was performed as described above. Base editing rates were quantified using CRISPResso2. Reads with average phred scores below 30 were removed, and remaining reads were aligned to the expected amplicon sequences.

### RNA-Seq

RNA was harvested as described above. Following analysis of RNA on a 1% agarose gel for RNA purity, RNA seq libraries were prepped with the TrueSeq® mRNA library prep kit (Illumina) and samples were sequenced with a NovaSeq 6000 system (Illumina). For analysis, a quality check performed using FASTQC showed that average per-base read quality scores in all samples were above 34 and no adapter sequences were found, indicating high quality reads, requiring no trimming. The reads were then mapped to the human genome (hg38) using HISAT2, with over 96% of reads being mapped uniquely. These mapped reads were counted across human genes with featureCounts, using annotations of hg38 obtained from GENCODE. About 60% of reads were assigned to genes. Differential expression analysis was performed using limma-voom which first calculates TMM normalized counts-per-million (CPM) to account for compositional bias. In addition, genes expressed at very low levels (CPM lower than 0.5) were filtered out before performing differential expression analysis. For DEG, a DEG was defined as a gene with a Benjamini-Hochberg adjusted p>0.05.

### RT-qPCR

RNA harvesting and cDNA synthesis was performed as described above. For qPCR, 50 ng of cDNA was used per sample. qPCR was performed with SsoFast™ EvaGreen® Supermix (Biorad) following manufacturer recommendations for both reaction setup and cycling conditions. 500 nM of qPCR primers were used per sample, and GAPDH was used as the housekeeping gene. Thermocycling and cycle threshold (ct) measurements was conducted on the CFX96™ qPCR instrument (Biorad). Gene expression levels were analyzed from ct values of the target genes and relative expression was quantified using the double-delta ct method.⁹² All primers used for qPCR are listed under Table 3.

### ELISA

To generate cells with editing at APP exon 17, BE(2)-M17 cells were transfected via reverse transfection with lipofectamine 2000 containing 1 μg of pEFS-SpRY-ABE8e-T2A-Puro, a plasmid which also contained a U6 driven sgRNA expression cassette. For samples expressing multiple sgRNAs, 500 ng of each plasmid was used. 24 hours after transfection, cell media was exchanged with media supplemented with 1 μg/mL puromycin and incubated for 36 hours.

For ELISA, cells were plated onto 10 cm dishes and grown to 100% confluency. After cells reached confluency, cell media was replaced and incubated for an additional 24 hours. Media was then replaced with 10 mL OPTI-MEM supplemented with 1% penicillin/streptomycin and cells were incubated for 48 hours. Cell supernatant was collected into 1.5 mL tubes, supplemented with 1× Halt™ Protease Inhibitor Cocktail (Thermo™ Scientific 78430) and centrifuged at 1400 rpm for 1 minute. Supernatant was collected, and an Aβ42 ELISA was performed on undiluted samples using the Ultrasensitive Aβ42 Human ELISA Kit (Thermo™ Scientific KHB3544) according to the manufacturer's instructions. Standards were diluted in OPTI-MEM supplemented with 1% penicillin/streptomycin and 1× Halt™ cocktail.

For total protein analysis, adhered cells were lysed with 1×RIPA buffer on ice with 1× Halt™ cocktail and incubated on ice for ten minutes. Cells were spun at 10,000 rpm for 10 minutes, and total protein content in the supernatant was quantified with the Pierce™ Bradford Protein Assay Kit (Thermo™ Scientific 23200) following manufacturer instructions.

After samples were quantified via ELISA, the concentration of Aβ42 was normalized to the total protein content of each sample by dividing each Aβ42 concentration by the total protein concentration. The value generated by each sample was normalized to its respective ROSA positive control.

### Stereotaxic Injections

All animal procedures were approved by the Illinois Institutional Animal Care and Use Committee at the University of Illinois and conducted in accordance with the National Institutes of Health (NIH) Guide for the Care and Use of Laboratory Animals. Four to six week old transgenic B6.129-Tg(APPsw)40Btla/Mmjax mice (Jackson Laboratory #0034831-JAX) were injected with 5×10⁹ vector genomes of each of AAVrh10-pAAV-CAG-N-ABE8e, pAAVrh10-CAG-C-ABE8e, and 5×10⁸ vector genomes of AAVrh10-pAAV-CAG-KASH in 2 μl of PBS with 0.001% Tween-20 into each half of the hippocampus at coordinates −1.8 AP, ±1.5 ML, and 0.45 DV.

### Tissue Harvesting and RNA Purification

Mice were anesthetized using 3% isoflurane delivered through vaporizer in a closed chamber and transcardially perfused using 1×PBS. The hippocampus was dissected, divided in 2 halves and each half was stored via flash freezing or in RNAlater (Invitrogen, AM7026).

For FACS, nuclei isolation was performed as previously described. Briefly, tissues were homogenized in NF1 buffer using the KIMBLE Dounce Tissue Grinder (Sigma-Aldrich) per the manufacturer's instructions, strained through a 70 μm strainer into a 50 mL conical tube underlayed with a 1.2 M sucrose cushion, and centrifuged at 3,900G for 30 minutes at 4° C. The supernatant was discarded and the pelleted nuclei were resuspended in 10 mL of NF1 buffer. Samples were spun 2× at 1,600G for 5 minutes at 4° C. and resuspended in 2 mL of FANS buffer. Cells were strained through a 35 μm strainer and incubated at 37° C. for 30 minutes with 0.5 μL Vybrant™ Dyecycle™ Ruby strain (Invitrogen, V10309) per mL of FANS buffer.

Harvested nuclei were sorted using a ThermoFisher Bigfoot Spectral Cell Sorter Cell Sorter (Roy J. Carver Biotechnology Center, University of Illinois, Urbana, IL). Cells were collected in FANS buffer. At least 10,000 cells or nuclei were sorted for each sample. Collected nuclei were diluted in FANS buffer with 1% wt/vol (g/mL) BSA and spun at 1,600G for 15 minutes at 4° C. Supernatant was aspirated, and pelleted nuclei were isolated via the DNeasy Blood and Tissue Kit

To perform downstream analysis on whole hippocampus samples, DNA and RNA samples were isolated with the DNeasy Blood and Tissue Kit and the RNeasy Plus Mini Kit per manufacturer's specifications, respectively. Prior to purification, brain tissue samples were homogenized in 1×PBS using a KIMBLE Dounce Tissue Grinder, diluted in the first buffer of each respective kit, and further isolated following the standard kit protocols.

For NGS on all DNA samples, Amplicons were generated by PCR with KAPA HiFi Hotstart using primers containing overhangs compatible with Nextera XT indexing primers listed in Table 3. Amplification, indexing, pooling, and analysis of base editing rates was performed as described in the “Next-Generation Sequencing for RNA Exon Skipping and DNA Amplicons” section. For RNA analysis of exon skipping, cDNA synthesis and amplification for NGS was performed as previously described above, however, libraries or cDNA were sequenced with the MiSeq Bulk flow cell for 275 cycles from each end of the fragment using a MiSeq Reagent Kit v3 (500-cycles). Exon skipping was quantified with RNA-STAR as described above.

### Statistical Analysis

GraphPad Prism version 9.1 (GraphPad Software, Inc.) software was used for statistical analysis. All experiments consisted of independent replicates. Test groups were compared using either student's t-test or a One-Way ANOVA with Tukey's post-hoc analysis. Statistical methods used for RNAseq are described within the section titled “RNA-Seq”.

### Example 2: Engineered Near-PAMless SpCas9 Variants Enable Targeting of SAs Inaccessible by the Native SpCas9

One limitation of SpCas9 for exon skipping is its reliance on the NGG PAM motif. The use of SaCas9 or SpCas9-VQR could alleviate this problem, but BEs comprised of these variants often exhibit lower activity than those composed of the wild-type (WT) variant. Several versions of SpCas9 with relaxed PAM preferences have been developed, including SpCas9-NG (NG PAM)², xCas9-NG (NG PAM), NAG-Cas9 (NRG PAM), SpCas9-NRNH (NRNH PAM), SpG Cas9 (NRN PAM), and SpRY Cas9 (near NNN PAMs). Most notably, SpRY Cas9 has demonstrated DNA editing activity at NRN and NYN PAM sites comparable to SpCas9 when targeted to its native NGG PAM. SpRY Cas9 was also demonstrated to edit a larger number of compatible targets compared to xCas9-NG, SpCas9-NG, and SpG Cas9. Importantly, SpRY Cas9 can install base edits using NDN PAMs, which is particularly convenient for targeting SAs, whose consensus sequence is A/T-rich and enable tiling of splicing sequences with multiple sgRNAs (FIG. 1A).

Given the broader targeting range observed with SpRY Cas9, we next tested the exon skipping ability of a BE toolbox comprised of this variant. To accomplish this, we targeted multiple exons by tiling SAs with eight sgRNAs per gene at six exons that contained either one NGG PAM or no NGG PAMs with the adenosine deaminase ABE8e (FIG. 1B, C) or the cytosine deaminase CBE4max (FIG. 1D, E) with either SpCas9 or SpRY Cas9 in HEK293T cells. Using ABE8e and APOBEC1-CBE4max, we identified at least one sgRNA that introduced the desired mutation for every target exon for SpRY Cas9, while BEs based on SpCas9 only introduced mutations in the SA of two exons (FIG. 1B-E). At target SAs with NGG PAMs both SpCas9 and SpRY Cas9 introduced the target change at similar rates (p>0.05, FIG. 1B, D). Overall, the mean editing rates for SpRY Cas9-ABE8e and SpRY Cas9-CBE4max were ~31% and ~35%, respectively, while the mean editing rates for WT ABE8e and CBE4max were ~3% for each. Thus, SpRY Cas9 provided a 9.5- and 9.7-fold increase in mean SA editing when comparing WT to SpRY for both ABE8e and CBE4max, respectively, (p<0.05; FIG. 1C, E).

Multiple new Cas9 BEs have been developed for various purposes. For example, in addition to CBE4max, CBEs composed of the evoFERNY, SsAPOBEC3B (R54Q), RrA3F (F130L), TadCBE, and PpAPOBEC1 (H122A) catalytic domains have expanded editing contexts, increasing overall editing efficiency, and decreasing non-specific deamination. In the case of ABEs, evolved variants such as the ABE8 family, including ABE8e and ABE8.20m have increased editing efficiency and different editing windows than the previous generation ABE7.10. Additionally, ABE9 was developed to minimize bystander editing by BEs of the ABE8 family. When fusing different deaminases to SpRY Cas9, we observed distinct profiles of edited SA sequences (FIG. 1F-I).

When comparing ABEs, ABE8e outperformed all other deaminases possessing a mean editing rate of ~47% compared to ~7%, ~28%, and ~0.3% for ABE7.1, ABE8.20m, and ABE9, respectively (p<0.01, FIG. 1F, G).

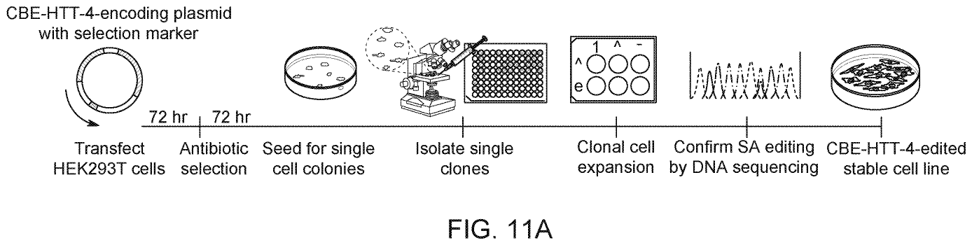

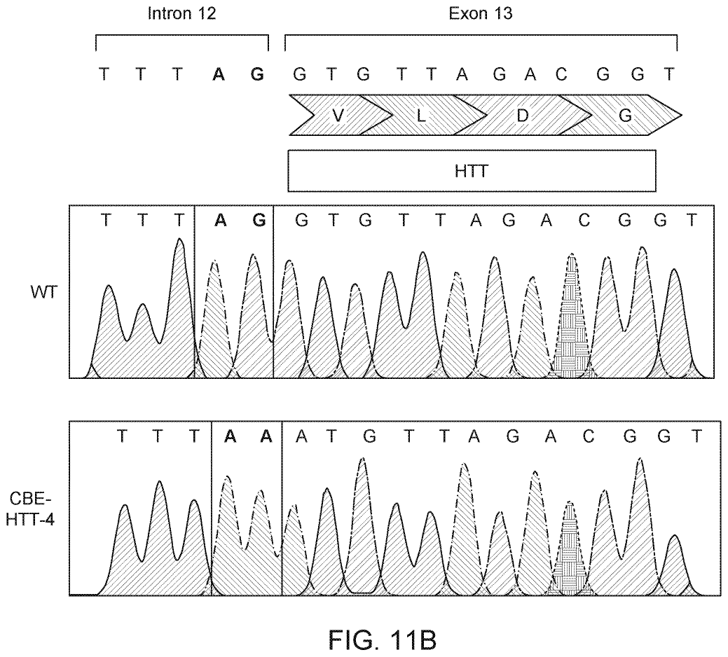

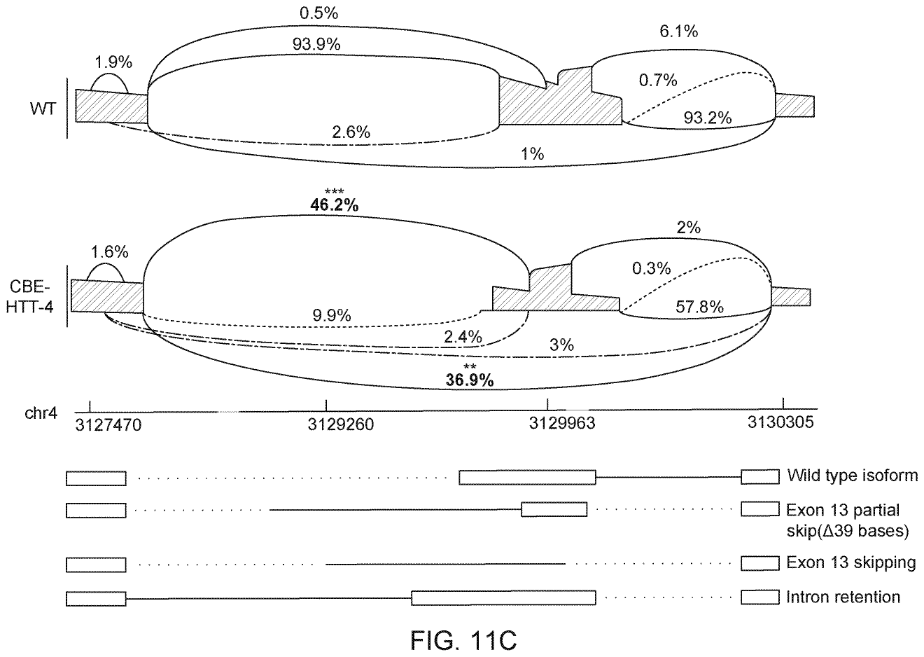

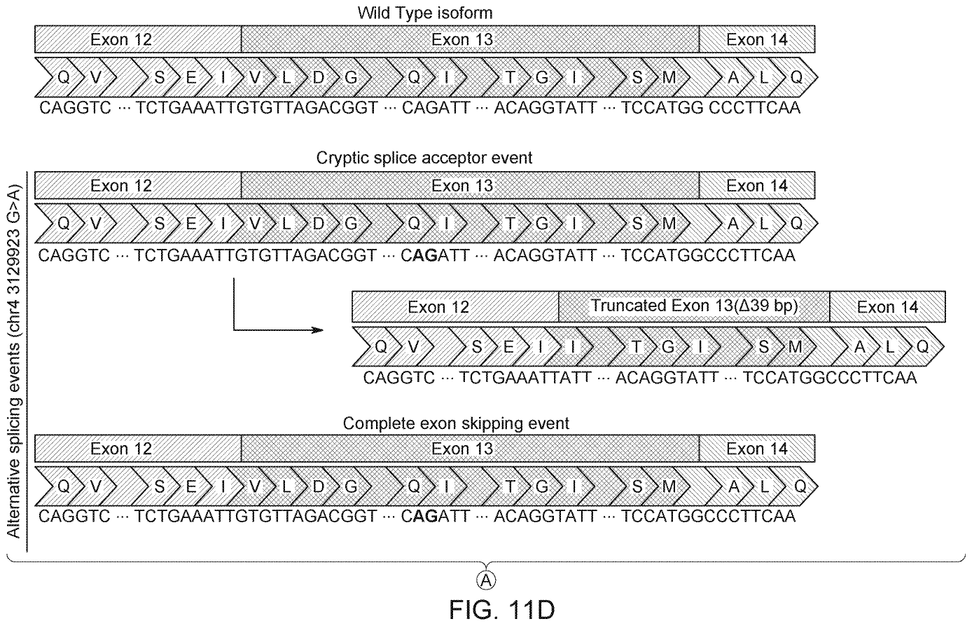

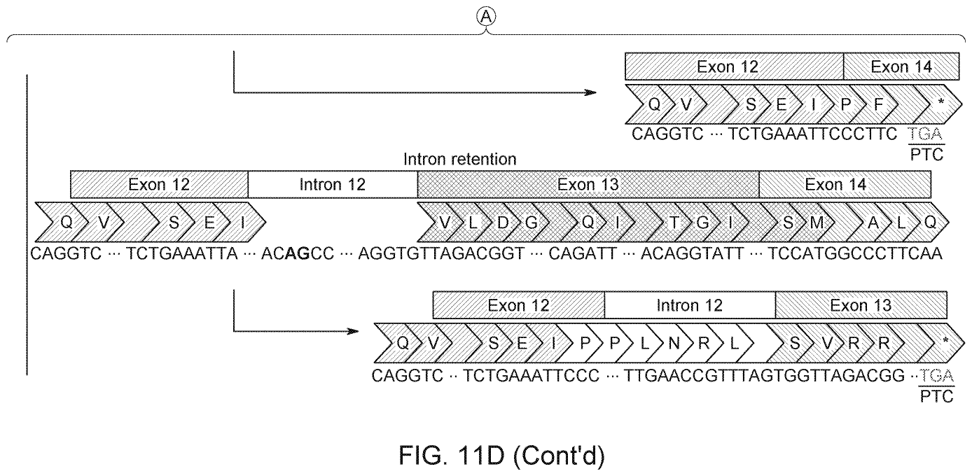

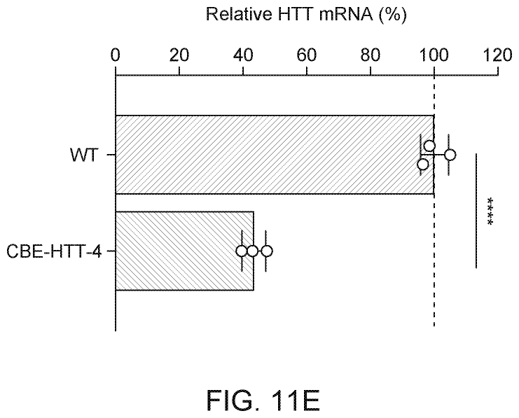

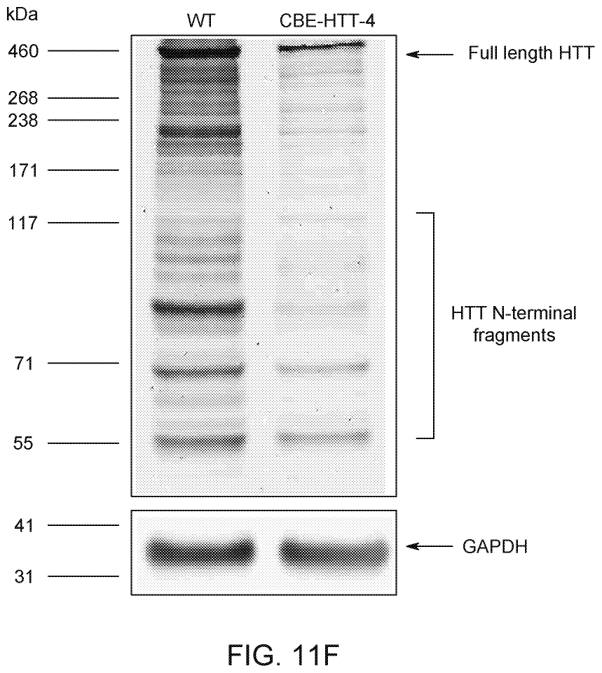

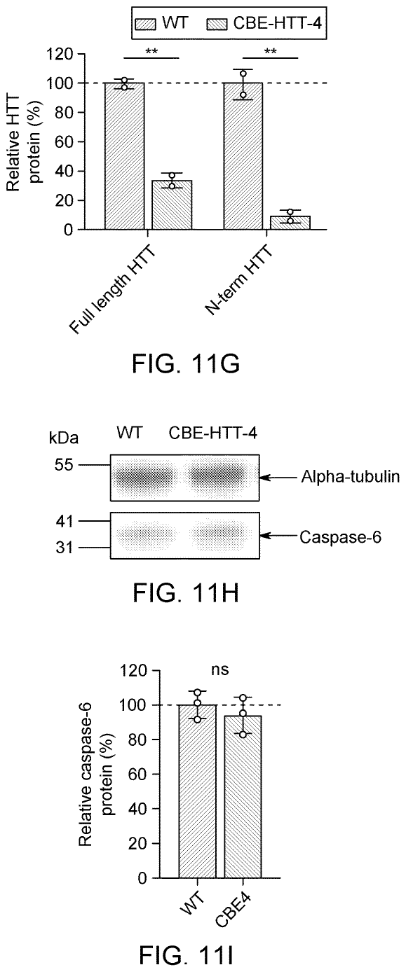

Comparisons of CBEs revealed different editing profiles, but we did not observe any significant difference in mean editing rates among the groups (p>0.05, FIG. 1H, I). The most efficient CBEs were TadCBEd, which successfully edited 75% of the targeted SAs (mean editing rate ~32%) followed by CBE4max (50%, mean editing rate ~40%), evoFERNY (50%, mean editing rate ~26%) and RrA3F (F130L) (50%, mean editing rate ~29%). SsAPOBEC3B (R54Q) and PpAPOBEC1 (H122A) only edited 1 and 2 target sites respectively at rates lower than ~22% (FIG. 1H, I). Interestingly, TadCBE and RrA3F (F130L) enable editing of target sequences that were resilient to modification by every other BE system tested (FIG. 11).

Thus, fusion of SpRY Cas9 with different deaminases significantly increased the number of exons that can be targeted with high efficiency.

### Example 3: Simultaneous Editing of SA and SD Sites Enhances Exon-Skipping

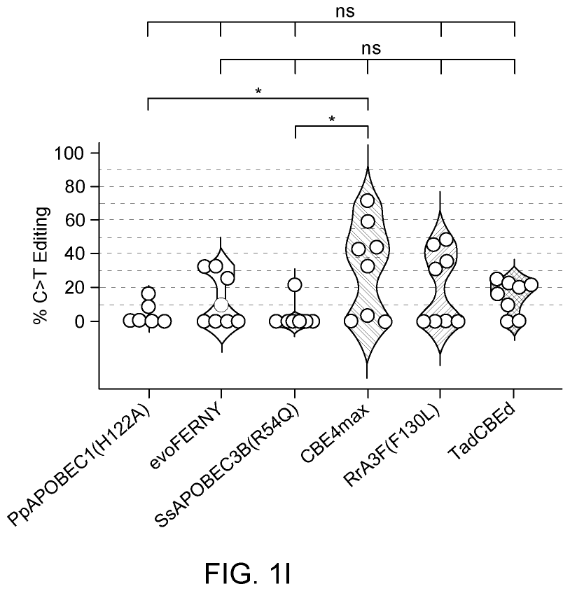

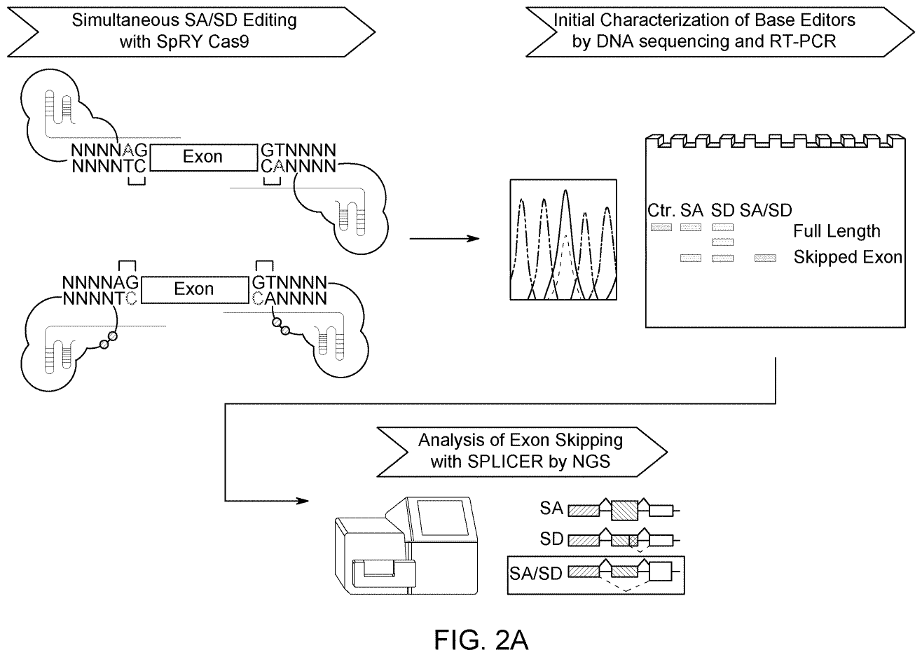

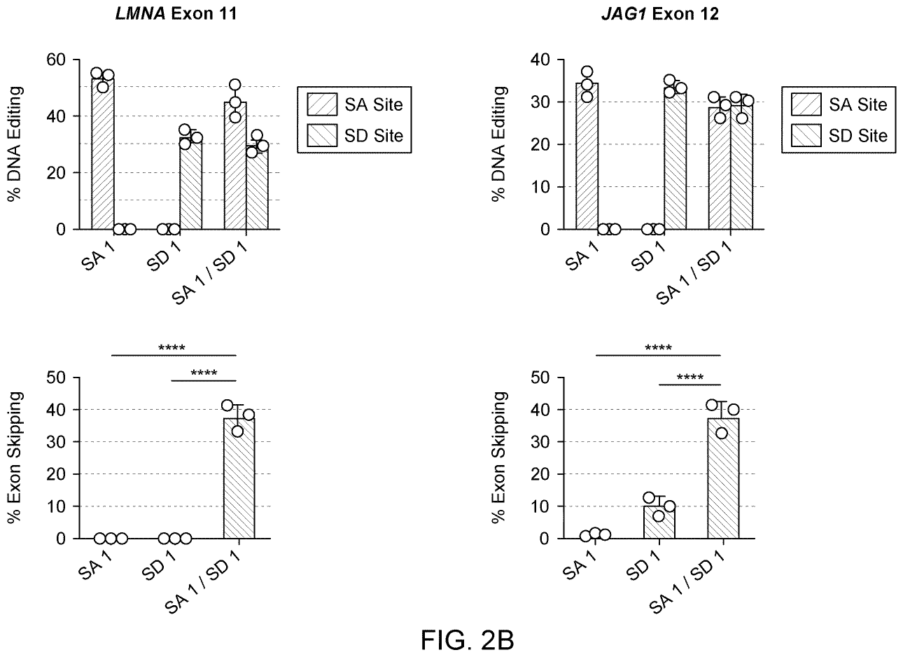

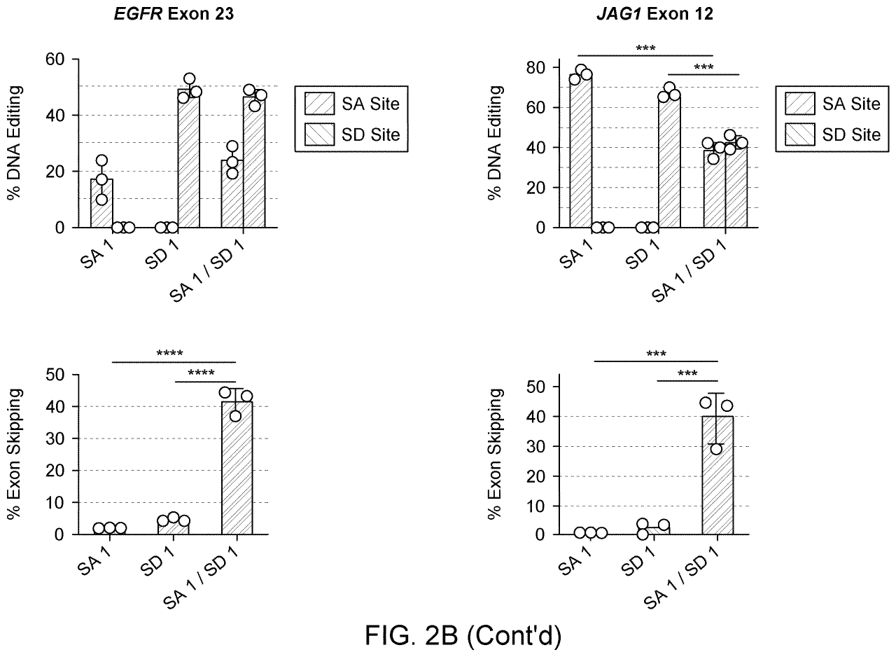

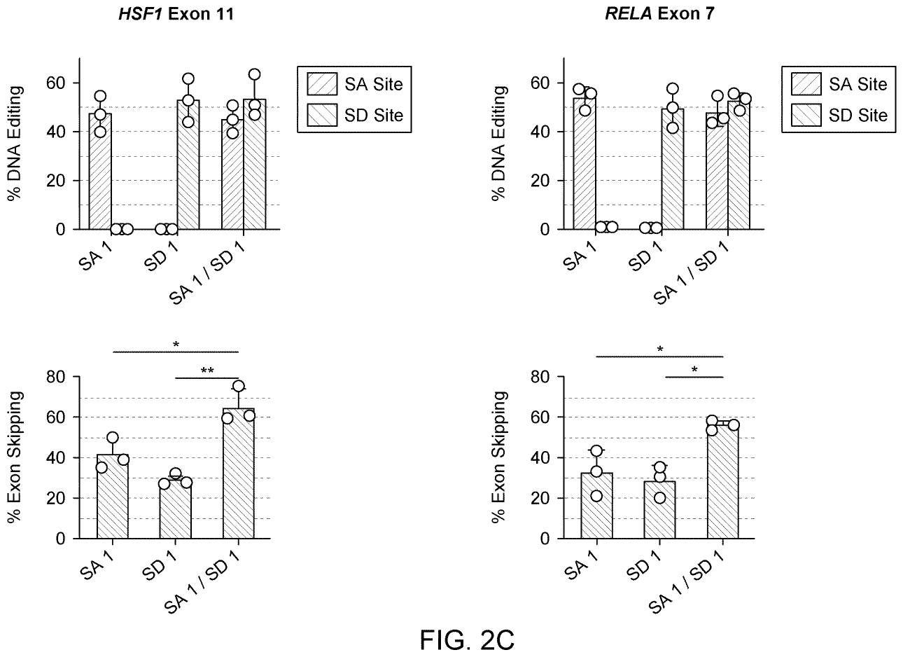

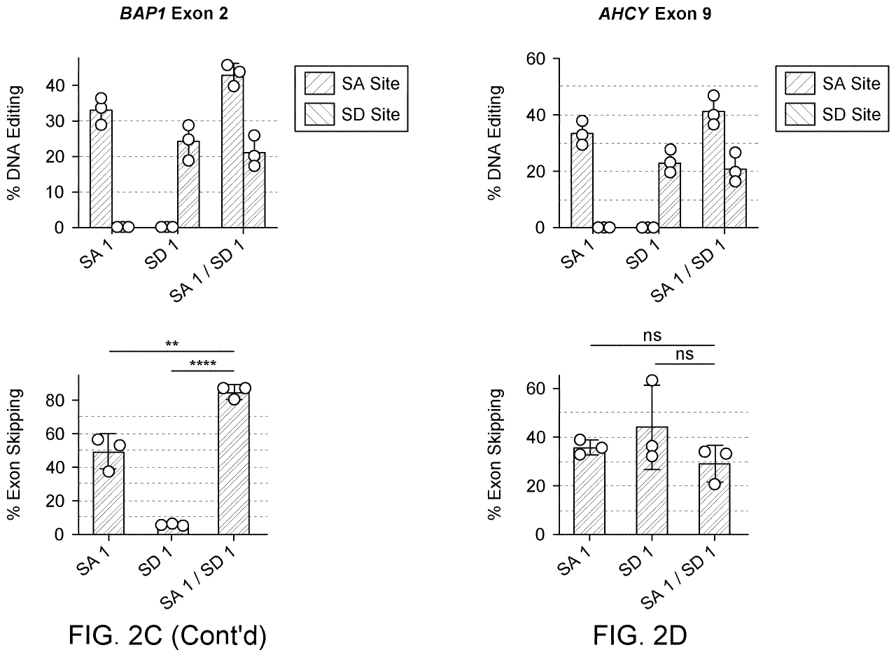

While there are different parameters that can influence exon splicing, a simplistic model of exon skipping would suggest that exon skipping rates are the result of the equilibrium between the DNA editing rate that disrupts different splicing elements favoring skipping and the persistence of non-edited functional splicing elements that favors exon inclusion, such as splicing enhancers or cryptic splicing elements. Thus, we tested whether exon skipping could be enhanced by simultaneously targeting multiple splicing elements, such as SA and SD sites (FIG. 2A), to shift the equilibrium further towards exon skipping.

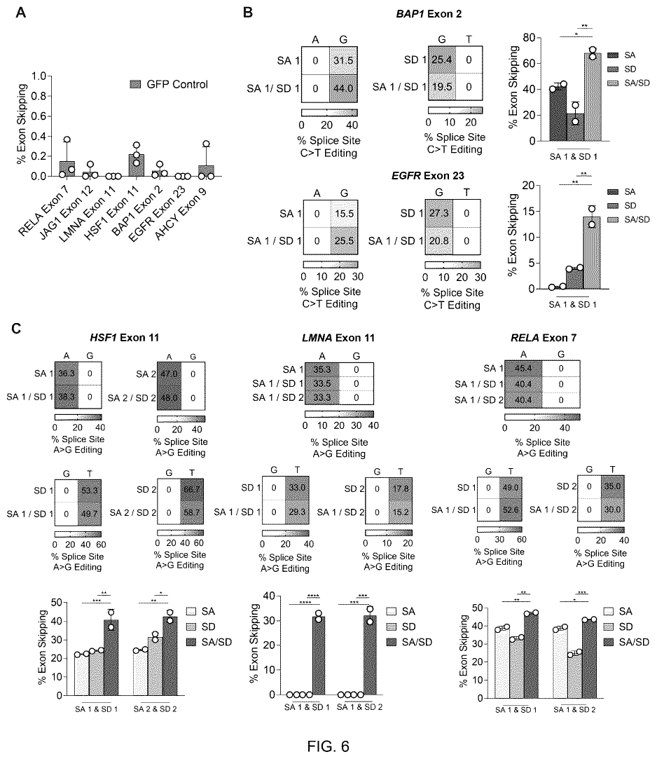

We targeted the SDs and SAs, referred to as dual splice site targeting, of 4 exons with SpRY Cas9-ABE8e or SpRY Cas9-CBE4max and performed high-throughput DNA sequencing to analyze splicing induced by selected sgRNA pairs (FIG. 2B, C, D). At steady state, we did not detect alternative splicing events at any of the targeted exons in control samples (FIG. 6A). Following treatment with ABEs, we observed that dual targeting improved exon skipping rates across all targets for multiple sets of sgRNAs (FIG. 2B, FIG. 6B, 6C), with effects ranging from synergistic (FIG. 2B) to additive (FIG. 2C) in comparison with targeting just the SA or just the SD. For example, when targeting HSF1, exon skipping improved from 40%, which was achieved by targeting the SD alone, to 58% when both splice sites were targeted (p=0.013, FIG. 2C). When targeting RELA exon 7 the improvement in exon skipping was additive, increasing from 32.6% and 25.3% skipping when targeting the individual SA and SD sites, respectively, to 56% when targeted simultaneously (SA/SD vs SA, p=0.031, SA/SD vs SD, p=0.014). Moreover, in other scenarios such as targeting exon 12 of JAG1, exon skipping improved from 0% and 9% when targeting the individual SA or SD sites, respectively, to 37% when both sites were targeted simultaneously (SA/SD vs SA, p<0.0001, SA/SD vs SD, p<0.0001, FIG. 2B), enhancing skipping synergistically. In some cases, exon skipping was accomplished despite limited skipping when targeting the individual single splicing sequences, such as for LMNA exon 11, where skipping increased from undetectable to 37% when editing both splice sites (SA/SD vs SA, p=0.0001, SA/SD vs SD, p<0.0001, FIG. 2B).

Dual splice site targeting of SAs and SDs with CBEs improved skipping in 75% of all targeted exons, with the improvements found to vary in a similar manner to the ABEs. Like for the ABEs, additive and synergistic trends in exon skipping were observed for multiple sets of sgRNAs targeting a specific exon (FIG. 6B, 6C). For example, the increase in exon skipping was additive for BAP1 exon 2 (SA/SD vs SA, p=0.0011, SA/SD vs SD, p<0.0001, FIG. 2C), while exon skipping with CBEs was synergistic for both EGFR exon 23 (SA/SD vs SA, p<0.0001, SA/SD vs SD, p<0.0001) and JAG1 exon 12 (SA/SD vs SA, p=0.0003, SA/SD vs SD, p=0.0004, FIG. 2B). Interestingly, when targeting JAG1 exon 12, exon skipping increased ~10-fold despite a decreased in DNA editing rates at the SA and SD sites by ~50% when simultaneously targeting both the SA and SD (FIG. 2B), which highlights the large impact that dual targeting of splice sites can have on exon skipping even when only relative low DNA editing is accomplished. Finally, for AHCY exon 9, simultaneous editing did not improve exon skipping which was ~28% (SA/SD vs SA, p=0.654, SA/SD vs SD, p=0.233, FIG. 2D), despite editing the SA and SD sites at efficiencies of ~44% and ~20% respectively.

Except for targeting of JAG1 exon 12 with CBEs, when comparing single and dual splice site editing scenarios, genomic DNA editing at the splice sites was not significantly different (p>0.05), indicating that the improvement in exon skipping was not due to higher editing rates but likely the effect of disrupting both SAs and SDs.

These results highlight that simultaneous targeting of multiple splicing sequences with SPLICER enhances full exon skipping outcomes. Further, they also suggest that one mechanism for the improvement of exon skipping rates is the suppression of aberrant splicing outcomes.

### Example 4: SPLICER Lowers Cryptic Splicing and Intron Retention

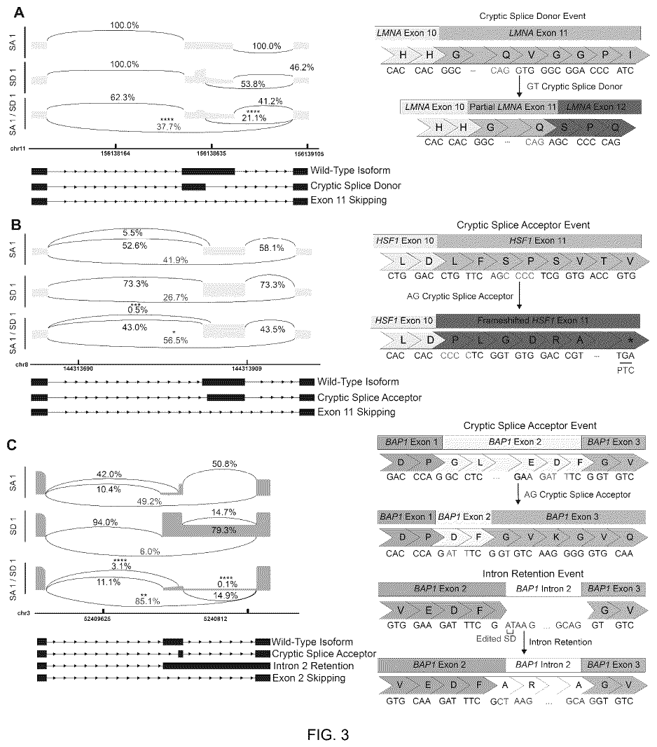

One problem frequently observed when inducing exon skipping is the presence of splicing aberrations, such as cryptic splicing and intron retention. Cryptic splicing occurs when the spliceosome machinery utilizes sites that closely resemble the canonical SA or SD sequences, resulting in a transcript in which the target exon is not fully skipped. Similarly, intron retention can occur when disruption of a SA or SD does not induce full exon skipping; instead, part of an intron or the entire intron is incorporated into the mature mRNA. While events such as these are complex and not fully understood, we and others have observed that ‘AG’ and ‘GT’ bases near the consensus SD and SAs are frequently interpreted by the spliceosome machinery as cryptic SAs or SDs respectively (FIG. 3).

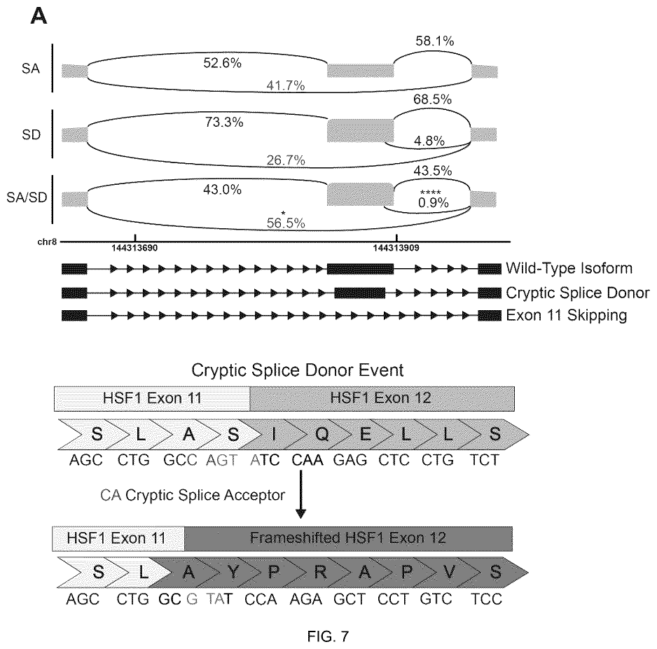

Since editing of both SA and SD sites increased full exon skipping, we hypothesized that one reason for the increase in full-length exon splicing could be a decrease in the rate of cryptic splicing. To test this hypothesis, we targeted the SA, the SD, or both in exon 11 of LMNA, where editing a single splicing sequence has been shown to induce cryptic splicing (FIG. 3A). While targeting the SA of LMNA exon 11 failed to induce exon skipping, targeting the SD led to ~53% partial exon 11 skipping, where a ‘GT’ dinucleotide that is normally part of the codon corresponding to residue V607 can be recognized as a cryptic SD (FIG. 3A), leading to partial exon skipping and a new reading frame for LMNA. However, the rate of full exon 11 skipping when targeting both the SD and SA was ~37%, while the rate of partial exon skipping was found to decrease ~3-fold (SA/SD vs SD, p=0.0001). This change in cryptic splicing was also observed at the SA for targets such as HSF1 exon 11 (FIG. 3B). In this instance, a cryptic ‘AG’ sequence present at S419 can be utilized, leading to a frameshifted transcript with a premature termination codon (PTC) within exon 11. We found that this cryptic splicing event was reduced ~10-fold upon simultaneous splice site disruption (SA/SD vs SA, p=0.0005). Additionally, in HSF1 exon 11, there exists a cryptic SD created by a ‘GT’ motif located at the 5′ of the canonical SD, which can be used as a splice-site, leading to a frameshift that extends through exons 12 and 13 and can result in a stop codon after the canonical stop codon in exon 13. When targeting the SD of HSF1 exon 11, the cryptic SD event was recognized in ~4.8% of cases. Further, this cryptic event was reduced by ~4.9-fold to 0.97% for dual splice site editing (p<0.0001, FIG. 7A). Similarly, when targeting the SA of BAP1 exon 2, the full exon skipping rate was measured to be ~49%, while ~42% of the transcripts were found to contain a partial exon 2 resulting from a cryptic SA (FIG. 3C). This cryptic splicing event leads to a transcript containing a truncated exon 2, although the reading frame was preserved. When targeting the SD, full exon skipping was only detected in ~6% of the transcripts, while ~79% of the transcripts retained intron 2 fully. Both aberrations were minimized by targeting both SD and SA sequences, which decreased cryptic splicing to ~3% (SA/SD vs SA, p=0.0003,), reduced intron retention to <1% (SA/SD vs SD, p<0.0001), and provided ~85% full length exon skipping. These results support that SPLICER improves full exon skipping by dual splice site DNA editing through the reduction of cryptic splicing events.

### Example 5: SPLICER Reduces In Vitro Aβ42 Expression and Skips APP Exon 17 In Vivo

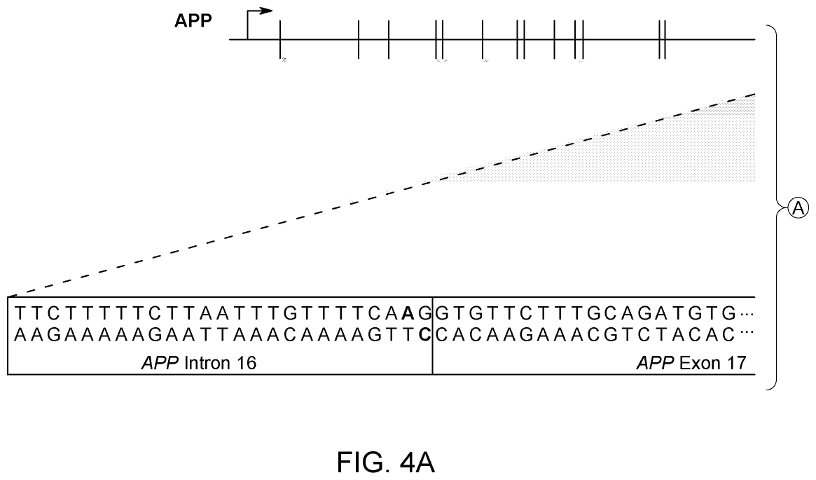

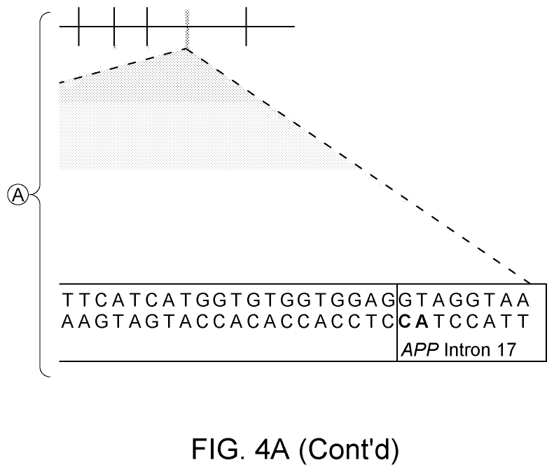

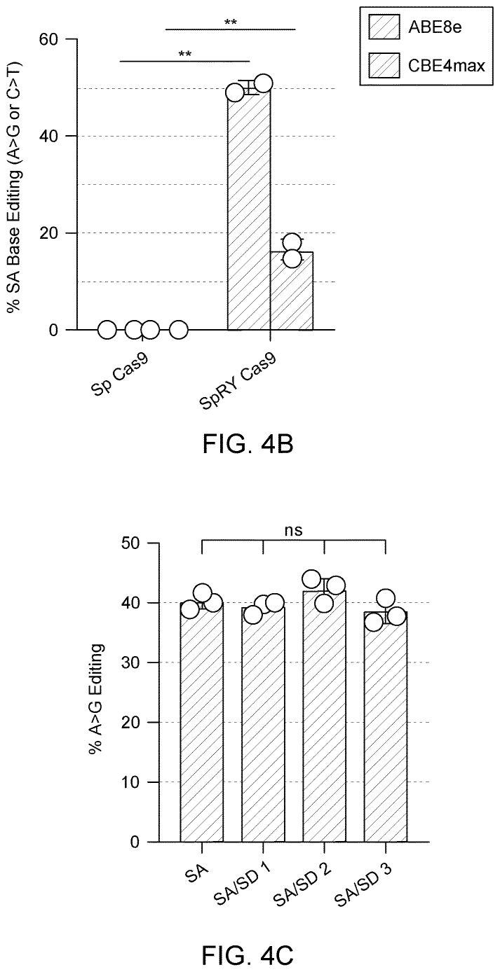

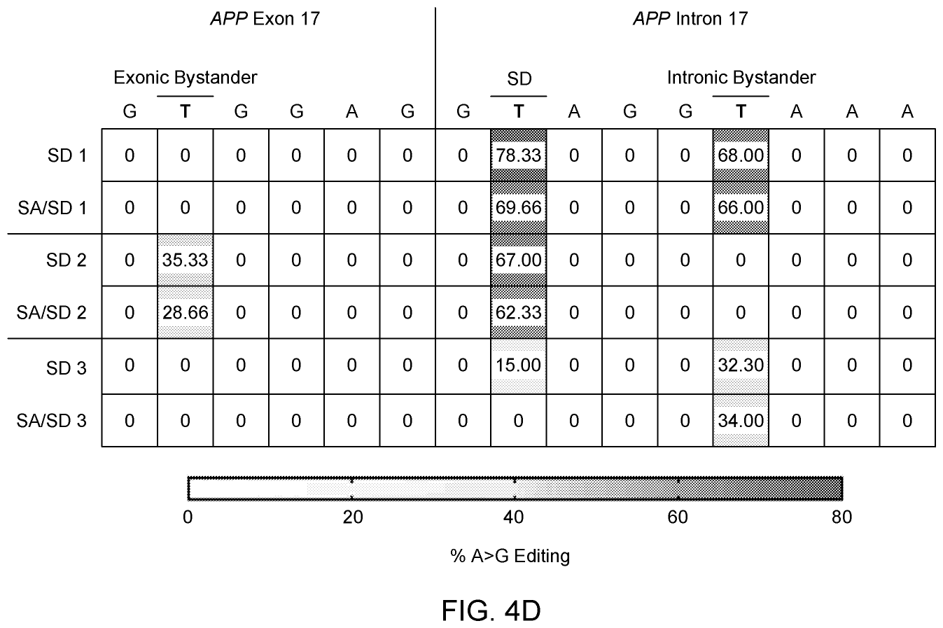

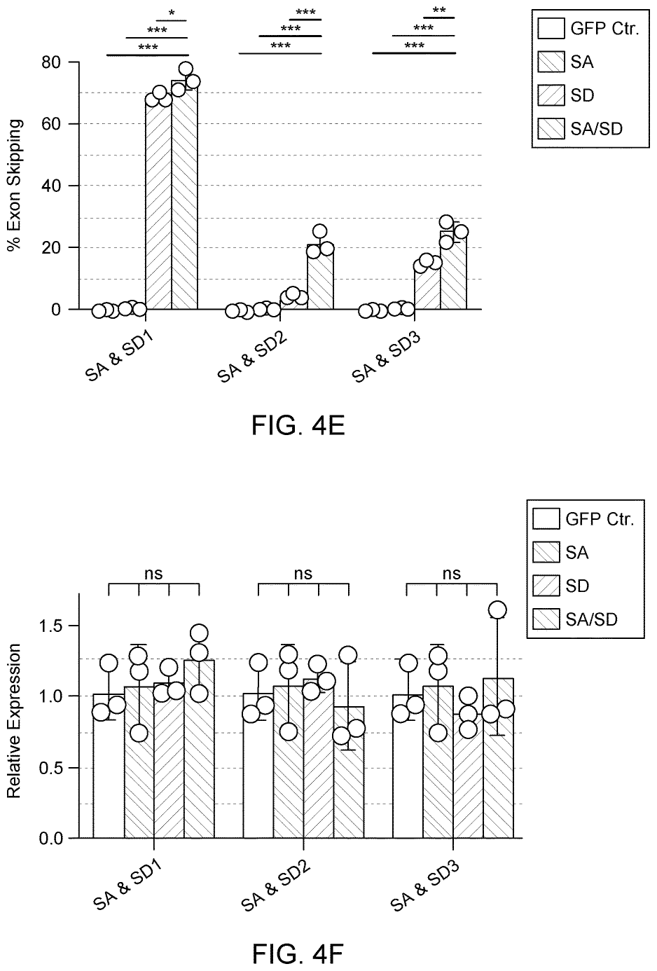

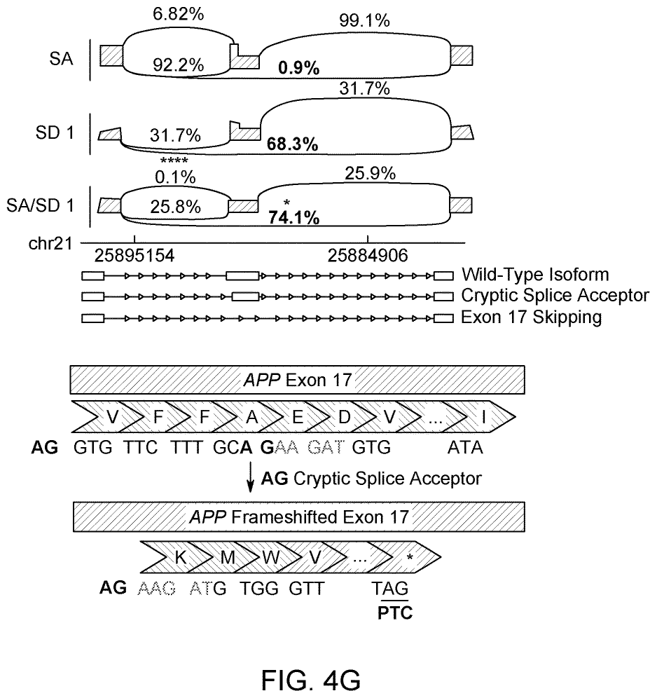

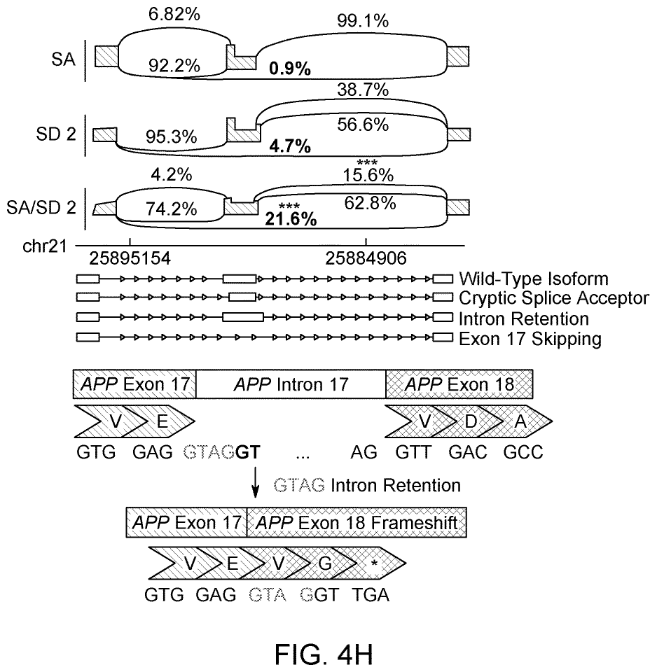

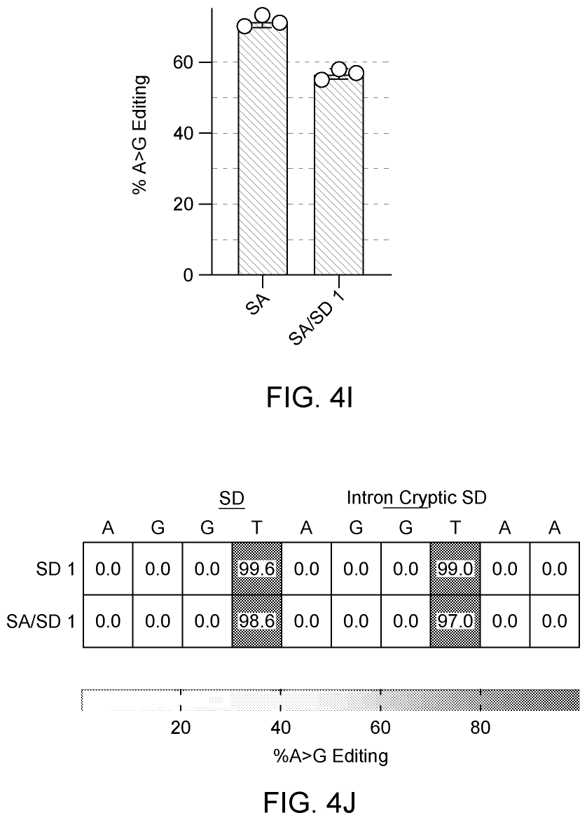

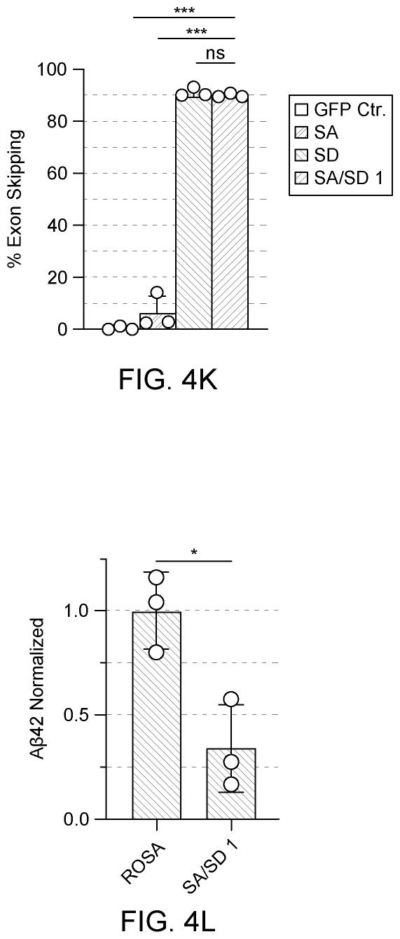

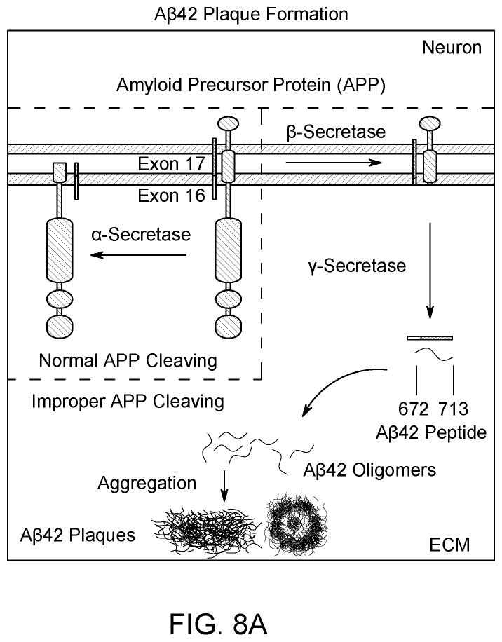

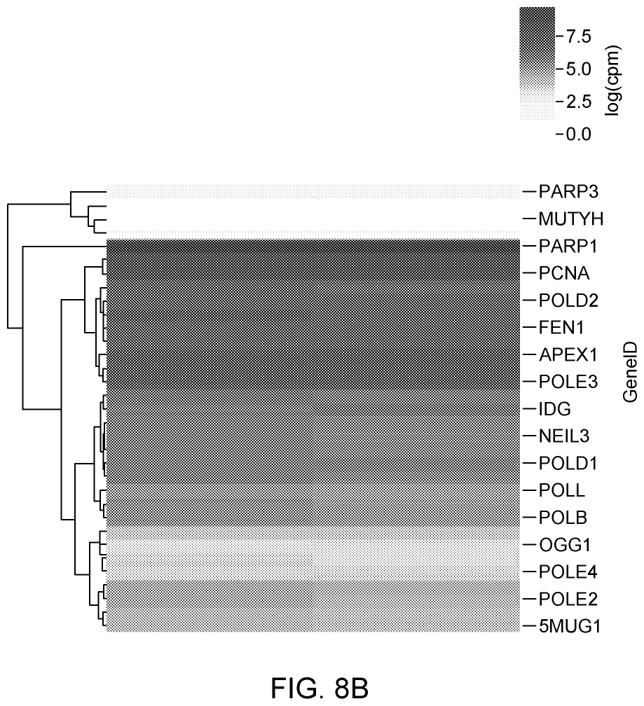

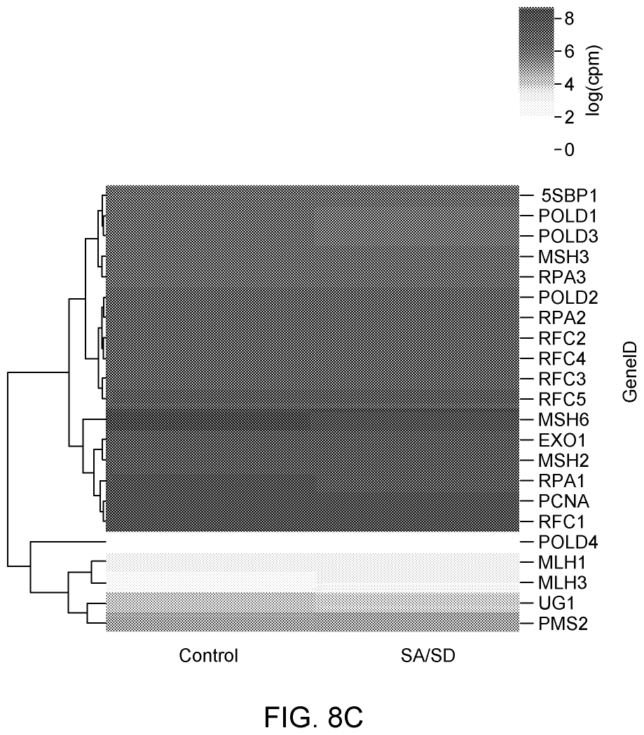

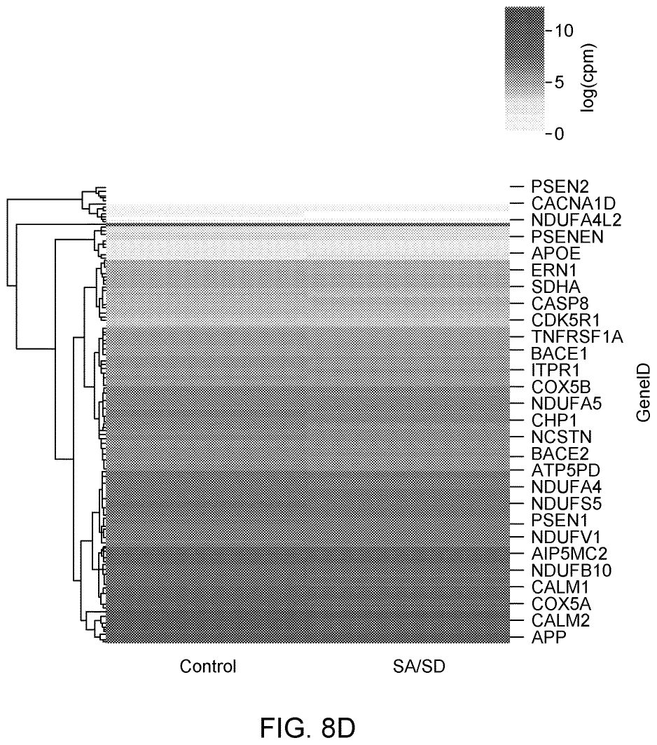

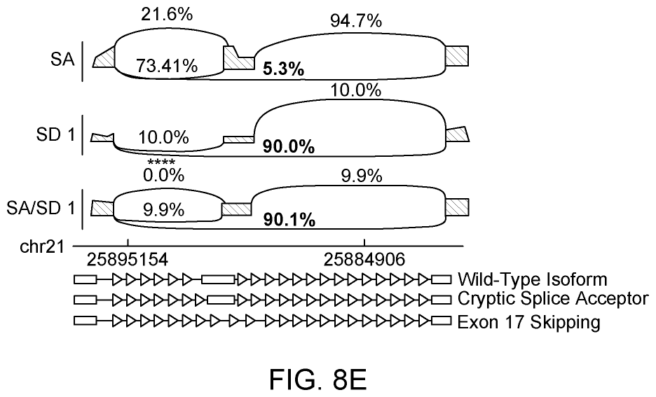

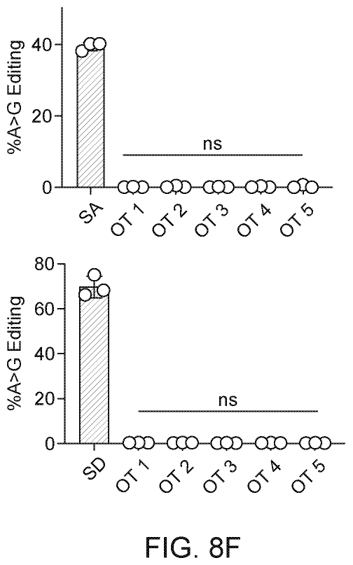

Given the efficiency of exon skipping accomplished with SPLICER, we next sought to determine whether it could be utilized to skip exons with therapeutic value, such as exon 17 in the APP gene which, in our experience, was resilient to skipping with CRISPR-SKIP. APP is located in chromosome 21 and encodes the amyloid precursor protein (FIG. 4A). APP digestion by β- and γ-secretase generates the Aβ42 peptide, which is hypothesized to contribute to the pathogenesis of Alzheimer's disease (AD) (FIG. 8A). Importantly, this cleavage site is encoded within exon 17, an exon whose skipping can reduce the formation of Aβ42 plaques. While previous attempts at editing the SA of APP exon 17 with SpCas9-derived BEs had failed in our laboratory, we found that the PAM flexibility afforded by SpRY Cas9 enabled efficient editing of the SA with both ABE8e (50%) and a CBE4max (16%) in HEK293T cells (FIG. 4B-C). We also utilized SpRY Cas9 to target the SD of APP exon 17, identifying three sgRNAs that edited it at efficiencies of 78%, 67%, and 16% (FIG. 4C-D).

Despite the high rates of editing observed at the SA site for APP exon 17, we observed limited skipping (~0.9%) when targeting the SA alone (FIG. 4E). However, when the SD was targeted with each of the 3 different sgRNAs described above, we observed exon skipping (FIG. 4D-E). In addition to editing at the SD, we identified two potential cryptic ‘GT’ splice donors that were edited, and their editing appeared to influence exon skipping (FIG. 4D-E). While editing at the canonical SDs for sgRNAs SD 1 and SD 2 were similar, sgRNA SD1 edited only the intronic cryptic SD while sgRNA SD2 edited only the exonic cryptic SD. The exon skipping rates were vastly different for sgRNAs SD 1 and 2, 69% and 6.5% respectively, demonstrating that the intronic cryptic SD plays a critical role in APP exon 17 splicing. Interestingly, when utilizing dual splice site editing with sgRNAs SA and SD 3 (FIG. 4D-E), there was editing at the cryptic intronic SD but not at the canonical SD, yet exon skipping was improved when editing the SA and the intronic ‘GT’ sequence. Furthermore, simultaneously targeting the SA and SD sites improved exon skipping rates for all sgRNA pairs tested, ranging from a slight increase to a synergistic improvement (69% to 75% for SA/SD 1, 6.5% to 24% for SA/SD 2, 15% to 27% for SA/SD 3, p<0.05 for all) (FIG. 4E). Importantly, splice site editing did not significantly (p>0.05) affect APP mRNA expression measured by qPCR, which is critical as APP has essential roles in multiple signaling pathways. (FIG. 4F). Notably, when characterizing the precision of the splicing events following editing of APP exon 17, we observed that simultaneous targeting of both the SA and SD again reduced cryptic splicing events (FIG. 4G-H). For example, when the SA was targeted individually, its modification caused a cryptic ‘AG’ motif located between V692 and E693 to be utilized for splicing, leading to a premature termination codon within exon 17 in ~7% of transcripts, an outcome that was almost completely abolished with dual splice site-editing (SA/SD 1 vs SA, p<0.0001). When editing the canonical SD with the sgRNA SD 2, a partial intron retention event, whereby the cryptic intronic SD functioned as a canonical SD, was found to occur in 39% of transcripts, further showing the importance of this intronic ‘GT’ site. This aberrant splicing was reduced by over 50% with simultaneous editing of SD and SA (SA/SD 2 vs SD 2, p=0.0004), which is critical as this cryptic splicing event preserves the protease cleavage sites for the β- and γ-secretases and, therefore, its presence is unlikely to reduce Aβ42. SA/SD 1 achieved the highest overall exon skipping rates and was thus used in subsequent experiments.

To determine if our dual-splice site editing platform influenced overall RNA expression on a transcriptome-wide scale, we performed RNA-seq and differential gene expression (DGE) analysis following targeting of APP exon 17. This analysis revealed differential expression of only 3 genes, HSPA6, ZCCHCH12, and RPL21P120 (Benjamini-Hochberg adjusted p<0.05, FIG. 4I). These genes were all upregulated, have no association with DNA damage repair or mismatch repair but have been linked to late-onset AD. No genes were found to be downregulated. Additionally, we analyzed gene expression pathways associated with mismatch repair (KEGG pathway hsa03430) and base excision repair (KEGG pathway hsa03410) and observed no changes in pathway expression for any genes when compared to control cells (DEG defined as a gene with a Benjamini-Hochberg adjusted p>0.05 for upregulated genes, FIG. 4 J, 8B,C), indicating SPLICER did not induce DNA damage repair pathways nor misregulate genes different from the intended target. When analyzing the transcriptomic profile for genes linked with AD (KEGG pathway hsa05010), we found no change in expression associated with pathways implicated in the pathogenesis of AD, including APP (DEG defined as a gene with a Benjamini-Hochberg adjusted p>0.05 for upregulated genes, FIG. 41-I, 8D), further demonstrating that simultaneous SA/SD targeting does not induce misregulation of gene expression.

Next, to evaluate the effect of dual-splice site editing on the formation of Aβ42, we targeted APP exon 17 in BE(2)-M17 cells, which are commonly used to model aspects of AD as they produce high levels of the Aβ42 peptide. Accordingly, we first sought to determine if APP could be effectively edited in BE(2)-M17 cells with the lead editing guide candidate identified in the experiments above. Following puromycin enrichment of transfected cells, DNA editing at the SA was ~60%, while the cryptic intron SD and canonical SD were both edited at ~100% (FIG. 4K-L) and exon skipping when simultaneously targeting the SA and SD sites was ~90%. When editing the SA alone, a cryptic ‘AG’ splice acceptor motif was recognized between V692 and E693, like in HEK293T cells, at a rate of 21%. This cryptic splicing was reduced to <1% with simultaneous editing (p<0.0001, FIG. 8E). No other alternative splicing was observed (FIG. 8E). We then performed ELISA to quantify expression of Aβ42, measuring a ~70% decrease in Aβ42 peptide following simultaneous SA/SD editing (SA/SD vs ROSA, p=0.015). Finally, an analysis of editing at potential off-target sites predicted by Cas-OFFinder revealed no appreciable A>G editing at any computationally predicted sites (p>0.05, FIG. 8F). These results demonstrate that SPLICER can enable APP exon 17 skipping and reduce expression of Aβ42.

To provide further validation of the potential of SPLICER, we sought to induce skipping of APP exon 17 in a mouse model of AD. We performed these experiments in APP^{K670N/M671L} mice, which harbor a yeast artificial chromosome expressing the full human APP gene, including introns, which are not typically included in other mouse strains. To enable delivery by AAV, we used a split-intein BE system as the size of a full-length BE exceeds the ~4.7 kb packaging capacity of the AAV vector genome. In this system, the base editor is split into two vectors at Cas9 amino acid residue 712 and the resulting N-terminus and C-terminus Cas9 domains are fused with the N and C Rhodothermus marinus inteins, respectively. Upon expression, the inteins dimerize and self-excise, which results in reconstitution of the full length BEs.

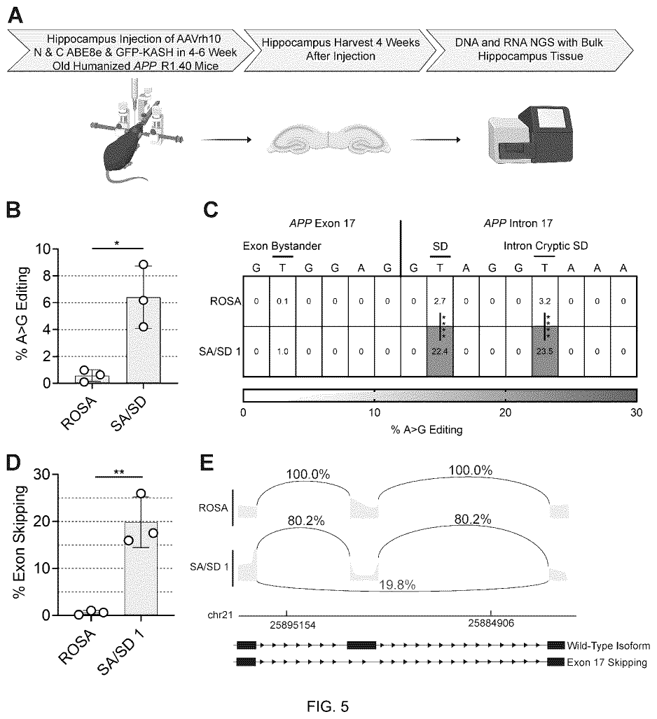

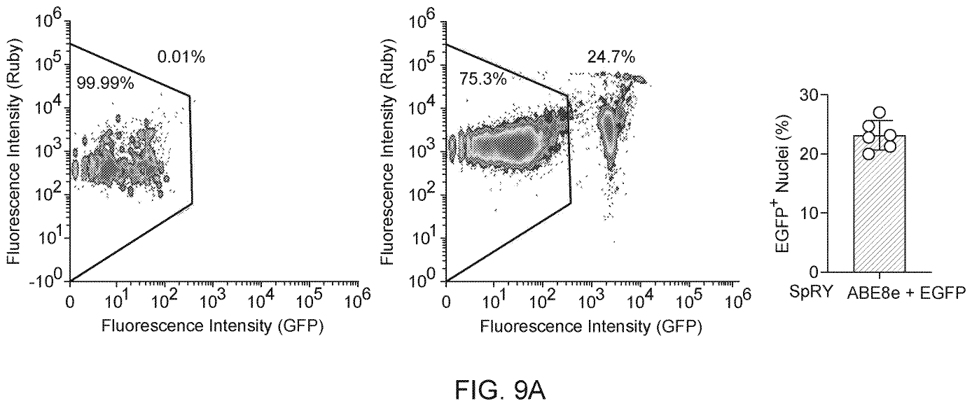

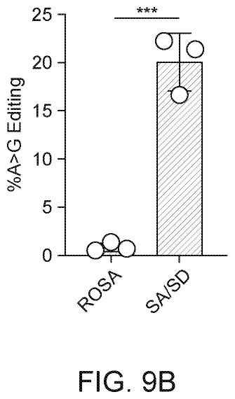

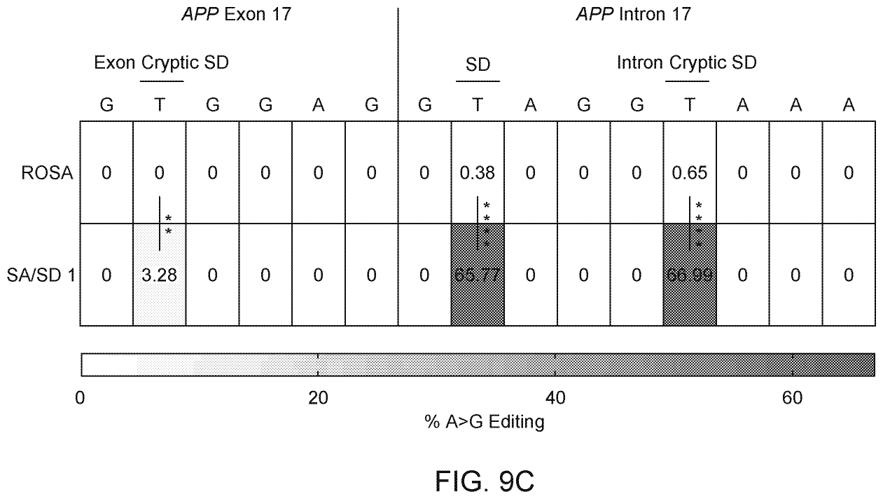

We delivered BE-encoding and EGFP-KASH encoding AAV to four-to seven week-old mice via a stereotaxic injection to the hippocampus, an area of the brain typically affected in AD by the formation of Aβ42 plaques, with AAVrh10, a serotype that has efficiently transduces the hippocampus (FIG. 5A). Expression of BEs and EGFP-KASH were driven by the CAG promoter. At one month after injection, the transduction efficiency measured by fluorescence activated cell sorting (FACS) in harvested hippocampus tissue was ~23.6% (FIG. 9A). In bulk tissue, our deep sequencing analysis revealed DNA editing rates of 6.4% for the SA (FIG. 5B), 22.4% for the SD, and 23.4% for the intron cryptic SD (FIG. 5C), which are consistent with our findings in cultured cells considering our transduction efficiency. Notably, this editing was ~3-fold higher in FACS sorted nuclei (FIG. 9B-C). Importantly, we observed ~20% skipping of APP exon 17 in bulk hippocampus tissue (SA/SD vs ROSA, p=0.003, unpaired t-test) (FIG. 5D). Our deep sequencing analysis further revealed that there was no aberrant cryptic splicing resulting from SA/SD DNA editing, indicating clean, full-length exon 17 skipping achieved with SPLICER editing in vivo (FIG. 5E).

In conclusion, we developed SPLICER, a method that improves full-length exon skipping by Cas base editors. We further demonstrate the therapeutic potential of SPLICER by targeting a high-value target for AD in a mouse model of the neurodegenerative disorder.

### Example 6: Discussion

In this work we developed SPLICER, a platform for exon skipping with base editors that overcomes the limitations of previously described technologies. This platform utilizes base editors with relaxed PAM requirements fused with different deaminases that, when targeted to both SD and SAs simultaneously, increase exon skipping efficiency and decrease splicing aberrations.

Potential applications of SPLICER include therapeutic exon skipping to restore protein expression, disruption of splicing sequences to interrogate and dissect exon splicing, gene knock out by altering the reading frame through exon skipping or massively parallel loss-of-function screens, which also take advantage of gene knock out by shifting the reading frame.

SPLICER provides significant advantages over other methods for exon skipping such as ASOs, which are currently used as gene therapies for treating Duchene muscular dystrophy or retinitis pigmentosa, because the transient nature and rapid clearance of ASO requires repeated administration and provides only a temporary benefit with limited activity in the periods of time leading up to the new dosage. The permanent nature of the modifications introduced in genomic DNA by base editors is advantageous, although the transient effects of ASOs are reversible, which could be beneficial if adverse effects occur.

Other gene editing technologies, such as traditional CRISPR-Cas9 nucleases or prime editors, can also be used to disrupt splicing sequences. However, as the knowledge about the potential deleterious effects of DSBs increases, it becomes critical to utilize technologies that minimize DSBs and introduce minimal genotoxicity, which can make base editors preferable over nucleases or prime editors.

One limitation of base editors is their capacity to introduce bystander mutations, which could hinder therapeutic applications that require precise correction of a single base mutation. This problem is not critical for exon skipping applications because undesired bystander mutations often occur in intronic sequences where they are unlikely to have a detrimental effect. Further, when they are found in exonic sequences, they are unlikely to have an impact on the protein products, as the exon is skipped.

Guide-dependent and guide-independent off-target effects are important concerns when using base editors. Since SPLICER targets sequences that are conserved in the genome, it is possible that the sgRNAs utilized may be at a higher risk for off-target effects. However, given that only 2 base pairs within SD or SAs are highly conserved, and the guides themselves almost invariably target partly exonic sequences, which are less conserved, we did not observe any off-target mutations at computationally predicted off-target sites for the sgRNAs targeting APP exon 17. Guide-independent off-target effects are derived from the DNA binding capability of the deaminases and occur more frequently when using CBE than ABEs. While newer generations of highly specific CBEs have been developed, we generally utilize ABEs whenever possible, which is advantageous not only because of their specificity but also because of their higher editing activity. Importantly, a CBE was recently developed from ABEs, which has improved specificity and has proven to be effective for inducing exon skipping in our work.

SPLICER was not sufficient to accomplish skipping of all targeted exons, including AHCY exon 9 (FIG. 1). One possibility for this lack of improved exon skipping is the presence of exon splice enhancer (ESE) sequences, which bind to Ser/Arg rich (SR) proteins that recruit spliceosome proteins. We studied the presence of ESE utilizing ESEfinder and determined that, within AHCY exon 9, there are motifs predicted to function as splice enhancers (high affinity to SF2/ASF: score 3.76, SC35: score 3.69, SC35: score 4.14, SRp40: score 3.88, SRP55, score 4.69), which may explain why this particular exon appeared resistant to exon skipping.

Lastly, we show the application of SPLICER for skipping APP exon 17, an approach with potential therapeutic value in Alzheimer's disease. By skipping exon 17, given that the binding site for the secretases is lost, generation of Aβ42 decreases. Previous attempts to skip this exon in our lab had been unsuccessful despite high editing rates at the SA, as a lack of tools for targeting additional splice sites prevented further optimization of the approach. Using SPLICER, we have identified multiple sgRNAs with different editing profiles that have contributed to our understanding of the splicing events governing this transcript. We observed that editing the canonical SD activates a cryptic SD that prevents exon skipping and when simultaneously editing both the canonical SD and cryptic SD sites with SPLICER we were able to effectively induce exon skipping. Additionally, APP exon 17 skipping was further enhanced by the simultaneous disruption of the SA, an event that reduced Aβ42 production in vitro. SPLICER had no effect on APP mRNA levels of expression, as determined by qPCR and RNA-seq, however, RNA-seq did reveal that APP exon 17 disruption affected the expression of the genes HSPA6, ZCCHCH12, and RPL21P120. Interestingly, the expression of these genes has been shown to be modulated in late-onset forms of AD, indicating that the Aβ42 may play a role in regulating expression of these genes, further illustrating the complex role of Aβ42. Critically, SPLICER functions efficiently in vivo for targeting APP exon 17, accomplishing 20% exon skipping in a humanized APP mouse model when delivered by AAVrh10. While the skipping rate in vivo was approximately three-fold lower than the observed in vitro efficiency, when taking into consideration the apparent transduction efficiency, which we measured as ~23.6%, this result emphasizes the potential of the approach.

In summary, we report the development of the SPLICER toolbox, which enables targeting of essentially any exon and improves skipping purity outcomes via simultaneous editing of multiple splicing elements. We demonstrate the in vivo applicability of SPLICER in a humanized mouse model of Alzheimer's disease and anticipate that this technique will enable a range of broad applications.

### REFERENCES

1. Anzalone, A. V., Koblan, L. W. & Liu, D. R. Genome editing with CRISPR-Cas nucleases, base editors, transposases and prime editors. Nature biotechnology 38, 824-844 (2020).
2. Adikusuma, F. et al. Large deletions induced by Cas9 cleavage. Nature 560, E8-E9 (2018).
3. Leibowitz, M. L. et al. Chromothripsis as an on-target consequence of CRISPR-Cas9 genome editing. Nature genetics 53, 895-905 (2021).
4. Haapaniemi, E., Botla, S., Persson, J., Schmierer, B. & Taipale, J. CRISPR-Cas9 genome editing induces a p53-mediated DNA damage response. Nature medicine 24, 927-930 (2018).
5. Tuladhar, R. et al. CRISPR-Cas9-based mutagenesis frequently provokes on-target mRNA misregulation. Nature communications 10, 4056 (2019).
6. Cox, D. B. T., Platt, R. J. & Zhang, F. Therapeutic genome editing: prospects and challenges. Nature medicine 21, 121-131 (2015).
7. Doudna, J. A. The promise and challenge of therapeutic genome editing. Nature 578, 229-236 (2020).
8. Komor, A. C., Kim, Y. B., Packer, M. S., Zuris, J. A. & Liu, D. R. Programmable editing of a target base in genomic DNA without double-stranded DNA cleavage. Nature 533, 420-424 (2016).
9. Gaudelli, N. M. et al. Programmable base editing of A•T to G•C in genomic DNA without DNA cleavage. Nature 551, 464-471 (2017).
10. Gapinske, M. et al. CRISPR-SKIP: programmable gene splicing with single base editors. Genome biology 19, 1-11 (2018).
11. Gapinske, M. et al. Targeting Duchenne Muscular Dystrophy by Skipping DMD Exon 45 with Base Editors. Molecular Therapy-Nucleic Acids (2023).
12. Chai, A. C. et al. Single-swap editing for the correction of common Duchenne muscular dystrophy mutations. Molecular Therapy-Nucleic Acids 32, 522-535 (2023).
13. Yuan, J. et al. Genetic modulation of RNA splicing with a CRISPR-guided cytidine deaminase. Molecular cell 72, 380-394. e7 (2018).
14. Webber, B. R. et al. Highly efficient multiplex human T cell engineering without double-strand breaks using Cas9 base editors. Nature communications 10, 5222 (2019).
15. Kluesner, M. G. et al. CRISPR-Cas9 cytidine and adenosine base editing of splice-sites mediates highly-efficient disruption of proteins in primary and immortalized cells. Nature Communications 12, 2437 (2021).
16. Jinek, M., Chylinski, K., Fonfara, I., Hauer, M., Doudna, J. A. & Charpentier, E. A programmable dual-RNA-guided DNA endonuclease in adaptive bacterial immunity. science 337, 816-821 (2012).
17. Yang, B., Yang, L. & Chen, J. Development and application of base editors. The CRISPR journal 2, 91-104 (2019).
18. Winter, J. et al. Targeted exon skipping with AAV-mediated split adenine base editors. Cell discovery 5, 1-12 (2019).
19. Sibley, C. R., Blazquez, L. & Ule, J. Lessons from non-canonical splicing. Nature Reviews Genetics 17, 407-421 (2016).
20. Liu, Y. et al. Adenine base editor-mediated splicing remodeling activates non-canonical splice sites. Journal of Biological Chemistry, 105442 (2023).
21. Jung, H. et al. Intron retention is a widespread mechanism of tumor-suppressor inactivation. Nature genetics 47, 1242-1248 (2015).
22. Dvinge, H. & Bradley, R. K. Widespread intron retention diversifies most cancer transcriptomes. Genome medicine 7, 1-13 (2015).
23. Hsu, T. Y.-T. et al. The spliceosome is a therapeutic vulnerability in MYC-driven cancer. Nature 525, 384-388 (2015).
24. Julia, T. & Goate, A. M. Genetics of β-amyloid precursor protein in Alzheimer's disease. Cold Spring Harbor perspectives in medicine 7(2017).
25. Chang, J. L. et al. Targeting amyloid-β precursor protein, APP, splicing with antisense oligonucleotides reduces toxic amyloid-β production. Molecular Therapy 26, 1539-1551 (2018).
26. Scheltens, P. et al. Alzheimer's disease. The Lancet 388, 505-517 (2016).
27. Gouras, G. K., Olsson, T. T. & Hansson, O. β-Amyloid peptides and amyloid plaques in Alzheimer's disease. Neurotherapeutics 12, 3-11 (2015).
28. Karran, E. & De Strooper, B. The amyloid hypothesis in Alzheimer disease: new insights from new therapeutics. Nature Reviews Drug Discovery 21, 306-318 (2022).
29. Duan, Y. et al. Brain-wide Cas9-mediated cleavage of a gene causing familial Alzheimer's disease alleviates amyloid-related pathologies in mice. Nature Biomedical Engineering 6, 168-180 (2022).
30. Hampel, H. et al. The amyloid-β pathway in Alzheimer's disease. Molecularpsychiatry 26, 5481-5503 (2021).
31. Kleinstiver, B. P. et al. Engineered CRISPR-Cas9 nucleases with altered PAM specificities. Nature 523, 481-485 (2015).
32. Hua, K., Tao, X., Yuan, F., Wang, D. & Zhu, J.-K. Precise A-T to G-C base editing in the rice genome. Molecular plant 11, 627-630 (2018).
33. Zhang, C. et al. Expanding the base editing scope to GA and relaxed NG PAM sites by improved xCas9 system. Plant Biotechnology Journal 18, 884 (2020).
34. Hu, J. H. et al. Evolved Cas9 variants with broad PAM compatibility and high DNA specificity. Nature 556, 57-63 (2018).
35. Goldberg, G. W. et al. Engineered dual selection for directed evolution of SpCas9 PAM specificity. Nature communications 12, 349 (2021).
36. Li, J., Xu, R., Qin, R., Liu, X., Kong, F. & Wei, P. Genome editing mediated by SpCas9 variants with broad non-canonical PAM compatibility in plants. Molecular plant 14, 352-360 (2021).
37. Walton, R. T., Christie, K. A., Whittaker, M. N. & Kleinstiver, B. P. Unconstrained genome targeting with near-PAMless engineered CRISPR-Cas9 variants. Science 368, 290-296 (2020).
38. Richter, M. F. et al. Phage-assisted evolution of an adenine base editor with improved Cas domain compatibility and activity. Nature biotechnology 38, 883-891 (2020).
39. Koblan, L. W. et al. Improving cytidine and adenine base editors by expression optimization and ancestral reconstruction. Nature biotechnology 36, 843-846 (2018).
40. Thuronyi, B. W. et al. Continuous evolution of base editors with expanded target compatibility and improved activity. Nature biotechnology 37, 1070-1079 (2019).
41. Yu, Y. et al. Cytosine base editors with minimized unguided DNA and RNA off-target events and high on-target activity. Nature Communications 11, 2052 (2020).
42. Neugebauer, M. E. et al. Evolution of an adenine base editor into a small, efficient cytosine base editor with low off-target activity. Nature biotechnology 41, 673-685 (2023).
43. Gaudelli, N. M. et al. Directed evolution of adenine base editors with increased activity and therapeutic application. Nature biotechnology 38, 892-900 (2020).
44. Chen, L. et al. Engineering a precise adenine base editor with minimal bystander editing. Nature Chemical Biology 19, 101-110 (2023).
45. Scotti, M. M. & Swanson, M. S. RNA mis-splicing in disease. Nature Reviews Genetics 17, 19-32 (2016).
46. Garcia-Blanco, M. A., Baraniak, A. P. & Lasda, E. L. Alternative splicing in disease and therapy. Nature biotechnology 22, 535-546 (2004).
47. Liu, Y. et al. Genome-wide screening for functional long noncoding RNAs in human cells by Cas9 targeting of splice sites. Nature Biotechnology 36, 1203-1210 (2018).
48. Grymove, T., Grodecke, L., Sou6ek, P. & Freiberger, T. SERPING1 exon 3 splicing variants using alternative acceptor splice sites. Molecular immunology 107, 91-96 (2019).
49. Jung, H., Lee, K. S. & Choi, J. K. Comprehensive characterisation of intronic mis-splicing mutations in human cancers. Oncogene 40, 1347-1361 (2021).
50. Li, R. et al. Amyloid p peptide load is correlated with increased β-secretase activity in sporadic Alzheimer's disease patients. Proceedings of the National Academy of Sciences 101, 3632-3637 (2004).
51. Zhang, Y.-w., Thompson, R., Zhang, H. & Xu, H. APP processing in Alzheimer's disease. Molecular brain 4, 1-13 (2011).
52. Law, C. W., Chen, Y., Shi, W. & Smyth, G. K. voom: Precision weights unlock linear model analysis tools for RNA-seq read counts. Genome biology 15, 1-17 (2014).
53. Shippy, D. C. & Ulland, T. K. Exploring the zinc-related transcriptional landscape in Alzheimer's disease. IBRO Neuroscience Reports 13, 31-37 (2022).
54. Annese, A. et al. Whole transcriptome profiling of Late-Onset Alzheimer's Disease patients provides insights into the molecular changes involved in the disease. Scientific reports 8, 4282 (2018).
55. Macias, M. P. et al. A cellular model of amyloid precursor protein processing and amyloid-β peptide production. Journal of Neuroscience Methods 223,114-122 (2014).
56. Bae, S., Park, J. & Kim, J.-S. Cas-OFFinder: a fast and versatile algorithm that searches for potential off-target sites of Cas9 RNA-guided endonucleases. Bioinformatics 30, 1473-1475 (2014).
57. Lamb, B. T. et al. Altered Metabolism of Familial Alzheimer's Disease-Linked Amyloid Precursor Protein Variants in Yeast Artificial Chromosome Transgenic Mice. Human Molecular Genetics 6, 1535-1541 (1997).
58. Kulnane, L. S. & Lamb, B. T. Neuropathological characterization of mutant amyloid precursor protein yeast artificial chromosome transgenic mice. Neurobiology of disease 8, 982-992 (2001).
59. Yokoyama, M., Kobayashi, H., Tatsumi, L. & Tomita, T. Mouse models of Alzheimer's disease. Frontiers in Molecular Neuroscience 15, 912995 (2022).
60. Lim, C. K. et al. Treatment of a mouse model of ALS by in vivo base editing. Molecular Therapy 28, 1177-1189 (2020).
61. Levy, J. M. et al. Cytosine and adenine base editing of the brain, liver, retina, heart and skeletal muscle of mice via adeno-associated viruses. Nature biomedical engineering 4, 97-110 (2020).
62. Cearley, C. N. & Wolfe, J. H. Transduction characteristics of adeno-associated virus vectors expressing cap serotypes 7, 8, 9, and Rh10 in the mouse brain. Molecular therapy 13, 528-537 (2006).
63. Thal, D. R., Capetillo-Zarate, E., Del Tredici, K. & Braak, H. The development of amyloid p protein deposits in the aged brain. Science of aging knowledge environment 2006, rel-rel (2006).
64. Gapinske, M. et al. Targeting Duchenne muscular dystrophy by skipping DMD exon 45 with base editors. Molecular Therapy-Nucleic Acids 33, 572-586 (2023).
65. Li, J. et al. Therapeutic exon skipping through a CRISPR-guided cytidine deaminase rescues dystrophic cardiomyopathy in vivo. Circulation 144, 1760-1776 (2021).
66. Kim, Y. J. et al. Exon-skipping antisense oligonucleotides for cystic fibrosis therapy. Proceedings of the National Academy of Sciences 119, e2114858118 (2022).
67. Sud, R., Geller, E. T. & Schellenberg, G. D. Antisense-mediated exon skipping decreases tau protein expression: a potential therapy for tauopathies. Molecular Therapy-Nucleic Acids 3(2014).
68. Yamamura, T. et al. Development of an exon skipping therapy for X-linked Alport syndrome with truncating variants in COL4A5. Nature communications 11, 2777 (2020).
69. Zhu, X.-x. et al. Adenine base-editing-mediated exon skipping induces gene knockout in cultured pig cells. Biotechnology Letters, 1-18 (2022).
70. Liu, J.-q. & Li, T. CRISPR-Cas9-mediated loss-of-function screens. Frontiers in Life Science 12, 1-13 (2019).
71. Aartsma-Rus, A. The future of exon skipping for Duchenne muscular dystrophy. Human Gene Therapy 34, 372-378 (2023).
72. Kole, R. & Krieg, A. M. Exon skipping therapy for Duchenne muscular dystrophy. Advanced drug delivery reviews 87, 104-107 (2015).
73. Xue, K. & MacLaren, R. E. Antisense oligonucleotide therapeutics in clinical trials for the treatment of inherited retinal diseases. Expert Opinion on Investigational Drugs 29, 1163-1170 (2020).
74. Dulla, K. et al. Antisense oligonucleotide-based treatment of retinitis pigmentosa caused by USH2A exon 13 mutations. Molecular Therapy 29, 2441-2455 (2021).
75. Gee, P. et al. Extracellular nanovesicles for packaging of CRISPR-Cas9 protein and sgRNA to induce therapeutic exon skipping. Nature communications 11, 1334 (2020).
76. Kenjo, E. et al. Low immunogenicity of LNP allows repeated administrations of CRISPR-Cas9 mRNA into skeletal muscle in mice. Nature communications 12, 7101 (2021).
77. Chemello, F. et al. Precise correction of Duchenne muscular dystrophy exon deletion mutations by base and prime editing. Science advances 7, eabg4910 (2021).
78. Happi Mbakam, C. et al. Prime editing strategies to mediate exon skipping in DMD gene. Frontiers in Medicine 10, 1128557 (2023).
79. Tao, J., Bauer, D. E. & Chiarle, R. Assessing and advancing the safety of CRISPR-Cas tools: from DNA to RNA editing. Nature Communications 14, 212 (2023).
80. Sharpe, J. J. & Cooper, T. A. Unexpected consequences: exon skipping caused by CRISPR-generated mutations. Genome Biology 18, 1-4 (2017).
81. Jeong, Y. K., Song, B. & Bae, S. Current status and challenges of DNA base editing tools. Molecular Therapy 28, 1938-1952 (2020).
82. Slesarenko, Y. S., Lavrov, A. V. & Smirnikhina, S. A. Off-target effects of base editors: what we know and how we can reduce it. Current Genetics 68, 39-48 (2022).
83. Park, S. & Beal, P. A. Off-target editing by CRISPR-guided DNA base editors. Biochemistry 58, 3727-3734 (2019).
84. Liang, P. et al. Genome-wide profiling of adenine base editor specificity by EndoV-seq. Nature communications 10, 67 (2019).
85. Jin, S. et al. Cytosine, but not adenine, base editors induce genome-wide off-target mutations in rice. Science 364, 292-295 (2019).
86. Zuo, E. et al. Cytosine base editor generates substantial off-target single-nucleotide variants in mouse embryos. Science 364, 289-292 (2019).
87. Blencowe, B. J. Exonic splicing enhancers: mechanism of action, diversity and role in human genetic diseases. Trends in biochemical sciences 25, 106-110 (2000).
88. Cartegni, L., Wang, J., Zhu, Z., Zhang, M. Q. & Krainer, A. R. ESEfinder: a web resource to identify exonic splicing enhancers. Nucleic acids research 31, 3568-3571 (2003).
89. Qiu, H. et al. Efficient exon skipping by base-editor-mediated abrogation of exonic splicing enhancers. Cell Reports 42(2023).
90. Lim, C. K. W. et al. Treatment of a mouse model of ALS by in vivo base editing. Molecular Therapy 28, 1177-1189 (2020).
91. Crosson, S. M., Dib, P., Smith, J. K. & Zolotukhin, S. Helper-free production of laboratory grade AAV and purification by iodixanol density gradient centrifugation. Molecular Therapy-Methods & Clinical Development 10, 1-7 (2018).
92. Livak, K. J. & Schmittgen, T. D. Analysis of relative gene expression data using real-time quantitative PCR and the 2-ΔΔCT method. methods 25, 402-408 (2001).
93. Nott, A., Schlachetzki, J. C., Fixsen, B. R. & Glass, C. K. Nuclei isolation of multiple brain cell types for omics interrogation. Nature protocols 16, 1629-1646 (2021).

### Example 7: HTT Targeting Methods

### Plasmids

Plasmids encoding BE3 (Addgene #73021), xCas9(3.7)-BE3 (Addgene #108380), xCas9(3.7)-ABE7.10 (Addgene #108382), SaKKH-BE3 (Addgene #85170), SpRY-CBE4max (Addgene #139999) and ABE8e (Addgene #138489) were obtained from Addgene. The plasmid encoding ABE7.10 was previously described. To construct SpRY-ABE8e, Gibson assembly was used to insert a gBlock (IDT) encoding the SpRY SpCas9 mutations (D1135L, S1136W, G1218K, E1219Q, N1317R, A1322R, R1333P, R1335Q, and T1337R) into the EcoRV and Agel restriction sites of pABE8e.

To construct the split-intein plasmid pAAV-CAG-N-SpCas9-CBE, Gibson assembly was used to insert a gBlock containing a CAG promoter and the N-terminal domain of the split-intein CBE (i.e., the V5 epitope tag, the rAPOBEC1 domain with a 16 residue linker, residues 1-712 of the SpCas9 protein, and the N-intein from the DnaB protein of R. marinus) with the U6 promoter and sgRNA into the XbaI and NotI restriction sites of pX602 (Addgene #61593) by Gibson Assembly. To construct pAAV-CAG-C-SpCas9-CBE, the C-terminal domain of split-intein CBE (i.e., the C-intein fragment from the DnaB protein, residues 713-1,371 of SpCas9, the uracil glycosylase inhibitor, three repeats of the HA epitope tag, and an SV40 nuclear localization signal [NLS] sequence) with the U6 promoter and sgRNA were inserted into the XbaI and NotI restriction sites of pX602 by Gibson assembly.

To construct the split-intein pAAV-CAG-N-SpRY-ABE8e, Gibson assembly was used to insert a gBlock encoding the N-terminal domain of the split-intein ABE (i.e., the V5 epitope tag, an NLS, the ABE8e deaminase domain and a 32 amino acid linker was inserted into the Agel and SalI restriction sites of the plasmid pAAV-CAG-N-SpCas9-CBE, which was described above. To construct pAAV-CAG-C-SpRY-ABE8e, Gibson assembly was used to insert a gBlock containing the C-terminal domain of the split-intein ABE (i.e., the DnaB protein of R. marinus and residues 713-1,088 of SpCas9) into the Agel and SalI restriction sites of pAAV-CAG-N-SpCas9-CBE. The ensuing plasmid was then used as a backbone to clone a gBlock containing residues 1,089-1,371 of SpCas9 with the SpRY mutations (D1135L, S1136W, G1218K, E1219Q, N1317R, A1322R, R1333P, R1335Q, and T1337R) with a 10 amino acid linker into the SalI and BamHI restriction sites. All amino acid sequences are provided in Table 4.

TABLE 4
Amino acid sequences of base editors constructed for this study

pAAV-CAG-N-SpCas9-CBE SEQ ID NO:173
ITR-CAG promoter-V5-nucleoplasmin NLS-rAPOBEC1-linker-1-712

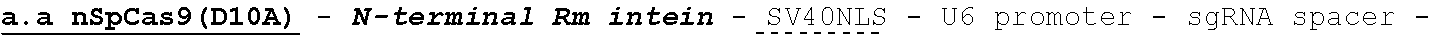

ITR
MGKPIPNPLLGLDSTAVKRPAATKKAGQAKKKKLDSSETGPVAVDPTLRRRIEPHEFEVFF
DPRELRKETCLLYEINWGGRHSIWRHTSQNTNKHVEVNFIEKFTTERYFCPNTRCSITWF
LSWSPCGECSRAITEFLSRYPHVTLFIYIARLYHHADPRNRQGLRDLISSGVTIQIMTEQES
GYCWRNFVNYSPSNEAHWPRYPHLWVRLYVLELYCIILGLPPCLNILRRKQPQLTFFTIAL
QSCHYQRLPPHILWATGLKSGSETPGTSESATPESDKKYSIGLAIGTNSVGWAVITDEYK
VPSKKFKVLGNTDRHSIKKNLIGALLFDSGETAEATRLKRTARRRYTRRKNRICYLQEIFS
NEMAKVDDSFFHRLEESFLVEEDKKHERHPIFGNIVDEVAYHEKYPTIYHLRKKLVDSTD
KADLRLIYLALAHMIKFRGHFLIEGDLNPDNSDVDKLFIQLVQTYNQLFEENPINASGVDA
KAILSARLSKSRRLENLIAQLPGEKKNGLFGNLIALSLGLTPNFKSNFDLAEDAKLQLSK
DTYDDDLDNLLAQIGDQYADLFLAAKNLSDAILLSDILRVNTEITKAPLSASMIKRYDEHH
QDLTLLKALVRQQLPEKYKEIFFDQSKNGYAGYIDGGASQEEFYKFIKPILEKMDGTEEL
LVKLNREDLLRKQRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKNLPNEKVLPK
HSLLYEYFTVYNELTKVKYVTEGMRKPAFLSGEQKKAIVDLLFKTNRKVTVKQLKEDYFK
KIECFDSVEISGVEDRFNASLGTYHDLLKIIKDKDFLDNEENEDILEDIVLTLTLFEDREMIE
ERLKTYAHLFDDKVMKQLKRRRYTGWGRLSRKLINGIRDKQSGKTILDFLKSDGFANRN
FMQLIHDDSLTFKEDIQKAQVCLAGDTLITLADGRRVPIRELVSQQNFSVWALNPQTYRL
ERARVSRAFCTGIKPVYRLTTRLGRSIRATANHRFLTPQGWKRVDELQPGDYLALPRRIP

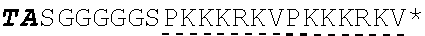

pAAV-CAG-C-SpCas9-CBE SEQ ID NO: 174
ITR-CAG promoter-C-terminal Rm intein-713-1371 a.a nSpCas9(D10A)-UGI-3x HA-

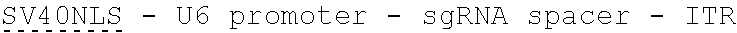

MAVKRPAATKKAGQAKKKKLDAAACPELRQLAQSDVYWDPIVSIEPDGVEEVFDLTVP
GPHNFVANDIIAHNSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNG
RDMYVDQELDINRLSDYDVDHIVPQSFLKDDSIDNKVLTRSDKNRGKSDNVPSEEVVKK
MKNYWRQLLNAKLITQRKFDNLTKAERGGLSELDKAGFIKRQLVETRQITKHVAQILDSR
MNTKYDENDKLIREVKVITLKSKLVSDFRKDFQFYKVREINNYHHAHDAYLNAVVGTALIK
KYPKLESEFVYGDYKVYDVRKMIAKSEQEIGKATAKYFFYSNIMNFFKTEITLANGEIRKR
PLIETNGETGEIVWDKGRDFATVRKVLSMPQVNIVKKTEVQTGGFSKESILPKRNSDKLIA
RKKDWDPKKYGGFDSPTVAYSVLVVAKVEKGKSKKLKSVKELLGITIMERSSFEKNPIDF
LEAKGYKEVKKDLIIKLPKYSLFELENGRKRMLASAGELQKGNELALPSKYVNFLYLASH
YEKLKGSPEDNEQKQLFVEQHKHYLDEIIEQISEFSKRVILADANLDKVLSAYNKHRDKPI
REQAENIIHLFTLTNLGAPAAFKYFDTTIDRKRYTSTKEVLDATLIHQSITGLYETRIDLSQL
GGDSGGSTNLSDIIEKETGKQLVIQESILMLPEEVEEVIGNKPESDILVHTAYDESTDENVM
LLTSDAPEYKPWALVIQDSNGENKIKMLSYPYDVPDYAYPYDVPDYAYPYDVPDYASGGS

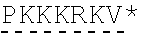

pCMV-ABE8e SpRY SEQ ID NO: 175
CMV promoter-SV40NLS-ABE8e-linker-SpRY (D1135L, S1136W, G1218K, E1219Q,
N1317R, A1322R, R1333P, R1335Q, and T1337R) nCas9(D10A)-linker-SV40NLS
MKRTADGSEFESPKKKRKVSEVEFSHEYWMRHALTLAKRARDEREVPVGAVLVLNNRV
IGEGWNRAIGLHDPTAHAEIMALRQGGLVMQNYRLIDATLYVTFEPCVMCAGAMIHSRIG
RVVFGVRNSKRGAAGSLMNVLNYPGMNHRVEITEGILADECAALLCDFYRMPRQVFNA
QKKAQSSINSGGSSGGSSGSETPGTSESATPESSGGSSGGSDKKYSIGLAIGTNSVGWA
VITDEYKVPSKKFKVLGNTDRHSIKKNLIGALLFDSGETAEATRLKRTARRRYTRRKNRICY
LQEIFSNEMAKVDDSFFHRLEESFLVEEDKKHERHPIFGNIVDEVAYHEKYPTIYHLRKKLV
DSTDKADLRLIYLALAHMIKFRGHFLIEGDLNPDNSDVDKLFIQLVQTYNQLFEENPINASGV
DAKAILSARLSKSRRLENLIAQLPGEKKNGLFGNLIALSLGLTPNFKSNFDLAEDAKLQLSK
DTYDDDLDNLLAQIGDQYADLFLAAKNLSDAILLSDILRVNTEITKAPLSASMIKRYDEHHQD
LTLLKALVRQQLPEKYKEIFFDQSKNGYAGYIDGGASQEEFYKFIKPILEKMDGTEELLVKL
NREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSLLYE
YFTVYNELTKVKYVTEGMRKPAFLSGEQKKAIVDLLFKTNRKVTVKQLKEDYFKKIECFDS
VEISGVEDRFNASLGTYHDLLKIIKDKDFLDNEENEDILEDIVLTLTLFEDREMIEERLKTYAH
LFDDKVMKQLKRRRYTGWGRLSRKLINGIRDKQSGKTILDFLKSDGFANRNFMQLIHDDSL
TFKEDIQKAQVSGQGDSLHEHIANLAGSPAIKKGILQTVKVVDELVKVMGRHKPENIVIEMA
RENQTTQKGQKNSRERMKRIEEGIKELGSQILKEHPVENTQLQNEKLYLYYLQNGRDMYV
DQELDINRLSDYDVDHIVPQSFLKDDSIDNKVLTRSDKNRGKSDNVPSEEVVKKMKNYWR
QLLNAKLITQRKFDNLTKAERGGLSELDKAGFIKRQLVETRQITKHVAQILDSRMNTKYDEN
DKLIREVKVITLKSKLVSDFRKDFQFYKVREINNYHHAHDAYLNAVVGTALIKKYPKLESEFV
YGDYKVYDVRKMIAKSEQEIGKATAKYFFYSNIMNFFKTEITLANGEIRKRPLIETNGETGEI
VWDKGRDFATVRKVLSMPQVNIVKKTEVQTGGFSKESILPKRNSDKLIARKKDWDPKKYG
GFLWPTVAYSVLVVAKVEKGKSKKLKSVKELLGITIMERSSFEKNPIDFLEAKGYKEVKKDL
IIKLPKYSLFELENGRKRMLASAKQLQKGNELALPSKYVNFLYLASHYEKLKGSPEDNEQK
QLFVEQHKHYLDEIIEQISEFSKRVILADANLDKVLSAYNKHRDKPIREQAENIIHLFTLTRLG
APRAFKYFDTTIDPKQYRSTKEVLDATLIHQSITGLYETRIDLSQLGGDSGGSKRTADGSEF
ESPKKKRKV*
pAAV-CAG-N-SpRY-ABE8e SEQ ID NO: 176
ITR-CAG promoter-V5-SV40NLS-ABE8e-linker-1-712 a.a. nCas9(D10A)-N-
terminal Rm intein-U6 promoter-sgRNA spacer-ITR
MGKPIPNPLLGLDSTKRTADGSEFESPKKKRKVSEVEFSHEYWMRHALTLAKRARDERE
VPVGAVLVLNNRVIGEGWNRAIGLHDPTAHAEIMALRQGGLVMQNYRLIDATLYVTFEPC
VMCAGAMIHSRIGRVVFGVRNSKRGAAGSLMNVLNYPGMNHRVEITEGILADECAALLC
DFYRMPRQVFNAQKKAQSSINSGGSSGGSSGSETPGTSESATPESSGGSSGGSDKKYS
IGLAIGTNSVGWAVITDEYKVPSKKFKVLGNTDRHSIKKNLIGALLFDSGETAEATRLKRT
ARRRYTRRKNRICYLQEIFSNEMAKVDDSFFHRLEESFLVEEDKKHERHPIFGNIVDEVAY
HEKYPTIYHLRKKLVDSTDKADLRLIYLALAHMIKFRGHFLIEGDLNPDNSDVDKLFIQLV
QTYNQLFEENPINASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAILLSDILRVNT
EITKAPLSASMIKRYDEHHQDLTLLKALVRQQLPEKYKEIFFDQSKNGYAGYIDGGASQE
EFYKFIKPILEKMDGTEELLVKLNREDLLRKQRTFDNGSIPHQIHLGELHAILRRQEDFYPF
LKDNREKIEKILTFRIPYYVGPLARGNSRFAWMTRKSEETITPWNFEEVVDKGASAQSFIE
RMTNFDKNLPNEKVLPKHSLLYEYFTVYNELTKVKYVTEGMRKPAFLSGEQKKAIVDLLF
KTNRKVTVKQLKEDYFKKIECFDSVEISGVEDRFNASLGTYHDLLKIIKDKDFLDNEENED
ILEDIVLTLTLFEDREMIEERLKTYAHLFDDKVMKQLKRRRYTGWGRLSRKLINGIRDKQS
GKTILDFLKSDGFANRNFMQLIHDDSLTFKEDIQKAQVCLAGDTLITLADGRRVPIRELVSQ
QNFSVWALNPQTYRLERARVSRAFCTGIKPVYRLTTRLGRSIRATANHRFLTPQGWKRVD
ELQPGDYLALPRRIPTAS*
pAAV-CAG-C-SpRY-ABE8e SEQ ID NO: 177
ITR-CAG promoter-C-terminal Rm intein-713-1371 a.a SpRY Cas9 (D1135L, S1136W,
G1218K, E1219Q, N1317R, A1322R, R1333P, R1335Q, and T1337R)-linker-3x UGI-
SV40NLS-U6 promoter-sgRNA spacer-ITR
MAAACPELRQLAQSDVYWDPIVSIEPDGVEEVFDLTVPGPHNFVANDIIAHNSGQGDSLH
EHIANLAGSPAIKKGILQTVKVVDELVKVMGRHKPENIVIEMARENQTTQKGQKNSRERM
KRIEEGIKELGSQILKEHPVENTQLQNEKLYLYYLQNGRDMYVDQELDINRLSDYDVDHIV
PQSFLKDDSIDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIREVKVITLKSKL
VSDFRKDFQFYKVREINNYHHAHDAYLNAVVGTALIKKYPKLESEFVYGDYKVYDVRKMI
AKSEQEIGKATAKYFFYSNIMNFFKTEITLANGEIRKRPLIETNGETGEIVWDKGRDFATV
RKVLSMPQVNIVKKTEVQTGGFSKESILPKRNSDKLIARKKDWDPKKYGGFLWPTVAYS
VLVVAKVEKGKSKKLKSVKELLGITIMERSSFEKNPIDFLEAKGYKEVKKDLIIKLPKYSLF
ELENGRKRMLASAKQLQKGNELALPSKYVNFLYLASHYEKLKGSPEDNEQKQLFVEQH
KHYLDEIIEQISEFSKRVILADANLDKVLSAYNKHRDKPIREQAENIIHLFTLTRLGAPRAFK
YFDTTIDPKQYRSTKEVLDATLIHQSITGLYETRIDLSQLGGDSGGSGGSGGSTNLSDIIEK
ETGKQLVIQESILMLPEEVEEVIGNKPESDILVHTAYDESTDENVMLLTSDAPEYKPWALV
IQDSNGENKIKMLSGGSKRTADGSEFEPKKKRKV*
pLVE-EF1α-EGFP (control for in vitro experiments) SEQ ID NO: 178
LTR-EF1α-EGFP-LTR
MVSKGEELFTGVVPILVELDGDVNGHKFSVSGEGEGDATYGKLTLKFICTTGKLPVPWP
TLVTTLTYGVQCFSRYPDHMKQHDFFKSAMPEGYVQERTIFFKDDGNYKTRAEVKFEGD
TLVNRIELKGIDFKEDGNILGHKLEYNYNSHNVYIMADKQKNGIKVNFKIRHNIEDGSVQL
ADHYQQNTPIGDGPVLLPDNH

To construct the sgRNA expression vectors, oligonucleotides encoding the sgRNA targeting sequences were acquired from IDT (Table 5) and hybridized, phosphorylated and ligated into BbsI and BsaI restriction sites of pSP-gRNA (Addgene #47108) and pAAV-CAG-split-intein-CBE/ABE, respectively. All plasmid sequences were verified by Sanger sequencing.

TABLE 5
Oligonucleotide sequences used to modify the human HTT gene

| Designation | Target site | Sequence | PAM | Base editor | Figure |
|---|---|---|---|---|---|
| Guide number 1 | Exon 12 SA | TTCACTGAGGCTGAAGACAG | AG | xCas9-BE3 | 15 |
| | | SEQ ID NO: 179 | | | |
| Guide number 2 | Exon 12 SA | CTCATCCTTCACTGAGGCTG | ACTGAT | SaKKH-BE3 | 15 |
| | | SEQ ID NO: 180 | | | |
| Guide number 3 | Exon 12 SA | TCATCCTTCACTGAGGCTGA | AG | xCas9-BE3 | 15 |
| | | SEQ ID NO: 181 | | | |
| Guide number 4 | Exon 12 SA | CCTTCACTGAGGCTGAAGAC | AG | xCas9-BE3 | 15 |
| | | SEQ ID NO: 182 | | | |
| Guide number 5 | Exon 12 SA | TTCACTGAGGCTGAAGACAG | AG | xCas9-BE3 | 15 |
| | | SEQ ID NO: 183 | | | |
| Guide number 6 | Exon 12 SA | TTCAGCCTCAGTGAAGGATG | AG | xCas9- | 15 |
| | | SEQ ID NO: 184 | | ABE7.10 | |
| Guide number 7 | Exon 12 SA | TCTTCAGCCTCAGTGAAGGA | TG | xCas9- | 15 |
| | | SEQ ID NO: 185 | | ABE7.10 | |
| Guide number 8 | Exon 12 SA | CTGTCTTCAGCCTCAGTGAA | GG | xCas9- | 15 |
| | | SEQ ID NO: 186 | | ABE7.10 | |
| Guide number 9 | Exon 12 SA | TCTGTCTTCAGCCTCAGTGA | AG | xCas9- | 15 |
| | | SEQ ID NO: 187 | | ABE7.10 | |
| Guide number 10 | Exon 12 SA | TTCTCTGTCTTCAGCCTCAG | TG | xCas9- | 15 |
| | | SEQ ID NO: 188 | | ABE7.10 | |
| Guide number 11 | Exon 12 SA | GCCTCAGTGAAGGATGAGAT | CAG | SpCas9- | 15 |
| | | SEQ ID NO: 189 | | ABE7.10 | |
| Guide number 12 | Exon 12 SA | TGTTTCTCTGTCTTCAGCCT | CAG | SpCas9- | 15 |
| | | SEQ ID NO: 190 | | ABE7.10 | |
| Guide number 13 | Exon 12 SA | CTCTGTCTTCAGCCTCAGTG | AAG | SpCas9- | 15 |
| | | SEQ ID NO: 191 | | ABE7.10 | |
| Guide number 14 | Exon 12 SA | CTTCAGCCTCAGTGAAGGAT | GAG | SpCas9- | 15 |
| | | SEQ ID NO: 192 | | ABE7.10 | |
| Guide number 15 | Exon 12 SA | GCCTCAGTGAAGGATGAGAT | CAG | SpCas9- | 15 |
| | | SEQ ID NO: 193 | | ABE7.10 | |
| Guide number 16 | Exon 12 SA | CTTCACTGAGGCTGAAGACA | GAG | SpCas9- | 15 |
| | | SEQ ID NO: 194 | | BE3 | |
| Guide number 17 | Exon 12 SA | TCCTTCACTGAGGCTGAAGA | CAG | SpCas9- | 15 |
| | | SEQ ID NO: 195 | | BE3 | |
| Guide number 18 | Exon 12 SA | CTCATCCTTCACTGAGGCTG | AAG | SpCas9- | 15 |
| | | SEQ ID NO: 196 | | BE3 | |
| Guide number 19 | Exon 12 SA | TCAGCCTCAGTGAAGGATGA | GAT | xCas9- | 15 |
| | | SEQ ID NO: 197 | | ABE7.10 | |
| Guide number 20 | Exon 12 SA | TGTCTTCAGCCTCAGTGAAG | GAT | xCas9- | 15 |
| | | SEQ ID NO: 198 | | ABE7.10 | |
| Guide number 21 | Exon 12 SA | TCTCTGTCTTCAGCCTCAGT | GAA | xCas9- | 15 |
| | | SEQ ID NO: 199 | | ABE7.10 | |
| Guide number 22 | Exon 12 SA | TCTCATCCTTCACTGAGGCT | GAA | xCas9-BE3 | 15 |
| | | SEQ ID NO: 200 | | | |
| Guide number 23 | Exon 12 SA | TCTGTCTTCAGCCTCAGTGA | AGG | SpCas9- | 15 |
| | | SEQ ID NO: 201 | | ABE7.10 | |
| Guide number 24 | ESE: | CACAGAACAGCCACGGTCAC | AG | xCas9-BE3 | 15 |
| | TCACAGC | SEQ ID NO: 202 | | | |
| | SEQIDNO: 395 | | | | |
| Guide number 25 | ESE: | GCAGTGTGTGCTGTGACCGT | GG | xCas9-BE3 | 15 |
| | TCACAGC | SEQ ID NO: 203 | | | |
| | SEQIDNO: 395 | | | | |
| Guide number 26 | ESE: | GTGTGTGCTGTGACCGTGGC | TG | xCas9-BE3 | 15 |
| | TCACAGC | SEQ ID NO: 204 | | | |
| | SEQIDNO: 395 | | | | |
| Guide number 27 | ESE: | TCACAGAACAGCCACGGTCA | CAG | SpCas9- | 15 |
| | TCACAGC | SEQ ID NO: 205 | | BE3 | |
| | SEQIDNO: 395 | | | | |
| Guide number 28 | ESE: | CACGGTCACAGCACACACTG | CAG | SpCas9- | 15 |
| | TCACAGC | SEQ ID NO: 206 | | BE3 | |
| | SEQIDNO: 395 | | | | |
| Guide number 29 | ESE: | CGGTCACAGCACACACTGCA | GG | xCas9-BE3 | 15 |
| | TCACAGC | SEQ ID NO: 207 | | | |
| | SEQIDNO: 395 | | | | |
| Guide number 30 | ESE: | TGCAGTGTGTGCTGTGACCG | TGG | SpCas9- | 15 |
| | TCACAGO | SEQ ID NO: 208 | | BE3 | |
| | SEQIDNO: 395 | | | | |
| Guide number 31 | ESE: | CACAGAACAGCCACGGTCAC | AG | xCas9- | 15 |
| | TCACAGC | SEQ ID NO: 209 | | ABE7.10 | |
| | SEQIDNO: 395 | | | | |
| Guide number 32 | ESE: | TCACAGAACAGCCACGGTCA | CAG | SpCas9- | 15 |
| | TCACAGC | SEQ ID NO: 210 | | ABE7.10 | |
| | SEQIDNO: 395 | | | | |
| Guide number 33 | ESE: | GTGTGTGCTGTGACCGTGGC | TG | xCas9- | 15 |
| | TCACAGC | SEQ ID NO: 211 | | ABE7.10 | |
| | SEQIDNO: 395 | | | | |
| Guide number 34 | ESE: | GCAGTGTGTGCTGTGACCGT | GG | xCas9- | 15 |
| | TCACAGC | SEQ ID NO: 212 | | ABE7.10 | |
| | SEQIDNO: 395 | | | | |
| Guide number 35 | ESE: | CACGGTCACAGCACACACTG | CAG | SpCas9- | 15 |
| | TCACAGC | SEQ ID NO: 213 | | ABE7.10 | |
| | SEQIDNO: 395 | | | | |
| Guide number 36 | ESE: | CGGTCACAGCACACACTGCA | GG | xCas9- | 15 |
| | TCACAGC | SEQ ID NO: 214 | | ABE7.10 | |
| | SEQIDNO: 395 | | | | |
| Guide number 37 | ESE: | CCGCCTGCAGTGTGTGCTGT | GAC | SpCas9- | 15 |
| | TCACAGC | SEQ ID NO: 215 | | ABE7.10 | |
| | SEQIDNO: 395 | | | | |
| Guide number 38 | ESE: | TGCAGTGTGTGCTGTGACCG | TGG | SpCas9- | 15 |
| | TCACAGC | SEQ ID NO: 216 | | ABE7.10 | |
| | SEQIDNO: 395 | | | | |
| Guide number 39 | ESE: | ACGGTCACAGCACACACTGC | AGG | SpCas9- | 15 |
| | TCACAGC | SEQ ID NO: 217 | | ABE7.10 | |
| | SEQIDNO: 395 | | | | |
| Guide number 40 | ESE: | GTCACAGCACACACTGCAGG | CGG | SpCas9- | 15 |
| | TCACAGC | SEQ ID NO: 218 | | ABE7.10 | |
| | SEQIDNO: 395 | | | | |
| Guide number 41 | ESE: | GTCACAGCACACACTGCAGG | CG | xCas9- | 15 |
| | TCACAGC | SEQ ID NO: 219 | | ABE7.10 | |
| | SEQIDNO: 395 | | | | |
| Guide number 42 | ESE: | ACGGTCACAGCACACACTGC | AGG | SpCas9- | 15 |
| | TCACAGC | SEQ ID NO: 220 | | BE3 | |
| | SEQIDNO: 395 | | | | |
| Guide number 43 | ESE: | GTCACAGCACACACTGCAGG | CGG | SpCas9- | 15 |
| | TCACAGC | SEQ ID NO: 221 | | BE3 | |
| | SEQIDNO: 395 | | | | |
| Guide number 44 | Caspase-3: | CACACTGCAGGCGGACTCAG | TGG | SpCas9- | 15 |
| | 510DSVD513 | SEQ ID NO: 222 | | ABE7.10 | |
| | SEQIDNO: 390 | | | | |
| Guide number 45 | Caspase-3: | CACACTGCAGGCGGACTCAG | TGG | SpCas9- | 15 |
| | 510DSVD513 | SEQ ID NO: 223 | | BE3 | |
| | SEQIDNO: 390 | | | | |
| Guide number 46 | Caspase-3: | GCAGGCGGACTCAGTGGATC | TGG | SpCas9- | 15 |
| | 510DSVD513 | SEQ ID NO: 224 | | ABE7.10 | |
| | SEQIDNO: 390 | | | | |
| Guide number 47 | Caspase-3: | GCAGGCGGACTCAGTGGATC | TGG | SpCas9- | 15 |
| | 510DSVD513 | SEQ ID NO: 225 | | BE3 | |
| | SEQIDNO: 390 | | | | |
| Guide number 48 | Caspase-3: | CTCTGCCACTGATGGGGATG | AGG | SpCas9- | 15 |
| | 527 DEED530 | SEQ ID NO: 226 | | ABE7.10 | |
| | SEQIDNO: 391 | | | | |
| Guide number 49 | Caspase-3: | TGCCACTGATGGGGATGAGG | AGG | SpCas9- | 15 |
| | 527DEED530 | SEQ ID NO: 227 | | ABE7.10 | |
| | SEQIDNO: 391 | | | | |
| Guide number 50 | Caspase-3: | CTGCCACTGATGGGGATGAG | GAG | SpCas9- | 15 |
| | 527DEED530 | SEQ ID NO: 228 | | ABE7.10 | |
| | SEQIDNO: 391 | | | | |
| Guide number 51 | Caspase-3: | TGCCACTGATGGGGATGAGG | AGG | SpCas9- | 15 |
| | 527DEED530 | SEQ ID NO: 229 | | ABE7.10 | |
| | SEQIDNO: 391 | | | | |
| Guide number 52 | Caspase-3: | GGACCTGAATGATGGGACCC | AGG | SpCas9- | 15 |
| | 549DLND552 | SEQ ID NO: 230 | | ABE7.10 | |
| | SEQIDNO: 392 | | | | |
| Guide number 53 | Caspase-3: | GGACCTGAATGATGGGACCC | AGG | SpCas9- | 15 |
| | 549DLND552 | SEQ ID NO: 231 | | BE3 | |
| | SEQIDNO: 392 | | | | |
| Guide number 54 | Caspase-3: | CTGCCATGGACCTGAATGAT | GGG | SpCas9- | 15 |
| | 549DLND552 | SEQ ID NO: 232 | | ABE7.10 | |
| | SEQIDNO: 392 | | | | |
| Guide number 55 | Caspase-3: | CTGCCATGGACCTGAATGAT | GGG | SpCas9- | 15 |
| | 549DLND552 | SEQ ID NO: 233 | | BE3 | |
| | SEQIDNO: 392 | | | | |
| Guide number 56 | Caspase-3: | CCTGCCATGGACCTGAATGA | TGG | SpCas9- | 15 |
| | 549DLND552 | SEQ ID NO: 234 | | ABE7.10 | |
| | SEQIDNO: 392 | | | | |
| Guide number 57 | Caspase-3: | CCTGCCATGGACCTGAATGA | TGG | SpCas9- | 15 |
| | 549DLND552 | SEQ ID NO: 235 | | BE3 | |
| | SEQIDNO: 392 | | | | |
| Guide number 58 | Caspase-3: | CATCATTCAGGTCCATGGCA | GGG | SpCas9- | 15 |
| | 549DLND552 | SEQ ID NO: 236 | | ABE7.10 | |
| | SEQIDNO: 392 | | | | |
| Guide number 59 | Caspase-3: | CATCATTCAGGTCCATGGCA | GGG | SpCas9- | 15 |
| | 549DLND552 | SEQ ID NO: 237 | | BE3 | |
| | SEQIDNO: 392 | | | | |
| Guide number 60 | Caspase-3: | CCATCATTCAGGTCCATGGC | AGG | SpCas9- | 15 |
| | 549DLND552 | SEQ ID NO: 238 | | ABE7.10 | |
| | SEQIDNO: 392 | | | | |
| Guide number 61 | Caspase-3: | CCATCATTCAGGTCCATGGC | AGG | SpCas9- | 15 |
| | 549DLND552 | SEQ ID NO: 239 | | BE3 | |
| | SEQIDNO: 392 | | | | |
| Guide number 62 | Caspase-3: | GGTCCCATCATTCAGGTCCA | TGG | SpCas9- | 15 |
| | 549DLND552 | SEQ ID NO: 240 | | ABE7.10 | |
| | SEQIDNO: 392 | | | | |
| Guide number 63 | Caspase-3: | GGTCCCATCATTCAGGTCCA | TGG | SpCas9- | 15 |
| | 549DLND552 | SEQ ID NO: 241 | | BE3 | |
| | SEQIDNO: 392 | | | | |
| Guide number 64 | Caspase-3: | AGGCCTGGGTCCCATCATTC | AGG | SpCas9- | 15 |
| | 549DLND552 | SEQ ID NO: 242 | | ABE7.10 | |
| | SEQIDNO: 392 | | | | |
| Guide number 65 | Caspase-3: | AGGCCTGGGTCCCATCATTC | AGG | SpCas9- | 15 |
| | 549DLND552 | SEQ ID NO: 243 | | BE3 | |
| | SEQIDNO: 392 | | | | |
| Guide number 66 | ESE: | AACAGCTGAATCAGGCCCTT | CGG | SpCas9- | 15 |
| | CAGCTGT | SEQ ID NO: 244 | | ABE7.10 | |
| Guide number 67 | ESE: | AACAGCTGAATCAGGCCCTT | CGG | SpCas9- | 15 |
| | CAGCTGT | SEQ ID NO: 245 | | BE3 | |
| Guide number 68 | ESE: | AGCTGAATCAGGCCCTTCGG | TGG | SpCas9- | 15 |
| | CAGCTGT | SEQ ID NO: 246 | | ABE7.10 | |
| Guide number 69 | ESE: | AGCTGAATCAGGCCCTTCGG | TGG | SpCas9- | 15 |
| | CAGCTGT | SEQ ID NO: 247 | | BE3 | |
| Guide number 70 | ESE: | CACCACCGAAGGGCCTGATT | CAG | SpCas9- | 15 |
| | CAGCTGT | SEQ ID NO: 248 | | ABE7.10 | |
| Guide number 71 | ESE: | CACCACCGAAGGGCCTGATT | CAG | SpCas9- | 15 |
| | CAGCTGT | SEQ ID NO: 249 | | BE3 | |
| Guide number 72 | Caspase-3: | CTAACACCTAAACGGTTCAA | GGG | SpCas9- | 15 |
| | 584VLDG587 | SEQ ID NO: 250 | | BE3 | |
| | SEQIDNO: 393 | | | | |
| Guide number 73 | Caspase-3: | CTAACACCTAAACGGTTCAA | GGG | xCas9-BE3 | 15 |
| | 584VLDG587 | SEQ ID NO: 251 | | | |
| | SEQIDNO: 393 | | | | |
| Guide number 74 | Caspase-3: | GGTACCGTCTAACACCTAAA | CGG | SpCas9- | 15 |
| | 584VLDG587 | SEQ ID NO: 252 | | BE3 | |
| | SEQIDNO: 393 | | | | |
| Guide number 75 | Caspase-3: | TCTAACACCTAAACGGTTCA | AGG | xCas9-BE3 | 15 |
| | 584VLDG587 | SEQ ID NO: 253 | | | |
| | SEQIDNO: 393 | | | | |
| Guide number 76 | Caspase-3: | GTACCGTCTAACACCTAAAC | GGT | xCas9-BE3 | 15 |
| | 584VLDG587 | SEQ ID NO: 254 | | | |
| | SEQIDNO: 393 | | | | |
| Guide number 77 | Caspase-3: | GGTACCGTCTAACACCTAAA | CGG | xCas9-BE3 | 15 |
| | 584VLDG587 | SEQ ID NO: 255 | | | |
| | SEQIDNO: 393 | | | | |
| Guide number 78 | Caspase-3: | GTTAGACGGTACCGACAACC | AGT | xCas9-BE3 | 15 |
| | 584VLDG587 | SEQ ID NO: 256 | | | |
| | SEQIDNO: 393 | | | | |
| Guide number 79 | Caspase-3: | GTCTAACACCTAAACGGTTC | AAG | SpCas9- | 15 |
| | 584VLDG587 | SEQ ID NO: 257 | | BE3 | |
| | SEQIDNO: 393 | | | | |
| Guide number 80 | Caspase-3: | GTCTAACACCTAAACGGTTC | AAG | xCas9-BE3 | 15 |
| | 584VLDG587 | SEQ ID NO: 258 | | | |
| | SEQIDNO: 393 | | | | |
| Guide number 81 | Caspase-3: | TGTTAGACGGTACCGACAAC | CAG | SpCas9- | 15 |
| | 584VLDG587 | SEQ ID NO: 259 | | BE3 | |
| | SEQIDNO: 393 | | | | |
| Guide number 82 | Caspase-3: | TGTTAGACGGTACCGACAAC | CAG | SpCas9- | 15 |
| | 584VLDG587 | SEQ ID NO: 260 | | ABE7.10 | |
| | SEQIDNO: 393 | | | | |
| Guide number 83 | Caspase-3: | TGTTAGACGGTACCGACAAC | CAG | xCas9-BE3 | 15 |
| | 584VLDG587 | SEQ ID NO: 261 | | | |
| | SEQIDNO: 393 | | | | |
| Guide number 84 | Caspase-3: | TGAACCGTTTAGGTGTTAGA | CGG | SpCas9- | 15 |
| | 584VLDG587 | SEQ ID NO: 262 | | ABE7.10 | |
| | SEQIDNO: 393 | | | | |
| Guide number 85 | Caspase-3: | TCTAACACCTAAACGGTTCA | AGG | SpCas9- | 15 |
| | 584VLDG587 | SEQ ID NO: 263 | | BE3 | |
| | SEQIDNO: 393 | | | | |
| CBE-HTT-1 | Exon 13 SA | AACACCTAAACGGTTCAAGG | GGG | SpCas9- | |
| | | SEQ ID NO: 264 | | BE3 | |
| CBE-HTT-2 | Exon 13 SA | GGTACCGTCTAACACCTAAA | CGG | SpCas9- | 10c |
| | | SEQ ID NO: 265 | | BE3 | |
| CBE-HTT-3 | Exon 13 SA | CTAACACCTAAACGGTTCAA | GGG | SpCas9- | 10c |
| | | SEQ ID NO: 266 | | BE3 | |
| CBE-HTT-4 | Exon 13 SA | ACACCTAAACGGTTCAAGGG | GGG | SpCas9- | 10c, |
| | | SEQ ID NO: 267 | | BE3 | 10e-g, |
| | | | | | 16, |
| | | | | | 11b-g, |
| | | | | | 19, |
| | | | | | 12b-i, |
| | | | | | 20 |
| CBE-HTT-5 | Exon 13 SA | TAACACCTAAACGGTTCAAG | GGG | SpCas9- | 10c |
| | | SEQ ID NO: 268 | | BE3 | |
| CBE-HTT-6 | Exon 13 SA | TCTAACACCTAAACGGTTCA | AGG | SpCas9- | 10c |
| | | SEQ ID NO: 269 | | BE3 | |
| CBE-HTT-7 | Exon 13 SA | AACACCTAAACGGTTCAAGG | GGG | xCas9-B3 | 10c |
| | | SEQ ID NO: 270 | | | |
| CBE-HTT-8 | Exon 13 SA | GGTACCGTCTAACACCTAAA | CGG | xCas9-B3 | 10c |
| | | SEQ ID NO: 271 | | | |
| CBE-HTT-9 | Exon 13 SA | TTGAACCGTTTAGGTGTTAG | GGG | xCas9-B3 | 10c |
| | | SEQ ID NO: 272 | | | |
| CBE-HTT-10 | Exon 13 SA | ACACCTAAACGGTTCAAGGG | GGG | xCas9-B3 | 10c |
| | | SEQ ID NO: 273 | | | |
| CBE-HTT-11 | Exon 13 SA | TAACACCTAAACGGTTCAAG | GGG | xCas9-B3 | 10c |
| | | SEQ ID NO: 274 | | | |
| CBE-HTT-12 | Exon 13 SA | TCTAACACCTAAACGGTTCA | AGG | xCas9-B3 | 10c |
| | | SEQ ID NO: 275 | | | |
| CBE-HTT-13 | Exon 13 SA | CACCTAAACGGTTCAAGGGG | GGC | xCas9-B3 | 10c |
| | | SEQ ID NO: 276 | | | |
| CBE-HTT-14 | Exon 13 SA | TCGGTACCGTCTAACACCTA | AAC | xCas9-B3 | 10c |
| | | SEQ ID NO: 277 | | | |
| CBE-HTT-15 | Exon 13 SA | GTACCGTCTAACACCTAAAC | GGT | xCas9-B3 | 10c |
| | | SEQ ID NO: 278 | | | |
| CBE-HTT-16 | Exon 13 SA | TCGGTACCGTCTAACACCTA | AACGGT | SaKKH-BE3 | 10c |
| | | SEQ ID NO: 279 | | | |
| ABE-HTT-1 | Exon 13 SA | TGAACCGTTTAGGTGTTAGA | CGG | SpCas9- | 10c |
| | | SEQ ID NO: 280 | | ABE7.10 | |
| ABE-HTT-2 | Exon 13 SA | CCTTGAACCGTTTAGGTGTT | AGA | xCas9- | 10c |
| | | SEQ ID NO: 281 | | ABE7.10 | |
| SpRY-CBE-HTT- | Exon 13 SA | GTCGGTACCGTCTAACACCT | AGA | SpRY- | 13b, |
| 1 | | SEQ ID NO: 282 | | CBE4max | 21a |
| SpRY-CBE-HTT- | Exon 13 SA | GTCTAACACCTAAACGGTTC | AAA | SpRY- | 13b, |
| 2 | | SEQ ID NO: 283 | | CBE4max | 21a |
| SpRY-CBE-HTT- | Exon 13 SA | TCTAACACCTAAACGGTTCA | AAG | SpRY- | 13b, 13 |
| 3 | | SEQ ID NO: 284 | | CBE4max | d, 21a |
| SpRY-CBE-HTT- | Exon 13 SA | CTAACACCTAAACGGTTCAA | AGG | SpRY- | 13b, |
| 4 | | SEQ ID NO: 285 | | CBE4max | 21a |
| SpRY-CBE-HTT- | Exon 13 SA | TAACACCTAAACGGTTCAAG | GGG | SpRY- | 13b, |
| 5 | | SEQ ID NO: 286 | | CBE4max | 21a |
| SpRY-CBE-HTT- | Exon 13 SA | AACACCTAAACGGTTCAAGG | GGG | SpRY- | 13b, |
| 6 | | SEQ ID NO: 287 | | CBE4max | 21a |
| SpRY-CBE-HTT- | Exon 13 SA | ACACCTAAACGGTTCAAGGG | GGG | SpRY- | 13b, |
| 7 | | SEQ ID NO: 288 | | CBE4max | 21a |
| SpRY-CBE-HTT- | Exon 13 SA | CACCTAAACGGTTCAAGGGG | GGG | SpRY- | 13b, |
| 8 | | SEQ ID NO: 289 | | CBE4max | 21a |
| SpRY-CBE-HTT- | Exon 13 SA | CTAAACGGTTCAAGGGGGGC | GGC | SpRY- | 13b, |
| 9 | | SEQ ID NO: 290 | | CBE4max | 21a |
| SpRY-CBE-HTT- | Exon 13 SA | TCGGTACCGTCTAACACCTA | TGT | SpRY- | 13b, |
| 10 | | SEQ ID NO: 291 | | CBE4max | 21a |
| SpRY-CBE-HTT- | Exon 13 SA | CGGTACCGTCTAACACCTAA | AAC | SpRY- | 13b, |
| 11 | | SEQ ID NO: 292 | | CBE4max | 21a |
| SpRY-CBE-HTT- | Exon 13 SA | GGTACCGTCTAACACCTAAA | ACG | SpRY- | 13b, |
| 12 | | SEQ ID NO: 293 | | CBE4max | 21a |
| SpRY-CBE-HTT- | Exon 13 SA | GTACCGTCTAACACCTAAAC | CGG | SpRY- | 13b, |
| 13 | | SEQ ID NO: 295 | | CBE4max | 21a |
| SpRY-CBE-HTT- | Exon 13 SA | TACCGTCTAACACCTAAACG | GGT | SpRY- | 13b, |
| 14 | | SEQ ID NO: 296 | | CBE4max | 21a |
| SpRY-CBE-HTT- | Exon 13 SA | ACCGTCTAACACCTAAACGG | GTT | SpRY- | 13b, |
| 15 | | SEQ ID NO: 297 | | CBE4max | 21a |
| SpRY-CBE-HTT- | Exon 13 SA | CCGTCTAACACCTAAACGGT | TTC | SpRY- | 13b, |
| 16 | | SEQ ID NO: 298 | | CBE4max | 21a |
| SpRY-CBE-HTT- | Exon 13 SA | CGTCTAACACCTAAACGGTT | TCA | SpRY- | 13b, |
| 17 | | SEQ ID NO: 299 | | CBE4max | 21a |
| SpRY-CBE-HTT- | Exon 13 SA | ACCTAAACGGTTCAAGGGGG | CAA | SpRY- | 13b, |
| 18 | | SEQ ID NO: 300 | | CBE4max | 21a |
| SpRY-CBE-HTT- | Exon 13 SA | CCTAAACGGTTCAAGGGGGG | GCT | SpRY- | 13b, |
| 19 | | SEQ ID NO: 301 | | CBE4max | 21a |
| SpRY-ABE-HTT- | Exon 13 SA | AGGTGTTAGACGGTACCGAC | CTG | SpRY- | 13c, |
| 1 | | SEQ ID NO: 302 | | ABE8e | 21b |
| SpRY-ABE-HTT- | Exon 13 SA | TAGGTGTTAGACGGTACCGA | AAC | SpRY- | 13c, |
| 2 | | SEQ ID NO: 303 | | ABE8e | 21b |
| SpRY-ABE-HTT- | Exon 13 SA | TTAGGTGTTAGACGGTACCG | CAA | SpRY- | 13c-d, |
| 3 | | SEQ ID NO: 304 | | ABE8e | 13f-i, |
| | | | | | 21b, |
| | | | | | 14b-k, |
| | | | | | 23, 24 |
| SpRY-ABE-HTT- | Exon 13 SA | TTTAGGTGTTAGACGGTACC | ACA | SpRY- | 13c, |
| 4 | | SEQ ID NO: 305 | | ABE8e | 21b |
| SpRY-ABE-HTT- | Exon 13 SA | GTTTAGGTGTTAGACGGTAC | GAC | SpRY- | 13c, |
| 5 | | SEQ ID NO: 306 | | ABE8e | 21b |
| SpRY-ABE-HTT- | Exon 13 SA | CGTTTAGGTGTTAGACGGTA | CGA | SpRY- | 13c, |
| 6 | | SEQ ID NO: 307 | | ABE8e | 21b |
| SpRY-ABE-HTT- | Exon 13 SA | CCGTTTAGGTGTTAGACGGT | CCG | SpRY- | 13c, |
| 7 | | SEQ ID NO: 308 | | ABE8e | 21b |
| SpRY-ABE-HTT- | Exon 13 SA | AACCGTTTAGGTGTTAGACG | ACC | SpRY- | 13c, |
| 8 | | SEQ ID NO: 309 | | ABE8e | 21b |
| SpRY-ABE-HTT- | Exon 13 SA | ACCGTTTAGGTGTTAGACGG | TAC | SpRY- | 13c, |
| 9 | | SEQ ID NO: 310 | | ABE8e | 21b |
| SpRY-ABE-HTT- | Exon 13 SA | GAACCGTTTAGGTGTTAGAC | GTA | SpRY- | 13c, |
| 10 | | SEQ ID NO: 311 | | ABE8e | 21b |
| SpRY-ABE-HTT- | Exon 13 SA | TGAACCGTTTAGGTGTTAGA | GGT | SpRY- | 13c, |
| 11 | | SEQ ID NO: 312 | | ABE8e | 21b |
| SpRY-ABE-HTT- | Exon 13 SA | TTGAACCGTTTAGGTGTTAG | CGG | SpRY- | 13c, |
| 12 | | SEQ ID NO: 313 | | ABE8e | 21b |
| SpRY-ABE-HTT- | Exon 13 SA | CTTGAACCGTTTAGGTGTTA | ACG | SpRY- | 13c, |
| 13 | | SEQ ID NO: 314 | | ABE8e | 21b |
| SpRY-ABE-HTT- | Exon 13 SA | CCTTGAACCGTTTAGGTGTT | GAC | SpRY- | 13c, |
| 14 | | SEQ ID NO: 315 | | ABE8e | 21b |
| SpRY-ABE-HTT- | Exon 13 SA | CCCTTGAACCGTTTAGGTGT | AGA | SpRY- | 13c, |
| 15 | | SEQ ID NO: 316 | | ABE8e | 21b |
| SpRY-ABE-HTT- | Exon 13 SA | CCCCTTGAACCGTTTAGGTG | TAG | SpRY- | 13c, |
| 16 | | SEQ ID NO: 317 | | ABE8e | 21b |
| SpRY-ABE-HTT- | Exon 13 SA | CCCCCTTGAACCGTTTAGGT | TTA | SpRY- | 13c, |
| 17 | | SEQ ID NO: 318 | | ABE8e | 21b |
| SpRY-ABE-HTT- | Exon 13 SA | CCCCCCTTGAACCGTTTAGG | GTT | SpRY- | 13c, |
| 18 | | SEQ ID NO: 319 | | ABE8e | 21b |
| SpRY-ABE-HTT- | Exon 13 SA | GCCCCCCTTGAACCGTTTAG | TGT | SpRY- | 13c, |
| 19 | | SEQ ID NO: 320 | | ABE8e | 21b |
| mRosa26 | ROSA26 | CATGGATTTCTCCGGTGAAT | AGG | SpCas9- | 12c-i, |
| | locus in mice | SEQ ID NO: 321 | | BE3/SpRY- | 20, |
| | | | | ABE8e | 14c-k, |
| | | | | | 24 |

TABLE 7
Guides found to edit HTT D586.

| | | | Mean HTT | |
|---|---|---|---|---|
| | | | Exon 13 SA | Mean D586 |
| Designation | Sequence | Base editor | Editing (%) | Editing (%) |
| Guide number 74 | GGTACCGTCTAACACCTAAA | SpCas9-BE3 | 5.71 | 14 |
| | SEQ ID NO: 252 | | | |
| Guide number 85 | TCTAACACCTAAACGGTTCA | SpCas9-BE3 | 8.78 | 14 |
| | SEQ ID NO: 253 | | | |
| SpRY-CBE-HTT-1 | GTCGGTACCGTCTAACACCT | SpRY-CBE4max | 14.33 | 17.33 |
| | SEQ ID NO: 282 | | | |
| SpRY-CBE-HTT-2 | GTCTAACACCTAAACGGTTC | SpRY-CBE4max | 3.67 | 16.67 |
| | SEQ ID NO: 257 | | | |
| SpRY-CBE-HTT-3 | TCTAACACCTAAACGGTTCA | SpRY-CBE4max | 19 | 26.67 |
| | SEQ ID NO: 253 | | | |
| SpRY-CBE-HTT-4 | CTAACACCTAAACGGTTCAA | SpRY-CBE4max | 42.33 | 35 |
| | SEQ ID NO: 250 | | | |
| SpRY-CBE-HTT-5 | TAACACCTAAACGGTTCAAG | SpRY-CBE4max | 42.67 | 30.33 |
| | SEQ ID NO: 268 | | | |
| SpRY-CBE-HTT-6 | AACACCTAAACGGTTCAAGG | SpRY-CBE4max | 50 | 53.67 |
| | SEQ ID NO: 264 | | | |
| SpRY-CBE-HTT-7 | ACACCTAAACGGTTCAAGGG | SpRY-CBE4max | 37.67 | 51.67 |
| | SEQ ID NO: 267 | | | |
| SpRY-CBE-HTT-8 | CACCTAAACGGTTCAAGGGG | SpRY-CBE4max | 45.33 | 33.33 |
| | SEQ ID NO: 276 | | | |
| SpRY-CBE-HTT-9 | CTAAACGGTTCAAGGGGGGC | SpRY-CBE4max | 8 | 2.67 |
| | SEQ ID NO: 290 | | | |
| SpRY-ABE-HTT-1 | AGGTGTTAGACGGTACCGAC | SpRY-ABE8e | 26 | 27 |
| | SEQ ID NO: 302 | | | |
| SpRY-ABE-HTT-2 | TAGGTGTTAGACGGTACCGA | SpRY-ABE8e | 53.67 | 50.67 |
| | SEQ ID NO: 303 | | | |
| SpRY-ABE-HTT-3 | TTAGGTGTTAGACGGTACCG | SpRY-ABE8e | 57.67 | 50.33 |
| | SEQ ID NO: 304 | | | |
| SpRY-ABE-HTT-4 | TTTAGGTGTTAGACGGTACC | SpRY-ABE8e | 47.67 | 28 |
| | SEQ ID NO: 305 | | | |
| SpRY-ABE-HTT-5 | GTTTAGGTGTTAGACGGTAC | SpRY-ABE8e | 7 | 4.33 |
| | SEQ ID NO: 306 | | | |
| SpRY-ABE-HTT-6 | CGTTTAGGTGTTAGACGGTA | SpRY-ABE8e | 36 | 28 |
| | SEQ ID NO: 307 | | | |
| SpRY-ABE-HTT-7 | CCGTTTAGGTGTTAGACGGT | SpRY-ABE8e | 22.67 | 28.33 |
| | SEQ ID NO: 308 | | | |
| SpRY-ABE-HTT-8 | AACCGTTTAGGTGTTAGACG | SpRY-ABE8e | 11.33 | 4.67 |
| | SEQ ID NO: 309 | | | |
| SpRY-ABE-HTT-9 | ACCGTTTAGGTGTTAGACGG | SpRY-ABE8e | 17 | 21.67 |
| | SEQ ID NO: 310 | | | |

### Cell Culture and Transfection

HEK293T cells were obtained from the American Type Culture Collection (ATCC) and maintained in Dulbecco's Modified Eagle Medium (DMEM) supplemented with 10% fetal bovine serum (FBS) and 1% penicillin/streptomycin at 37° C. with 5% C02. HEK293T cells were transfected in 24-well plates using Lipofectamine 2000 (Thermo Fisher Scientific) according to the manufacturer's instructions, with 500 ng of base editor-encoding plasmid and 500 ng of sgRNA-encoding plasmid used per well. For experiments using the split-intein versions of a base editor, 500 ng each of the N- and C-terminal plasmids was used.

### Clonal Cell Line Generation

HEK293T cells were transfected with 500 ng of plasmid encoding pCMV-N-CBE-HTT-4 and 500 ng pCMV-C-CBE-HTT-4 using Lipofectamine 2000. At 24 hr post-transfection, cells were re-plated on a 10-cm dish at a 1:100 ratio and grown as colonies from single cells, with Sanger sequencing used to confirm the target edit in PCR amplicons generated from genomic DNA from randomly selected clones, as described below.

### Analysis of DNA Editing

Genomic DNA was isolated using a DNeasy Blood and Tissue Kit (Qiagen) with PCR amplification performed using a KAPA2G Robust PCR Kit (KAPA Biosystems) using 20-100 ng of DNA, Buffer A (1×), Enhancer (1×), dNTPs (0.2 mM), forward primer (0.5 μM), reverse primer (0.5 μM), KAPA2G Robust DNA Polymerase (0.5 U) and water (up to 25 μL). Cycling parameters were used as recommended by the manufacturer.

Sanger sequencing of the PCR amplicons was performed by the W. M. Keck Center for Comparative and Functional Genomics at the University of Illinois Urbana-Champaign. Base editing efficiencies were estimated by analyzing sequencing chromatograms using EditR with the primer sequences provided in Table 6.

TABLE 6
Primer sequences used in this study.

| Designation | Sequence (5′→3′) |
|---|---|
| HTT exon 12 amplicon | GTTGTTTCTGACCACTGCTTGC SEQ ID NO: 322 |
| gDNA F | |
| HTT exon 12 amplicon | AAAAAGATGTCAGGCCCCTCTG SEQ ID NO: 323 |
| gDNA R | |
| HTT exon 12 gDNA | TTGAACTTCGGTTTTTGCAG SEQ ID NO: 324 |
| sequencing | |
| HTT exon 12 RT-PCR | CCTTTCAAGAAAACAAAAAG SEQ ID NO: 325 |
| amplicon F | |
| HTT exon 12 RT-PCR | ATACCTGTGGCTTCCTCATC SEQ ID NO: 326 |
| amplicon R | |
| HTT exon 13 amplicon | GTGATGTGGAATCATGCCCCAAGC SEQ ID NO: 327 |
| gDNA F | |
| HTT exon 13 amplicon | CCACTTTGTAGCGCATCACCTG SEQ ID NO: 328 |
| gDNA R | |
| HTT exon 13 gDNA | CTTAGCAGTTTCCATGCGTGCA SEQ ID NO: 329 |
| sequencing | |
| HTT exon 13 RT-PCR | GTGACTTGACAAGCTCTGCCAC SEQ ID NO: 330 |
| amplicon F | |
| HTT exon 13 RT-PCR | ATGTCACCTTTGATGCGGCAAG SEQ ID NO: 331 |
| amplicon R | |
| GAPDH (housekeeping | GACAGTCAGCCGCATCTTCT SEQ ID NO: 332 |
| gene mRNA) qPCR F | |
| GAPDH (housekeeping | ATCCGTTGACTCCGACCTTC SEQ ID NO: 333 |
| gene mRNA) qPCR R | |
| HTT (target mRNA) qPCR | AAAGAATGGTGCCCCTCGGAGT SEQ ID NO: 334 |
| F | |
| HTT (target mRNA) qPCR | TTGCTTGTTCGAGTCAGGCACG SEQ ID NO: 335 |
| R | |
| HTT exon 13 amplicon | TCGTCGGCAGCGTCAGATGTGTATAAGAGACAGGGAGGGCTTGTCTCT |
| gDNA NGS F | TGGTGAT SEQ ID NO: 336 |
| HTT exon 13 amplicon | GTCTCGTGGGCTCGGAGATGTGTATAAGAGACAGCCACTTTGTAGCGC |
| gDNA NGS R | ATCACCTG SEQ ID NO: 337 |
| HTT exon 13 amplicon | TCGTCGGCAGCGTCAGATGTGTATAAGAGACAGATCTCTCAACACAAAT |
| cDNA NGS F | TTATCAAC SEQ ID NO: 338 |
| HTT exon 13 amplicon | GTCTCGTGGGCTCGGAGATGTGTATAAGAGACAGCACTCCAGGGTCAG |
| cDNA NGS R | CA SEQ ID NO: 339 |
| AAV titer (bGH polyA) | GCCTTCTAGTTGCCAGCCAT SEQ ID NO: 340 |
| qPCR F | |
| AAV titer (bGH polyA) | GGCACCTTCCAGGGTCAAG SEQ ID NO: 341 |
| qPCR R | |
| CBE-HTT-4 OT1 gDNA | TCGTCGGCAGCGTCAGATGTGTATAAGAGACAGAAGTGTGGGGTTCAG |
| Nextera amplicon F | GCCCTGA SEQ ID NO: 342 |
| CBE-HTT-4 OT1 gDNA | GTCTCGTGGGCTCGGAGATGTGTATAAGAGACAGGGAGTAGCATCAG |
| Nextera amplicon R | GCCCGTGGA SEQ ID NO: 343 |
| CBE-HTT-4 OT2 gDNA | TCGTCGGCAGCGTCAGATGTGTATAAGAGACAGCCCTCTCACACACAG |
| Nextera amplicon F | TAGCAGACCA SEQ ID NO: 344 |
| CBE-HTT-4 OT2 gDNA | GTCTCGTGGGCTCGGAGATGTGTATAAGAGACAGGCCCACCTTCCTCT |
| Nextera amplicon R | CAGAGGCA SEQ ID NO: 345 |
| CBE-HTT-4 OT3 gDNA | TCGTCGGCAGCGTCAGATGTGTATAAGAGACAGGGCAACTGCTGGAAC |
| Nextera amplicon F | CAAAGCA SEQ ID NO: 346 |
| CBE-HTT-4 OT3 gDNA | GTCTCGTGGGCTCGGAGATGTGTATAAGAGACAGTTCAGGTGCTTGGC |
| Nextera amplicon R | CGGGATTT SEQ ID NO: 347 |
| CBE-HTT-4 OT4 gDNA | TCGTCGGCAGCGTCAGATGTGTATAAGAGACAGATGAGTGCACAGGCC |
| Nextera amplicon F | TCCCCTT SEQ ID NO: 348 |
| CBE-HTT-4 OT4 gDNA | GTCTCGTGGGCTCGGAGATGTGTATAAGAGACAGAGGGTGAACAGGG |
| Nextera amplicon R | CCAGAAGCT SEQ ID NO: 349 |
| CBE-HTT-4 OT5 gDNA | TCGTCGGCAGCGTCAGATGTGTATAAGAGACAGCCCGGATTGCTGTGA |
| Nextera amplicon F | TGCTGCA SEQ ID NO: 350 |
| CBE-HTT-4 OT5 gDNA | GTCTCGTGGGCTCGGAGATGTGTATAAGAGACAGTCAGGCTCCCCTAC |
| Nextera amplicon R | CTCCCACT SEQ ID NO: 351 |
| CBE-HTT-4 OT6 gDNA | TCGTCGGCAGCGTCAGATGTGTATAAGAGACAGTCACACTGTCACTGT |
| Nextera amplicon F | GCTCCCA SEQ ID NO: 352 |
| CBE-HTT-4 OT6 gDNA | GTCTCGTGGGCTCGGAGATGTGTATAAGAGACAGGGTGTCCACGATG |
| Nextera amplicon R | GCCTTCAGC SEQ ID NO: 353 |
| CBE-HTT-4 OT7 gDNA | TCGTCGGCAGCGTCAGATGTGTATAAGAGACAGTGTGCTGCTCCATTG |
| Nextera amplicon F | TTAATGCA SEQ ID NO: 354 |
| CBE-HTT-4 OT7 gDNA | GTCTCGTGGGCTCGGAGATGTGTATAAGAGACAGGCATCATACAACTA |
| Nextera amplicon R | GAGTGAGGGGCA SEQ ID NO: 355 |
| CBE-HTT-4 OT8 gDNA | TCGTCGGCAGCGTCAGATGTGTATAAGAGACAGACAAACTGCTCCAGG |
| Nextera amplicon F | CACACCG SEQ ID NO: 356 |
| CBE-HTT-4 OT8 gDNA | GTCTCGTGGGCTCGGAGATGTGTATAAGAGACAGACTTGGCACCAAGC |
| Nextera amplicon R | TGGTTGCA SEQ ID NO: 357 |
| SpRY-ABE-HTT-3 OT1 | TCGTCGGCAGCGTCAGATGTGTATAAGAGACAG |
| gDNA Nextera amplicon F | GGACATAAGCCAGCCCAGATAA SEQ ID NO: 358 |
| SpRY-ABE-HTT-3 OT1 | GTCTCGTGGGCTCGGAGATGTGTATAAGAGACAG |
| gDNA Nextera amplicon R | TTTCATCACCTTCTTCCTCTTCCT SEQ ID NO: 359 |
| SpRY-ABE-HTT-3 OT2 | TCGTCGGCAGCGTCAGATGTGTATAAGAGACAG |
| gDNA Nextera amplicon F | TGCTGCAGTGAACATAGGCATG SEQ ID NO: 360 |
| SpRY-ABE-HTT-3 OT2 | GTCTCGTGGGCTCGGAGATGTGTATAAGAGACAG |
| gDNA Nextera amplicon R | AAAGCTCAACGTCACTGATCATT SEQ ID NO: 361 |
| SpRY-ABE-HTT-3 OT3 | TCGTCGGCAGCGTCAGATGTGTATAAGAGACAG |
| gDNA Nextera amplicon F | TGGCCAGATATGTTCATGCCAG SEQ ID NO: 362 |
| SpRY-ABE-HTT-3 OT3 | GTCTCGTGGGCTCGGAGATGTGTATAAGAGACAG |
| gDNA Nextera amplicon R | GAAGTCCACTGATTAGCCTGCA SEQ ID NO: 363 |
| SpRY-ABE-HTT-3 OT4 | TCGTCGGCAGCGTCAGATGTGTATAAGAGACAG |
| gDNA Nextera amplicon F | AACAGGGCACCAGATAACCAGG SEQ ID NO: 364 |
| SpRY-ABE-HTT-3 OT4 | GTCTCGTGGGCTCGGAGATGTGTATAAGAGACAG |
| gDNA Nextera amplicon R | AATTCACAACCAAGAACCTTAA SEQ ID NO: 365 |
| SpRY-ABE-HTT-3 OT5 | TCGTCGGCAGCGTCAGATGTGTATAAGAGACAG |
| gDNA Nextera amplicon F | ATGGCTTCCTCATTGTGGTCCT SEQ ID NO: 366 |
| SpRY-ABE-HTT-3 OT5 | GTCTCGTGGGCTCGGAGATGTGTATAAGAGACAGGTCTTGATCCCCTG |
| gDNA Nextera amplicon R | ACCTCG SEQ ID NO: 367 |
| NeuN (target mRNA) qPCR | CACCACTCTCTTGTCCGTTTGC SEQ ID NO: 468 |
| F | |
| NeuN (target mRNA) qPCR | GGCTGAGCATATCTGTAAGCTGC SEQ ID NO: 469 |
| R | |
| GFAP (target mRNA) qPCR | CACCTACAGGAAATTGCTGGAGG SEQ ID NO: 470 |
| F | |
| GFAP (target mRNA) qPCR | CCACGATGTTCCTCTTGAGGT SEQ ID NO: 480 |
| R | |
| IBA1 (target mRNA) qPCR | TCTGCCGTCCAAACTTGAAGCC SEQ ID NO: 481 |
| F | |
| HTT Exon 1 cDNA | CTGTGGCTGAGGAGCCGCTG SEQ ID NO: 482 |
| synthesis F | |
| HTT Exon 67 cDNA | CCTGCGGTCGAGCTCCTCCTCTAT SEQ ID NO: 483 |
| synthesis R | |
| HTT Exon 1 Long-read | TGGCTGAGGAGCCGCTGCACCG SEQ ID NO: 484 |
| amplicon F | |
| HTT Exon 48 Long-read | TGGCTGAGGAGCCGCTGCACCG SEQ ID NO: 485 |
| amplicon R | |

### RT-PCR

RNA was harvested using the RNeasy Plus Mini Kit (Qiagen) according to manufacturer's instructions. cDNA synthesis was performed using the qScript cDNA Synthesis Kit (Quanta Biosciences) with 1 μg of RNA. PCR was then performed using the KAPA2G Robust PCR Kit using 25 ng of cDNA, Buffer A (1×), Enhancer (1×), dNTPs (0.2 mM), forward primer (0.5 μM), reverse primer (0.5 μM), KAPA2G Robust DNA Polymerase (0.5 U) and water (up to 25 μL). Cycling parameters were used as recommended by the manufacturer.

PCR products were visualized using a 2% agarose gel stained with ethidium bromide and imaged using a ChemiDoc-It2 (UVP). The sequences for the primers used for each target are provided in Table 6.

### Densitometry Analysis

Exon skipping efficiencies were determined by densitometry analysis. The PCR products obtained from RT-PCR were analyzed by agarose gel electrophoresis, with the intensity of the bands measured using ImageJ. After subtracting background, band intensity was calculated using the following formula: % exon skipping=(skipped band intensity)/(non-skipped band intensity+skipped band intensity), where band intensity is the sum of each pixel grayscale value within the selected area of the band.

qPCR

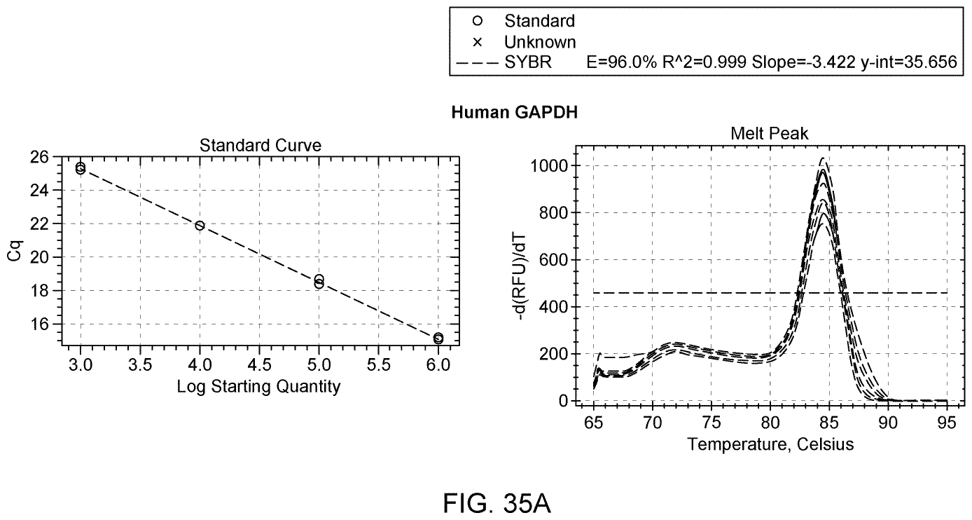

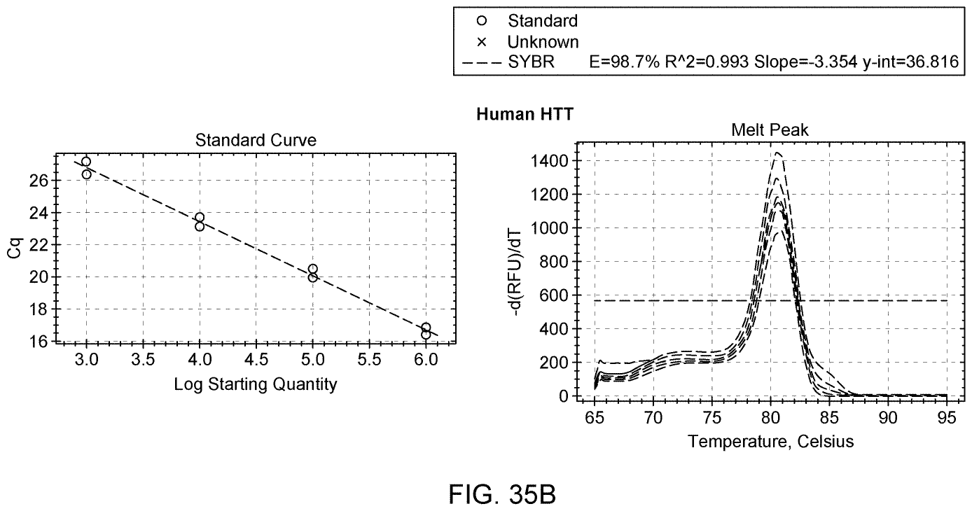

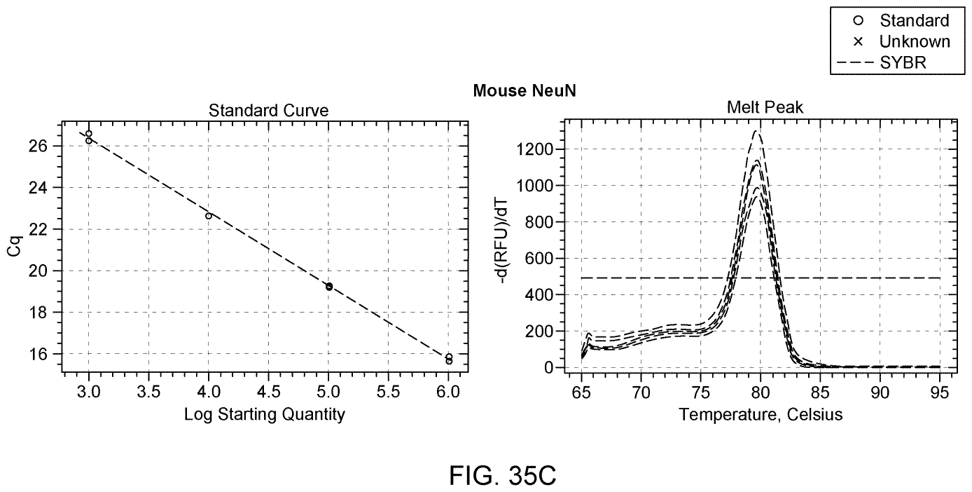

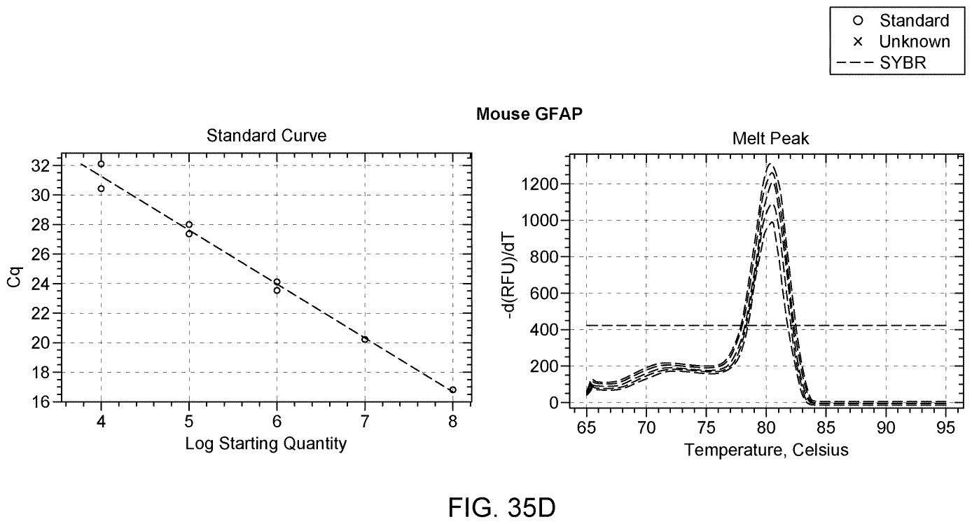

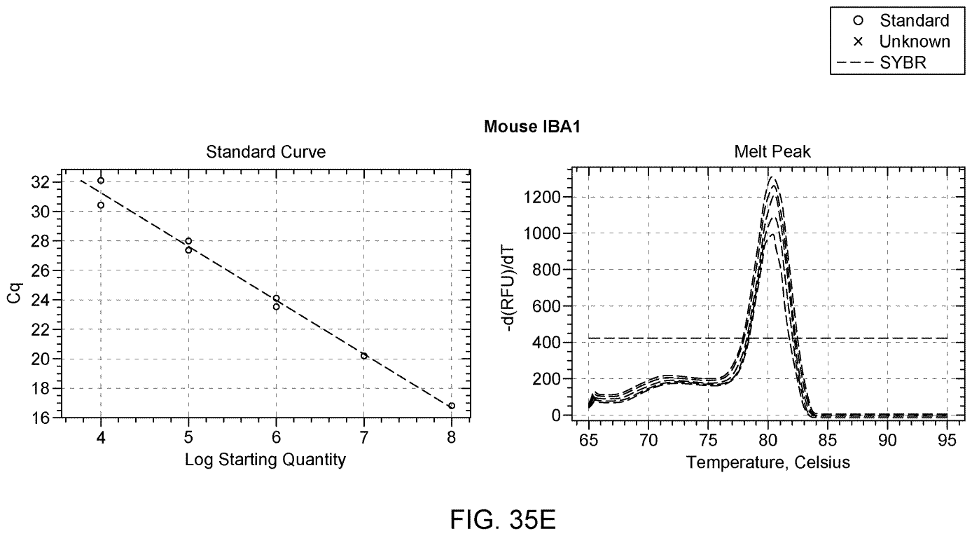

qPCR was performed using the SsoFast EvaGreen Supermix (Bio-Rad) according to the manufacturer recommendations. 50 ng of cDNA and 500 nM of each qPCR primer were used per sample, with GAPDH used as the housekeeping gene. Thermocycling and cycle threshold (CT) measurements were conducted using the CFX96 Touch Real-Time PCR Detection System (Bio-Rad). Standard and melt curve analyses were performed to validate qPCR primers and cycling parameters (FIG. 35). Relative gene expression was determined using the delta-delta C_{T} method. All primers used for qPCR are provided in Table 6.

### Caspase-6 Cleavage Assay

500,000 cells were resuspended in lysis buffer (10 mM DTT, 50 mM HEPES, 50 mM NaCl, 10 mM EDTA and 0.1% CHAPS, pH 7.5) and subjected to two freeze-thaw cycles using liquid nitrogen. 5 units of human recombinant caspase-6 protein (Millipore-Sigma #218799) was then added to the resulting supernatant and incubated at 37° C. for 5 hr. Reactions were halted with 1× NuPAGE LDS Sample Buffer (Thermo Fisher Scientific) with 2.5% (v/v) β-mercaptoethanol. Samples were then subjected to western blot as described below.

### Western Blot

Cells were lysed using radioimmunoprecipitation assay (RIPA) buffer (10 mM Tris-HCl pH 8.0, 140 mM NaCl, 1 mM EDTA, 1% Triton X-100, 0.1% SDS, 0.5% sodium deoxycholate, 2.5% β-mercaptoethanol) or 100 μL 1× NuPAGE LDS Sample Buffer (Thermo Fisher Scientific) containing 2.5% (v/v) β-mercaptoethanol and subsequently boiled at 95° C. for 5 min. Protein lysates were then electrophoresed on a NuPAGE Bis-Tris Protein Gel (4-12%) using NuPAGE SDS Running Buffer (Thermo Fisher Scientific) for 2 hr at 130 V before transfer to a nitrocellulose membrane in Towbin Buffer (20 mM Tris-HCl pH 8.3, 192 mM glycine, and 10% [v/v]methanol, 1% SDS) for 2.5 hr at 75 V in an ice chamber with constant agitation.

Membranes were then blocked for 1 hr with 0.5% (v/v) bovine serum albumin fraction V in Tris-buffered saline (TBS; 20 mM Tris-HCl pH 7.5, 150 mM NaCl) with 0.1%, Tween-20 (TBS-T). Membranes were subsequently incubated overnight at 4° C. with the following primary antibodies: rabbit anti-GAPDH (1:1,000, Cell Signaling Technology #2118) or rabbit anti-HTT, clone EPR5526 (1:1,000, Abcam #109115) in blocking solution. After overnight incubation, the membranes were washed three times with 1×TBS-T and incubated with HRP-conjugated goat anti-rabbit antibodies (1:2,500, Cell Signaling Technology #7074P2) in blocking solution for 1 hr at room temperature. Membranes were then washed three times with 1×TBS-T and developed using Clarity Western ECL Substrate (Bio-Rad) and visualized using an Odyssey Imager (LI-COR). Band intensity was quantitated using ImageJ and normalized to GAPDH.

### Next-Generation Sequencing

Genomic DNA and cDNA were isolated as described above. Amplicons for sequencing were generated by PCR using KAPA HiFi HotStart (Roche) using primers with overhangs compatible with Nextera XT indexing (IDT) (Table 6). Following validation by agarose gel electrophoresis, PCR products were isolated using AMPure XP PCR purification beads (Beckman Coulter). Indexed amplicons were generated using a Nextera XT DNA Library Prep Kit (Illumina) and subsequently quantified and pooled. Libraries were then sequenced using a MiSeq Nano Flow Cell for 251 cycles from each end using the MiSeq Reagent Kit v2 (500-cycles). FASTQ files were created and demultiplexed using bcl2fastq v2.17.1.14 Conversion Software (Illumina). All NGS was performed by the Roy J. Carver Biotechnology Center at the University of Illinois Urbana-Champaign (Urbana, IL).

Base editing rates were quantified using CRISPResso2. Reads with average Phred scores below 30 were removed, and remaining reads were aligned to the expected amplicon sequences.

Exon skipping rates were quantified using the STAR RNA-Seq aligner by Galaxy. Forward and reverse reads were combined and aligned to the human reference genome (GRCh38) using STAR. 2-pass mapping was then used for splice junction analysis with a MAPQ value of 60 for .bam files. Splice junctions were determined from the STAR SJ.out.tab files, where the percentage of a junction event was defined as the number of reads for the target junction divided by the total number of events at the specified junction. Sashimi plots for splice junctions were generated by the Integrative Genomics Viewer (IGV's) using the .bam and .bai files produced by RNA-STAR. The Sashimi plots presented herein were traces of the images generated by IGV.

### RNA-Seq

Following RNA isolation, RNA-seq libraries were prepared using the TruSeq mRNA library prep kit (Illumina). Samples were sequenced with a NovaSeq 6000 system (Illumina). For all analysis, a quality check was performed using FASTQC. All samples showed an average per-base read quality scores above 34, indicating high quality reads that required no trimming. The reads were then mapped to the GRCh38 reference human genome using HISAT2. Mapped reads were counted across human genes with featureCounts using annotations of GRCh38 obtained from GENCODE. About 60% of reads were assigned to genes. Differential expression analysis was then performed using Limma-voom [139] which first calculates TMM normalized counts-per-million (CPM) to account for compositional bias. Genes expressed at very low levels (CPM lower than 0.5) were filtered out before differential expression analysis. A differentially expressed gene was defined as a gene with a Benjamin-Hochberg adjusted P<0.05.

### Long-Read Sequencing

RNA was harvested using the RNeasy Plus Mini Kit (Qiagen) according to manufacturer's instructions. cDNA was synthesized using the qScript Ultra Flex cDNA Kit (Quanta Biosciences) and sequence specific primers from 2.5 pg of RNA. Long-read PCR was conducted using the repliQa HiFi ToughMix (Quanta Biosciences) with 1 μL of cDNA, ToughMix (1×), forward primer (0.3 μM), reverse primer (0.3 μM), and water (up to 25 μL). Cycling parameters were used as recommended by the manufacturer. All primers used are in Table 6. The samples were sequenced with a PacBio REVIO system.

Long-read sequencing data was aligned to the human reference genome (hg38) using minimap2 (v2.28, splice:hq preset) on Galaxy. Splice junctions were extracted from BAM files by parsing CIGAR N operators to obtain splice donor and acceptor coordinates for each junction. For every read, junctions were ordered sequentially to create a splicing pattern. Reads sharing identical junction patterns were grouped together. To minimize the number of fragmented reads analyzed, reads containing less than ten junctions were excluded. Junctions were annotated against the MANE-Select reference transcript for HTT (GENCODE v42) with a ±5 bp tolerance at splice sites to assign each splice donor and acceptor to its corresponding exon.

To quantify each splicing event, splice junctions were identified from each long read, and the number of reads containing each junction was counted. Reads with more than one alternative splicing event were counted separately from reads containing a single alternative splicing event. This ensured that distinct patterns containing multiple alternative splice junctions were not combined with sequences showing only one instance of alternative splicing. The frequency of a splicing event was calculated as the number of reads containing that event divided by all reads at that junction site.

### AAV Vector Production

HEK293T cells were seeded onto 15-cm plates and maintained in DMEM supplemented with 10% (v/v) FBS and 1% (v/v) penicillin/streptomycin. After 16 hr, cells were transfected with 65 pg total of pAAV-CAG-N/C-CBE-HTT-4 or pAAV-CAG-N/C-SpRY-ABE-HTT-3 alongside pAAV9 and pHelper in a 1:1:1 ratio using PEI MAX (pH 8). Cells were harvested 72 hr after transfection by manual dissociation using a cell scraper and centrifuged at 2,000 RPM for 5 min at room temperature. The supernatant was collected into a fresh tube and mixed with 40% PEG 8000 (Thermo Fisher Scientific) in a 4:1 ratio (v/v) and stored overnight at 4° C. Cell pellets were then resuspended in 2 mL of lysis buffer (50 mM Tris-HCl pH 8.0 and 150 mM NaCl) per plate. The following day, the solution was centrifuged at 3,000 RPM for 30 min at 4° C., with the resulting pellets resuspended in lysis buffer and subjected to three consecutive freeze-thaw cycles. Supernatants were subsequently treated with 0.5% Triton X-100 (Thermo Fisher Scientific) and 50 units/mL Benzonase (Millipore Sigma) and shaken at 37° C. for 1 hr. The lysate was then centrifuged at 4,000 RPM for 15 min at room temperature. The resulting supernatant was overlaid onto an iodixanol density gradient using 15%, 25%, 40% and 60% Opti-Prep solution (Sigma-Aldrich) and the AAV vector was isolated by ultracentrifugation at 58,400 RPM for 2 hr at 18° C. This step was repeated using a second iodixanol density gradient using fractions of 30%, 40% and 60%. Following the second extraction, AAV vector was filter-dialyzed using an Amicon Ultra 100 kDa MWCO column (Millipore Sigma) with PBS containing 0.001% Tween-20. Following treatment with DNase I (Millipore Sigma), AAV vector titer was determined by qPCR, with the vector stored at −80° C.

Stereotaxic injections

All animal procedures were approved by the Illinois Institutional Animal Care and Use Committee (IACUC) at the University of Illinois Urbana-Champaign and conducted in accordance with the National Institutes of Health (NIH) Guide for the Care and Use of Laboratory Animals.

YAC128 mice were generated by breeding female FVB/N mice with male YAC128 mice obtained from the Jackson Laboratory (Stock #004938). Genotype was determined by qPCR using transgene specific probes (TransnetYX).

For studies involving the CBE, two-month-old YAC128 mice were injected with 1×10⁹ VGs each of AAV9-CAG-N-CBE-sgRNA and AAV9-CAG-C-CBE-sgRNA in 2 μl of PBS with 0.001% Tween-20, per hemisphere. The coordinates for the injection were: anterior-posterior (AP)=0.86 mm, medial-lateral (ML)=±1.80 mm, and dorsal-ventral (DL)=−3.75 mm.

For studies involving the ABE, one-month old YAC128 mice were injected with 1×10⁹, 1×10¹⁰ and 1×10¹¹ VGs of each AAV9-CAG-N-SpRY-ABE-sgRNA and AAV9-CAG-C-SpRY-ABE-sgRNA in 2 μl of PBS with 0.001% Tween-20, per hemisphere. The coordinates for each injection were: AP=0.86 mm, MV=±1.80 mm, and DL=−3.75 mm, −3.55 mm, and −3.35 mm.

All groups for each study were sex-balanced and litter-matched.

### Behavior

All measurements and scoring were performed either by a researcher blinded to the genotype or by automated software.

Mice were weighed each week.

At 12 months of age, mice were analyzed using an observational scoring system for hindlimb clasping. Mice were suspended by holding base of the tail and scored based on appearance of hindlimbs as follows: 0=legs splayed outwards; 1=hindlimbs are drawn towards each other (without touching) or one leg is drawn into the body; 2=both legs are pulled in tightly, either touching each other or touching the body.

Grip strength was measured using BIO-GS3 (BioSEB) grip strength meter. Animals were placed over the rod such that both forelimbs grasped it. The animal was then pulled from the midpoint of the tail and the force exerted in Newtons (N) was recorded.

Motor function was determined using an accelerating Rotamex-5 (Columbus Instruments). Animals were placed on an apparatus that gradually accelerated from 4 to 40 rpm over 5 min and latency to fall time was recorded across three independent sessions that were averaged. Mice that turned twice on the rod were marked as fallen.

For the open field test, mice were placed at the center of a square in a white box and allowed to freely move for 10 min while being recorded by an overhead camera. Total distance traveled was determined by an automated tracking system software (Panlab Smart 3.0).

For the elevated plus maze test, mice were placed on a continuous plus-shaped platform with two closed and two open regions for 5 min. The time spent exploring the open regions was quantified by ANY-maze software (Stoelting, Wood Dale, IL, USA).

Prior to all measurements, mice were habituated to the testing room for at least 30 min.

### Tissue Harvesting and RNA Purification

Mice were anesthetized using 3% isoflurane delivered by vaporizer in a closed chamber and transcardially perfused using PBS. The striatum and cortex were dissected and divided in two halves, with one half stored in RNAlater Stabilization Solution (Thermo Fisher Scientific) and the other flash frozen. DNA and RNA were isolated using the DNeasy Blood and Tissue Kit (Qiagen) and RNeasy Plus Mini Kit (Qiagen), respectively. Prior to purification, tissues were homogenized in PBS using a KIMBLE Dounce Tissue Grinder.

### Western Blot for Tissues

Protein was isolated from bulk striatal tissue using RIPA buffer supplemented 10 μL/mL of Halt Phosphatase Inhibitor Cocktail (Thermo Fisher Scientific 78420). Samples were then boiled at 95° C. for 5 min and electrophoresed using a NuPAGE Bis-Tris Gel (4-12%) with NuPAGE SDS Running Buffer (Thermo Fisher Scientific) for 2 hr at 130 V, before a electrophoretic transfer to a nitrocellulose membrane in Towbin buffer with 0.5% SDS, for 2.5 hr at 75 V. Membranes were then blocked with 1% (v/v) non-fat dry milk in TBS-T for 1 hr and incubated overnight at 4° C. with primary antibodies in blocking solution. The following primary antibodies were used: mouse anti-HTT antibody, clone 5HU-1H6 (1:1,000, Sigma Aldrich MAB5490) and rabbit anti-GAPDH (1:1,000, Cell Signaling Technology #2118). After the overnight incubation, membranes were washed three times with TBS-T and incubated with HRP-conjugated goat anti-mouse (1:1,000, Cell Signaling Technology #7056) or goat anti-rabbit (1:1,000, Cell Signaling Technology #7074P2) antibodies in blocking solution for 1 hr at room temperature. Membranes were washed three times with TBS-T, developed using Clarity Western ECL Substrate (Bio-Rad) and visualized using an Odyssey Imager (LI-COR). Band intensity was quantitated using ImageJ and normalized to GAPDH.

### Immunohistochemistry

Dissected striatum and cortex were fixed in 4% (v/v) paraformaldehyde (PFA) at 4° C. and embedded in OCT for generation of 15 μm coronal sections using a CM3050 S cryostat (Leica).

For immunostaining, sections were washed three times with PBS for 15 min and incubated in blocking solution (PBS with 10% [v/v], goat serum [Abcam #7481] and 0.5% Triton X-100) for 2 hr at room temperature, at which point the sections were stained with primary antibodies in blocking solution overnight at 4° C. Afterwards, sections were washed three times with PBS and incubated with secondary antibodies for 1 hr at room temperature, before three final washes with PBS. Sections were then incubated with 1 μg/mL of DAPI (Sigma-Aldrich) for 10 min and mounted onto slides using VECTASHIELD HardSet Antifade Mounting Medium (Vector Laboratories). Sections were imaged using a Leica TCS SP8 confocal microscope (Beckman Institute Imaging Technology Microscopy Suite, University of Illinois Urbana-Champaign, Urbana, IL), with all images analyzed using ImageJ.

The following primary antibodies were used: rabbit anti-V5 (1:100, Cell Signaling Technology #13202), mouse anti-NeuN (1:400, Cell Signaling Technology #94403), rabbit anti-Iba1 (1:500; Wako Pure Chemicals Industries 019-19741), and mouse anti-GFAP (1:200; Cell Signaling Technology, #3670)).

The following secondary antibodies were used: goat anti-rabbit Alexa Fluor 555 (1:400, Cell Signaling Technology #4413) and goat anti-mouse Alexa Fluor 488 (1:400, Abcam #150113) and goat anti-chicken Alexa Fluor 647 (1:200, Abcam #150175).

mHTT Inclusion Staining

Floating sections (30 μm) from the striatum and cortex were washed three times in 12-well plates with PBS and incubated with blocking solution (0.3% Triton X-100 and 1% goat serum in PBS) for 2 hr at room temperature. Sections were then incubated with the primary antibody EM48 (1:80, Millipore MAB5374) for 72 hr. Afterwards, sections were washed three times with PBS and incubated with the secondary antibody (goat anti-mouse Alexa Fluor 488, 1:400, Abcam 150113) for 2 hr at room temperature. Following a subsequent 10-min incubation with 1 μg/mL of DAPI (Sigma-Aldrich), floating sections were mounted onto microscope slides using ProLong Gold Antifade Mountant (Cell Signaling Technology #9071). Sections were then imaged using a Leica TCS SP8 confocal microscope (Beckman Institute Imaging Technology Microscopy Suite, University of Illinois Urbana-Champaign, Urbana, IL).

### MRI Analysis

A 9.4T/20-cm horizontal bore animal MRI system with a cryoprobe and ParaVision v6.0.1 software (Bruker BioSpin) was used to analyze 12-month-old YAC128 and FVB/N mice. The scanner was equipped with a gradient coil set of 660 mT/m per axis and a 4570 T/m/s slew rate, allowing for high resolution imaging. Body temperature and respiratory function were monitored using a three-dimensional (3D) T2-weighted fast spin echo (FSE) sequence using a 1030 MRI-compatible small animal monitoring and gating system (SA Instruments). Mice were anesthetized with 2% isoflurane, which was delivered by a vaporizer.

Post-processing of MRI files in the form of DICOM images was conducted using 3D Slicer. Briefly, anatomical images were first merged to generate an average of all anatomical images. This template was then segmented into different brain regions using the Allen Mouse Brain Atlas. Transformations of the atlas were projected onto the images of the mouse brain and corresponding segmentation of brain anatomical structures were made. Striatal and cortical volumes were measured in mm³ and normalized to the volume of age-matched wild-type FVB/NJ mice.

### Statistical Analysis

Statistical analysis was performed using GraphPad Prism v9.1 (GraphPad Software, Inc.). All experiments consisted of independent biological replicates. Groups were compared using an unpaired one-tailed t-test or a one-way ANOVA with Tukey's post-hoc analysis.

### Example 7: Identification of CRISPR Base Editors to Modify the SA of HTT Exon 13

The human HTT gene consists of 67 exons, with the proteolytic cleavage sites implicated in the formation of the N-terminal fragment of the HTT protein located in exons 12 and 13. To disrupt the proteolysis of HTT without fully ablating its expression, we sought to use exon skipping to remove these cleavage sites, which we hypothesized would create HTT protein isoforms resistant to proteolysis. To accomplish this goal, we utilized CRISPR base editing, a gene-editing technology that can facilitate exon skipping through its targeted editing of splice sites.

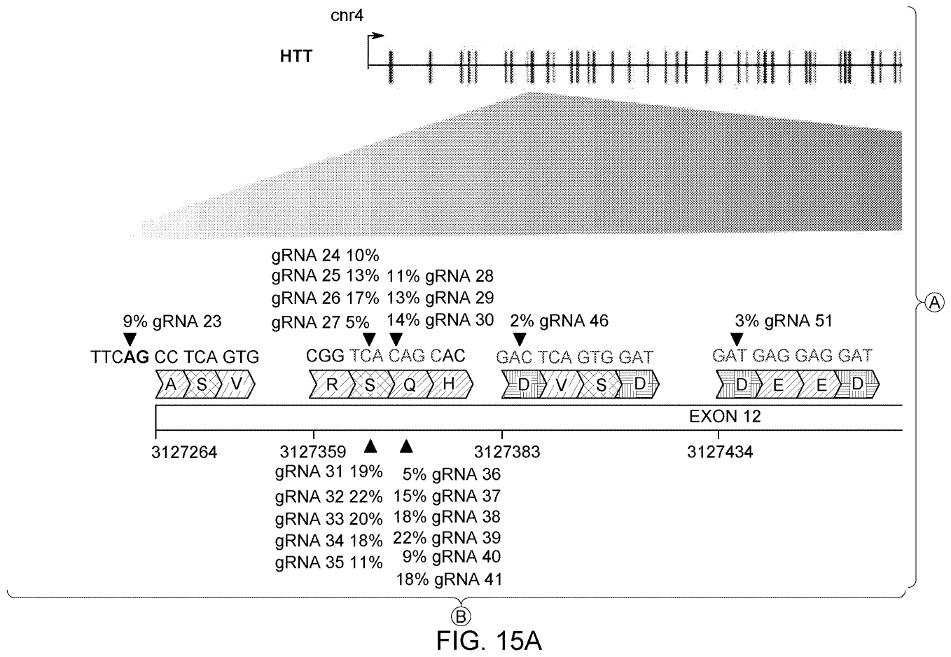

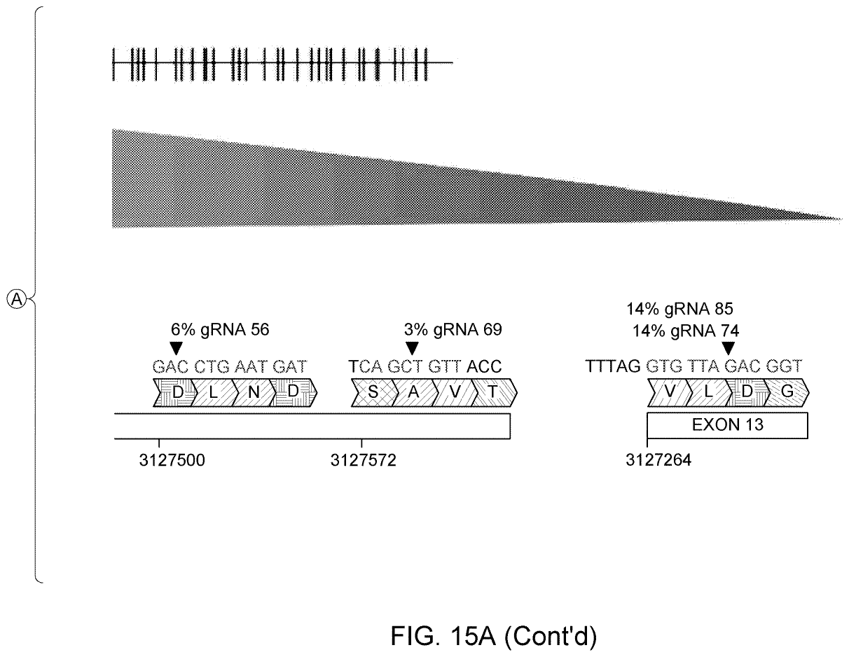

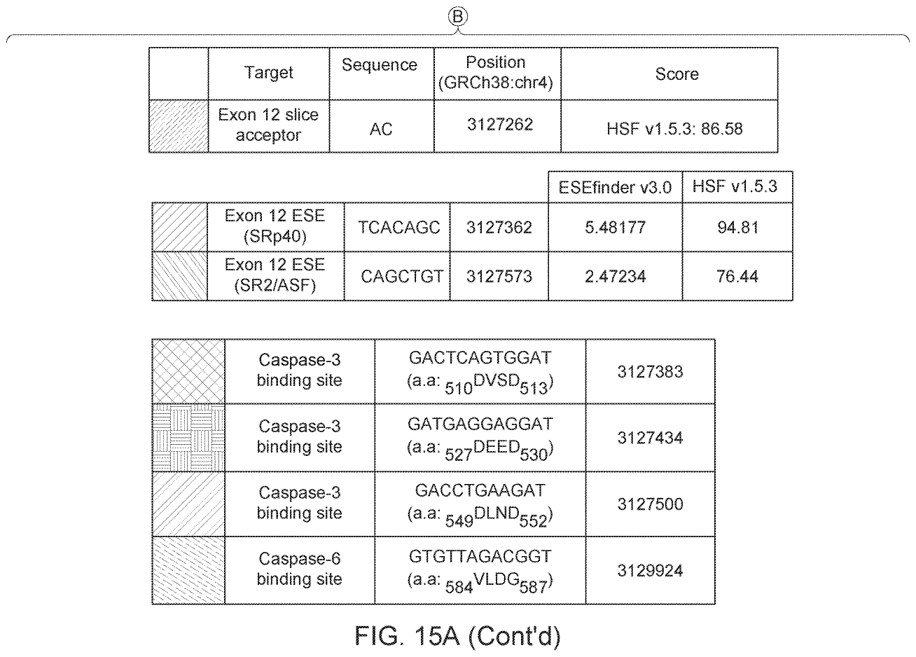

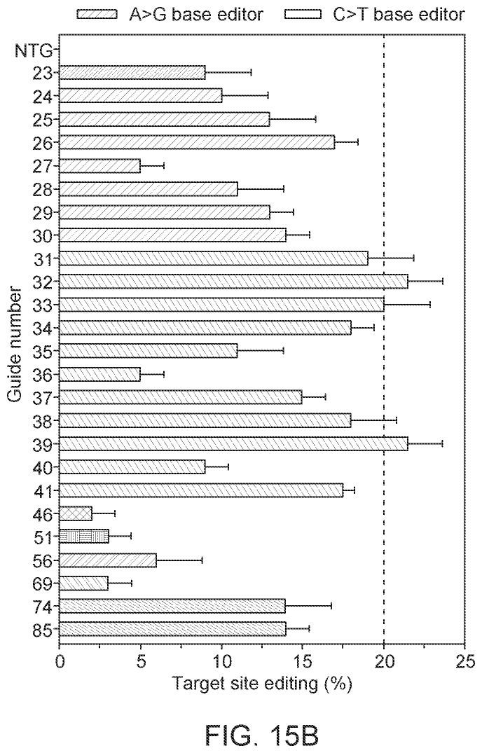

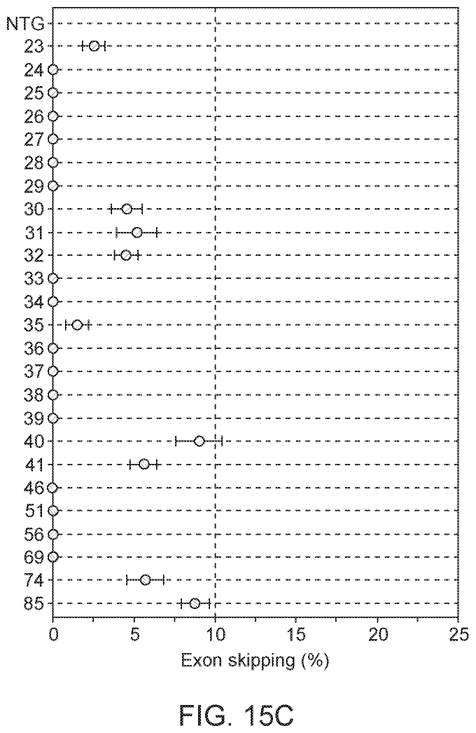

First, we designed cytosine base editors (CBEs) and adenine base editors (ABEs)—initially centered on BE3 and ABE 7.10 respectively—to target the SAs for exons 12 and 13, as well as two putative splice enhancers for exon 12, which we identified using ESEfinder and Human Splicing Finder. In conjunction with these four splice sites, we also designed base editors to target the predicted caspase-3 and caspase-6 cleavage sites in exons 12 and 13 (FIG. 15a). To enable targeting, we utilized a suite of Cas9 variants, including the prototypical Cas9 nuclease from Streptococcus pyogenes (SpCas9), which recognizes a 5′-NGG-3′ and to a lesser degree a 5′-NAG-3′ protospacer adjacent motif (PAM) as well as the engineered variants xCas9 and SaCas9-KKH, which each recognize non-NGG PAM sequences (Table 5). At the time this study was initiated, xCas9 and SaCas9-KKH were among the state-of-the-art for PAM-flexible Cas9 variants. BE3 and ABE7.10 also represented among the most efficient deaminase domains available.

Each CBE and ABE variant was transfected to human embryonic kidney (HEK) 293T cells and evaluated for their ability to modify the HTT gene. DNA sequencing revealed that, of the 103 variants analyzed, 40 of them were able to modify their target base (FIG. 15b). Though 25 of the 40 variants targeted the exon 12 SA, its splice enhancer or its putative protease cleavage sites, only two of these variants edited with an efficiency over 20% (FIG. 15b), with a further analysis found to reveal that neither of these two variants induced skipping at a rate that exceeded 10% (FIG. 15c). The other 15 variants with detectable activity targeted the SA for HTT exon 13. This included CBE-HTT-4, which had the highest activity and edited with ~27% efficiency (P=0.0001; FIG. 10C).

We next compared the DNA editing capabilities of the most efficient CBE system identified above, CBE-HTT-4, against a split-intein version of it intended for AAV delivery, as the carrying capacity of an AAV vector limits its ability to deliver full-length versions of these editors within a single viral particle. Our split-intein CBE is encoded across two vectors: one vector expresses the APOBEC deaminase and the first 712 residues of the SpCas9 protein fused to the N-terminus DnaB intein from Rhodothermus marinus, which mediates a protein trans-splicing reaction with its C-terminal counterpart, and the second vector expresses the C-terminal DnaB intein fused to residues 713-1,371 of the SpCas9 protein fused with an uracil glycosylase inhibitor (UGI) domain. These inteins perform similarly as the DnaE inteins from Nostoc punctiforme. Each vector further encodes an sgRNA expression cassette (FIG. 10D).

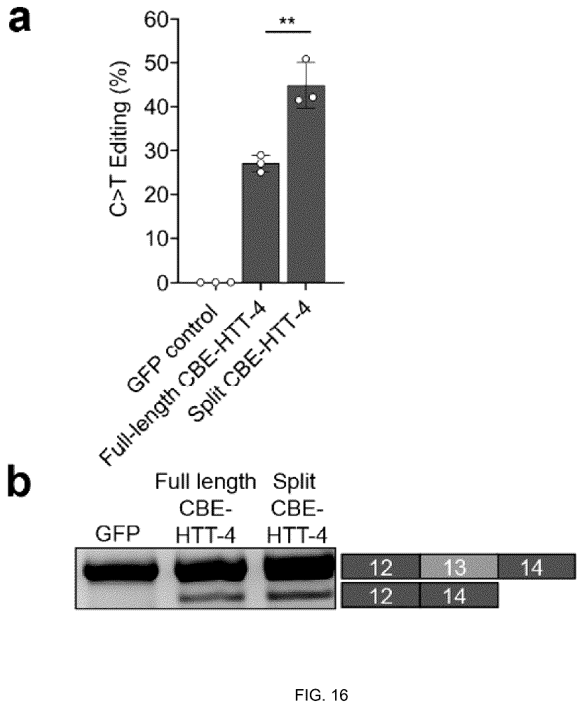

This split-intein system introduced the target C>T edit in the SA of HTT exon 13 at an efficiency of ~45% in HEK293T cells, though we found that it also created a G>A bystander mutation +1-bp from the SA at an efficiency of ~46%, with this edit expected to create a missense mutation in HTT [GTG>ATG: Val584>Met](FIG. 10e and FIG. 16). Nonetheless, we note that exonic mutations that occur simultaneously with the target modification in the SA are not expected to have a biological effect, as the exon would be skipped.

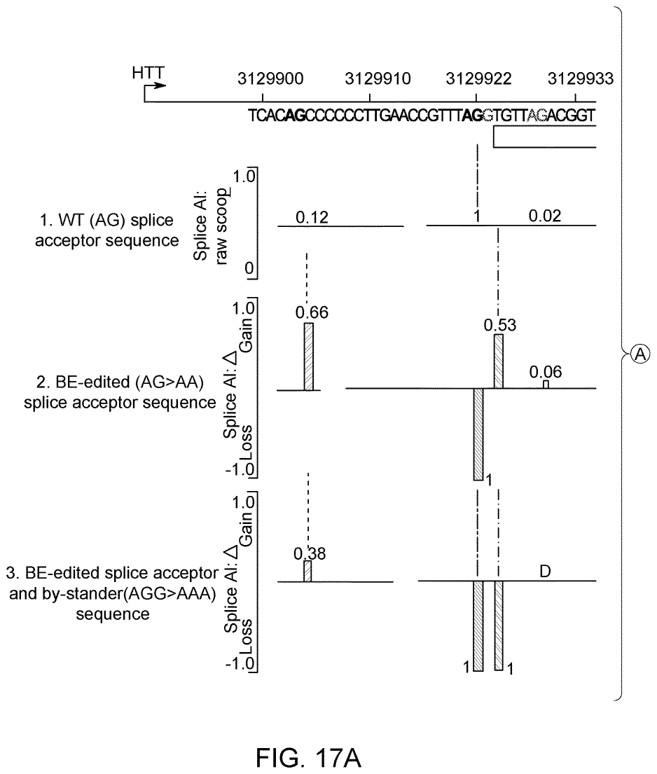

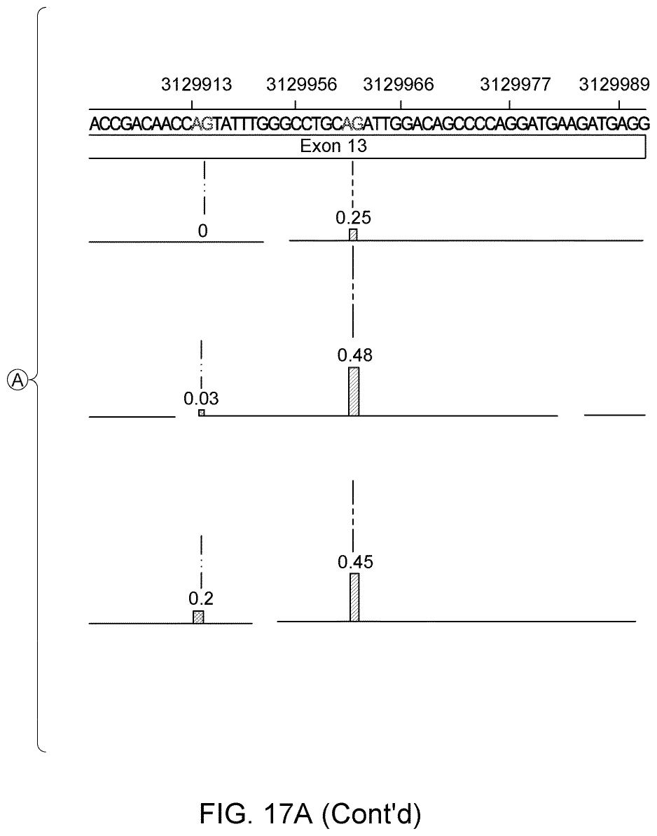

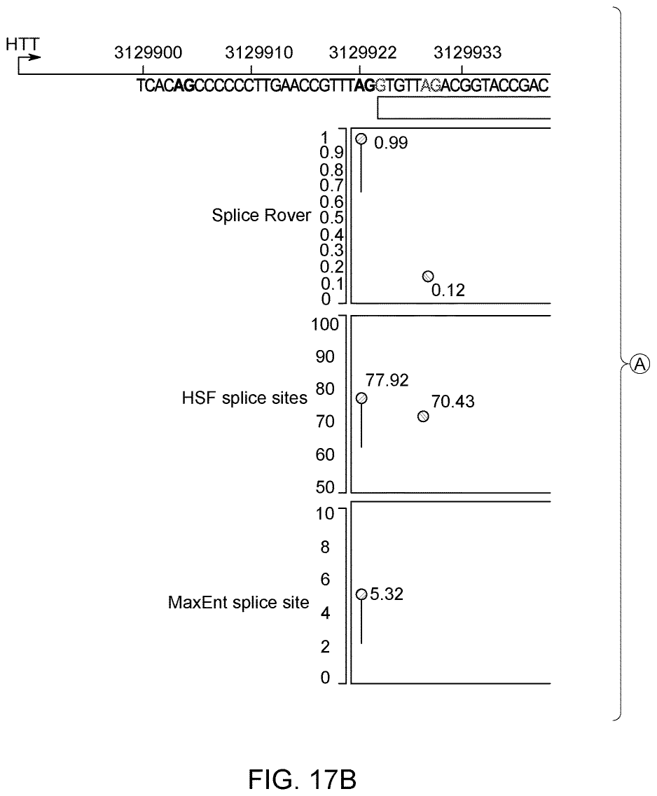

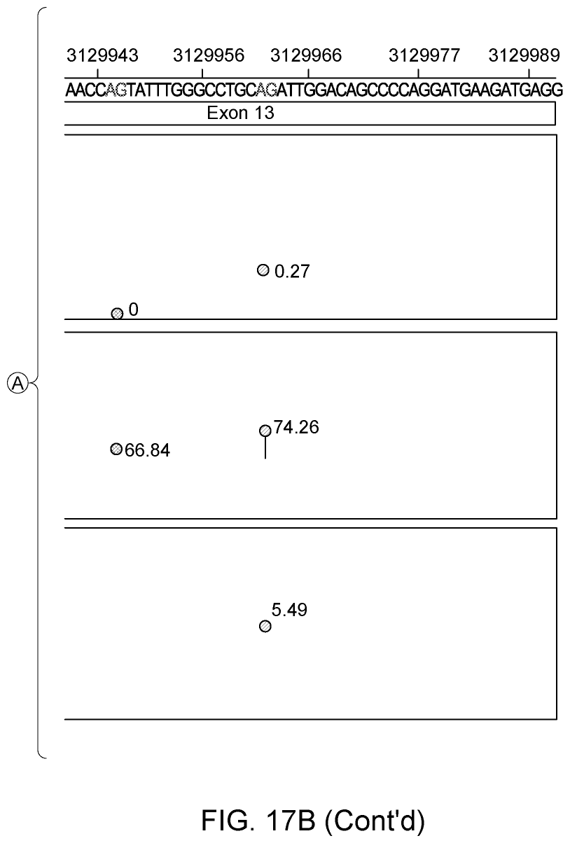

Interestingly, ablating the SA for HTT exon 13 is predicted to result in a frameshift mutation in the HTT transcript, which is expected to trigger nonsense mediated decay (NMD) and a reduction in HTT expression. However, according to SpliceAI, MaxEntScan and Human Splicing Finder, there exists an alternative SA in exon 13, located 39 bps from the canonical SA, whose utilization is predicted to produce a 13-residue truncation in the HTT protein that would disrupt the caspase-6 cleavage site but not alter its reading frame (FIGS. 17a and 17b).

We next quantified the rate of exon skipping induced by this split-intein CBE in HEK293T cells. Based on an NGS analysis of PCR amplicons generated from cDNA using primers that targeted HTT exons 11 and 14 (FIG. 16b), a method that provides a high-throughput means to measure relative differences in transcript abundance. Based on NGS, we found that the split-intein version of CBE-HTT-4 induced more than three-fold higher relative skipping of exon 13 compared to its FL equivalent (~33% vs ~11.6%; P=0.0006; FIG. 10f). This analysis further revealed that editing of the SA in HTT exon 13 did in fact generate two major products, with ~16.7% of the analyzed HTT transcripts found to lack the entirety of exon 13, and ~16.9% of the transcripts found to lack only the first 39-bps of exon 13 (P<0.05 for both; FIG. 10g).

Last, we determined the specificity of this split-intein CBE. Using NGS, we measured C>T

TABLE 8
Guide-dependent off-target sites tested in this study.

| | | Genomic location | |
|---|---|---|---|
| ID | Sequence (5′→3′) | (GRCh38) | Gene |
| CBE4 (on-target) | ACACCTAAACGGTTCAAGGG | chr 4: 3129908 | HTT |
| | SEQ ID NO: 267 | | (ENSG00000197386) |
| CBE4 OT1 | ACAGCTATTGGGTTCAAGGG | chr 5: | Intergenic |
| | SEQ ID NO: 454 | +138604309 | |
| CBE4 OT2 | ACAGTCAAAGGGTTCAAGGG | chr 4: | Intergenic |
| | SEQ ID NO: 455 | +140082204 | |
| CBE4 OT3 | ACATTTAAATGGTTCAAGGT- | chr 13: | Intergenic |
| | SEQ ID NO: 486 | +108744040 | |
| CBE4 OT4 | ACATTTGAACCGTTCAAGGG | chr 2: | Intergenic |
| | SEQ ID NO: 457 | +149527162 | |
| CBE4 OT5 | AGACACAAACTGTTCAAGGG | chr12: | Intergenic |
| | SEQ ID NO: 458 | +2359583 | |
| | ACAGGTTAACGTTTCAAGGG | chr 6: | MTCO3P1 |
| CBE4 OT6 | SEQ ID NO: 459 | +151466540 | (ENSG00000235040) |
| CBE4 OT7 | ACATTTGAACCGTTCAAGGG | chr 2: | Intergenic |
| | SEQ ID NO: 457 | +5267170 | |
| CBE4 OT8 | AATCCTAAAAGGTTGAAGGG | chr 13: | Intergenic |
| | SEQ ID NO: 461 | +40651917 | |
| SpRY-ABE-HTT-3 | TTAGGTGTTAGACGGTACCG | chr 4: | HTT |
| (on-target) | SEQ ID NO: 304 | +3129920 | (ENSG00000197386) |
| | | | Intergenic |
| SpRY-ABE-HTT-3 | TTACAGGTGTTAGACGGTAGCA | chr 15: 58077695 | Intergenic |
| OT1 | SEQ ID NO: 487 | | |
| SpRY-ABE-HTT-3 | TTACTGGTGTTAGAtGGTACCT | chr X: 122595733 | Intergenic |
| OT2 | SEQ ID NO: 488 | | |
| SpRY-ABE-HTT-3 | TTAGGTGTTACACGCTACCC | chr X: 109694073 | Intergenic |
| OT3 | SEQ ID NO: 465 | | |
| SpRY-ABE-HTT-3 | TTAGGGGTTAGACGGTCACCT | chr 11: | Intergenic |
| OT4 | SEQ ID NO: 489 | 130525045 | |
| SpRY-ABE-HTT-3 | TTAGGTGTAAGAAGGTCCACCG | chr 3: 16959807 | PLCL2 |
| OT5 | SEQ ID NO: 490 | | (ENST00000615277) |
| SpRY-ABE-HTT-3 | TGTAGGTGTGAGATGGTACCG | chr 7: 133911706 | Intergenic |
| OT6 | SEQ ID NO: 491 | | |
| SpRY-ABE-HTT-3 | TTAGGGGTTAGACGGTcACCT | chr 11: | Intergenic |
| OT7 | SEQ ID NO: 489 | 130525045 | |
| SpRY-ABE-HTT-3 | TTAGGTGTTAGTGATACCGGAT | chr 5: 52932372 | Intergenic |
| OT8 | SEQ ID NO: 492 | | |
| SpRY-ABE-HTT-3 | TTAGGTGTTGACTTTACCGAGT | chr 1: 99462410 | Intergenic |
| OT9 | SEQ ID NO: 493 | | |
| SpRY-ABE-HTT-3 | TTAGGTGTTAGAGGTACAAGAG | chr 1: 75915876 | Intergenic |
| OT10 | SEQ ID NO: 494 | | |
| SpRY-ABE-HTT-3 | TTAGATGTTAGACGGTCCAATG | chr 1: 202288544 | Intergenic |
| OT11 | SEQ ID NO: 495 | | |
| SpRY-ABE-HTT-3 | TTGATGTTAGACGGCACCGAAC | chr 7: 150688538 | Intergenic |
| OT12 | SEQ ID NO: 496 | | |
| SpRY-ABE-HTT-3 | TTAGGTGTTAACGGTAAGGTAA | chr 2: 25827912 | Intergenic |
| OT13 | SEQ ID NO: 497 | | |
| SpRY-ABE-HTT-3 | TTAGGTGTTGAGGGTACAGCAA | chr 12: 88502532 | Intergenic |
| OT14 | SEQ ID NO: 498 | | |
| SpRY-ABE-HTT-3 | TTAGGTGTTAGACTGTAAGTAA | chr 12: 13604712 | Intergenic |
| OT15 | SEQ ID NO: 499 | | |
| SpRY-ABE-HTT-3 | TAGGTGTTAGACAGTACTGTTG | chr 4: 47586613 | Intergenic |
| OT16 | SEQ ID NO: 500 | | |
| SpRY-ABE-HTT-3 | TTAGGTGTTAGAGGTAGAG | chr 7: 134849500 | Intergenic |
| OT17 | SEQ ID NO: 501 | | |
| SpRY-ABE-HTT-3 | TTAGGTGTTGACGTCACCG | chr X: 39552937 | Intergenic |
| OT18 | SEQ ID NO: 502 | | |
| SpRY-ABE-HTT-3 | TTAGGTGTTAAGGGTACTG | chr X: 46466516 | Intergenic |
| OT19 | SEQ ID NO: 503 | | |
| SpRY-ABE-HTT-3 | TTAGGTGGAGTCGGTACCG | chr 16: 12879910 | Intergenic |
| OT20 | SEQ ID NO: 504 | | |
| SpRY-ABE-HTT-3 | TTAGGTGTTAGAAGGAACAG | chr 17: 21190320 | Intergenic |
| OT21 | SEQ ID NO: 505 | | |
| SpRY-ABE-HTT-3 | ATAGGTGTTAGACAGTACTG | chr 4: 47586614 | Intergenic |
| OT22 | SEQ ID NO: 506 | | |
| SpRY-ABE-HTT-3 | TAAGGTGTTAGACGGTCCCT | chr 2: 130259427 | Intergenic |
| OT23 | SEQ ID NO: 507 | | |

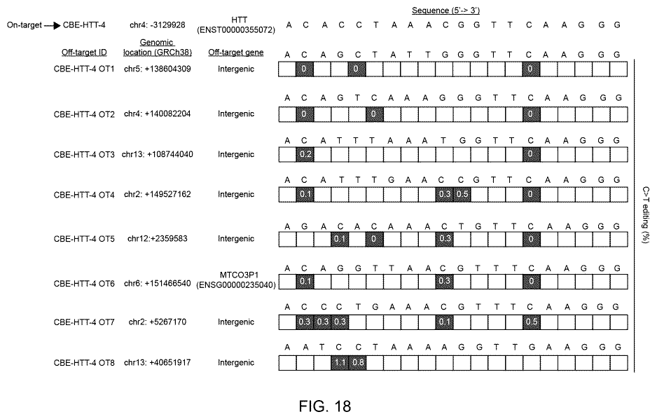

No editing was observed in HEK293T cells at any of the candidate OT sites (P>0.05; FIG. 18).

### Example 8: Editing the HTT Exon 13 SA Site Alters HTT Splicing

To further characterize the expression patterns resulting from the disruption of the SA site for HTT exon 13, we generated clonal HEK293T cell lines edited by the split-intein version of CBE-HTT-4 (FIG. 16a). After confirming the presence of the target edit in each clone (FIG. 16b), we conducted an NGS analysis on PCR products from cDNA prepared from the base editor-modified cell lines, as well as naive unedited HEK293T cells. In total, ~37% of the HTT transcripts in the modified clones lacked exon 13 (P=0.001; FIG. 16c), while ~46% of transcripts lacked only the first 39-bps of it (P=0.0003; FIG. 16c). Interestingly, ~10% of all transcripts retained a fragment of intron 12, which we hypothesized occurred through the utilization of an ‘AG’ sequence in an intergenic region 18-bp upstream of the targeted SA site (FIG. 16d), which is consistent with the model generated by Splice AI that predicted that disrupting the exon 13 SA and the concurrent introduction of a G>A mutation +1-bp of the SA would create cryptic SAs at nearby exonic and intronic sequences (FIG. 17a).

As the skipping of exon 13 and the retention of intron 12 is expected to result in a frameshift mutation in HTT, which in turn is anticipated to decrease its expression by NMD, we also measured the relative abundance of HTT mRNA in the isolated clones. Using primers that bind the exon 4/5 junction and exon 6, we found that the base editor-modified cell lines had on average a ~57% decrease in HTT mRNA compared to the naive, unedited HEK293T cells (P<0.0001; FIG. 11e).

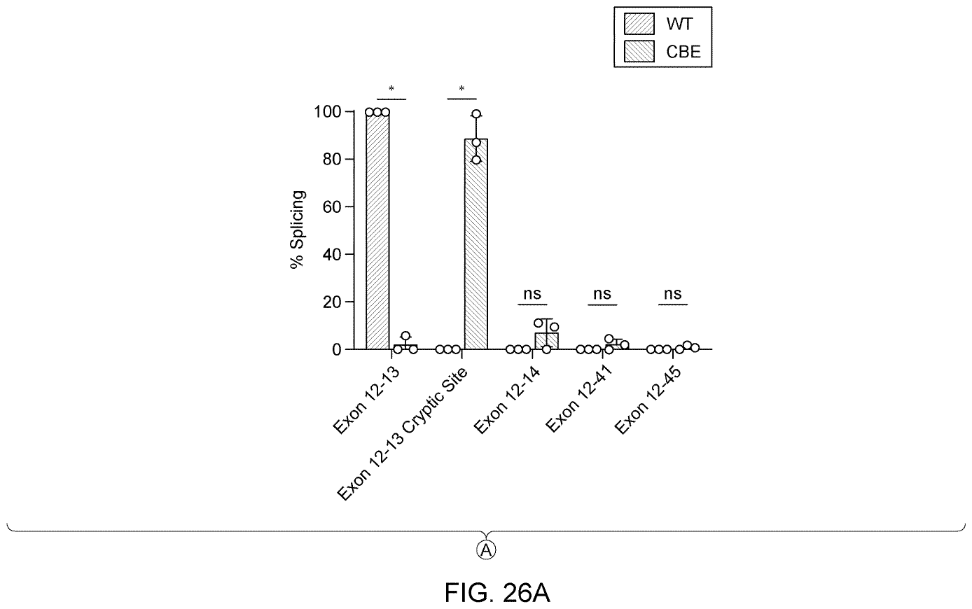

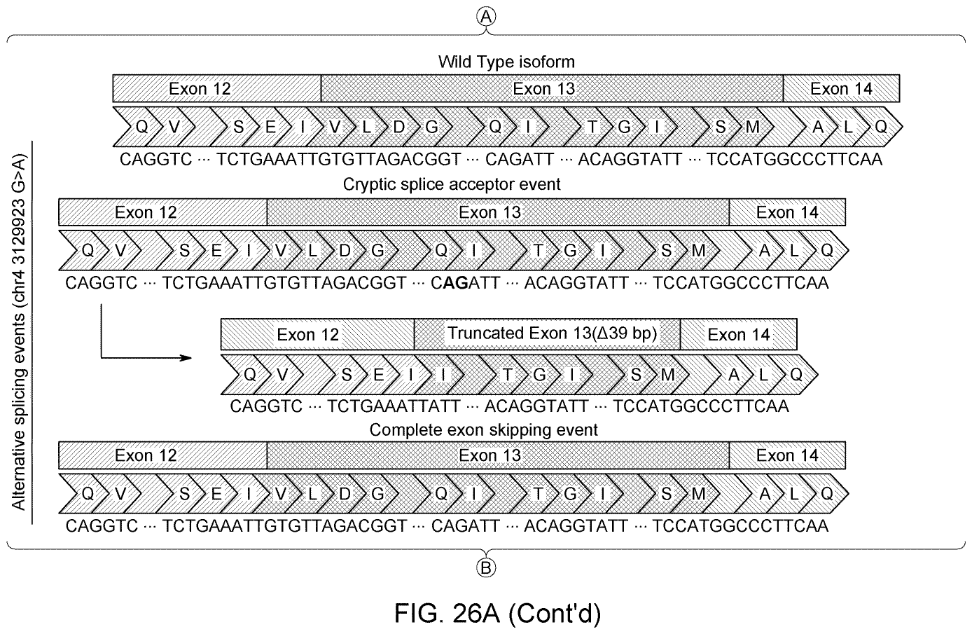

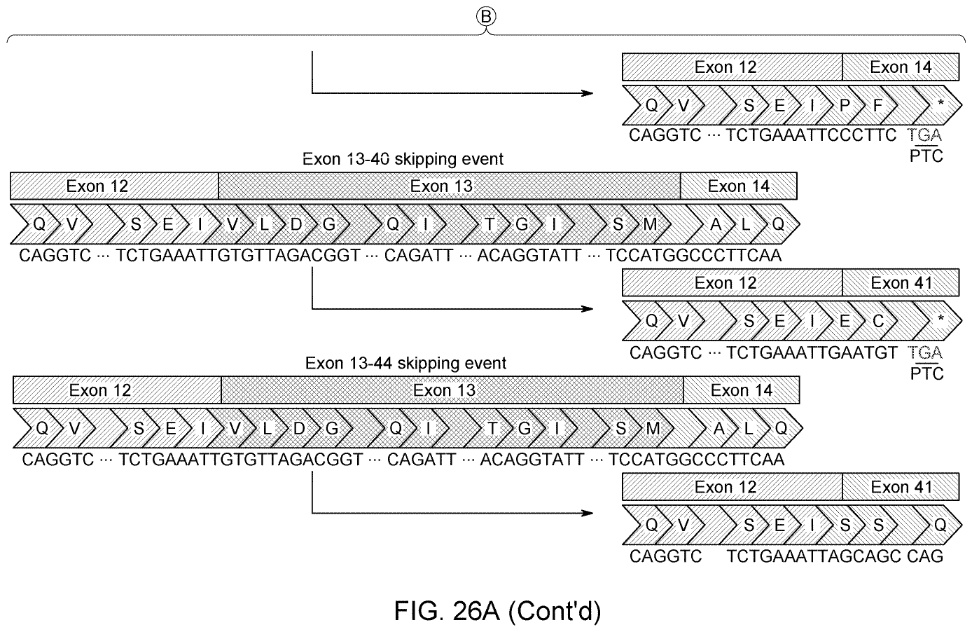

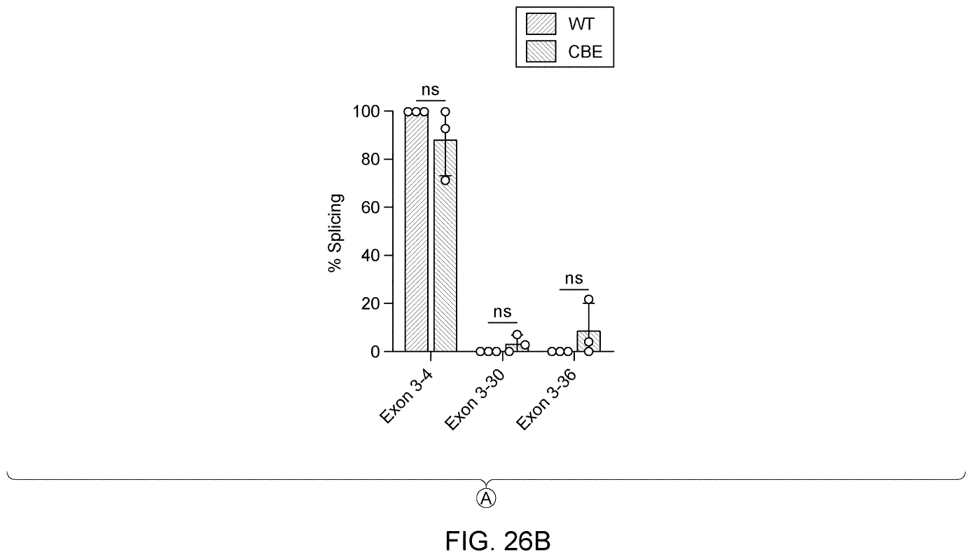

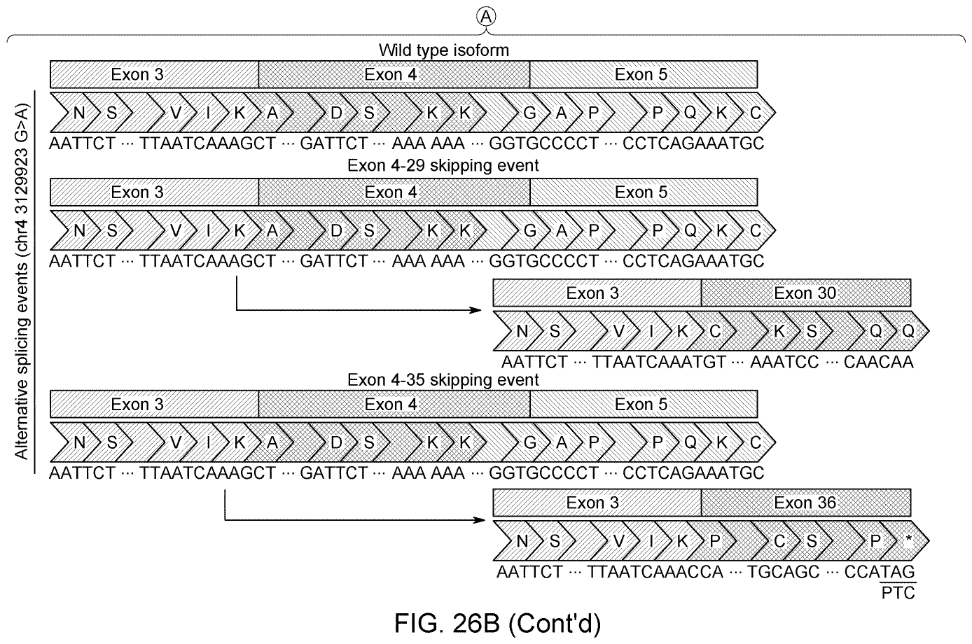

To determine if the HTT transcript reservoir was otherwise affected by editing of the exon 13 SA, we used long-read sequencing to analyze PCR amplicons generated with primers that bind HTT exon 1 and exon 48 (FIG. 26a, b). ~90% of the HTT transcripts had the expected products with no detectable alternative splicing (FIG. 26a). Notably, only minor alternative splicing was detected in the CBE-modified cells, chiefly at exons 3 (FIG. 26b; P>0.05) and 12 (FIG. 26a; P>0.05), which showed up to ~8% alternative splicing with exons 36 and 14, respectively.

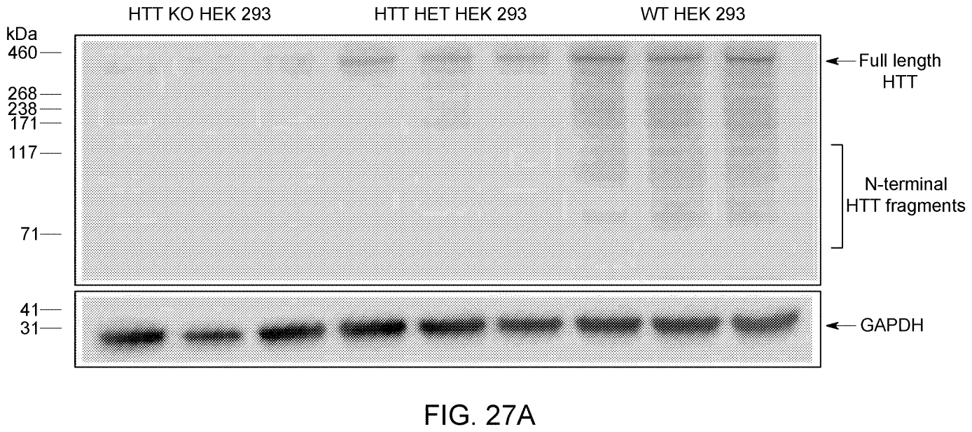

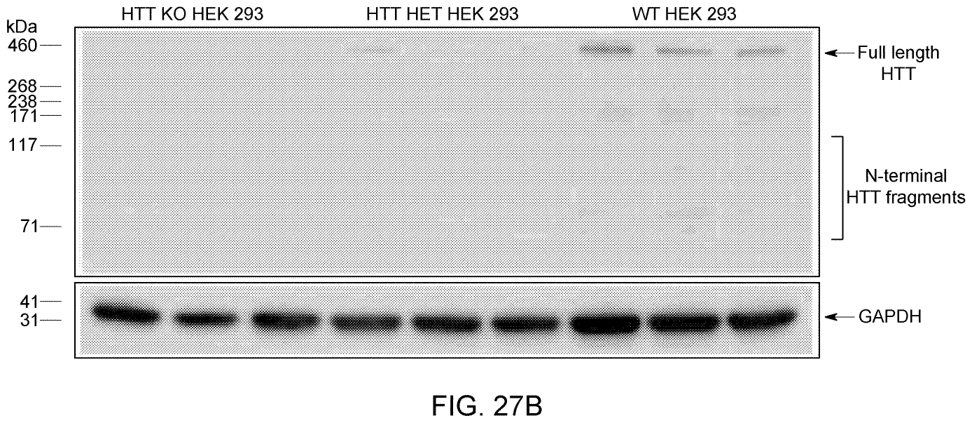

To determine whether the HTT protein isoforms produced in the modified cell lines were resistant to proteolysis by caspase-6, we incubated their lysates with purified caspase-6 enzyme (FIG. 11f, g). We then analyzed HTT protein by western blot using an anti-HTT antibody (EPR5526) that recognizes a motif between residues 49 and 148 of the protein and has been previously used to detect both FL and cleaved HTT protein products [49, 50] although the identity of the N-terminal fragments was not validated by mass-spectrometry. We validated the specificity in lysates from HEK293T cells modified to have either a knockout or partial reduction of HTT (FIG. 27a). According to western blot, the treated lysates originating from the base editor-modified clones showed a ~67% decrease in FL HTT protein compared to the naive cells (P=0.002, FIG. 11g). Importantly, we observed ~90% (P=0.0038, FIG. 11g) and a ~82% (P=0.001, FIG. 11g) decrease in N- and C-terminal HTT protein fragments, respectively, compared to their naive counterparts, indicating that the proteolytically resistant HTT isoform reduced N-terminal fragment formation by an additional ~23%. To rule out the possibility that cell line-specific differences in caspase-6 expression contributed to this result, we used western blot to measure caspase-6 protein in the cell lysates used in this experiment. The CBE-modified clones and native cells had equivalent amounts of caspase-6 (FIG. 11h, i).

Thus, our results demonstrate that disrupting the SA for HTT exon 13 by CRISPR base editing can create HTT protein isoforms resistant to cleavage by caspase-6.

### Example 9: In Vivo Exon Skipping of HTT Improves HD-Related Deficits

We next evaluated the effectiveness of the split-intein CBE in a transgenic rodent model of HD, specifically the YAC128 mouse model, which expresses a mutant form of the FL human HTT gene with intervening intronic sequences. YAC128 mice recapitulate pathological hallmarks of HD, including the formation of mHTT inclusions and brain atrophy.

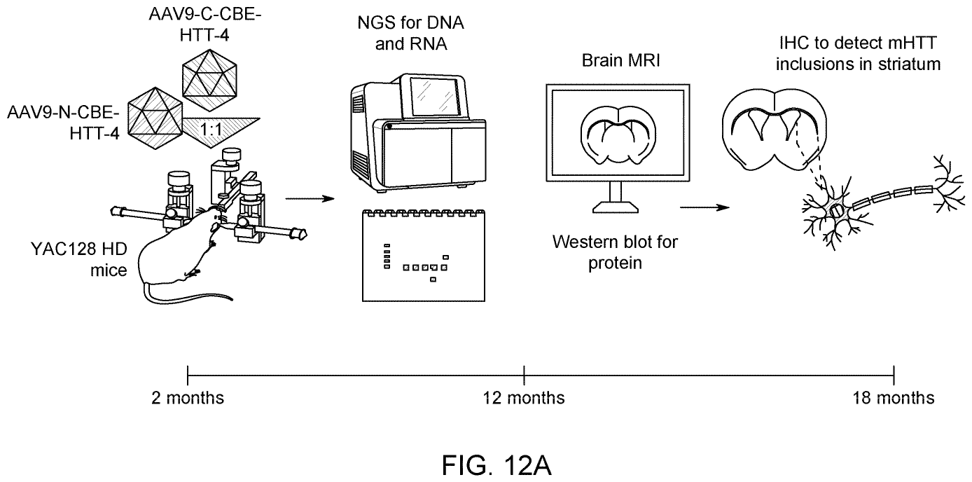

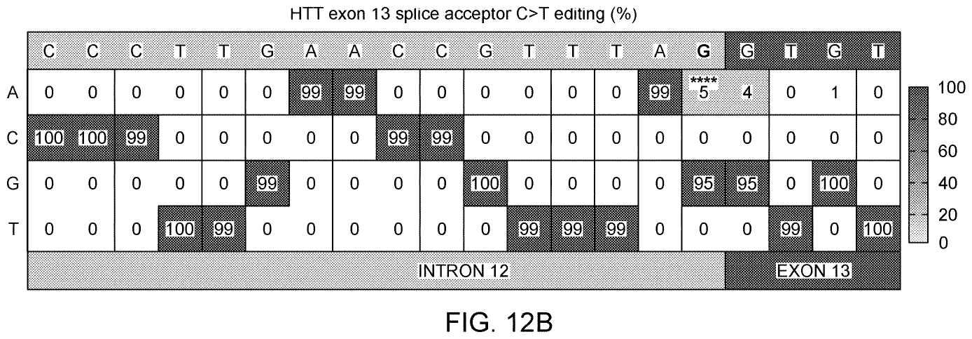

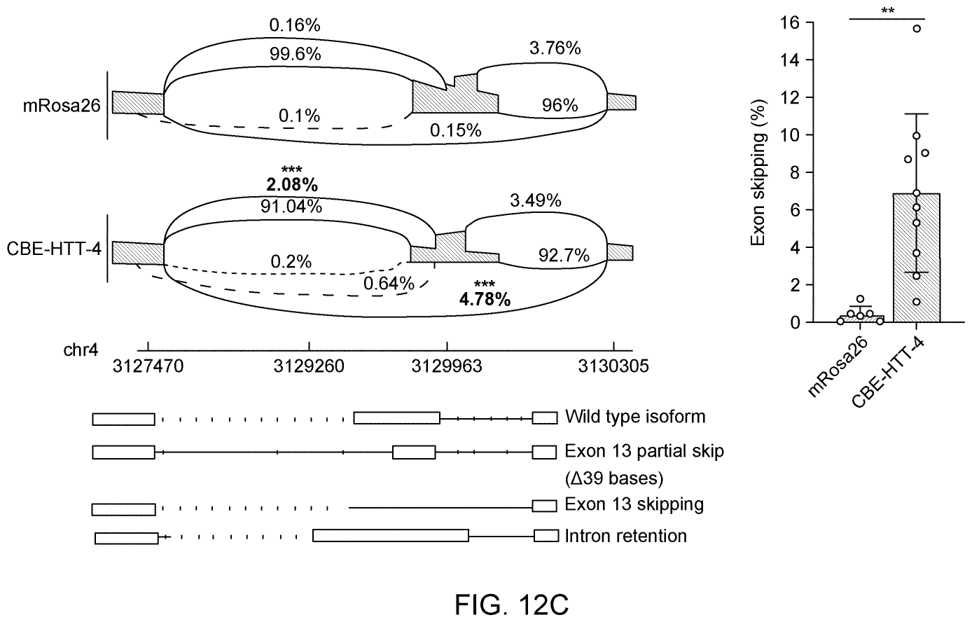

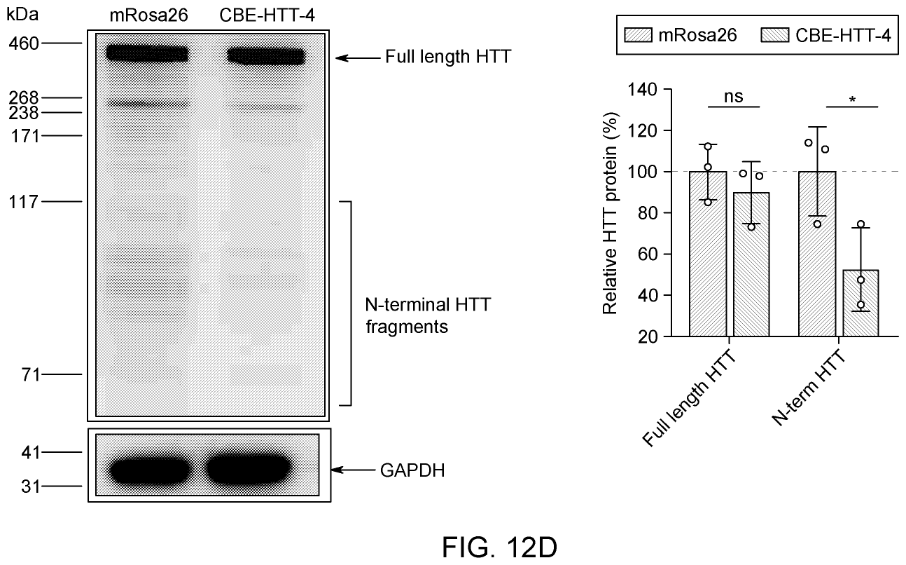

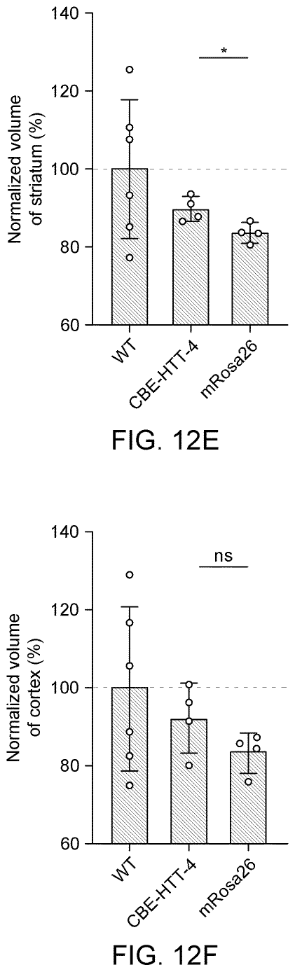

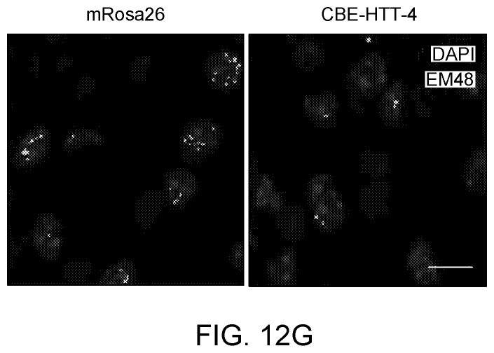

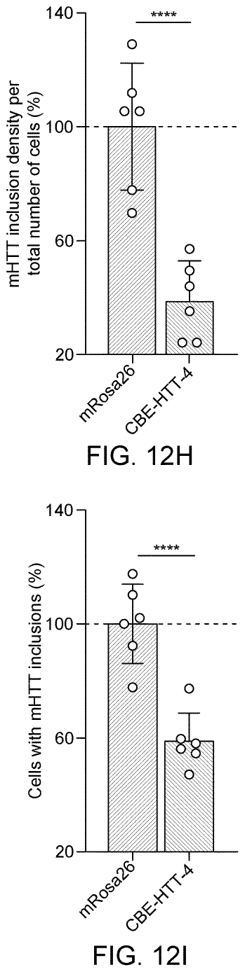

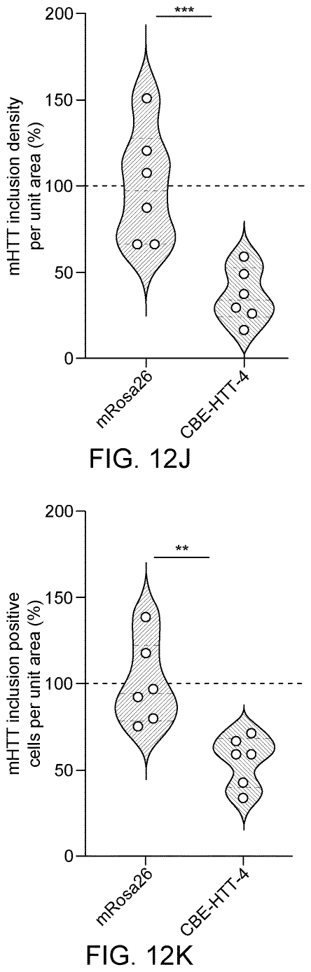

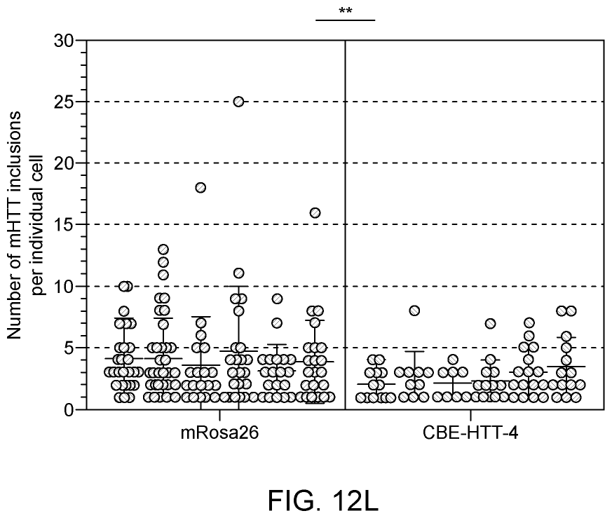

To mediate the delivery of the HTT-targeting split-intein CBE, we used AAV9, which can transduce neurons in the striatum and has demonstrated effectiveness in various studies. For this experiment, we bilaterally injected the striatum of two-month-old YAC128 mice with 1×10⁹ vector genomes (VGs) total, per hemisphere, of two AAV9 vectors encoding the split-intein version of CBE-HTT-4, with the N- and C-terminal CBE-encoding AAV vectors injected at a 1:1 ratio (FIG. 12a). As negative control, we injected YAC128 mice with an AAV formulation encoding split-intein CBEs targeting the mRosa26 locus, a safe harbor region in the mouse genome. To express the CBE, we used a minimal version of the CAG promoter, which can accommodate packaging of the split-intein CBE into dual-AAV vectors and is functional in neurons.

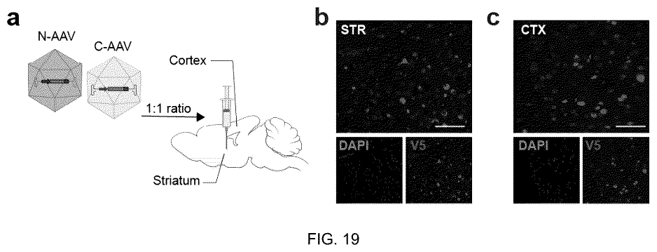

At four weeks post-injection, we conducted an immunofluorescence-based analysis to determine the expression of the CBE using an antibody that recognizes a V5 epitope contained on its N-terminus, which revealed the CBE was predominantly expressed within the striatum and the cortex (FIG. 19). Using NGS, we next measured DNA editing in bulk striatal tissue from 12-month-old YAC128 mice, observing the target C>T modification in the SA for exon 13 in ~5% of the reads analyzed from mice injected with the HTT-targeting CBE (P<0.0001; FIG. 12b). The previously observed G>A bystander mutation, located +1-bp of the target SA, was also found in ~4.8% of the analyzed reads (P<0.0001; FIG. 12b). Notably, no editing was observed in tissue from YAC128 mice injected with AAV encoding the mRosa26-targeting CBE.

To determine if modifying the exon 13 SA influenced HTT mRNA splicing, we conducted an NGS analysis on RT-PCR products from mRNA isolated from bulk striatal tissue of 12-month-old YAC128 mice. From this analysis, we found that ~4.8% of all HTT transcripts from mice treated with the HTT-targeting CBE lacked exon 13 (P=0.001) and that ~2% of all transcripts lacked the first 39-bps of exon 13 (P=0.001; FIG. 12c). Importantly, no skipping was observed in samples from mice injected with the mRosa26-targeting CBE.

Using western blot, we next measured the abundance of the HTT protein in 12-month-old YAC128 mice with an anti-HTT antibody clone (5HU-1 H6) that recognizes the N-terminal domain (amino acids 115-129) of the human protein whose specificity we validated on lysates from HEK293T cells modified to have either a knockout or partial reduction of HTT (FIG. 27b). Within the striatum, we found that YAC128 mice treated by the split-intein CBE had ~48% less N-terminal HTT protein (P=0.0254; FIG. 12d) and 10% less FL HTT protein (P=0.2; FIG. 12d) compared to animals injected with the mRosa26-targeting CBE.

We next determined if disrupting the SA for exon 13 improved HD-related deficits in YAC128. Using magnetic resonance imaging (MRI), we measured volumetric changes of the striatum and cortex of 12-month-old YAC128 mice, observing mice treated by base editing had striatal volumes that trended towards an increase compared to the controls (P=0.04; FIG. 12e; FIG. 33). As expected, YAC128 mice injected with the mRosa26-targeting CBE had reduced striatal volume compared to their wild-type littermates (FIG. 12e); however, in YAC128 mice treated by base editing the striatum was ~10% larger than in mice injected with the mRosa26-targeting CBE (P=0.04; FIG. 12e). No perseveration in cortical volumes were observed for the mice treated by base editing (P>0.05; FIG. 12f).

A hallmark of HD is the accumulation of intraneuronal inclusions consisting of the mHTT protein, which can consist of N-terminal fragments of the mHTT protein. We thus conducted an immunofluoresence-based analysis to determine if disrupting the exon 13 SA also reduced the abundance of mHTT-containing inclusions in 18-month-old YAC128 mice. Notably, at this time point, mHTT inclusions in YAC128 mice cluster predominately in the nucleus of cells.

Using an anti-HTT antibody (EM48) that recognizes the N-terminal domain of the protein (amino acids 1-256) and is selective for mHTT-containing inclusions (FIG. 12g), we determined the relative density of mHTT inclusions in the striatum, a ratio that we defined as the number of mHTT inclusions per the total number of DAPI⁺ cells per section. From this analysis, we measured that mice treated by base editing had a ~61% decrease in inclusion density compared to mice injected with the mRosa26-targeting CBE (P<0.0001; FIG. 12h). We further measured that mice treated by base editing had a ~41% decrease in the percentage of cells with detectable mHTT inclusions (p<0.0001; FIG. 12i), a ~63% decrease in mHTT inclusion density per area unit (P=0.0008; FIG. 12j) and a ~45% decrease and the number of cells with detectable mHTT inclusions per area unit (P=0.0015; FIG. 12k). Additionally, for those cells with detectable mHTT inclusions, we measured a ~50% decrease in their total number of inclusions per cell (P=0.0027; FIG. 12I).

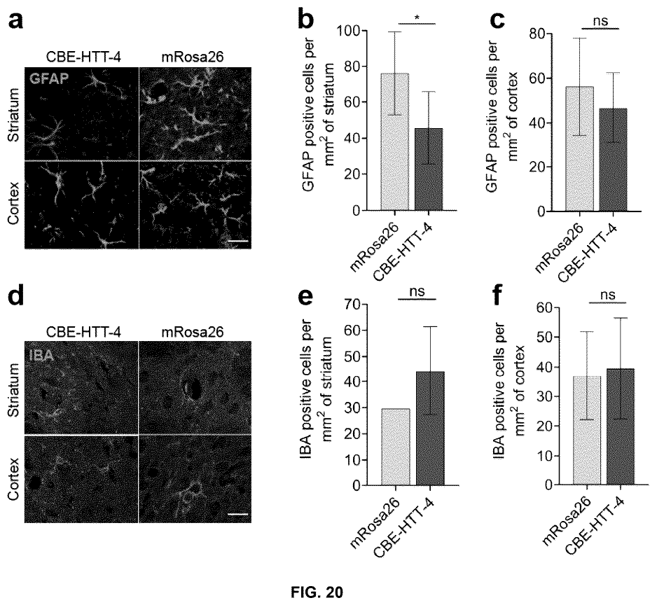

Last, we evaluated if disrupting the SA for exon 13 influenced astrogliosis and microgliosis, two neuroinflammatory responses that are suspected to play a role in HD. Within the striatum, YAC128 mice treated by the CBE had a ~42% decrease in the number of cells positive for the reactive astrocyte marker glial fibrillary acidic protein (GFAP) compared to the controls (P=0.03; FIG. 20b). However, no difference was observed in the number of GFAP⁺ cells in the cortex for animals treated by base editing compared to controls (FIG. 20c). Similarly, we observed no difference between either group in the number of cells positive for the reactive microglia marker ionized calcium-binding adaptor molecule 1 (Iba-1) in either the striatum or cortex (FIG. 20e, f).

Collectively, these findings demonstrate that CRISPR base editors can be delivered in vivo to disrupt the SA for HTT exon 13 and that their action can attenuate the formation of mHTT inclusions and preserve the striatum of YAC128 mice.

### Example 10: Near-PAMless Base Editors Enhance Editing of the SA for HTT Exon 13

While an early-generation SpCas9-based CBE enabled editing of the SA site for HTT exon 13, the emergence of a large catalog of base editor variants with improved efficiency, increased precision, and greater targeting range prompted us to examine whether their utilization could enhance the skipping of HTT exon 13.

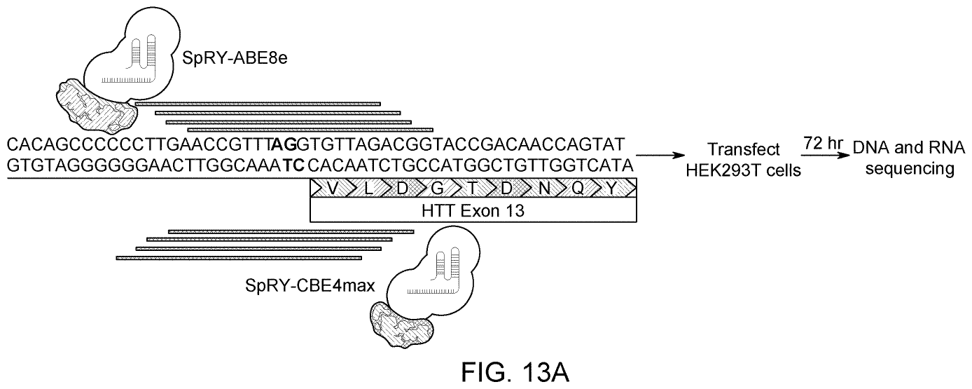

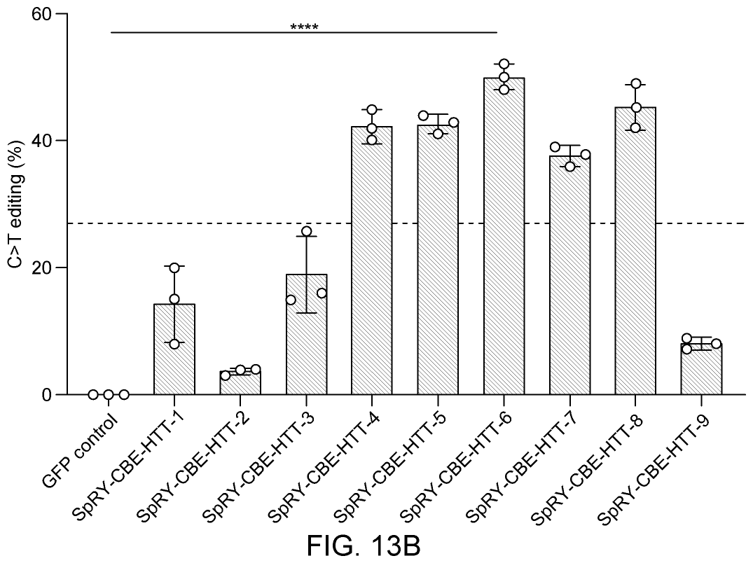

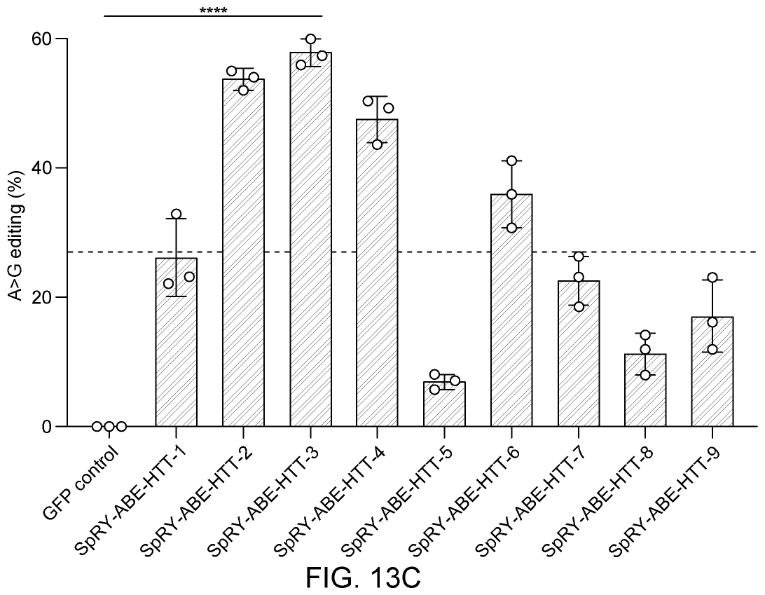

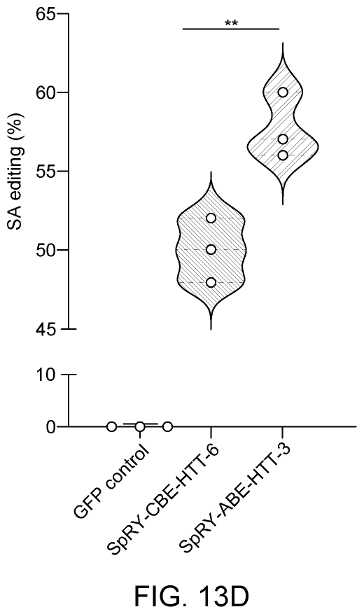

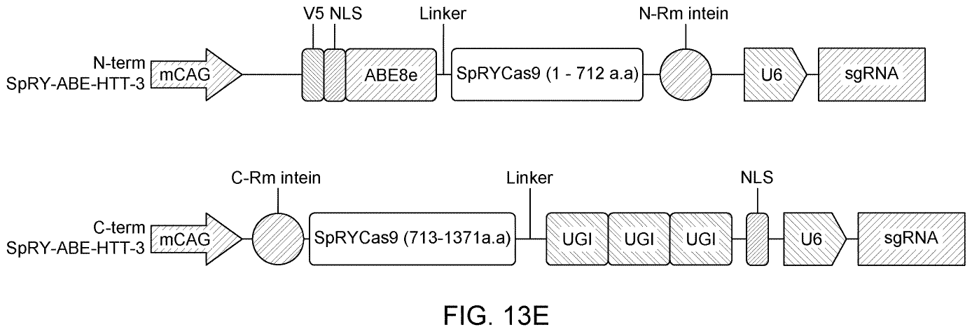

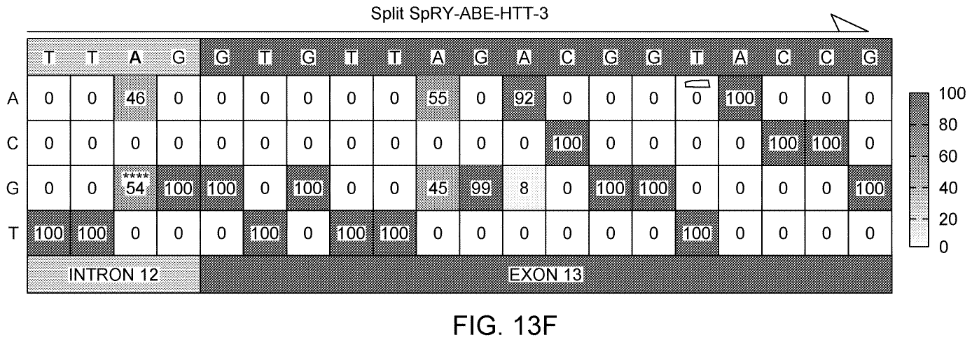

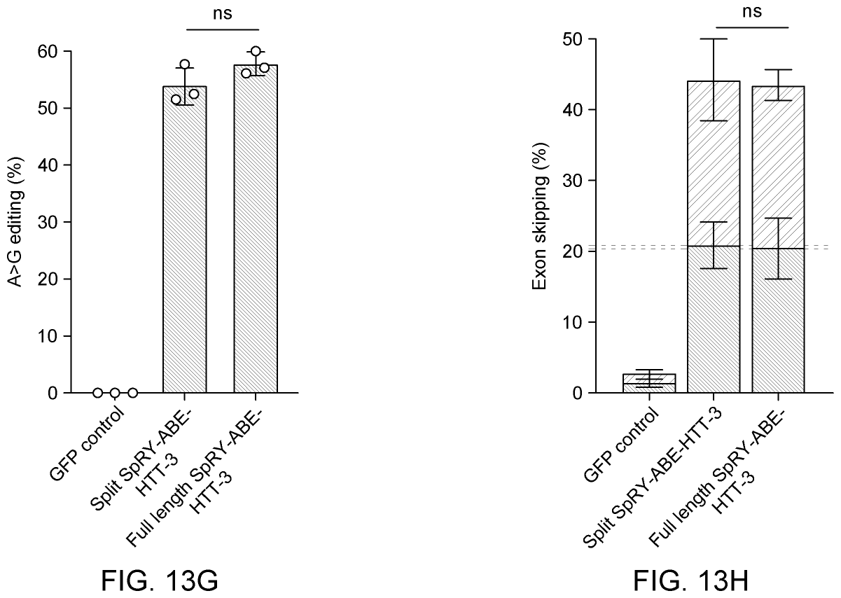

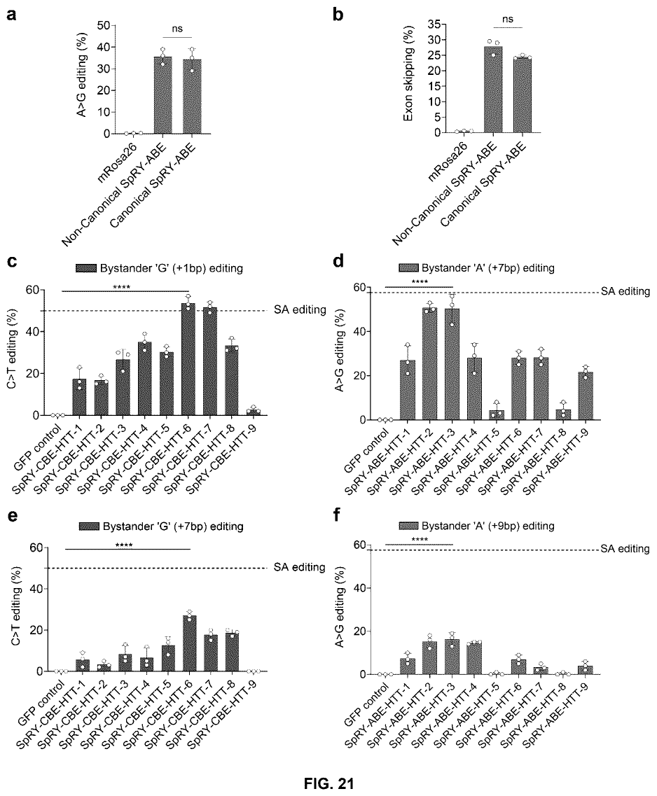

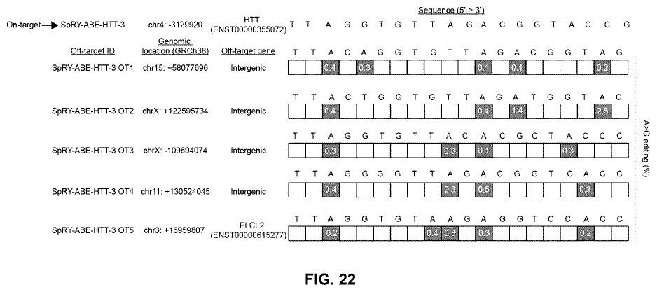

To determine this, we designed 19 new CBEs comprised of the deaminase domain from CBE4max and 19 new ABEs based on ABE8e. To enable a more systemic tiling of the SA for HTT exon 13, we also utilized a variant of the near PAM-less Cas9 variant SpRY for DNA targeting (FIG. 13a) which has equivalent DNA targeting activity compared to the originally engineered form of the enzyme (FIG. 21a,b). In our experience, SpRY is generally as efficient or more efficient than the PAM-flexible enzymes SpCas9-NG or SpG for the type of PAMs targeted in HTT exon 13. Similarly, even though seven of the 38 sites contain 5′-NGG-3′ PAM sequences, we chose to use SpRY for targeting as, in our experience, it functions with equivalent efficiency as the wild-type SpCas9 protein. Thus, for simplicity purposes, SpRY was the lone Cas9 architecture used for this analysis.

Using NGS, we evaluated the editing capabilities of these 38 variants in HEK293T cells, finding that nine of the 38 editors modified the SA for HTT exon 13 with efficiencies that exceeded those for the SpCas9-based CBE variant used in our initial study (FIG. 13b, c). This included SpRY-CBE-HTT-6 and SpRY-ABE-HTT-3, two variants that edited the exon 13 SA with efficiencies of ~50% and ~57%, respectively (P<0.0001 for both, FIG. 13d), rates that corresponded to a nearly two-fold improvement to the originally identified SpCas9-based CBE variant. Though variants that targeted D586 were also evaluated, each editor modified the exon 13 SA at a near equal efficiency (Table 7). Additionally, no variant that targeted D586 was found to edit more efficiently than SpRY-ABE-HTT-3.

Following this screen, we next determined the editing capabilities of the split-intein version of SpRY-ABE-HTT-3 (FIG. 13e), which we utilized for all subsequent studies given its increased efficiency compared to SpRY-CBE-HTT-6. Of note, the split-intein version of this ABE contained a C-terminal uracil DNA glycosylase inhibitor (UGI) domain can boast exon skipping. According to an NGS analysis of editing outcomes in HEK293T cells, the split-intein equivalent of this ABE induced the target A>G edit in the SA site for HTT exon 13 at an efficiency of ~54% (P<0.0001; FIG. 13f), a number that was largely indistinguishable from its FL counterpart, though both variants were found to edit a bystander ‘A’ +7-bps downstream of the SA at an efficiency of ~45% (FIG. 13f, 21). Fortuitously, this edit was expected to create only a silent mutation in HTT [TTA>TTG: Leu585>Leu]. Additionally, the split SpRY-ABE-HTT-3 installed another bystander mutation +9-bps downstream of the SA in 8% of the reads (FIG. 13f), which causes a missense mutation in residue D586 [GAC>GGC: Asp586>Gly].

Using NGS, we next measured the rate of exon skipping for each ABE via the RT-PCR products from mRNA extracted from transfected HEK293T cells. We measured no difference in relative efficiency of HTT exon skipping for the split-intein ABE versus its full-length equivalent (P>0.05), with both variants found to induce ~43% skipping (FIG. 13g). In the case of the split-intein ABE, ~47% of these skipping events led to the removal of the entirety of exon 13, while ~53% of them involving the skipping of just the first 39-bps of exon 13 (P>0.05 for both events; FIG. 13h, i).

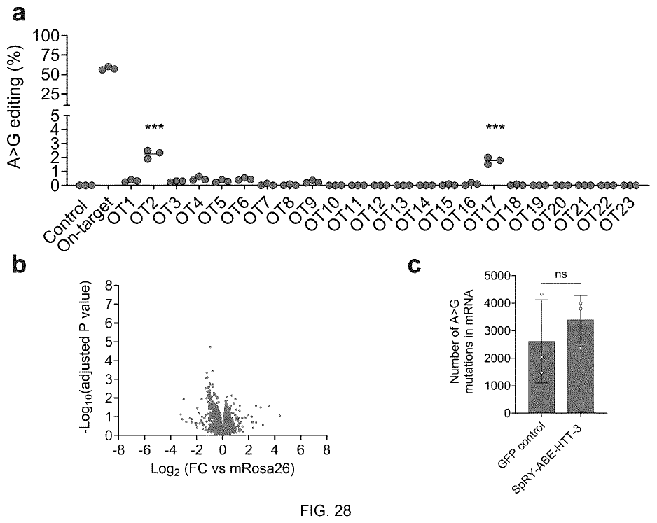

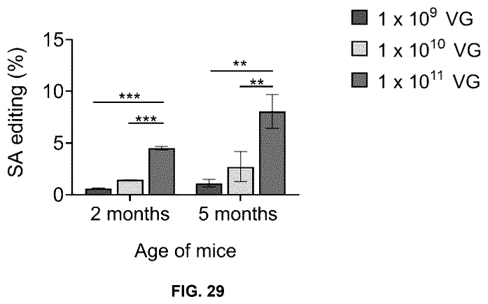

Next, we analyzed OT editing by the ABE in HEK293T cells at 23 candidate OT sites, which we identified by Cas-OFFinder and COSMID (Table 8). These candidate OT sites had up to 4-bps of mismatch to the sgRNA target and up to 2-bps of bulge sequence. We measured no editing at 21 of the 23 sites compared to the GFP-control samples (FIG. 28), however, a low percentage of A>G editing was observed at OT2 (2.27%, P>0.0001) and OT17 (1.767%, P>0.0001) (FIG. 28a), two sites located in intergenic regions.

To determine transcriptome-wide impact of HTT exon 13 SA editing on gene expression, RNA-seq was conducted on HEK293T cells transfected with SpRY-ABE-HTT-3 or SpRY-ABE-mRosa26, which served as the control for this study. No differentially expressed genes were observed for SpRY-ABE-HTT-3 versus SpRY-ABE-mRosa26 at 72-hours post transfection (fold-change (FC)>1; [FDR]-adjusted P<0.05; FIG. 28b). We also measured the RNA editing activity of SpRY-ABE8e. Compared to HEK293T cells transfected with a GFP-expressing control plasmid, we observed an elevated but not significantly increased number of A>G edits in RNA from cells transfected with SpRY-ABE8e-HTT-3 (3394 SNVs for SpRY-ABE8e versus 2613 SNVs for GFP; P>0.05; FIG. 28c).

### Example 11: Near-PAMless Base Editors Edit HTT Exon 13 SA In Vivo and Improve HD-Related Deficits

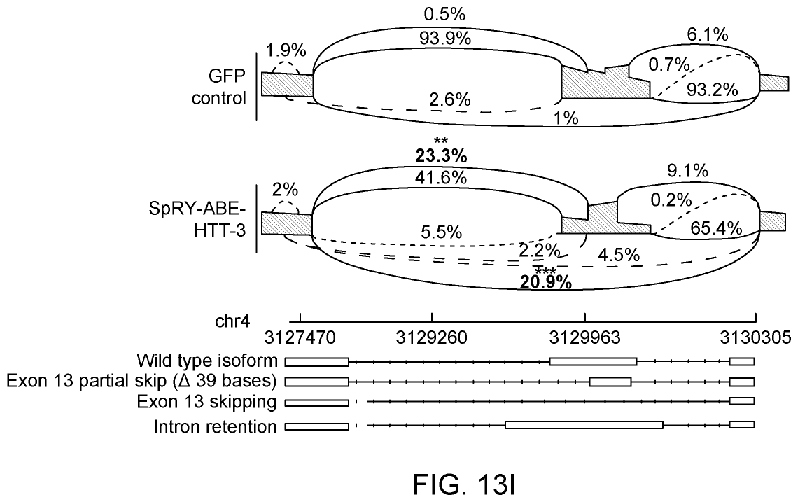

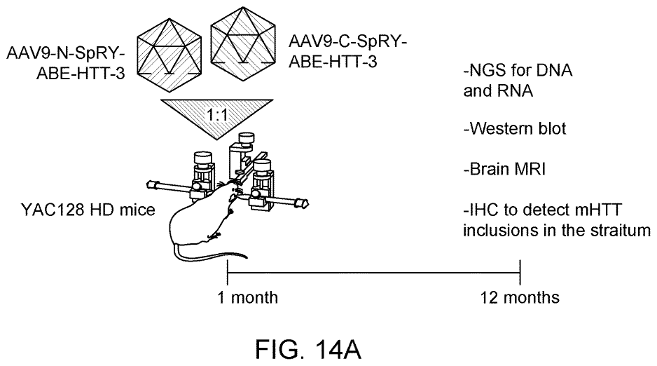

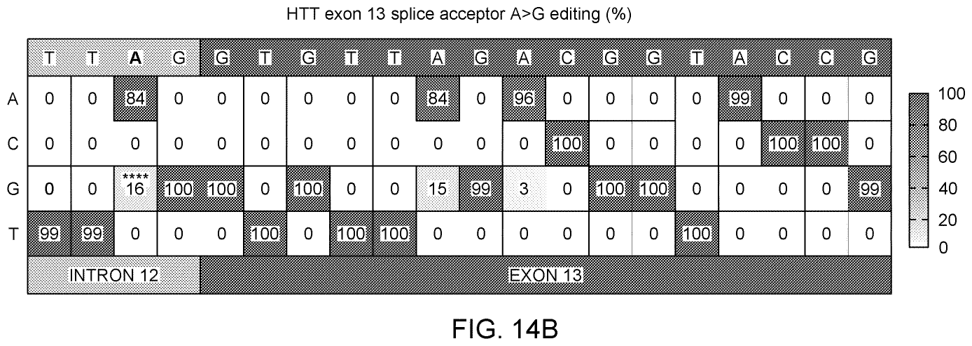

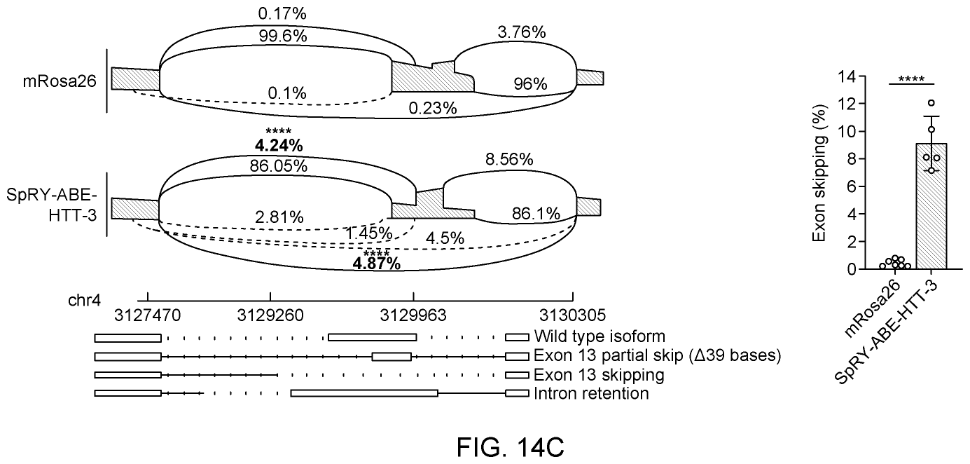

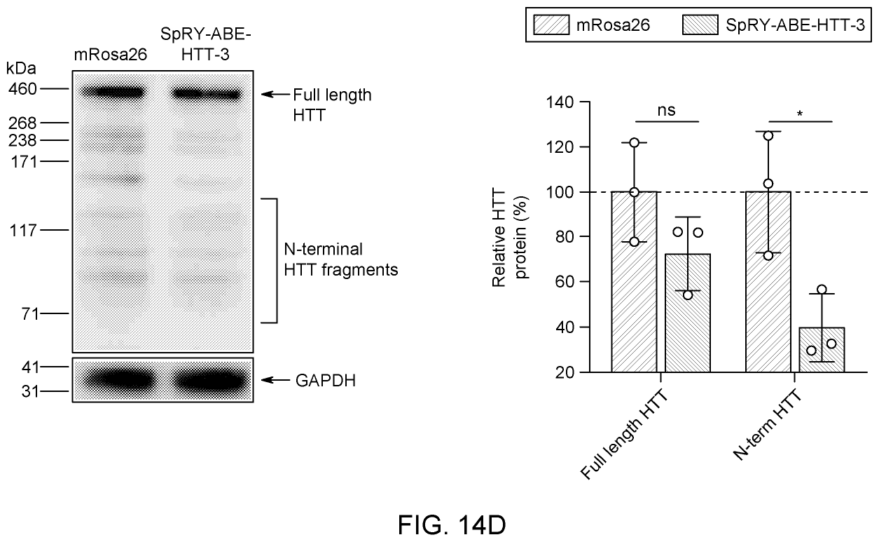

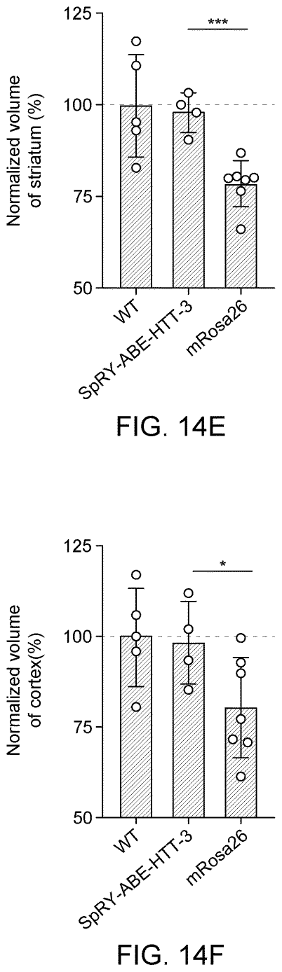

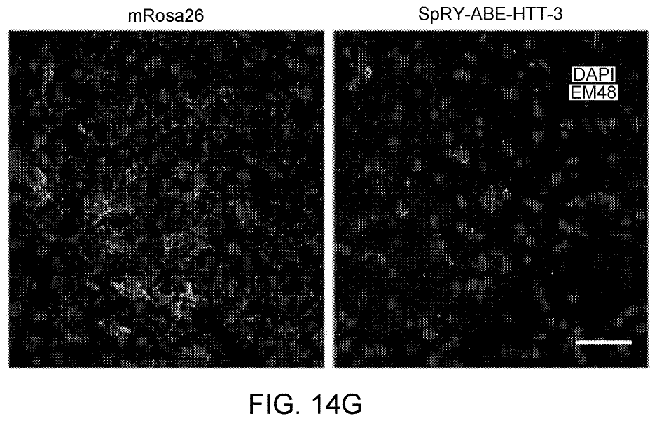

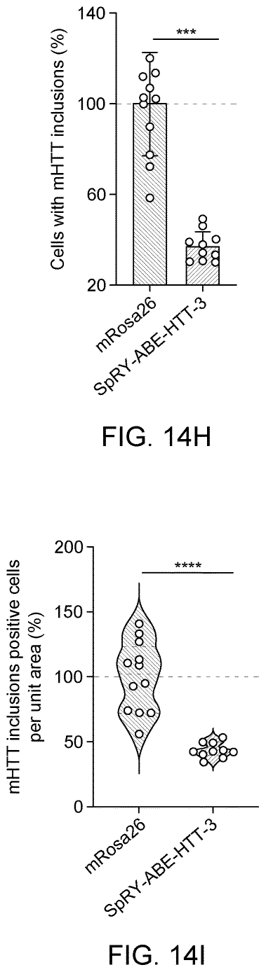

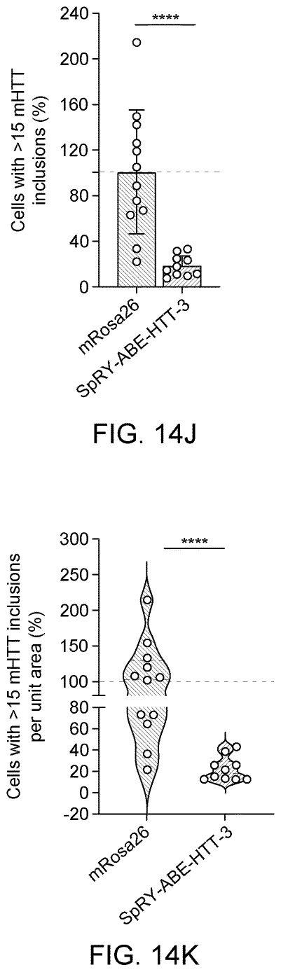

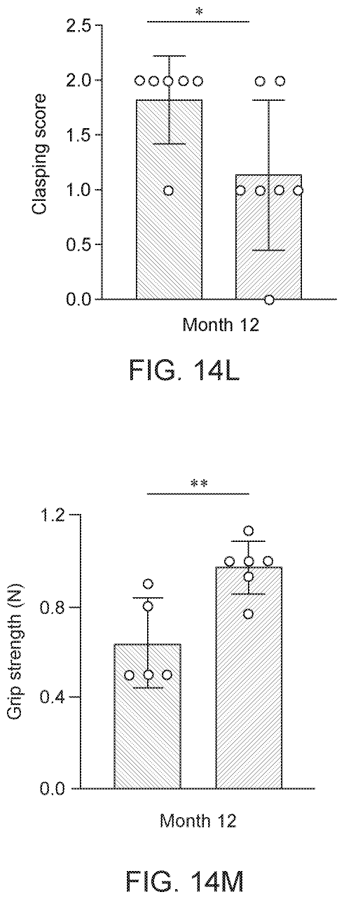

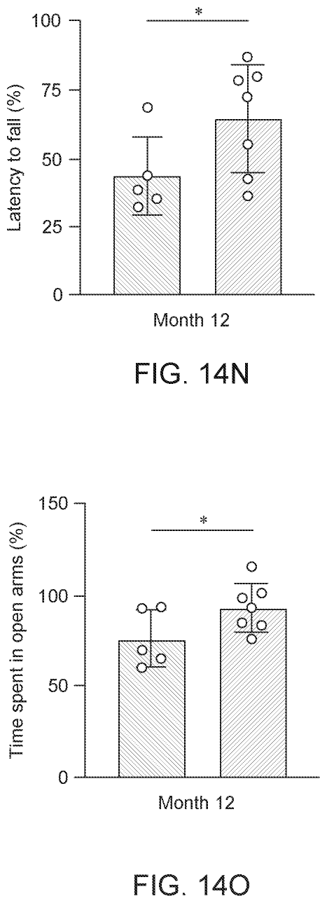

Given its improved editing capabilities compared to the SpCas9-based CBE used in our initial study, we next evaluated the ability for SpRY-ABE-HTT-3 to edit the SA for HTT exon 13 in YAC128 mice (FIG. 14a). AAV9 vectors encoding the split-intein version of this editor, whose expression was again driven by a minimal CAG promoter, were bilaterally injected to the striatum of one-month-old YAC128 mice at three doses: 1×10⁹, 1×10¹⁰, and 1×10¹¹ VGs total per hemisphere, with the N- and C-terminal AAV vectors injected at a 1:1 ratio.

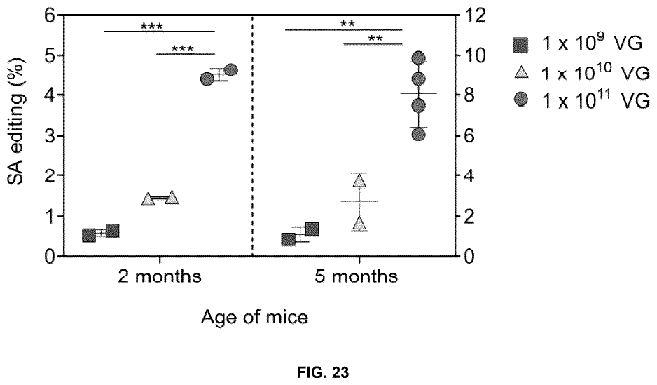

Using NGS, we analyzed editing outcomes in bulk striatal tissue at two time-points: one-month and four-months post-injection. From this analysis, we observed a dose- and time-dependent increase in A>G editing at the exon 13 SA (FIG. 23), with editing rates of ~0.6%, ~1.4%, and ~4.5% observed at one-month post-injection for mice injected with 1×10⁹, 1×10¹⁰, and 1×10¹¹ VGs per hemisphere, respectively (P<0.001; FIG. 23), and editing rates of ~1.1%, ~2.7%, and ~8.1% observed at four-month post-injection for mice injected with 1×10⁹, 1×10¹⁰, and 1×10¹¹ VGs per hemisphere, respectively (P<0.01; FIG. 23). Because the editing rate for SpRY-ABE-HTT-3 was approximately four-fold higher when delivered at 1×10¹¹ VGs versus 1×10¹⁰ VGs (P<0.01), we elected to use the higher dosage for all subsequent studies.

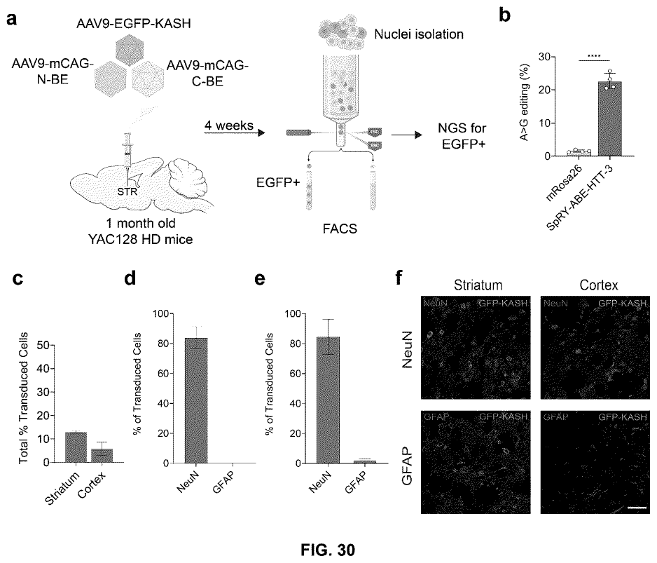

As bulk editing analyses are influenced by the presence of non-transduced cells, we next analyzed editing within enriched cell populations. YAC128 mice were thus injected with 1×10¹¹ VGs of dual AAV9 vectors encoding SpRY-ABE-HTT-3 alongside 1×10¹⁰ VGs of third AAV9 encoding an mCAG-driven EGFP variant fused to the Klarsicht/ANC-1/Syne-1 homology (KASH) domain (AAV9-mCAG-EGFP-KASH), which localizes to the outer nuclear membrane of cells [81] and can enable enrichment of AAV-transduced nuclei (FIG. 30a). We then used NGS to analyze editing at the HTT exon 13 SA from FACS-enriched EGFP-KASH⁺ cells harvested from the striatum at four-weeks post-injection, which revealed ~23% A>G editing (FIG. 30b). An immunofluorescence analysis further indicated that EGFP-KASH was expressed primarily in neurons, as >80% of all EGFP-KASH⁺ cells in injected YAC128 mice were NeuN⁺, both in the striatum and cortex (FIG. 30c-f).

Following this dose-escalation, we used NGS to analyze editing outcomes in bulk striatal tissue from 12-month-old YAC128 mice injected with 1×10¹¹ VGs of the split-intein ABE, which revealed the target edit in ~16% of all reads (FIG. 14b, P<0.0001). Notably, ~15% and ~3% of all reads were also found to carry the above-described bystander edit +7-bps and +9-bps downstream of the target SA, respectively (FIG. 14b). Using NGS, we also measured the relative frequency of exon skipping on PCR products from cDNA prepared from bulk striatal tissue, observing an overall skipping rate of ~9.1% (P<0.0001; FIG. 14c), with ~4.9% of all transcripts found to lack all of exon 13 (P<0.0001) and ~4.2% of transcripts found to lack only the first 39-bps of exon 13 (P<0.0001; FIG. 14c), which was altogether consistent with our findings from HEK293T cells.

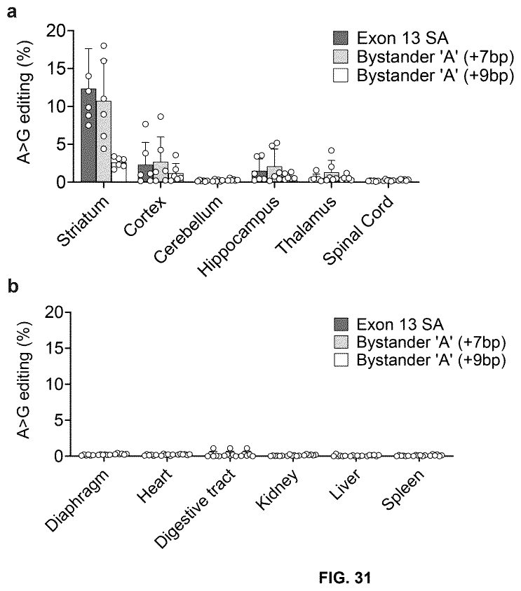

Using NGS, we also analyzed editing in the cortex, cerebellum, hippocampus, thalamus and spinal cord, as well as in the heart, liver, skeletal muscle, and spleen. No detectable A>G editing was observed in any brain region outside the striatum (FIG. 31a) nor in any peripheral tissue (FIG. 31b).

Using the anti-HTT antibody clone 5HU-1 H6, which recognize residues 115-129 of the human protein, we determined the effects of SpRY-ABE-HTT-3 on the HTT protein by western blot. Within the striatum, we found that 12-month-old YAC128 mice treated with SpRY-ABE-HTT-3 had ~60% less N-terminal HTT protein (P=0.013; FIG. 14d) and ~28% less FL HTT protein (P=0.07; FIG. 14d) compared to the controls.

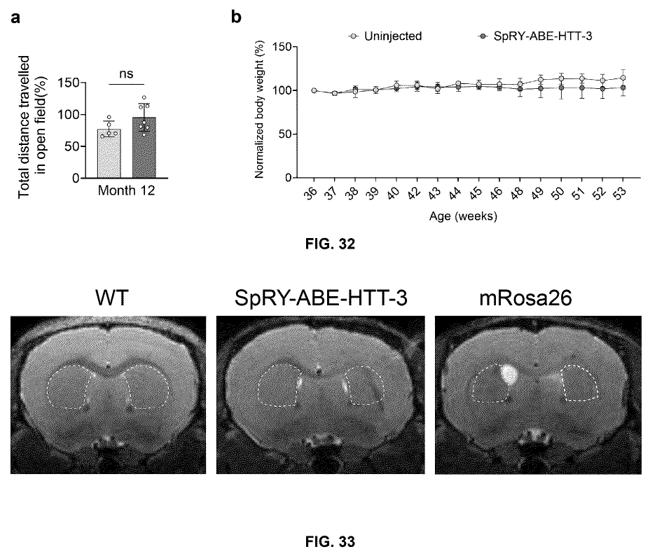

We next determined if the split-intein ABE improved HD-related deficits. Compared to control mice, 12-month-old YAC128 mice treated with SpRY-ABE-HTT-3 had a ~62% decrease in clasping (P=0.028; FIG. 14l), a ~38% increase in forelimb grip strength (P=0.034; FIG. 14m), a ~33% increase in latency to fall time on an accelerating rotarod (P=0.037; FIG. 14n), and spent ~25% more time in the open arms of an elevated plus maze (P=0.035; FIG. 14o), which indicates decreased anxiety-like behavior. However, no difference in distance traveled in the open field test (FIG. 32a) and no difference in body weight (FIG. 32b) was observed between the groups.

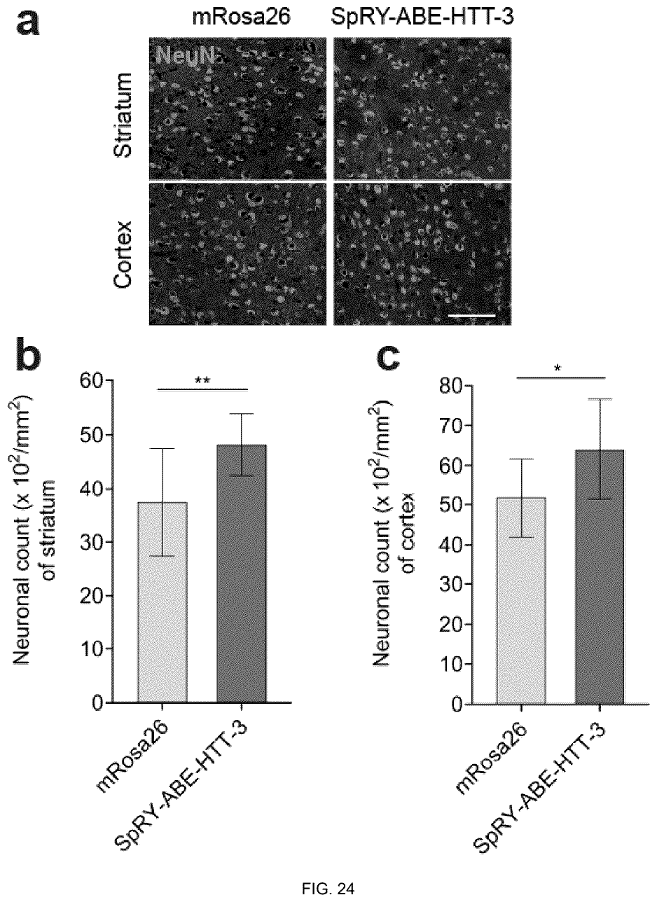

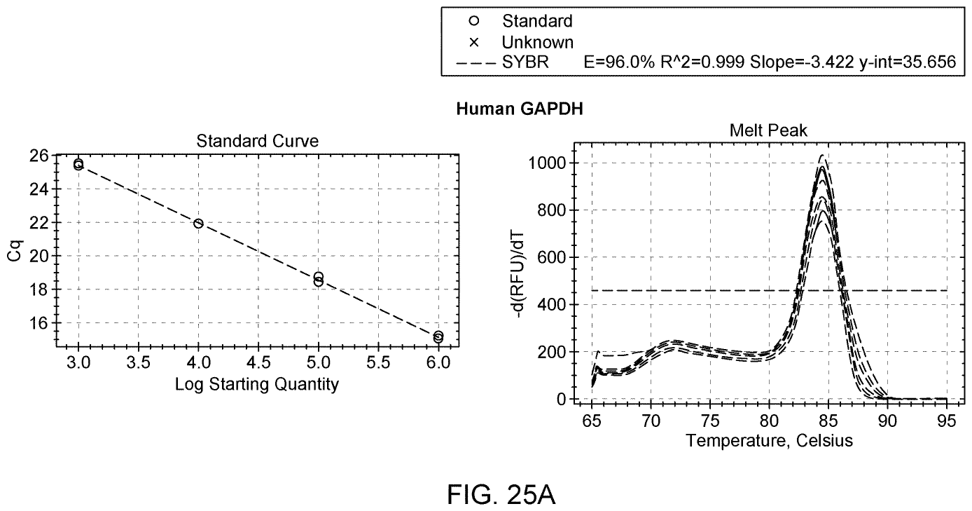

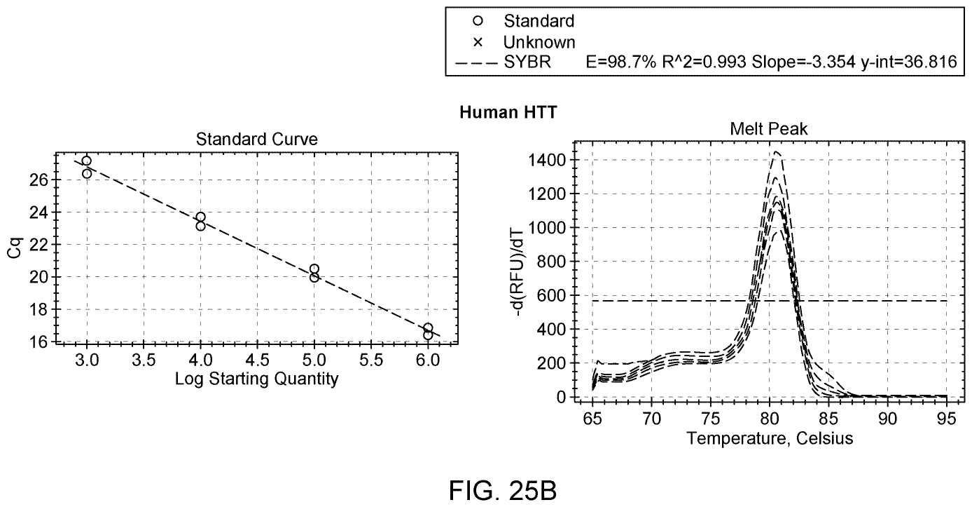

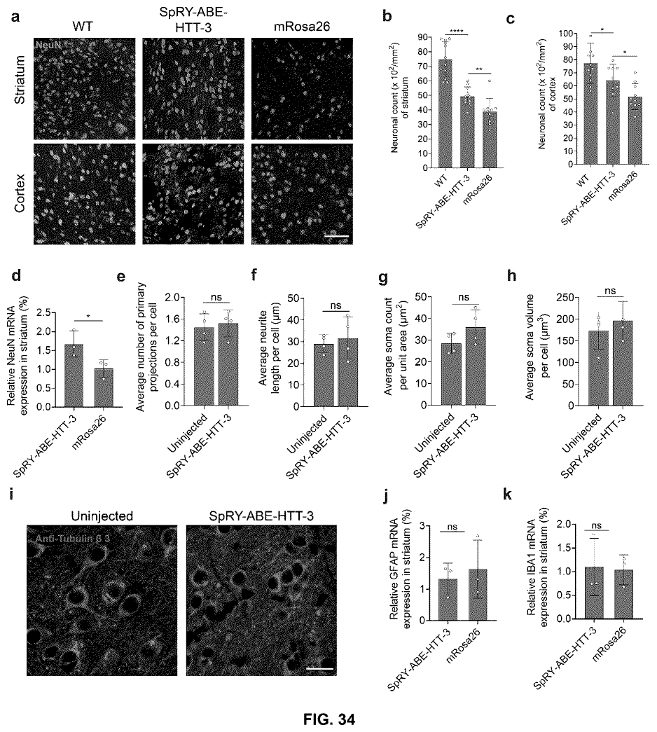

To determine if the split-intein ABE improved HD-related deficits, we used MRI to monitor volumetric changes in the striatum and cortex of YAC128 mice. While 12-month-old YAC128 mice injected with the mRosa26-targeting ABE had a ~22% and ~20% decrease in their striatal and cortical volumes, respectively, compared to the wild-type littermates (FIG. 14e, f), YAC128 mice treated with the split-intein ABE had only a ~3% and ~2% reduction in these respective volumes (P<0.05 for both; FIG. 14e, f) compared to wild-type mice. We further quantified the density of neurons in the striatum and cortex of 12-month-old YAC128 mice (FIG. 24a), measuring that animals injected with SpRY-ABE-HTT-3 had ~22% and ~19% more NeuN⁺ cells in the striatum (P=0.006; FIG. 24b) and cortex (P=0.015; FIG. 24c) compared to the age-matching controls injected with an mRosa26-targeting ABE. qPCR further revealed a ~64% increase in NeuN mRNA in the ABE-treated mice compared to the controls (P=0.0274; FIG. S16d). Though we observed a number of increased NeuN⁺ cells and an increase in NeuN expression, we measured no difference in the number of projections per NeuN⁺ cell (FIG. 34e), neurite length per NeuN⁺ cell (FIG. 34f), soma count (FIG. 34g) and soma volume (FIG. 34h) between the ABE-treated YAC128 mice and the controls. qPCR also showed no difference in relative GFAP and Iba1 expression in treated mice versus the controls (FIG. 34j,k).

Finally, using the EM48 antibody, which recognizes aggregated forms of the mHTT protein, we conducted an immunofluorescence-based analysis to determine if the ABE reduced mHTT inclusions in the striatum of 12-month-old YAC128 mice. Compared to controls, mice treated by base editing had a ~63% decrease in the percentage of cells with detectable mHTT inclusions (P<0.0001; FIG. 14h), an ~82% decrease in the number of inclusions per cell (P<0.0001, FIG. 14i), and a ~56% decrease in the number of cells with inclusions per area unit (P<0.001; FIG. 14j). Additionally, among cells with detectable mHTT inclusions, we measured a ~78% decrease in the total number of cells with >15 inclusions for mice treated by base editing (P<0.0001; FIG. 14k).

In conclusion, we have developed CRISPR base editing platforms capable of disrupting the SA for HTT exon 13, which we show can induce its skipping to produce HTT protein isoforms resistant to proteolysis by caspase-6. These platforms were delivered by AAV to the striatum of a transgenic rodent model, where their editing decreased the accumulation of mHTT inclusions. Improved functional deficits, and led to the preservation of striatal and cortical volumes. Our results demonstrate the potential of base editing for treatment of HD.

### Example 12: Discussion

In this work, we describe an approach to treat HD using CRISPR base editing, which we find can improve two hallmarks of the disease: the accumulation of mHTT aggregates in the brain, and striatal and cortical volume loss. Given the critical role that the mHTT protein plays in HD and that its genetic depletion from cells can reverse features of HD, both antisense oligonucleotides (ASOs) and RNA interference (RNAi) have been used to lower the mHTT protein, with tominersen, an ASO developed by lonis Therapeutics, and AMT-130, a microRNA-based gene therapy developed by uniQure, in clinical trials. However, tominersen and AMT-130 are non-allele-specific and thus target both the mutant and wild-type HTT transcripts, which has raised concerns about tolerability, as HTT has been found to play an important role in numerous physiological processes. As an alternative, allele-specific approaches that target single nucleotide polymorphisms (SNPs) associated with the mHTT allele have been explored, including several ASOs developed by Wave Sciences that have advanced to clinical testing (SELECT-HD; NCT05032196). However, these strategies are limited not just by the number of targetable SNPs available within the HD patient population, but also the financial and technical demands associated with individualized therapies. More broadly, these efforts suffer from other limitations, including the transient lifecycle of ASOs, which require a lifetime of administrations that could pose a burden on patients and lead to periods of diminished activity. Further, tominersen, the non-allele-specific ASO for HTT developed by lonis, was found to trigger a dose-dependent ventricular enlargement, a safety concern that could lead to life-threatening side effects. Conversely, while miRNAs and shRNAs can be expressed for an extended period of time from a viral vector to continuously engage with a target mRNA, these modalities rely on endogenous RNA processing pathways, whose saturation carries risk for dysregulating certain cellular functions. Thus, there is a major need for new approaches that not only have the ability to permanently lower the mHTT protein but can do so without fully depleting the reservoir of the wild-type protein by a manner applicable to the entire HD patient population.

To overcome the limitations of existing gene therapies for HD, we explored the potential for CRISPR base editing technology to skip exon 13 of the HTT gene, both in cell culture models and a transgenic rodent model of HD. While HTT has been targeted and disrupted using CRISPR-Cas9 nucleases, we used base editors in this study given that: (1) feature more homogeneous and predictable editing outcomes compared to traditional nucleases and (2) possess a more favorable safety profile. In particular, as base editors do not rely on DSBs nor NHEJ to initiate editing, they carry a reduced risk for inducing various genotoxic effects, including chromosomal deletions, translocations and chromothripsis.

Unlike most other strategies developed to date for HD, the approach described here is both haplotype-agnostic and expected to preserve some function of the HTT protein even in the absence of allele-specificity, as the 39-bp in-frame deletion generated by exon skipping removes the caspase-6 cleavage site but maintains the reading frame. This is in contrast to other non-allele-specific approaches, including tominersen and AMT-130, which lower both mutant and wild-type HTT. Additionally, this approach induces a permanent effect. Thus, unlike ASOs, it only needs to be administered once. An alternative approach with the potential to decrease the toxicity of the mHTT protein is to edit the CAG repeat, which was recently accomplished with base editors. Though this approach holds the potential for preventing somatic expansion and delaying the onset of disease, it's not expected to affect the function of the mHTT protein. Beyond our current work, base editors have also been used to edit the CAG repeat in the HTT gene. However, while editing the repeat holds potential for preventing somatic expansion and could be used to delay the onset of HD, it's not expected to affect the function of the mHTT protein, as it does not alter the amino acid sequence of the repeat. More broadly, genome editing has been used to contract the CAG repeat and to inactivate the mutant HTT allele via haplotype targeting. Though promising, these approaches utilize CRISPR nucleases, which induce DSBs to stimulate editing and thus carry risk for mosaic repair outcomes. Additionally, haplotype-specific targeting of the mutant HTT allele is limited by the prevalence of the haplotype. By contrast, base editing of the HTT exon 13 SA is broadly applicable across genotypes.

While expressing a mHTT variant with a mutated caspase-6 cleavage site protects against neurotoxicity in YAC128 mice, knockout of capase-6 in HdhQ150 knock-in mice and BACHD mice failed to prevent the cleavage of the mHTT protein. While the reason for this remains unknown, it's been suggested that other proteases may also cleave mHTT at position 586, which, if the case, raises questions about the potential of the caspase-6 protein as a therapeutic target for HD. Our approach, however, targets the caspase-6 cleavage site itself, which ensures that mHTT protein cleavage is blocked no matter which protease cleaves at or near position 586. By targeting the cleavage site in the mHTT protein, this approach also avoids the challenges inherit to the pharmacological inhibition of proteases like caspase-6, which contribute to numerous critical physiological functions and whose inhibition is known to cause side-effects. Beyond the current work, ASOs have also been used to block HTT proteolysis by disrupting the caspase-6 cleavage site via the skipping of exon 12. This ASO reduced HTT proteolysis, normalized body weight, rescued hypoactivity and preserved striatal volume in YAC128 mice and produced an HTT isoform. However, it remains unknown if this ASO can reduce mHTT inclusion burden.

HD is marked by neuronal loss and brain atrophy in the striatum and cortex. Interestingly, despite observing DNA editing and exon skipping rates lower than 20% in bulk tissue from the striatum, we observed a robust preservation of both the striatum and cortex in YAC128 mice, as animals treated by our most effective platform, SpRY-ABE-HTT-3, had only a ~3% decrease in striatal volume at 12 months of age. We observed a similar beneficial effect for the mHTT inclusions, finding that animals treated by our approach had ~63% less inclusions at 12 months of age. This could be explained in part by the observation that the mHTT protein is able to spread via cell-to-cell and through the late endosomal secretory pathway, which can lead to the accumulation of mHTT aggregates in neighboring cells and, in some cases, trigger the aggregation of the wild-type HTT protein. We hypothesize that modulating the abundance of the mHTT protein at a relatively early-stage of disease may have inhibited a feed-forward control mechanism, which resulted in an effect that is not entirely commensurate with the degree of editing measured in our bulk tissue analyses. Consistent with nucleation-dependent aggregation models, even relatively modest levels of editing, such as the ~5% editing observed in bulk tissue with the CBE, was accompanied at later stages by a disproportionately larger decrease in mHTT protein fragments (~48%) and EM48-reactive inclusions (~41-61%). These data suggest that removing the caspase-6 cleavage site or inducing NMD at even low levels can be therapeutically meaningful in the longer term.

By the current approach, HTT exon 13 SA editing yields two products expected to suppress formation of N-terminal aggregates: (1) an in-frame variant with a 39-bp deletion that removes the D586NLDG caspase-6 binding motif and (2) a variant with a fully skipped exon 13 that is predicted to be degraded by NMD. Of these two variants, we expect that the partially skipped transcript that maintains its reading frame but lacks the caspase-6 cleavage site is the more beneficial, as it prevents proteolysis but preserves a reservoir of HTT.

Though the current approach does lower the FL HTT protein to some extent, heterozygous Hdh (+/−) mice do not manifest a Hdh-deficient phenotype. Additionally, heterozygous individuals with only one functional copy of the HTT gene do not develop a neurological phenotype, suggesting that the partial suppression of HTT may be tolerated. The strategy developed here thus may offer a potentially safer alternative to the non-allele-specific approaches developed for silencing mHTT, though, in the future, we expect that it should be further optimizable to prevent the full skipping of exon 13.

In summary, we demonstrate that exon skipping with CRISPR base editing can be used to create new HTT protein isoforms resistant to proteolysis by caspase-6, which we found mitigated the development of HD-related deficits in a transgenic rodent model of the disease. Our results demonstrate the broad potential of CRISPR base technology for HD and illustrate promise for exon skipping to permanently produce forms of the mHTT protein with decreased toxicity.

### REFERENCES

1. Tabrizi S J, Estevez-Fraga C, van Roon-Mom W M C, Flower M D, Scahill R I, Wild E J, Munoz-Sanjuan I, Sampaio C, Rosser A E, Leavitt B R: Potential disease-modifying therapies for Huntington's disease: lessons learned and future opportunities. Lancet Neurol 2022, 21:645-658.
2. Bates G P, Dorsey R, Gusella J F, Hayden M R, Kay C, Leavitt B R, Nance M, Ross C A, Scahill R I, Wetzel R, et al: Huntington disease. Nat Rev Dis Primers 2015, 1:15005.
3. Nopoulos P C: Huntington disease: a single-gene degenerative disorder of the striatum. Dialogues Clin Neurosci 2016, 18:91-98.
4. Lunkes A, Lindenberg K S, Ben-Haiem L, Weber C, Devys D, Landwehrmeyer G B, Mandel J-L, Trottier Y: Proteases Acting on Mutant Huntingtin Generate Cleaved Products that Differentially Build Up Cytoplasmic and Nuclear Inclusions. Molecular Cell 2002, 10:259-269.
5. Mangiarini L, Sathasivam K, Seller M, Cozens B, Harper A, Hetherington C, Lawton M, Trottier Y, Lehrach H, Davies S W, Bates G P: Exon 1 of the H D Gene with an Expanded CAG Repeat Is Sufficient to Cause a Progressive Neurological Phenotype in Transgenic Mice. Cell 1996, 87:493-506.
6. Wang C E, Tydlacka S, Orr A L, Yang S H, Graham R K, Hayden M R, Li S, Chan A W, Li X J: Accumulation of N-terminal mutant huntingtin in mouse and monkey models implicated as a pathogenic mechanism in Huntington's disease. Hum Mol Genet 2008, 17:2738-2751.
7. Neueder A, Landles C, Ghosh R, Howland D, Myers R H, Faull R L M, Tabrizi S J, Bates G P: The pathogenic exon 1 HTT protein is produced by incomplete splicing in Huntington's disease patients. Scientific Reports 2017, 7:1307.
8. Sathasivam K, NeuederA, Gipson T A, Landles C, Benjamin A C, Bondulich M K, Smith D L, Faull R L, Roos R A, Howland D, et al: Aberrant splicing of HTT generates the pathogenic exon 1 protein in Huntington disease. Proc Natl Acad Sci USA 2013, 110:2366-2370.
9. Fienko S, Landles C, Sathasivam K, McAteer S J, Milton R E, Osborne G F, Smith E J, Jones S T, Bondulich M K, Danby E C E, et al: Alternative processing of human HTT mRNA with implications for Huntington's disease therapeutics. Brain 2022, 145:4409-4424.
10. Graham R K, Ehrnhoefer D E, Hayden M R: Caspase-6 and neurodegeneration. Trends in Neurosciences 2011, 34:646-656.
11. Wong B K Y, Ehrnhoefer D E, Graham R K, Martin D D O, Ladha S, Uribe V, Stanek L M, Franciosi S, Qiu X, Deng Y, et al: Partial rescue of some features of Huntington Disease in the genetic absence of caspase-6 in YAC128 mice. Neurobiology of Disease 2015, 76:24-36.
12. Ehrnhoefer D E, Skotte N H, Reinshagen J, Qiu X, Windshugel B, Jaishankar P, Ladha S, Petina O, Khankischpur M, Nguyen Y T N, et al: Activation of Caspase-6 Is Promoted by a Mutant Huntingtin Fragment and Blocked by an Allosteric Inhibitor Compound. Cell Chemical Biology 2019, 26:1295-1305.el296.
13. Wellington C L, Hayden M R: Of molecular interactions, mice and mechanisms. Current Opinion in Neurology 1997, 10:291-298.
14. Waldron-Roby E, Ratovitski T, Wang X, Jiang M, Watkin E, Arbez N, Graham R K, Hayden M R, Hou Z, Mori S, et al: Transgenic mouse model expressing the caspase 6 fragment of mutant huntingtin. J Neurosci 2012, 32:183-193.
15. Graham R K, Deng Y, Slow E J, Haigh B, Bissada N, Lu G, Pearson J, Shehadeh J, Bertram L, Murphy Z, et al: Cleavage at the caspase-6 site is required for neuronal dysfunction and degeneration due to mutant huntingtin. Cell 2006, 125:1179-1191.
16. Wellington C L, Singaraja R, Ellerby L, Savill J, Roy S, Leavitt B, Cattaneo E, Hackam A, Sharp A, Thornberry N, et al: Inhibiting Caspase Cleavage of Huntingtin Reduces Toxicity and Aggregate Formation in Neuronal and Nonneuronal Cells. Journal of Biological Chemistry 2000, 275:19831-19838.
17. Graham R K, Deng Y, Carroll J, Vaid K, Cowan C, Pouladi M A, Metzler M, Bissada N, Wang L, Faull R L, et al: Cleavage at the 586 amino acid caspase-6 site in mutant huntingtin influences caspase-6 activation in vivo. J Neurosci 2010, 30:15019-15029.
18. Evers M M, Tran H D, Zalachoras I, Meijer O C, den Dunnen J T, van Ommen G J, Aartsma-Rus A, van Roon-Mom W M: Preventing formation of toxic N-terminal huntingtin fragments through antisense oligonucleotide-mediated protein modification. Nucleic Acid Ther 2014, 24:4-12.
19. Komor A C, Kim Y B, Packer M S, Zuris J A, Liu D R: Programmable editing of a target base in genomic DNA without double-stranded DNA cleavage. Nature 2016, 533:420-424.
20. Gaudelli N M, Komor A C, Rees H A, Packer M S, Badran A H, Bryson D I, Liu D R: Programmable base editing of A•T to G•C in genomic DNA without DNA cleavage. Nature 2017, 551:464-471.
21. Gapinske M, Luu A, Winter J, Woods W S, Kostan K A, Shiva N, Song J S, Perez-Pinera P: CRISPR-SKIP: programmable gene splicing with single base editors. Genome Biol 2018, 19:107.
22. Gapinske M, Winter J, Swami D, Gapinske L, Woods W S, Shirguppe S, Miskalis A, Busza A, Joulani D, Kao C J, et al: Targeting Duchenne muscular dystrophy by skipping DMD exon 45 with base editors. Molecular Therapy—Nucleic Acids 2023, 33:572-586.
23. Miskalis A, Shirguppe S, Winter J, Elias G, Swami D, Nambiar A, Stilger M, Woods W S, Gosstola N, Gapinske M, et al: SPLICER: a highly efficient base editing toolbox that enables in vivo therapeutic exon skipping. Nature Communications 2024, 15.
24. Chai A C, Chemello F, Li H, Nishiyama T, Chen K, Zhang Y, Senchez-Ortiz E, Alomar A, Xu L, Liu N, et al: Single-swap editing for the correction of common Duchenne muscular dystrophy mutations. Mol Ther Nucleic Acids 2023, 32:522-535.
25. Yuan J, Ma Y, Huang T, Chen Y, Peng Y, Li B, Li J, Zhang Y, Song B, Sun X, et al: Genetic Modulation of RNA Splicing with a CRISPR-Guided Cytidine Deaminase. Mol Cell 2018, 72:380-394.e387.
26. Kluesner M G, Lahr W S, Lonetree C-I, Smeester B A, Qiu X, Slipek N J, Claudio Vezquez P N, Pitzen S P, Pomeroy E J, Vignes M J, et al: CRISPR-Cas9 cytidine and adenosine base editing of splice-sites mediates highly-efficient disruption of proteins in primary and immortalized cells. Nature Communications 2021, 12:2437.
27. Xiang-Xing Z, Jia-Sheng P, Tao L, Ye-Cheng Y, Qiu-Yan H, Shuai-Peng Y, Zi-Xiao Q, Z-Sheng L, Jian-Cong W, Ai-Fen Y, et al: Adenine base-editing-mediated exon skipping induces gene knockout in cultured pig cells—PubMed. Biotechnology Letters 2022 January, 44.
28. Qiu H, Li G, Yuan J, Yang D, Ma Y, Wang F, Dai Y, Chang X: Efficient exon skipping by base-editor-mediated abrogation of exonic splicing enhancers. Cell Reports 2023 November, 42.
29. Martin D D O, Schmidt M E, Nguyen Y T, Lazic N, Hayden M R: Identification of a novel caspase cleavage site in huntingtin that regulates mutant huntingtin clearance. Fasebj2019, 33:3190-3197.
30. Wellington C L, Ellerby L M, Hackam A S, Margolis R L, Trifiro M A, McCutcheon K, Salvesen G S, Propp S S, Bromm M, Rowland K J: Caspase cleavage of gene products associated with triplet expansion disorders generates truncated fragments containing the polyglutamine tract. Journal of Biological Chemistry 1998, 273:9158-9167.
31. Wellington C L, Ellerby L M, Gutekunst C-A, Rogers D, Warby S, Graham R K, Loubser O, van Raamsdonk J, Singaraja R, Yang Y-Z, et al: Caspase Cleavage of Mutant Huntingtin Precedes Neurodegeneration in Huntington's Disease. The Journal of Neuroscience 2002, 22:7862-7872.
32. Cartegni L: ESEfinder: a web resource to identify exonic splicing enhancers. Nucleic Acids Research 2003, 31:3568-3571.
33. Desmet F-O, Hamroun D, Lalande M, Collod-Beroud G, Claustres M, Beroud C: Human Splicing Finder: an online bioinformatics tool to predict splicing signals. Nucleic Acids Research 2009, 37:e67-e67.
34. Deltcheva E, Chylinski K, Sharma C M, Gonzales K, Chao Y, Pirzada Z A, Eckert M R, Vogel J, Charpentier E: CRISPR RNA maturation by trans-encoded small RNA and host factor RNase III. Nature 2011, 471:602-607.
35. Hsu P D, Scott D A, Weinstein J A, Ran F A, Konermann S, Agarwala V, Li Y, Fine E J, Wu X, Shalem O, et al: DNA targeting specificity of RNA-guided Cas9 nucleases. Nature Biotechnology 2013, 31:827-832.
36. Hu J H, Miller S M, Geurts M H, Tang W, Chen L, Sun N, Zeina C M, Gao X, Rees H A, Lin Z, Liu D R: Evolved Cas9 variants with broad PAM compatibility and high DNA specificity. Nature 2018, 556:57-63.
37. Kim Y B, Komor A C, Levy J M, Packer M S, Zhao K T, Liu D R: Increasing the genome-targeting scope and precision of base editing with engineered Cas9-cytidine deaminase fusions. Nature Biotechnology 2017, 35:371-376.
38. Kleinstiver B P, Prew M S, Tsai S Q, Nguyen N T, Topkar V V, Zheng Z, Joung J K: Broadening the targeting range of Staphylococcus aureus CRISPR-Cas9 by modifying PAM recognition. Nature Biotechnology 2015, 33:1293-1298.
39. Gaj T, Epstein B E, Schaffer D V: Genome Engineering Using Adeno-associated Virus: Basic and Clinical Research Applications. Molecular Therapy 2016, 24:458-464.
40. Lim C K W, Gapinske M, Brooks A K, Woods W S, Powell J E, Zeballos C M, Winter J, Perez-Pinera P, Gaj T: Treatment of a Mouse Model of ALS by In Vivo Base Editing. Mol Ther 2020, 28:1177-1189.
41. Lim C K W, McCallister T X, Saporito-Magrina C, McPheron G D, Krishnan R, Zeballos C M, Powell J E, Clark L V, Perez-Pinera P, Gaj T: CRISPR base editing of cis-regulatory elements enables the perturbation of neurodegeneration-linked genes. Mol Ther 2022, 30:3619-3631.
42. Arbab M, Matuszek Z, Kray K M, Du A, Newby G A, Blatnik A J, Raguram A, Richter M F, Zhao K T, Levy J M, et al: Base editing rescue of spinal muscular atrophy in cells and in mice. Science 2023, 380:eadg6518.
43. Levy J M, Yeh W-H, Pendse N, Davis J R, Hennessey E, Butcher R, Koblan L W, Comander J, Liu Q, Liu D R: Cytosine and adenine base editing of the brain, liver, retina, heart and skeletal muscle of mice via adeno-associated viruses. Nature Biomedical Engineering 2020, 4:97-110.
44. Anastassov S, Filo M, Khammash M: Inteins: A Swiss army knife for synthetic biology. Biotechnology Advances 2024/07/01, 73.
45. Ellils S, Jurvansuu J M, Iwai H: Evaluation and comparison of protein splicing by exogenous inteins with foreign exteins in Escherichia coli. FEBS Letters 2011, 585.
46. Jaganathan K, Kyriazopoulou Panagiotopoulou S, McRae J F, Darbandi S F, Knowles D, Li Y I, Kosmicki J A, Arbelaez J, Cui W, Schwartz G B, et al: Predicting Splicing from Primary Sequence with Deep Learning. Cell 2019, 176:535-548.e524.
47. Maximum Entropy Modeling of Short Sequence Motifs with Applications to RNA Splicing Signals. Journal of Computational Biology 2004, 11:377-394.
48. Zuallaert J, Godin F, Kim M, Soete A, Saeys Y, De Neve W: SpliceRover: interpretable convolutional neural networks for improved splice site prediction. Bioinformatics 2018, 34:4180-4188.
49. Schut M H, Pepers B A, Klooster R, van der Maarel S M, El Khatabi M, Verrips T, den Dunnen J T, van Ommen G J, van Roon-Mom W M: Selection and characterization of llama single domain antibodies against N-terminal huntingtin. Neurol Sci 2015, 36:429-434.
50. Schut M H, Patassini S, Kim E H, Bullock J, Waldvogel H J, Faull R L M, Pepers B A, den Dunnen J T, van Ommen G B, van Roon-Mom WMC: Effect of post-mortem delay on N-terminal huntingtin protein fragments in human control and Huntington disease brain lysates. PLoS One 2017, 12:e0178556.
51. Slow E J, van Raamsdonk J, Rogers D, Coleman S H, Graham R K, Deng Y, Oh R, Bissada N, Hossain S M, Yang Y Z, et al: Selective striatal neuronal loss in a YAC128 mouse model of Huntington disease. Hum Mol Genet 2003, 12:1555-1567.
52. Petrella L I, Castelhano J M, Ribeiro M, Sereno J V, Gongalves S I, Lago M N, Hayden M R, Rego A C, Castelo-Branco M: A whole brain longitudinal study in the YAC128 mouse model of Huntington's disease shows distinct trajectories of neurochemical, structural connectivity and volumetric changes. Human Molecular Genetics 2018, 27:2125-2137.
53. Van Raamsdonk J M, Murphy Z, Slow E J, Leavitt B R, Hayden M R: Selective degeneration and nuclear localization of mutant huntingtin in the YAC128 mouse model of Huntington disease. Human Molecular Genetics 2005, 14:3823-3835.
54. Cearley C N, Wolfe J H: A Single Injection of an Adeno-Associated Virus Vector into Nuclei with Divergent Connections Results in Widespread Vector Distribution in the Brain and Global Correction of a Neurogenetic Disease. The Journal of Neuroscience 2007, 27:9928-9940.
55. Cearley C N, Wolfe J H: Transduction characteristics of adeno-associated virus vectors expressing cap serotypes 7, 8, 9, and Rh10 in the mouse brain. Molecular Therapy 2006, 13:528-537.
56. Foust K D, Nurre E, Montgomery C L, Hernandez A, Chan C M, Kaspar B K: Intravascular AAV9 preferentially targets neonatal neurons and adult astrocytes. Nature biotechnology 2009, 27:59-65.
57. Keeler A M, Sapp E, Chase K, Sottosanti E, Danielson E, Pfister E, Stoica L, DiFiglia M, Aronin N, Sena-Esteves M: Cellular Analysis of Silencing the&nbsp; Huntington's Disease Gene Using AAV9&nbsp; Mediated Delivery of Artificial Micro&nbsp; RNA into the Striatum of&nbsp; Q140/Q140&nbsp; Mice. Journal of Huntington's Disease 2016, 5:239-248.
58. Mondo E, Moser R, Gao G, Mueller C, Sena-Esteves M, Sapp E, Pfister E, O'Connell D, Takle K, Erger K E, et al: Selective Neuronal Uptake and Distribution of AAVrh8, AAV9, and AAVrh10 in Sheep After Intra-Striatal Administration. Journal of Huntington's Disease 2018, 7:309-319.
59. Soriano P: Generalized lacZ expression with the ROSA26 Cre reporter strain. Nat Genet 1999, 21:70-71.
60. Zambrowicz B P, Imamoto A, Fiering S, Herzenberg L A, Kerr W G, Soriano P: Disruption of overlapping transcripts in the ROSA pgeo 26 gene trap strain leads to widespread expression of β-galactosidase in mouse embryos and hematopoietic cells. Proceedings of the National Academy of Sciences 1997, 94:3789-3794.
61. Smith E J, Sathasivam K, Landles C, Osborne G F, Mason M A, Gomez-Paredes C, Taxy B A, Milton R E, Ast A, Schindler F, et al: Early detection of exon 1 huntingtin aggregation in zQ175 brains by molecular and histological approaches. Brain Communications 2023, 5.
62. Thompson L M, Aiken C T, Kaltenbach L S, Agrawal N, Illes K, Khoshnan A, Martinez-Vincente M, Arrasate M, O'Rourke J G, Khashwji H, et al: IKK phosphorylates Huntingtin and targets it for degradation by the proteasome and lysosome. Journal of Cell Biology 2009, 187:1083-1099.
63. Hickman R A, Faust P L, Marder K, Yamamoto A, Vonsattel J-P: The distribution and density of Huntingtin inclusions across the Huntington disease neocortex: regional correlations with Huntingtin repeat expansion independent of pathologic grade. Acta Neuropathologica Communications 2022, 10:55.
64. Gutekunst C-A, Li S-H, Yi H, Mulroy J S, Kuemmerle S, Jones R, Rye D, Ferrante R J, Hersch S M, Li X-J: Nuclear and Neuropil Aggregates in Huntington's Disease: Relationship to Neuropathology. The Journal of Neuroscience 1999, 19:2522-2534.
65. DiFiglia M, Sapp E, Chase K O, Davies S W, Bates G P, Vonsattel J P, Aronin N: Aggregation of huntingtin in neuronal intranuclear inclusions and dystrophic neurites in brain. Science 1997, 277:1990-1993.
66. Riguet N, Mahul-Mellier A-L, Maharjan N, Burtscher J, Croisier M, Knott G, Hastings J, Patin A, Reiterer V, Farhan H, et al: Nuclear and cytoplasmic huntingtin inclusions exhibit distinct biochemical composition, interactome and ultrastructural properties. Nature Communications 2021, 12.
67. Carty N, Berson N, Tillack K, Thiede C, Scholz D, Kottig K, Sedaghat Y, Gabrysiak C, Yohrling G, von der Kammer H, et al: Characterization of HTT inclusion size, location, and timing in the zQ175 mouse model of Huntington's disease: an in vivo high-content imaging study. PLoS One 2015, 10:e0123527.
68. Slow E J, Graham R K, Osmand A P, Devon R S, Lu G, Deng Y, Pearson J, Vaid K, Bissada N, Wetzel R, et al: Absence of behavioral abnormalities and neurodegeneration<i>in vivo</i>despite widespread neuronal huntingtin inclusions. Proceedings of the National Academy of Sciences 2005, 102:11402-11407.
69. Bayram-Weston Z, Stone T C, Giles P, Elliston L, Janghra N, Higgs G V, Holmans P A, Dunnett S B, Brooks S P, Jones L: Similar striatal gene expression profiles in the striatum of the YAC128 and HdhQ150 mouse models of Huntington's disease are not reflected in mutant Huntingtin inclusion prevalence. BMC Genomics 2015, 16:1079.
70. Tong X, Ao Y, Faas G C, Nwaobi S E, Xu J, Haustein M D, Anderson M A, Mody I, Olsen M L, Sofroniew M V, Khakh B S: Astrocyte Kir4.1 ion channel deficits contribute to neuronal dysfunction in Huntington's disease model mice. Nature Neuroscience 2014, 17:694-703.
71. Kraft A D, Kaltenbach L S, Lo D C, Harry G J: Activated microglia proliferate at neurites of mutant huntingtin-expressing neurons. Neurobiol Aging 2012, 33:621.e617-633.
72. Hol E M, Pekny M: Glial fibrillary acidic protein (GFAP) and the astrocyte intermediate filament system in diseases of the central nervous system. Current Opinion in Cell Biology 2015, 32:121-130.
73. Sasaki Y, Ohsawa K, Kanazawa H, Kohsaka S, Imai Y: Iba1 is an actin-cross-linking protein in macrophages/microglia. Biochem Biophys Res Commun 2001, 286:292-297.
74. Koblan L W, Doman J L, Wilson C, Levy J M, Tay T, Newby G A, Maianti J P, Raguram A, Liu D R: Improving cytidine and adenine base editors by expression optimization and ancestral reconstruction. Nat Biotechnol 2018, 36:843-846.
75. Richter M F, Zhao K T, Eton E, Lapinaite A, Newby G A, Thuronyi B W, Wilson C, Koblan L W, Zeng J, Bauer D E, et al: Phage-assisted evolution of an adenine base editor with improved Cas domain compatibility and activity. Nature Biotechnology 2020, 38:883-891.
76. Walton R T, Christie K A, Whittaker M N, Kleinstiver B P: Unconstrained genome targeting with near-PAMless engineered CRISPR-Cas9 variants. Science 2020, 368:290-296.
77. Nishimasu H, Shi X, Ishiguro S, Gao L, Hirano S, Okazaki S, Noda T, Abudayyeh 00, Gootenberg J S, Mori H, et al: Engineered CRISPR-Cas9 nuclease with expanded targeting space. Science (New York, N Y) 2018 Aug. 30, 361.
78. Winter J, Luu A, Gapinske M, Manandhar S, Shirguppe S, Woods W S, Song J S, Perez-Pinera P: Targeted exon skipping with AAV-mediated split adenine base editors. Cell Discovery 2019, 5.
79. Bae S, Park J, Kim J-S: Cas-OFFinder: a fast and versatile algorithm that searches for potential off-target sites of Cas9 RNA-guided endonucleases. Bioinformatics 2014, 30:1473-1475.
80. Cradick T J, Qiu P, Lee C M, Fine E J, Bao G: COSMID: A Web-based Tool for Identifying and Validating CRISPR/Cas Off-target Sites. Mol Ther Nucleic Acids 2014, 3:e214.
81. Ostlund C, Folker E S, Choi J C, Gomes E R, Gundersen G G, Worman H J: Dynamics and molecular interactions of linker of nucleoskeleton and cytoskeleton (LINC) complex proteins. J Cell Sci 2009, 122:4099-4108.
82. Yamamoto A, Lucas, J. J., and Hen, R.: Reversal of neuropathology and motor dysfunction in a conditional model of Huntington's disease. Cell 2000, 101:57-66.
83. Diaz-Hernendez M, Torres-Peraza, J., Salvatori-Abarca, A., Moren, M. A., G6mez-Ramos, P., Alberch, J., and Lucas, J. J.: Full motor recovery despite striatal neuron loss and formation of irreversible amyloid-like inclusions in a conditional mouse model of Huntington's disease. J Neurosci 2005, 25:9773-9781.
84. Kordasiewicz H B, Stanek L M, Wancewicz E V, Mazur C, McAlonis M M, Pytel K A, Artates J W, Weiss A, Cheng S H, Shihabuddin L S, et al: Sustained therapeutic reversal of Huntington's disease by transient repression of huntingtin synthesis. Neuron 2012, 74:1031-1044.
85. Harper S Q, Staber P D, He X, Eliason S L, Martins I H, Mao Q, Yang L, Kotin R M, Paulson H L, Davidson B L: RNA interference improves motor and neuropathological abnormalities in a Huntington's disease mouse model. Proc Natl Acad Sci USA 2005, 102:5820-5825.
86. Rodriguez-Lebron E, Denovan-Wright E M, Nash K, Lewin A S, Mandel R J: Intrastriatal rAAV-mediated delivery of anti-huntingtin shRNAs induces partial reversal of disease progression in R6/1 Huntington's disease transgenic mice. Mol Ther 2005, 12:618-633.
87. Wang G, Liu X, Gaertig M A, Li S, Li X J: Ablation of huntingtin in adult neurons is nondeleterious but its depletion in young mice causes acute pancreatitis. Proc Natl Acad Sci USA 2016, 113:3359-3364.
88. Carroll J B, Warby S C, Southwell A L, Doty C N, Greenlee S, Skotte N, Hung G, Bennett C F, Freier S M, Hayden M R: Potent and selective antisense oligonucleotides targeting single-nucleotide polymorphisms in the Huntington disease gene/allele-specific silencing of mutant huntingtin. Mol Ther 2011, 19:2178-2185.
89. Southwell A L, Skotte N H, Kordasiewicz H B, Ostergaard M E, Watt A T, Carroll J B, Doty C N, Villanueva E B, Petoukhov E, Vaid K, et al: In vivo evaluation of candidate allele-specific mutant huntingtin gene silencing antisense oligonucleotides. Mol Ther 2014, 22:2093-2106.
90. Dragatsis I, Dietrich P, Ren H, Deng Y P, Del Mar N, Wang H B, Johnson I M, Jones K R, Reiner A: Effect of early embryonic deletion of huntingtin from pyramidal neurons on the development and long-term survival of neurons in cerebral cortex and striatum. Neurobiology of Disease 2018, 111:102-117.
91. Vogt T, Dietrich P, Johnson I M, Alli S, Dragatsis I: Elimination of huntingtin in the adult mouse leads to progressive behavioral deficits, bilateral thalamic calcification, and altered brain iron homeostasis. PLOS Genetics 2017, 13.
92. Monteys A M, Ebanks S A, Keiser M S, Davidson B L: CRISPR/Cas9 editing of the mutant huntingtin allele in vitro and in vivo. Molecular therapy 2017, 25:12-23.
93. Shin J W, Kim K H, Chao M J, Atwal R S, Gillis T, MacDonald M E, Gusella J F, Lee J M: Permanent inactivation of Huntington's disease mutation by personalized allele-specific CRISPR/Cas9. Hum Mol Genet 2016, 25:4566-4576.
94. Oikemus S R, Pfister E L, Sapp E, Chase K O, Kennington L A, Hudgens E, Miller R, Zhu L J, Chaudhary A, Mick E O, et al: Allele-Specific Knockdown of Mutant Huntingtin Protein via Editing at Coding Region Single Nucleotide Polymorphism Heterozygosities. Hum Gene Ther 2022, 33:25-36.
95. Grimm D, Streetz K L, Jopling C L, Storm T A, Pandey K, Davis C R, Marion P, Salazar F, Kay M A: Fatality in mice due to oversaturation of cellular microRNA/short hairpin RNA pathways. Nature 2006, 441:537-541.
96. Ekman F K, Ojala D S, Adil M M, Lopez P A, Schaffer D V, Gaj T: CRISPR-Cas9-Mediated Genome Editing Increases Lifespan and Improves Motor Deficits in a Huntington& #x2019;s Disease Mouse Model. Molecular Therapy—Nucleic Acids 2019, 17:829-839.
97. Yang S, Chang R, Yang H, Zhao T, Hong Y, Kong H E, Sun X, Qin Z, Jin P, Li S, Li X-J: CRISPR/Cas9-mediated gene editing ameliorates neurotoxicity in mouse model of Huntington's disease. The Journal of Clinical Investigation 2017, 127:2719-2724.
98. Doudna J A: The promise and challenge of therapeutic genome editing. Nature 2020, 578:229-236.
99. Kosicki M, Tomberg K, Bradley A: Repair of double-strand breaks induced by CRISPR-Cas9 leads to large deletions and complex rearrangements. Nature Biotechnology 2018, 36:765-771.
100. Alvarez M M, Biayna J, Supek F: TP53-dependent toxicity of CRISPR/Cas9 cuts is differential across genomic loci and can confound genetic screening. Nature Communications 2022, 13:4520.
101. Tsuchida C A, Brandes N, Bueno R, Trinidad M, Mazumder T, Yu B, Hwang B, Chang C, Liu J, Sun Y, et al: Mitigation of chromosome loss in clinical CRISPR-Cas9-engineered T cells. bioRxiv 2023.
102. Dagbay K B, Hardy J A: Multiple proteolytic events in caspase-6 self-activation impact conformations of discrete structural regions. Proceedings of the National Academy of Sciences 2017, 114:E7977-E7986.
103. Zheng M, Karki R, Vogel P, Kanneganti T D: Caspase-6 Is a Key Regulator of Innate Immunity, Inflammasome Activation, and Host Defense. Cell 2020, 181:674-687.e613.
104. Wang X J, Cao Q, Zhang Y, Su X D: Activation and regulation of caspase-6 and its role in neurodegenerative diseases. Annu Rev Pharmacol Toxicol 2015, 55:553-572.
105. Riedl S J, Shi Y: Molecular mechanisms of caspase regulation during apoptosis. Nat Rev Mol Cell Biol 2004, 5:897-907.
106. Baskin-Bey E, Washburn K, Feng S, Oltersdorf T, Shapiro D, Huyghe M, Burgart L, Garrity-Park M, Vilsteren F V, Oliver L, et al: Clinical Trial of the Pan-Caspase Inhibitor, IDN-6556, in Human Liver Preservation Injury. American Journal of Transplantation 2007/01/01, 7.
107. Sarah H MacKenzie JLS, A Clay Clark The potential for caspases in drug discovery. Curr Opin Drug Discov Dev 2010, 13:68-76.
108. Vonsattel J-P, Myers R H, Stevens T J, Ferrante R J, Bird E D, Richardson E P: Neuropathological Classification of Huntington's Disease. Journal of Neuropathology and Experimental Neurology 1985, 44:559-577.
109. Landles C, Weiss A, Franklin S, Howland D, Bates G: Caspase-6 does not contribute to the proteolysis of mutant huntingtin in the HdhQ150 knock-in mouse model of Huntington's disease. PLoS Currents 2012 Jul. 16, 4.
110. Gafni J, Papanikolaou T, DeGiacomo F, Holcomb J, Chen S, Menalled L, Kudwa A, Fitzpatrick J, Miller S, Ramboz S, et al: Caspase-6 Activity in a BACHD Mouse Modulates Steady-State Levels of Mutant Huntingtin Protein But Is Not Necessary for Production of a 586 Amino Acid Proteolytic Fragment. The Journal of Neuroscience 2012 May 30, 32.
111. Kim H, Lenoir S, Helfricht A, Jung T, Karneva Z K, Lee Y, Beumer W, Horst GBvd, Anthonijsz H, Buil L C M, et al: A pathogenic proteolysis-resistant huntingtin isoform induced by an antisense oligonucleotide maintains huntingtin function. JCI Insight 2022/09/08, 7.
112. Reddy P H, Williams M, Charles V, Garrett L, Pike-Buchanan L, Whetsell W O, Jr., Miller G, Tagle D A: Behavioural abnormalities and selective neuronal loss in H D transgenic mice expressing mutated full-length H D cDNA. Nat Genet 1998, 20:198-202.
113. Martindale D, Hackam A, Wieczorek A, Ellerby L, Wellington C, McCutcheon K, Singaraja R, Kazemi-Esfarjani P, Devon R, Kim S U, et al: Length of huntingtin and its polyglutamine tract influences localization and frequency of intracellular aggregates. Nature Genetics 1998, 18:150-154.
114. Hodgson J G, Agopyan N, Gutekunst C A, Leavitt B R, LePiane F, Singaraja R, Smith D J, Bissada N, McCutcheon K, Nasir J, et al: A YAC mouse model for Huntington's disease with full-length mutant huntingtin, cytoplasmic toxicity, and selective striatal neurodegeneration. Neuron 1999, 23:181-192.
115. Kinnunen K M, Schwarz A J, Turner E C, Pustina D, Gantman E C, Gordon M F, Joules R, Mullin A P, Scahill R I, Georgiou-Karistianis N: Volumetric MRI-Based Biomarkers in Huntington's Disease: An Evidentiary Review. Frontiers in Neurology 2021, 12.
116. Squitieri F, Cannella M, Simonelli M, Sassone J, Martino T, Venditti E, Ciammola A, Colonnese C, Frati L, Ciarmiello A: Distinct Brain Volume Changes Correlating with Clinical Stage, Disease Progression Rate, Mutation Size, and Age at Onset Prediction as Early Biomarkers of Brain Atrophy in Huntington's Disease. CNS Neuroscience &amp; Therapeutics 2009, 15:1-11.
117. Reiner A, Dragatsis I, Dietrich P: Genetics and neuropathology of Huntington's disease. Int Rev Neurobiol 2011, 98:325-372.
118. Costanzo M, Abounit S, Marzo L, Danckaert A, Chamoun Z, Roux P, Zurzolo C: Transfer of polyglutamine aggregates in neuronal cells occurs in tunneling nanotubes. Journal of Cell Science 2013.
119. Trajkovic K, Jeong H, Krainc D: Mutant Huntingtin Is Secreted via a Late Endosomal/Lysosomal Unconventional Secretory Pathway. The Journal of Neuroscience 2017, 37:9000-9012.
120. Jarosinska O D, Ruidiger S G D: Molecular Strategies to Target Protein Aggregation in Huntington's Disease. Frontiers in Molecular Biosciences 2021, 8.
121. R. Bonavita G S, R. Di Martino, S. Nuzzo, E. Polishchuk, M. Di Gennarol, S. V. Williams, M. G. Caporaso, C. Caiazza, R. Polishchuk, M. D'Agostino, A. Fleming and M. Renna: The HSPB1-p62/SQSTM1 functional complex regulates the unconventional secretion and transcellular spreading of the H D-associated mutant huntingtin protein. Human Molecular Genetics 2023, 32:2269-2291.
122. Chen S, Ferrone F A, Wetzel R, Chen S, Ferrone F A, Wetzel R: Huntington's disease age-of-onset linked to polyglutamine aggregation nucleation. Proceedings of the National Academy of Sciences 2002-9-3, 99.
123. Slepko N, Bhattacharyya A M, Jackson G R, Steffan J S, Marsh J L, Thompson L M, Wetzel R, Slepko N, Bhattacharyya A M, Jackson G R, et al: Normal-repeat-length polyglutamine peptides accelerate aggregation nucleation and cytotoxicity of expanded polyglutamine proteins. Proceedings of the National Academy of Sciences 2006-9-26, 103.
124. Marchionini D M, Liu J-P, Ambesi-Impiombato A, Kerker K, Cirillo K, Bansal M, Mushlin R, Brunner D, Ramboz S, Kwan M, et al: Benefits of global mutant huntingtin lowering diminish over time in a Huntington's disease mouse model. JCI Insight 2022 Oct. 24, 7.
125. Stanek Lisa M. SSP, Mastis Bryan, Richards Amy R. Treleaven Christopher M., Taksir Tatyana, Misra Kuma, Cheng Seng H., Shihabuddin Lamya S.: Silencing Mutant Huntingtin by Adeno-Associated Virus-Mediated RNA Interference Ameliorates Disease Manifestations in the YAC128 Mouse Model of Huntington's Disease. Human Gene Therapy 2014-01-31, 25.
126. Zeitlin S, Liu J-P, Chapman D L, Papaioannou V E, Efstratiadis A: Increased apoptosis and early embryonic lethality in mice nullizygous for the Huntington's disease gene homologue. Nature Genetics 1995, 11:155-163.
127. Slesarenko Y S, Lavrov A V, Smirnikhina S A: Off-target effects of base editors: what we know and how we can reduce it. Current Genetics 2022, 68:39-48.
128. Park S, Beal P A: Off-target editing by CRISPR-guided DNA base editors. Biochemistry 2019, 58:3727-3734.
129. Chatterjee P, Lee J, Nip L, Koseki S R T, Tysinger E, Sontheimer E J, Jacobson J M, Jakimo N: A Cas9 with PAM recognition for adenine dinucleotides. Nat Commun 2020, 11:2474.
130. Zhao L, Koseki S R T, Silverstein R A, Amrani N, Peng C, Kramme C, Savic N, Pacesa M, Rodriguez T C, Stan T, et al: PAM-flexible genome editing with an engineered chimeric Cas9. Nature Communications 2023, 14:6175.
131. Neugebauer M E, Hsu A, Arbab M, Krasnow N A, McElroy A N, Pandey S, Doman J L, Huang T P, Raguram A, Banskota S, et al: Evolution of an adenine base editor into a small, efficient cytosine base editor with low off-target activity. Nature Biotechnology 2022 41:5 2022-11-10, 41.
132. Lam D K, Feliciano P R, Arif A, Bohnuud T, Fernandez T P, Gehrke J M, Grayson P, Lee K D, Ortega M A, Sawyer C, et al: Improved cytosine base editors generated from TadA variants. Nature Biotechnology 2023 41:5 2023-01-09, 41.
133. Zhang E, Neugebauer M E, Krasnow N A, Liu D R, Zhang E, Neugebauer M E, Krasnow N A, Liu D R: Phage-assisted evolution of highly active cytosine base editors with enhanced selectivity and minimal sequence context preference. Nature Communications 2024 15:1 2024-02-24, 15.
134. Liang P, Xie X, Zhi S, Sun H, Zhang X, Chen Y, Chen Y, Xiong Y, Ma W, Liu D, et al: Genome-wide profiling of adenine base editor specificity by EndoV-seq. Nature Communications 2019, 10:67.
135. Southwell A L, Skotte N H, Villanueva E B, Ostergaard M E, Gu X, Kordasiewicz H B, Kay C, Cheung D, Xie Y, WaltI S, et al: A novel humanized mouse model of Huntington disease for preclinical development of therapeutics targeting mutant huntingtin alleles. Human Molecular Genetics 2017:ddx021.
136. Gapinske M, Tague N, Winter J, Underhill G H, Perez-Pinera P: Targeted Gene Knock Out Using Nuclease-Assisted Vector Integration: Hemi- and Homozygous Deletion of JAG1. Methods Mol Biol 2018, 1772:233-248.
137. Kluesner M G, Nedveck D A, Lahr W S, Garbe J R, Abrahante J E, Webber B R, Moriarity B S: EditR: A Method to Quantify Base Editing from Sanger Sequencing. The CRISPR Journal 2018, 1:239-250.
138. Livak K J, Schmittgen T D: Analysis of relative gene expression data using real-time quantitative PCR and the 2(-Delta Delta C(T)) Method. Methods 2001, 25:402-408.
139. Law C W, Chen Y, Shi W, Smyth G K, Law C W, Chen Y, Shi W, Smyth G K: voom: precision weights unlock linear model analysis tools for RNA-seq read counts. Genome Biology 2014 15:2 2014-02-03, 15.
140. S M C, P D, J K S, S Z: Helper-free Production of Laboratory Grade AAV and Purification by lodixanol Density Gradient Centrifugation—PubMed. Molecular therapy Methods & clinical development 2018, 10.
141. Fedorov A, Beichel R, Kalpathy-Cramer J, Finet J, Fillion-Robin J-C, Pujol S, Bauer C, Jennings D, Fennessy F, Sonka M, et al: 3D Slicer as an image computing platform for the Quantitative Imaging Network. Magnetic Resonance Imaging 2012, 30:1323-1341.
142. Lein E S, Hawrylycz M J, Ao N, Ayres M, Bensinger A, Bernard A, Boe A F, Boguski M S, Brockway K S, Byrnes E J, et al: Genome-wide atlas of gene expression in the adult mouse brain. Nature 2007, 445:168-176.

1. A method of modifying RNA splicing of a pre-mRNA molecule to produce a modified mRNA molecule comprising delivering to a cell one or more vectors or proteins comprising:
(i) (a) a nucleic acid molecule encoding one or more PAM-less or nearly PAM-less Cas nickases and one or more cytosine or adenosine deaminases; or (b) one or more PAM-less or nearly PAM-less Cas proteins and one or more cytosine or adenosine deaminase proteins; and
(ii) a nucleic acid molecule encoding one or more single guide RNA (sgRNA) molecules targeting both a splice acceptor site and a splice donor site of a DNA molecule encoding the pre-mRNA;
wherein one or more exons or one or more portions of an exon are excluded during RNA splicing, resulting in the production of the modified mRNA molecule.

2. The method of claim 1, wherein the nucleic acid molecule encoding one or more PAM-less or nearly PAM-less Cas nickases and the nucleic acid molecule encoding the one or more cytosine or adenosine deaminases are linked on one vector such that a fusion protein of the one or more PAM-less or nearly PAM-less Cas nickases and the one or more cytosine or adenosine deaminases can be expressed or wherein the or one or more PAM-less or nearly PAM-less Cas proteins and one or more cytosine or adenosine deaminase proteins are present in a fusion protein.

3. (canceled)

4. (canceled)

5. (canceled)

6. The method of claim 1, wherein an amount of cryptic splicing is reduced as compared to a cell not receiving the one or more vectors or proteins.

7. The method of claim 1, wherein intron retention is reduced as compared to a control.

8. The method of claim 1, wherein full exon skipping is increased as compared to a control.

9. The method of claim 1, wherein the one or more vectors are Adeno-associated virus (AAV) vectors.

10. (canceled)

11. The method of claim 1, wherein the modified mRNA molecule is an amyloid precursor protein (APP) modified mRNA molecule.

12. The method of claim 1, wherein the one or more exons or one or more portions of an exon that are removed are from exon 17 of an amyloid precursor protein (APP) gene.

13. The method of claim 1, wherein the modified mRNA molecule is a huntingtin (HTT) modified mRNA molecule.

14. The method of claim 1, wherein the one or more exons or one or more portions of an exon that are removed are from exon 12 or 13 of a huntingtin (HTT) gene.

15. The method of claim 1, wherein the one or more vectors comprise:
(i) a first vector comprising an inverted terminal repeat (ITR) sequence, a promoter, a nucleic acid molecule encoding one or more cytosine or adenosine deaminases, and a nucleic acid molecule encoding an N-terminal portion of a PAM-less or nearly PAM-less Cas nickase, an N-terminal fragment of a dimerization protein, and an ITR sequence; and
(ii) a second vector comprising an ITR sequence, a promoter, a C-terminal fragment of a dimerization protein, a nucleic acid molecule encoding a C-terminal portion of a PAM-less or nearly PAM-less Cas nickase, a nucleic acid molecule encoding a uracil glycosylase inhibitor, and an ITR sequence,
wherein the first vector, the second vector, or both the first vector and second vector further comprise a nucleic acid molecule encoding one or more sgRNA molecules targeting a splice acceptor site and/or a splice donor site of a DNA molecule encoding the pre-mRNA.

16. (canceled)

17. The method of claim 15, wherein the first vector, the second vector, or both the first vector and second vector further comprise one or more nuclear localization signal (NLS) sequences.

18. (canceled)

19. The method of claim 15, wherein the N-terminal fragment of a dimerization protein is an N-terminal fragment of an intein, and wherein the C-terminal fragment of a dimerization protein is a C-terminal fragment of an intein.

20. (canceled)

21. (canceled)

22. (canceled)

23. (canceled)

24. (canceled)

25. (canceled)

26. (canceled)

27. The method of claim 15, wherein the N-terminal fragment of the Cas nickase and the C-terminal fragment of the Cas nickase form a full-length Cas nickase when combined.

28. The method of claim 15, wherein the PAM-less or nearly PAM-less Cas nickase is split into a N-terminal fragment and a C-terminal fragment at a split point.

29. The method of claim 28, wherein:
(a) the split point is localized at any amino acid between position 564 and 584, and the N-terminal fragment of PAM-less or nearly PAM-less Cas nickase comprises nucleotides from position 1 of the PAM-less or nearly PAM-less Cas nickase to the split point and the C-terminal fragment of a PAM-less or nearly PAM-less Cas nickase comprises nucleotides from the split point to position 1371 of the PAM-less or nearly PAM-less Cas nickase;
(b) the split point is localized at any amino acid between position 249 and 269, and the N-terminal fragment of the PAM-less or nearly PAM-less Cas nickase comprises nucleotides from position 1 of the PAM-less or nearly PAM-less Cas nickase to the split point and the C-terminal fragment of the PAM-less or nearly PAM-less Cas nickase comprises nucleotides from the split point to position 1371 of the PAM-less or nearly PAM-less Cas nickase; or
(c) the split point is localized at any amino acid between position 265 and 285, and the N-terminal fragment of the PAM-less or nearly PAM-less Cas nickase comprises nucleotides from position 1 of the PAM-less or nearly PAM-less Cas nickase to the split point and the C-terminal fragment of the PAM-less or nearly PAM-less Cas nickase comprises nucleotides from the split point to position 1371 of the PAM-less or nearly PAM-less Cas nickase.

30. The method of claim 28, wherein:
(a) the split point is localized at any amino acid between position 703 and 723, and the N-terminal fragment of the PAM-less or nearly PAM-less Cas nickase comprises nucleotides from position 1 of the PAM-less or nearly PAM-less Cas nickase to the split point and the C-terminal fragment of the PAM-less or nearly PAM-less Cas nickase comprises nucleotides from the split point to position 1371 of the PAM-less or nearly PAM-less Cas nickase;
(b) the split point is localized at any amino acid between position 935 and 965, and the N-terminal fragment of the PAM-less or nearly PAM-less Cas nickase comprises nucleotides from position 1 of the PAM-less or nearly PAM-less Cas nickase to the split point and the C-terminal fragment of the PAM-less or nearly PAM-less Cas nickase comprises nucleotides from the split point to position 1371 of the PAM-less or nearly PAM-less Cas nickase;
(c) the split point is localized at any amino acid between position 1044 and 1064 and, the N-terminal fragment of the PAM-less or nearly PAM-less Cas nickase comprises nucleotides from position 1 of the PAM-less or nearly PAM-less Cas nickase to the split point and the C-terminal fragment of the PAM-less or nearly PAM-less Cas nickase comprises nucleotides from the split point to position 1371 of the PAM-less or nearly PAM-less Cas nickase; or
(d) the split point is localized at any amino acid between position 1105 and 1125, and the N-terminal fragment of the PAM-less or nearly PAM-less Cas nickase comprises nucleotides from position 1 of the PAM-less or nearly PAM-less Cas nickase to the split point and the C-terminal fragment of the PAM-less or nearly PAM-less Cas nickase comprises nucleotides from the split point to position 1371 of the PAM-less or nearly PAM-less Cas nickase.

31. A method of treating Alzheimer's disease comprising delivering to a patient in need thereof one or more vectors or proteins comprising:
(i) a nucleic acid molecule encoding one or more PAM-less or nearly PAM-less Cas nickases and one or more cytosine or adenosine deaminases or one or more PAM-less or nearly PAM-less Cas proteins and one or more cytosine or adenosine deaminase proteins; and
(ii) a nucleic acid molecule encoding one or more single guide RNA (sgRNA) molecules targeting a splice acceptor site, a splice donor site, or both a splice acceptor site and a splice donor site of a target amyloid precursor protein gene (APP),
wherein one or more exons or one or more portions of an exon of the target amyloid precursor protein gene (APP) are excluded during RNA splicing.

32. (canceled)

33. (canceled)

34. (canceled)

35. (canceled)

36. (canceled)

37. (canceled)

38. (canceled)

39. A method of treating Huntington's disease comprising delivering one or more vectors or proteins to a patient in need thereof:
(i) a nucleic acid molecule encoding one or more PAM-less or nearly PAM-less Cas nickases and one or more cytosine or adenosine deaminases or one or more PAM-less or nearly PAM-less Cas proteins and one or more cytosine or adenosine deaminase proteins; and
(ii) a nucleic acid molecule encoding one or more single guide RNA (sgRNA) molecules targeting a splice acceptor site, a splice donor site, or both a splice acceptor site and splice donor site of a target huntingtin gene (HTT);
wherein one or more exons or one or more portions of an exon of the huntingtin gene (HTT) are excluded during RNA splicing.

40. The method of claim 39, wherein a caspase-6 cleavage site is disrupted when one or more exons or one or more portions of an exon of the huntingtin gene (HTT) are excluded during RNA splicing.

41. (canceled)

42. (canceled)

43. (canceled)

44. (canceled)

45. (canceled)

46. (canceled)

47. (canceled)

48. (canceled)


SEQUENCE LISTING (530 sequences)

SEQ ID NO: 1 - protein, 1605 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGDSGGSKRTADGSEFEPKKKRKV

SEQ ID NO: 2 - protein, 2117 aa, synthetic construct
MKRTADGSEFESPKKKRKVSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSDKKYSIGLAIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKVGGGGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 3 - protein, 1031 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVCLAGDTLITLADGRRVPIRELVSQQNFSVWALNPQTYRLERARVSRAFCTGIKPVYRLTTRLGRSIRATANHRFLTPQGWKRVDELQPGDYLALPRRIPTAS

SEQ ID NO: 4 - protein, 821 aa, synthetic construct
MAAACPELRQLAQSDVYWDPIVSIEPDGVEEVFDLTVPGPHNFVANDIIAHNSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 5 - protein, 1031 aa, synthetic construct; identical to SEQ ID NO: 3

SEQ ID NO: 6 - protein, 821 aa, synthetic construct
MAAACPELRQLAQSDVYWDPIVSIEPDGVEEVFDLTVPGPHNFVANDIIAHNSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 7 - protein, 1230 aa, synthetic construct
MKRTADGSEFESPKKKRKVSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDASGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVCLAGDTLITLADGRRVPIRELVSQQNFSVWALNPQTYRLERARVSRAFCTGIKPVYRLTTRLGRSIRATANHRFLTPQGWKRVDELQPGDYLALPRRIPTAS

SEQ ID NO: 8 - protein, 1605 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGDSGGSKRTADGSEFEPKKKRKV

SEQ ID NO: 9 - protein, 1031 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRQSKRGAAGSLMNVLNYPGMNHRVEITEGILADECAALTCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVCLAGDTLITLADGRRVPIRELVSQQNFSVWALNPQTYRLERARVSRAFCTGIKPVYRLTTRLGRSIRATANHRFLTPQGWKRVDELQPGDYLALPRRIPTAS

SEQ ID NO: 10 - protein, 1107 aa, synthetic construct
MGKPIPNPLLGLDSTKRTADGSEFESPKKKRKVSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVCLAGDTLITLADGRRVPIRELVSQQNFSVWALNPQTYRLERARVSRAFCTGIKPVYRLTTRLGRSIRATANHRFLTPQGWKRVDELQPGDYLALPRRIPTAS

SEQ ID NO: 11 - protein, 931 aa, synthetic construct
MAAACPELRQLAQSDVYWDPIVSIEPDGVEEVFDLTVPGPHNFVANDIIAHNSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLYPYDVPDYAYPYDVPDYAYPYDVPDYASGGSPKKKRKV

SEQ ID NO: 12 - protein, 1107 aa, synthetic construct; identical to SEQ ID NO: 10

SEQ ID NO: 13 - protein, 833 aa, synthetic construct
MAAACPELRQLAQSDVYWDPIVSIEPDGVEEVFDLTVPGPHNFVANDIIAHNSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSTNLSDIIEKETGKQLVIQESILMLPEEVEEVIGNKPESDILVHTAYDESTDENVMLLTSDAPEYKPWALVIQDSNGENKIKMLSYPYDVPDYAYPYDVPDYAYPYDVPDYASGGSPKKKRKV

SEQ ID NO: 14 - protein, 1116 aa, synthetic construct
MGKPIPNPLLGLDSTKRTADGSEFESPKKKRKVTSEKGPSTGDPTLRRRIESWEFDVFYDPRELRKETCLLYEIKWGMSRKIWRSSGKNTTNHVEVNFIKKFTSERRFHSSISCSITWFLSWSPCWECSQAIREFLSQHPGVTLVIYVARLFWAMDQRNRQGLRDLVNSGVTIQIMRASEYYHCWRNFVNYPPGDEAHWPQYPPLWMMLYALELHCIILSLPPCLKISRRWQNHLAFFRLHLQNCHYQTIPPHILLATGLIHPSVTWRLKSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVCLAGDTLITLADGRRVPIRELVSQQNFSVWALNPQTYRLERARVSRAFCTGIKPVYRLTTRLGRSIRATANHRFLTPQGWKRVDELQPGDYLALPRRIPTAS

SEQ ID NO: 15 - protein, 1041 aa, synthetic construct
MGKPIPNPLLGLDSTKRTADGSEFESPKKKRKVSFERNYDPRELRKETYLLYEIKWGKSGKLWRHWCQNNRTQHAEVYFLENIFNARRFNPSTHCSITWYLSWSPCAECSQKIVDFLKEHPNVNLEIYVARLYYPENERNRQGLRDLVNSGVTIRIMDLPDYNYCWKTFVSDQGGDEDYWPGHFAPWIKQYSLKLSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVCLAGDTLITLADGRRVPIRELVSQQNFSVWALNPQTYRLERARVSRAFCTGIKPVYRLTTRLGRSIRATANHRFLTPQGWKRVDELQPGDYLALPRRIPTAS

SEQ ID NO: 16 - protein, 1158 aa, synthetic construct
MGKPIPNPLLGLDSTKRTADGSEFESPKKKRKVDPQRLRQWPGPGPASRGGYGQRPRIRNPEEWFHELSPRTFSFHFRNLRFASGQNRSYICCQVEGKNCFFQGIFQNQVPPDPPCHAELCFLSWFQSWGLSPDEHYYVTWFISWSPCCECAAKVAQFLEENRNVSLSLSAARLYYFWKSESREGLRRLSDLGAQVGIMSFQDFQHCWNNFVHNLGMPFQPWKKLHKNYQRLVTELKQILREEPATYGSPQAQGKVRIGSTAAGLRHSHSHTRSEAHLRPNHSSRQHRILNPPREARARTCVLVDASWICYRSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVCLAGDTLITLADGRRVPIRELVSQQNFSVWALNPQTYRLERARVSRAFCTGIKPVYRLTTRLGRSIRATANHRFLTPQGWKRVDELQPGDYLALPRRIPTAS

SEQ ID NO: 17 - protein, 1072 aa, synthetic construct
MGKPIPNPLLGLDSTKRTADGSEFESPKKKRKVKPQIRDHRPNPMEAMYPHIFYFHFENLEKAYGRNETWLCFTVEIIKQYLPVPWKKGVFRNQVDPETHCHAEKCFLSWFCNNTLSPKKNYQVTWYTSWSPCPECAGEVAEFLAEHSNVKLTIYTARLYYLWDTDYQEGLRSLSEEGASVEIMDYEDFQYCWENFVYDDGEPFKRWKGLKYNFQSLTRRLREILQSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVCLAGDTLITLADGRRVPIRELVSQQNFSVWALNPQTYRLERARVSRAFCTGIKPVYRLTTRLGRSIRATANHRFLTPQGWKRVDELQPGDYLALPRRIPTAS

SEQ ID NO: 18 - protein, 1045 aa, synthetic construct
MGKPIPNPLLGLDSTKRTADGSEFESPKKKRKVSEVEFSHEYWMRHALTLAKRARDERKAPVGAVLVLNNRVIGEGWNRAIGLHDPTAHAEIIALRQGGLVMQNYRLIDATLYVTFEPCVMCAGAMINSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVCLAGDTLITLADGRRVPIRELVSQQNFSVWALNPQTYRLERARVSRAFCTGIKPVYRLTTRLGRSIRATANHRFLTPQGWKRVDELQPGDYLALPRRIPTAS

SEQ ID NO: 19 - protein, 1045 aa, synthetic construct
MGKPIPNPLLGLDSTKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVCLAGDTLITLADGRRVPIRELVSQQNFSVWALNPQTYRLERARVSRAFCTGIKPVYRLTTRLGRSIRATANHRFLTPQGWKRVDELQPGDYLALPRRIPTAS

SEQ ID NO: 20 - protein, 821 aa, synthetic construct; identical to SEQ ID NO: 4

SEQ ID NO: 21 - protein, 327 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGLRSREEEEETDSRMPHLDSPGSSQPRRSFLSRVIRAALPLQLLLLLLLLLACLLPASEDDYSCTQANNFARSFYPMLRYTNGPPPT

SEQ ID NO: 22 - RNA, 20 nt, synthetic construct
  source 1..20: mol_type = other RNA
gtaggtgtttatacgggagc

SEQ ID NO: 23 - RNA, 20 nt, synthetic construct
  source 1..20: mol_type = other RNA
tgtaggtgtttatacgggag

SEQ ID NO: 24 - RNA, 20 nt, synthetic construct
  source 1..20: mol_type = other RNA
ctgtaggtgtttatacggga

SEQ ID NO: 25 - RNA, 20 nt, synthetic construct
  source 1..20: mol_type = other RNA
cctgtaggtgtttatacggg

SEQ ID NO: 26 - RNA, 20 nt, synthetic construct
  source 1..20: mol_type = other RNA
tcctgtaggtgtttatacgg

SEQ ID NO: 27 - RNA, 20 nt, synthetic construct
  source 1..20: mol_type = other RNA
ttcctgtaggtgtttatacg

SEQ ID NO: 28 - RNA, 20 nt, synthetic construct
  source 1..20: mol_type = other RNA
gtcaggtgagaatgtccgag

SEQ ID NO: 29 - RNA, 20 nt, synthetic construct
  source 1..20: mol_type = other RNA
tgtcaggtgagaatgtccga

SEQ ID NO: 30 - RNA, 20 nt, synthetic construct
  source 1..20: mol_type = other RNA
gtgtcaggtgagaatgtccg

SEQ ID NO: 31 - RNA, 20 nt, synthetic construct
  source 1..20: mol_type = other RNA
tgtgtcaggtgagaatgtcc

SEQ ID NO: 32 - RNA, 20 nt, synthetic construct
  source 1..20: mol_type = other RNA
actcaggtgatgagatatgt

SEQ ID NO: 33 - RNA, 20 nt, synthetic construct
  source 1..20: mol_type = other RNA
tactcaggtgatgagatatg

SEQ ID NO: 34 - RNA, 20 nt, synthetic construct
  source 1..20: mol_type = other RNA
tcttactcaggtgatgagat

SEQ ID NO: 35 - RNA, 20 nt, synthetic construct
  source 1..20: mol_type = other RNA
ttttcaaggtgttctttgca

SEQ ID NO: 36 - RNA, 20 nt, synthetic construct
  source 1..20: mol_type = other RNA
actgcctcaggtgcccccaa

SEQ ID NO: 37 - RNA, 20 nt, synthetic construct
  source 1..20: mol_type = other RNA
tcactgcctcaggtgccccc

SEQ ID NO: 38 - RNA, 20 nt, synthetic construct
  source 1..20: mol_type = other RNA
tcactgcctcaggtgccccc

SEQ ID NO: 39 - RNA, 20 nt, synthetic construct
  source 1..20: mol_type = other RNA
tatacctttctgcaccttgt

SEQ ID NO: 40 - RNA, 20 nt, synthetic construct
  source 1..20: mol_type = other RNA
ttctaggatttggttaatgg

SEQ ID NO: 41 - RNA, 20 nt, synthetic construct
  source 1..20: mol_type = other RNA
tcacctgacagaggtttcca

SEQ ID NO: 42 - RNA, 20 nt, synthetic construct
  source 1..20: mol_type = other RNA
cgcagctgttcagcccctcg

SEQ ID NO: 43 - RNA, 20 nt, synthetic construct
  source 1..20: mol_type = other RNA
ccgcagctgttcagcccctc

SEQ ID NO: 44 - RNA, 20 nt, synthetic construct
  source 1..20: mol_type = other RNA
tacgcacactggccaggctg

SEQ ID NO: 45 - RNA, 20 nt, synthetic construct
  source 1..20: mol_type = other RNA
ctacgcacactggccaggct

SEQ ID NO: 46 - RNA, 20 nt, synthetic construct
  source 1..20: mol_type = other RNA
tcccagggctcccactgcag

SEQ ID NO: 47 - RNA, 20 nt, synthetic construct
  source 1..20: mol_type = other RNA
ttcccagggctcccactgca

SEQ ID NO: 48 - RNA, 20 nt, synthetic construct
  source 1..20: mol_type = other RNA
gacaactcacctgggttcgg

SEQ ID NO: 49 - RNA, 20 nt, synthetic construct
  source 1..20: mol_type = other RNA
agacaactcacctgggttcg

SEQ ID NO: 50 - RNA, 20 nt, synthetic construct
  source 1..20: mol_type = other RNA
aagtttacctacctccacca

SEQ ID NO: 51 - RNA, 20 nt, synthetic construct
  source 1..20: mol_type = other RNA
acctacctccaccacaccat

SEQ ID NO: 52 - RNA, 20 nt, synthetic construct
  source 1..20: mol_type = other RNA
agtttacctacctccaccac

SEQ ID NO: 53 - RNA, 20 nt, synthetic construct
  source 1..20: mol_type = other RNA
ctagaagaggagaaggggag

SEQ ID NO: 54 - RNA, 20 nt, synthetic construct
  source 1..20: mol_type = other RNA
tcctagaagaggagaagggg

SEQ ID NO: 55 - RNA, 20 nt, synthetic construct
  source 1..20: mol_type = other RNA
atcctagaagaggagaaggg

SEQ ID NO: 56 - RNA, 20 nt, synthetic construct
  source 1..20: mol_type = other RNA
aatcctagaagaggagaagg

SEQ ID NO: 57 - RNA, 20 nt, synthetic construct
  source 1..20: mol_type = other RNA
aaatcctagaagaggagaag

SEQ ID NO: 58 - RNA, 22 nt, synthetic construct
  source 1..22: mol_type = other RNA
accattaaccaaatcctagaag

SEQ ID NO: 59 - RNA, 20 nt, synthetic construct
  source 1..20: mol_type = other RNA
caaatcctagaagaggagaa

SEQ ID NO: 60 - RNA, 20 nt, synthetic construct
  source 1..20: mol_type = other RNA
cacctgaggcagtgaaaaca

SEQ ID NO: 61 - RNA, 20 nt, synthetic construct
  source 1..20: mol_type = other RNA
gcacctgaggcagtgaaaac

SEQ ID NO: 62 - RNA, 20 nt, synthetic construct
  source 1..20: mol_type = other RNA
gaggcctgggtggggcgaca

SEQ ID NO: 63 - RNA, 20 nt, synthetic construct
  source 1..20: mol_type = other RNA
cttaccgaaatcttccacga

SEQ ID NO: 64 - RNA, 20 nt, synthetic construct
  source 1..20: mol_type = other RNA
ctcttaccgaaatcttccac

SEQ ID NO: 65 - RNA, 20 nt, synthetic construct
  source 1..20: mol_type = other RNA
cggtccacctacacgcaggc

SEQ ID NO: 66 - RNA, 20 nt, synthetic construct
  source 1..20: mol_type = other RNA
cccaccttcttgggcaggaa

SEQ ID NO: 67 - RNA, 20 nt, synthetic construct
  source 1..20: mol_type = other RNA
acccctgagaggatgaagca

SEQ ID NO: 68 - RNA, 20 nt, synthetic construct
  source 1..20: mol_type = other RNA
cacacttgaccatgatcatg

SEQ ID NO: 69 - RNA, 20 nt, synthetic construct
  source 1..20: mol_type = other RNA
actcacacttgaccatgatc

SEQ ID NO: 70 - RNA, 20 nt, synthetic construct
  source 1..20: mol_type = other RNA
ttgccaccactcacctgaca

SEQ ID NO: 71 - RNA, 20 nt, synthetic construct
  source 1..20: mol_type = other RNA
acaccttgaaaacaaattaa

SEQ ID NO: 72 - RNA, 20 nt, synthetic construct
  source 1..20: mol_type = other RNA
ttttaaaggtgttctttgcg

SEQ ID NO: 73 - RNA, 20 nt, synthetic construct
  source 1..20: mol_type = other RNA
ttttgaaggtgttctttaca

SEQ ID NO: 74 - RNA, 20 nt, synthetic construct
  source 1..20: mol_type = other RNA
tttaccaggtgttctttgct

SEQ ID NO: 75 - RNA, 20 nt, synthetic construct
  source 1..20: mol_type = other RNA
ttatcaaggtgttctataca

SEQ ID NO: 76 - RNA, 20 nt, synthetic construct
  source 1..20: mol_type = other RNA
ctttcaagttgttctgtgca

SEQ ID NO: 77 - RNA, 20 nt, synthetic construct
  source 1..20: mol_type = other RNA
aagtttccctaccgccacca

SEQ ID NO: 78 - RNA, 20 nt, synthetic construct
  source 1..20: mol_type = other RNA
aaggttaccttcctccacca

SEQ ID NO: 79 - RNA, 20 nt, synthetic construct
  source 1..20: mol_type = other RNA
aagtttccctacatccacca

SEQ ID NO: 80 - RNA, 20 nt, synthetic construct
  source 1..20: mol_type = other RNA
aagtctacgtacctccacca

SEQ ID NO: 81 - RNA, 20 nt, synthetic construct
  source 1..20: mol_type = other RNA
tagtttacctccctccaccc

SEQ ID NO: 82 - DNA, 20 bp, synthetic construct
  source 1..20: mol_type = other DNA
aacgatcacttgagcctggg

SEQ ID NO: 83 - DNA, 20 bp, synthetic construct
  source 1..20: mol_type = other DNA
tgagggaggtacctggatgg

SEQ ID NO: 84 - DNA, 20 bp, synthetic construct
  source 1..20: mol_type = other DNA
gcaacatagcgagaccctgt

SEQ ID NO: 85 - DNA, 20 bp, synthetic construct
  source 1..20: mol_type = other DNA
ggctctgctgtgacactctt

SEQ ID NO: 86 - DNA, 20 bp, synthetic construct
  source 1..20: mol_type = other DNA
agtagggggcatccagtctt

SEQ ID NO: 87 - DNA, 20 bp, synthetic construct
  source 1..20: mol_type = other DNA
agctctggaggaatgagggt

SEQ ID NO: 88 - DNA, 20 bp, synthetic construct
  source 1..20: mol_type = other DNA
ttgggtccttacagcaatcc

SEQ ID NO: 89 - DNA, 22 bp, synthetic construct
  source 1..22: mol_type = other DNA
gatgcaaaggcctcagctgttt

SEQ ID NO: 90 - DNA, 22 bp, synthetic construct
  source 1..22: mol_type = other DNA
gaggtagactgaggcttccagc

SEQ ID NO: 91 - DNA, 22 bp, synthetic construct
  source 1..22: mol_type = other DNA
gctcaactggtgtgtgcagatc

SEQ ID NO: 92 - DNA, 22 bp, synthetic construct
  source 1..22: mol_type = other DNA
tcacggaactttgggcgactat

SEQ ID NO: 93 - DNA, 55 bp, synthetic construct
  source 1..55: mol_type = other DNA
tcgtcggcagcgtcagatgtgtataagagacaggctcaactggtgtgtgcagatc

SEQ ID NO: 94 - DNA, 56 bp, synthetic construct
  source 1..56: mol_type = other DNA
gtctcgtgggctcggagatgtgtataagagacagtcacggaactttgggcgactat

SEQ ID NO: 95 - DNA, 20 bp, synthetic construct
  source 1..20: mol_type = other DNA
gctgaagccctgtgtttgtg

SEQ ID NO: 96 - DNA, 20 bp, synthetic construct
  source 1..20: mol_type = other DNA
gctgtagggactgccaatgt

SEQ ID NO: 97 - DNA, 21 bp, synthetic construct
  source 1..21: mol_type = other DNA
tcgtcagtatctccttccacc

SEQ ID NO: 98 - DNA, 22 bp, synthetic construct
  source 1..22: mol_type = other DNA
cggatttaagtgtgtgtgcccc

SEQ ID NO: 99 - DNA, 22 bp, synthetic construct
  source 1..22: mol_type = other DNA
ttctggcagggattaggctcac

SEQ ID NO: 100 - DNA, 55 bp, synthetic construct
  source 1..55: mol_type = other DNA
tcgtcggcagcgtcagatgtgtataagagacagcggatttaagtgtgtgtgcccc

SEQ ID NO: 101 - DNA, 56 bp, synthetic construct
  source 1..56: mol_type = other DNA
gtctcgtgggctcggagatgtgtataagagacagttctggcagggattaggctcac

SEQ ID NO: 102 - DNA, 20 bp, synthetic construct
  source 1..20: mol_type = other DNA
tgaataagggctggctggag

SEQ ID NO: 103 - DNA, 20 bp, synthetic construct
  source 1..20: mol_type = other DNA
attgtgtgaccggggtcttc

SEQ ID NO: 104 - DNA, 20 bp, synthetic construct
  source 1..20: mol_type = other DNA
gtagggttcctggcactgtc

SEQ ID NO: 105 - DNA, 20 bp, synthetic construct
  source 1..20: mol_type = other DNA
gtctgtgtgtgggactgagg

SEQ ID NO: 106 - DNA, 20 bp, synthetic construct
  source 1..20: mol_type = other DNA
gttgggtatcagctggtggg

SEQ ID NO: 107 - DNA, 53 bp, synthetic construct
  source 1..53: mol_type = other DNA
tcgtcggcagcgtcagatgtgtataagagacaggtctgtgtgtgggactgagg

SEQ ID NO: 108 - DNA, 54 bp, synthetic construct
  source 1..54: mol_type = other DNA
gtctcgtgggctcggagatgtgtataagagacaggttgggtatcagctggtggg

SEQ ID NO: 109 - DNA, 20 bp, synthetic construct
  source 1..20: mol_type = other DNA
agagacgggctttcactgtg

SEQ ID NO: 110 - DNA, 20 bp, synthetic construct
  source 1..20: mol_type = other DNA
ttagacacgtgacccttggc

SEQ ID NO: 111 - DNA, 20 bp, synthetic construct
  source 1..20: mol_type = other DNA
cggccagctcaacatttctt

SEQ ID NO: 112 - DNA, 22 bp, synthetic construct
  source 1..22: mol_type = other DNA
catctttgtcaccaccacaggc

SEQ ID NO: 113 - DNA, 22 bp, synthetic construct
  source 1..22: mol_type = other DNA
aggtactgggcttgcttctcag

SEQ ID NO: 114 - DNA, 55 bp, synthetic construct
  source 1..55: mol_type = other DNA
tcgtcggcagcgtcagatgtgtataagagacaggtcaagtggctcaacgagaacg

SEQ ID NO: 115 - DNA, 56 bp, synthetic construct
  source 1..56: mol_type = other DNA
gtctcgtgggctcggagatgtgtataagagacagtccaagaccactgagctcatgg

SEQ ID NO: 116 - DNA, 22 bp, synthetic construct
  source 1..22: mol_type = other DNA
cagattggcacccactggacta

SEQ ID NO: 117 - DNA, 22 bp, synthetic construct
  source 1..22: mol_type = other DNA
cctggtcccgtgaaatacacct

SEQ ID NO: 118 - DNA, 22 bp, synthetic construct
  source 1..22: mol_type = other DNA
gggtcagtgtgtctaaccctcc

SEQ ID NO: 119 - DNA, 22 bp, synthetic construct
  source 1..22: mol_type = other DNA
cccacgagcttgtaggaaagga

SEQ ID NO: 120 - DNA, 22 bp, synthetic construct
  source 1..22: mol_type = other DNA
cctggtcccgtgaaatacacct

SEQ ID NO: 121 - DNA, 55 bp, synthetic construct
  source 1..55: mol_type = other DNA
tcgtcggcagcgtcagatgtgtataagagacagcccacgagcttgtaggaaagga

SEQ ID NO: 122 - DNA, 56 bp, synthetic construct
  source 1..56: mol_type = other DNA
gtctcgtgggctcggagatgtgtataagagacagcctggtcccgtgaaatacacct

SEQ ID NO: 123 - DNA, 20 bp, synthetic construct
  source 1..20: mol_type = other DNA
gtccccaagtcttcctgagc

SEQ ID NO: 124 - DNA, 20 bp, synthetic construct
  source 1..20: mol_type = other DNA
tcctacccctcgatgaccag

SEQ ID NO: 125 - DNA, 20 bp, synthetic construct
  source 1..20: mol_type = other DNA
tgtcttccctctcctcctcc

SEQ ID NO: 126 - DNA, 20 bp, synthetic construct
  source 1..20: mol_type = other DNA
acccatctcctctggctctt

SEQ ID NO: 127 - DNA, 22 bp, synthetic construct
  source 1..22: mol_type = other DNA
gacgacgaggatgaggatggag

SEQ ID NO: 128 - DNA, 22 bp, synthetic construct
  source 1..22: mol_type = other DNA
cacccctttcccttggcttcta

SEQ ID NO: 129 - DNA, 53 bp, synthetic construct
  source 1..53: mol_type = other DNA
tcgtcggcagcgtcagatgtgtataagagacagacggctctcatcaactccac

SEQ ID NO: 130 - DNA, 52 bp, synthetic construct
  source 1..52: mol_type = other DNA
gtctcgtgggctcggagatgtgtataagagacagtgaggaggacgcaggaag

SEQ ID NO: 131 - DNA, 22 bp, synthetic construct
  source 1..22: mol_type = other DNA
ctgttctgacttccctccctcc

SEQ ID NO: 132 - DNA, 22 bp, synthetic construct
  source 1..22: mol_type = other DNA
tgggacttggctcacctgaatc

SEQ ID NO: 133 - DNA, 22 bp, synthetic construct
  source 1..22: mol_type = other DNA
tgggacttggctcacctgaatc

SEQ ID NO: 134 - DNA, 22 bp, synthetic construct
  source 1..22: mol_type = other DNA
tgcctggacaagaatgagctca

SEQ ID NO: 135 - DNA, 22 bp, synthetic construct
  source 1..22: mol_type = other DNA
ctctaggagacagtggggtcct

SEQ ID NO: 136 - DNA, 55 bp, synthetic construct
  source 1..55: mol_type = other DNA
tcgtcggcagcgtcagatgtgtataagagacagtgcctggacaagaatgagctca

SEQ ID NO: 137 - DNA, 56 bp, synthetic construct
  source 1..56: mol_type = other DNA
gtctcgtgggctcggagatgtgtataagagacagctctaggagacagtggggtcct

SEQ ID NO: 138 - DNA, 22 bp, synthetic construct
  source 1..22: mol_type = other DNA
ccaaccagttgggcagagaata

SEQ ID NO: 139 - DNA, 22 bp, synthetic construct
  source 1..22: mol_type = other DNA
acctggttgtattgagaggcca

SEQ ID NO: 140 - DNA, 26 bp, synthetic construct
  source 1..26: mol_type = other DNA
agttcttagcaaaaagctaagcctaa

SEQ ID NO: 141 - DNA, 22 bp, synthetic construct
  source 1..22: mol_type = other DNA
gaagttgagcctgttgatgccc

SEQ ID NO: 142 - DNA, 22 bp, synthetic construct
  source 1..22: mol_type = other DNA
cgttctgctgcatcttggacag

SEQ ID NO: 143 - DNA, 55 bp, synthetic construct
  source 1..55: mol_type = other DNA
tcgtcggcagcgtcagatgtgtataagagacaggaagttgagcctgttgatgccc

SEQ ID NO: 144 - DNA, 56 bp, synthetic construct
  source 1..56: mol_type = other DNA
gtctcgtgggctcggagatgtgtataagagacagcgttctgctgcatcttggacag

SEQ ID NO: 145 - DNA, 52 bp, synthetic construct
  source 1..52: mol_type = other DNA
tcgtcggcagcgtcagatgtgtataagagacagatccaaatgtcccctgcat

SEQ ID NO: 146 - DNA, 54 bp, synthetic construct
  source 1..54: mol_type = other DNA
gtctcgtgggctcggagatgtgtataagagacagcatggaagcacactgattcg

SEQ ID NO: 147 - DNA, 54 bp, synthetic construct
  source 1..54: mol_type = other DNA
tcgtcggcagcgtcagatgtgtataagagacagacacagaaaacgaagttgagc

SEQ ID NO: 148 - DNA, 56 bp, synthetic construct
  source 1..56: mol_type = other DNA
gtctcgtgggctcggagatgtgtataagagacaggcgataatgagtaaatcataaa

SEQ ID NO: 149 - DNA, 54 bp, synthetic construct
  source 1..54: mol_type = other DNA
tcgtcggcagcgtcagatgtgtataagagacagtttgaccagatccctctctca

SEQ ID NO: 150 - DNA, 54 bp, synthetic construct
  source 1..54: mol_type = other DNA
gtctcgtgggctcggagatgtgtataagagacaggggtctggtccctaccctta

SEQ ID NO: 151 - DNA, 53 bp, synthetic construct
  source 1..53: mol_type = other DNA
tcgtcggcagcgtcagatgtgtataagagacagcaaccttccaaccaagcaat

SEQ ID NO: 152 - DNA, 54 bp, synthetic construct
  source 1..54: mol_type = other DNA
gtctcgtgggctcggagatgtgtataagagacagcagtgtgacccttcaagcaa

SEQ ID NO: 153 - DNA, 53 bp, synthetic construct
  source 1..53: mol_type = other DNA
tcgtcggcagcgtcagatgtgtataagagacagacagccaaaccaaaaattgc

SEQ ID NO: 154 - DNA, 54 bp, synthetic construct
  source 1..54: mol_type = other DNA
gtctcgtgggctcggagatgtgtataagagacagtgggtggaattgacaatgaa

SEQ ID NO: 155 - DNA, 53 bp, synthetic construct
  source 1..53: mol_type = other DNA
tcgtcggcagcgtcagatgtgtataagagacaggtgcatgggatgttcatcag

SEQ ID NO: 156 - DNA, 54 bp, synthetic construct
  source 1..54: mol_type = other DNA
gtctcgtgggctcggagatgtgtataagagacagctcagccagactgccaactt

SEQ ID NO: 157 - DNA, 53 bp, synthetic construct
  source 1..53: mol_type = other DNA
tcgtcggcagcgtcagatgtgtataagagacagtgcaaggcccaagtaatagc

SEQ ID NO: 158 - DNA, 54 bp, synthetic construct
  source 1..54: mol_type = other DNA
gtctcgtgggctcggagatgtgtataagagacagcattttcagcaccagaacca

SEQ ID NO: 159 - DNA, 53 bp, synthetic construct
  source 1..53: mol_type = other DNA
tcgtcggcagcgtcagatgtgtataagagacagcctgtttcttagccgtcctg

SEQ ID NO: 160 - DNA, 54 bp, synthetic construct
  source 1..54: mol_type = other DNA
gtctcgtgggctcggagatgtgtataagagacagcacgtgggaaaacgtgatta

SEQ ID NO: 161 - DNA, 53 bp, synthetic construct
  source 1..53: mol_type = other DNA
tcgtcggcagcgtcagatgtgtataagagacagccccaagatatgggatgttg

SEQ ID NO: 162 - DNA, 54 bp, synthetic construct
  source 1..54: mol_type = other DNA
gtctcgtgggctcggagatgtgtataagagacagttagcaagccccttttgttc

SEQ ID NO: 163 - DNA, 53 bp, synthetic construct
  source 1..53: mol_type = other DNA
tcgtcggcagcgtcagatgtgtataagagacagatggacttggtcccaaacag

SEQ ID NO: 164 - DNA, 54 bp, synthetic construct
  source 1..54: mol_type = other DNA
gtctcgtgggctcggagatgtgtataagagacagagctccaccatatcgtgtcc

SEQ ID NO: 165 - DNA, 53 bp, synthetic construct
  source 1..53: mol_type = other DNA
tcgtcggcagcgtcagatgtgtataagagacagcggctggccatacactttta

SEQ ID NO: 166 - DNA, 54 bp, synthetic construct
  source 1..54: mol_type = other DNA
gtctcgtgggctcggagatgtgtataagagacagtgcctgagatgtgagtttgc

SEQ ID NO: 167 - DNA, 53 bp, synthetic construct
  source 1..53: mol_type = other DNA
tcgtcggcagcgtcagatgtgtataagagacagctgggacatgaagctcgtct

SEQ ID NO: 168 - DNA, 54 bp, synthetic construct
  source 1..54: mol_type = other DNA
gtctcgtgggctcggagatgtgtataagagacaggcactccagcctaggtgaca

SEQ ID NO: 169 - DNA, 22 bp, synthetic construct
  source 1..22: mol_type = other DNA
gaggtacccactgatggtaatg

SEQ ID NO: 170 - DNA, 21 bp, synthetic construct
  source 1..21: mol_type = other DNA
ctgaatcccacttcccattct

SEQ ID NO: 171 - DNA, 20 bp, synthetic construct
  source 1..20: mol_type = other DNA
caatgaccccttcattgacc

SEQ ID NO: 172 - DNA, 20 bp, synthetic construct
  source 1..20: mol_type = other DNA
ttgattttggagggatctcg

SEQ ID NO: 173 - protein, 1113 aa, synthetic construct
MGKPIPNPLLGLDSTAVKRPAATKKAGQAKKKKLDSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGSETPGTSESATPESDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVCLAGDTLITLADGRRVPIRELVSQQNFSVWALNPQTYRLERARVSRAFCTGIKPVYRLTTRLGRSIRATANHRFLTPQGWKRVDELQPGDYLALPRRIPTASGGGGGSPKKKRKVPKKKRKV

SEQ ID NO: 174 - protein, 853 aa, synthetic construct
MAVKRPAATKKAGQAKKKKLDAAACPELRQLAQSDVYWDPIVSIEPDGVEEVFDLTVPGPHNFVANDIIAHNSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSTNLSDIIEKETGKQLVIQESILMLPEEVEEVIGNKPESDILVHTAYDESTDENVMLLTSDAPEYKPWALVIQDSNGENKIKMLSYPYDVPDYAYPYDVPDYAYPYDVPDYASGGSPKKKRKV

SEQ ID NO: 175 - protein, 1606 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGDSGGSKRTADGSEFESPKKKRKV

SEQ ID NO: 176 - protein, 1045 aa, synthetic construct; identical to SEQ ID NO: 19

SEQ ID NO: 177 - protein, 821 aa, synthetic construct; identical to SEQ ID NO: 4

SEQ ID NO: 178 - protein, 200 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNH

SEQ ID NO: 179 - RNA, 20 nt, synthetic construct
  source 1..20: mol_type = other RNA
ttcactgaggctgaagacag

SEQ ID NO: 180 - RNA, 20 nt, synthetic construct
  source 1..20: mol_type = other RNA
ctcatccttcactgaggctg

SEQ ID NO: 181 - RNA, 20 nt, synthetic construct
  source 1..20: mol_type = other RNA
tcatccttcactgaggctga

SEQ ID NO: 182 - RNA, 20 nt, synthetic construct
  source 1..20: mol_type = other RNA
ccttcactgaggctgaagac

SEQ ID NO: 183 - RNA, 20 nt, synthetic construct
  source 1..20: mol_type = other RNA
ttcactgaggctgaagacag

SEQ ID NO: 184 - RNA, 20 nt, synthetic construct
  source 1..20: mol_type = other RNA
ttcagcctcagtgaaggatg

SEQ ID NO: 185 - RNA, 20 nt, synthetic construct
  source 1..20: mol_type = other RNA
tcttcagcctcagtgaagga

SEQ ID NO: 186 - RNA, 20 nt, synthetic construct
  source 1..20: mol_type = other RNA
ctgtcttcagcctcagtgaa

SEQ ID NO: 187 - RNA, 20 nt, synthetic construct
  source 1..20: mol_type = other RNA
tctgtcttcagcctcagtga

SEQ ID NO: 188 - RNA, 20 nt, synthetic construct
  source 1..20: mol_type = other RNA
ttctctgtcttcagcctcag

SEQ ID NO: 189 - RNA, 20 nt, synthetic construct
  source 1..20: mol_type = other RNA
gcctcagtgaaggatgagat

SEQ ID NO: 190 - RNA, 20 nt, synthetic construct
  source 1..20: mol_type = other RNA
tgtttctctgtcttcagcct

SEQ ID NO: 191 - RNA, 20 nt, synthetic construct
  source 1..20: mol_type = other RNA
ctctgtcttcagcctcagtg

SEQ ID NO: 192 - RNA, 20 nt, synthetic construct
  source 1..20: mol_type = other RNA
cttcagcctcagtgaaggat

SEQ ID NO: 193 - RNA, 20 nt, synthetic construct
  source 1..20: mol_type = other RNA
gcctcagtgaaggatgagat

SEQ ID NO: 194 - RNA, 20 nt, synthetic construct
  source 1..20: mol_type = other RNA
cttcactgaggctgaagaca

SEQ ID NO: 195 - RNA, 20 nt, synthetic construct
  source 1..20: mol_type = other RNA
tccttcactgaggctgaaga

SEQ ID NO: 196 - RNA, 20 nt, synthetic construct
  source 1..20: mol_type = other RNA
ctcatccttcactgaggctg

SEQ ID NO: 197 - RNA, 20 nt, synthetic construct
  source 1..20: mol_type = other RNA
tcagcctcagtgaaggatga

SEQ ID NO: 198 - RNA, 20 nt, synthetic construct
  source 1..20: mol_type = other RNA
tgtcttcagcctcagtgaag

SEQ ID NO: 199 - RNA, 20 nt, synthetic construct
  source 1..20: mol_type = other RNA
tctctgtcttcagcctcagt

SEQ ID NO: 200 - RNA, 20 nt, synthetic construct
  source 1..20: mol_type = other RNA
tctcatccttcactgaggct

SEQ ID NO: 201 - RNA, 20 nt, synthetic construct
  source 1..20: mol_type = other RNA
tctgtcttcagcctcagtga

SEQ ID NO: 202 - RNA, 19 nt, synthetic construct
  source 1..19: mol_type = other RNA
cacagaacagccacggtca

SEQ ID NO: 203 - RNA, 20 nt, synthetic construct
  source 1..20: mol_type = other RNA
gcagtgtgtgctgtgaccgt

SEQ ID NO: 204 - RNA, 20 nt, synthetic construct
  source 1..20: mol_type = other RNA
gtgtgtgctgtgaccgtggc

SEQ ID NO: 205 - RNA, 20 nt, synthetic construct
  source 1..20: mol_type = other RNA
tcacagaacagccacggtca

SEQ ID NO: 206 - RNA, 20 nt, synthetic construct
  source 1..20: mol_type = other RNA
cacggtcacagcacacactg

SEQ ID NO: 207 - RNA, 20 nt, synthetic construct
  source 1..20: mol_type = other RNA
cggtcacagcacacactgca

SEQ ID NO: 208 - RNA, 20 nt, synthetic construct
  source 1..20: mol_type = other RNA
tgcagtgtgtgctgtgaccg

SEQ ID NO: 209 - RNA, 20 nt, synthetic construct
  source 1..20: mol_type = other RNA
cacagaacagccacggtcac

SEQ ID NO: 210 - RNA, 20 nt, synthetic construct
  source 1..20: mol_type = other RNA
tcacagaacagccacggtca

SEQ ID NO: 211 - RNA, 20 nt, synthetic construct
  source 1..20: mol_type = other RNA
gtgtgtgctgtgaccgtggc

SEQ ID NO: 212 - RNA, 20 nt, synthetic construct
  source 1..20: mol_type = other RNA
gcagtgtgtgctgtgaccgt

SEQ ID NO: 213 - RNA, 20 nt, synthetic construct
  source 1..20: mol_type = other RNA
cacggtcacagcacacactg

SEQ ID NO: 214 - RNA, 20 nt, synthetic construct
  source 1..20: mol_type = other RNA
cggtcacagcacacactgca

SEQ ID NO: 215 - RNA, 20 nt, synthetic construct
  source 1..20: mol_type = other RNA
ccgcctgcagtgtgtgctgt

SEQ ID NO: 216 - RNA, 20 nt, synthetic construct
  source 1..20: mol_type = other RNA
tgcagtgtgtgctgtgaccg

SEQ ID NO: 217 - RNA, 20 nt, synthetic construct
  source 1..20: mol_type = other RNA
acggtcacagcacacactgc

SEQ ID NO: 218 - RNA, 20 nt, synthetic construct
  source 1..20: mol_type = other RNA
gtcacagcacacactgcagg

SEQ ID NO: 219 - RNA, 20 nt, synthetic construct
  source 1..20: mol_type = other RNA
gtcacagcacacactgcagg

SEQ ID NO: 220 - RNA, 20 nt, synthetic construct
  source 1..20: mol_type = other RNA
acggtcacagcacacactgc

SEQ ID NO: 221 - RNA, 20 nt, synthetic construct
  source 1..20: mol_type = other RNA
gtcacagcacacactgcagg

SEQ ID NO: 222 - RNA, 20 nt, synthetic construct
  source 1..20: mol_type = other RNA
cacactgcaggcggactcag

SEQ ID NO: 223 - RNA, 20 nt, synthetic construct
  source 1..20: mol_type = other RNA
cacactgcaggcggactcag

SEQ ID NO: 224 - RNA, 20 nt, synthetic construct
  source 1..20: mol_type = other RNA
gcaggcggactcagtggatc

SEQ ID NO: 225 - RNA, 20 nt, synthetic construct
  source 1..20: mol_type = other RNA
gcaggcggactcagtggatc

SEQ ID NO: 226 - RNA, 20 nt, synthetic construct
  source 1..20: mol_type = other RNA
ctctgccactgatggggatg

SEQ ID NO: 227 - RNA, 20 nt, synthetic construct
  source 1..20: mol_type = other RNA
tgccactgatggggatgagg

SEQ ID NO: 228 - RNA, 20 nt, synthetic construct
  source 1..20: mol_type = other RNA
ctgccactgatggggatgag

SEQ ID NO: 229 - RNA, 20 nt, synthetic construct
  source 1..20: mol_type = other RNA
tgccactgatggggatgagg

SEQ ID NO: 230 - RNA, 20 nt, synthetic construct
  source 1..20: mol_type = other RNA
ggacctgaatgatgggaccc

SEQ ID NO: 231 - RNA, 20 nt, synthetic construct
  source 1..20: mol_type = other RNA
ggacctgaatgatgggaccc

SEQ ID NO: 232 - RNA, 20 nt, synthetic construct
  source 1..20: mol_type = other RNA
ctgccatggacctgaatgat

SEQ ID NO: 233 - RNA, 20 nt, synthetic construct
  source 1..20: mol_type = other RNA
ctgccatggacctgaatgat

SEQ ID NO: 234 - RNA, 20 nt, synthetic construct
  source 1..20: mol_type = other RNA
cctgccatggacctgaatga

SEQ ID NO: 235 - RNA, 20 nt, synthetic construct
  source 1..20: mol_type = other RNA
cctgccatggacctgaatga

SEQ ID NO: 236 - RNA, 20 nt, synthetic construct
  source 1..20: mol_type = other RNA
catcattcaggtccatggca

SEQ ID NO: 237 - RNA, 20 nt, synthetic construct
  source 1..20: mol_type = other RNA
catcattcaggtccatggca

SEQ ID NO: 238 - RNA, 20 nt, synthetic construct
  source 1..20: mol_type = other RNA
ccatcattcaggtccatggc

SEQ ID NO: 239 - RNA, 20 nt, synthetic construct
  source 1..20: mol_type = other RNA
ccatcattcaggtccatggc

SEQ ID NO: 240 - RNA, 20 nt, synthetic construct
  source 1..20: mol_type = other RNA
ggtcccatcattcaggtcca

SEQ ID NO: 241 - RNA, 20 nt, synthetic construct
  source 1..20: mol_type = other RNA
ggtcccatcattcaggtcca

SEQ ID NO: 242 - RNA, 20 nt, synthetic construct
  source 1..20: mol_type = other RNA
aggcctgggtcccatcattc

SEQ ID NO: 243 - RNA, 20 nt, synthetic construct
  source 1..20: mol_type = other RNA
aggcctgggtcccatcattc

SEQ ID NO: 244 - RNA, 20 nt, synthetic construct
  source 1..20: mol_type = other RNA
aacagctgaatcaggccctt

SEQ ID NO: 245 - RNA, 20 nt, synthetic construct
  source 1..20: mol_type = other RNA
aacagctgaatcaggccctt

SEQ ID NO: 246 - RNA, 20 nt, synthetic construct
  source 1..20: mol_type = other RNA
agctgaatcaggcccttcgg

SEQ ID NO: 247 - RNA, 20 nt, synthetic construct
  source 1..20: mol_type = other RNA
agctgaatcaggcccttcgg

SEQ ID NO: 248 - RNA, 20 nt, synthetic construct
  source 1..20: mol_type = other RNA
caccaccgaagggcctgatt

SEQ ID NO: 249 - RNA, 20 nt, synthetic construct
  source 1..20: mol_type = other RNA
caccaccgaagggcctgatt

SEQ ID NO: 250 - RNA, 20 nt, synthetic construct
  source 1..20: mol_type = other RNA
ctaacacctaaacggttcaa

SEQ ID NO: 251 - RNA, 20 nt, synthetic construct
  source 1..20: mol_type = other RNA
ctaacacctaaacggttcaa

SEQ ID NO: 252 - RNA, 20 nt, synthetic construct
  source 1..20: mol_type = other RNA
ggtaccgtctaacacctaaa

SEQ ID NO: 253 - RNA, 20 nt, synthetic construct
  source 1..20: mol_type = other RNA
tctaacacctaaacggttca

SEQ ID NO: 254 - RNA, 20 nt, synthetic construct
  source 1..20: mol_type = other RNA
gtaccgtctaacacctaaac

SEQ ID NO: 255 - RNA, 20 nt, synthetic construct
  source 1..20: mol_type = other RNA
ggtaccgtctaacacctaaa

SEQ ID NO: 256 - RNA, 20 nt, synthetic construct
  source 1..20: mol_type = other RNA
gttagacggtaccgacaacc

SEQ ID NO: 257 - RNA, 20 nt, synthetic construct
  source 1..20: mol_type = other RNA
gtctaacacctaaacggttc

SEQ ID NO: 258 - RNA, 20 nt, synthetic construct
  source 1..20: mol_type = other RNA
gtctaacacctaaacggttc

SEQ ID NO: 259 - RNA, 20 nt, synthetic construct
  source 1..20: mol_type = other RNA
tgttagacggtaccgacaac

SEQ ID NO: 260 - RNA, 20 nt, synthetic construct
  source 1..20: mol_type = other RNA
tgttagacggtaccgacaac

SEQ ID NO: 261 - RNA, 20 nt, synthetic construct
  source 1..20: mol_type = other RNA
tgttagacggtaccgacaac

SEQ ID NO: 262 - RNA, 20 nt, synthetic construct
  source 1..20: mol_type = other RNA
tgaaccgtttaggtgttaga

SEQ ID NO: 263 - RNA, 20 nt, synthetic construct
  source 1..20: mol_type = other RNA
tctaacacctaaacggttca

SEQ ID NO: 264 - RNA, 20 nt, synthetic construct
  source 1..20: mol_type = other RNA
aacacctaaacggttcaagg

SEQ ID NO: 265 - RNA, 20 nt, synthetic construct
  source 1..20: mol_type = other RNA
ggtaccgtctaacacctaaa

SEQ ID NO: 266 - RNA, 20 nt, synthetic construct
  source 1..20: mol_type = other RNA
ctaacacctaaacggttcaa

SEQ ID NO: 267 - RNA, 20 nt, synthetic construct
  source 1..20: mol_type = other RNA
acacctaaacggttcaaggg

SEQ ID NO: 268 - RNA, 20 nt, synthetic construct
  source 1..20: mol_type = other RNA
taacacctaaacggttcaag

SEQ ID NO: 269 - RNA, 20 nt, synthetic construct
  source 1..20: mol_type = other RNA
tctaacacctaaacggttca

SEQ ID NO: 270 - RNA, 20 nt, synthetic construct
  source 1..20: mol_type = other RNA
aacacctaaacggttcaagg

SEQ ID NO: 271 - RNA, 20 nt, synthetic construct
  source 1..20: mol_type = other RNA
ggtaccgtctaacacctaaa

SEQ ID NO: 272 - RNA, 20 nt, synthetic construct
  source 1..20: mol_type = other RNA
ttgaaccgtttaggtgttag

SEQ ID NO: 273 - RNA, 20 nt, synthetic construct
  source 1..20: mol_type = other RNA
acacctaaacggttcaaggg

SEQ ID NO: 274 - RNA, 20 nt, synthetic construct
  source 1..20: mol_type = other RNA
taacacctaaacggttcaag

SEQ ID NO: 275 - RNA, 20 nt, synthetic construct
  source 1..20: mol_type = other RNA
tctaacacctaaacggttca

SEQ ID NO: 276 - RNA, 20 nt, synthetic construct
  source 1..20: mol_type = other RNA
cacctaaacggttcaagggg

SEQ ID NO: 277 - RNA, 20 nt, synthetic construct
  source 1..20: mol_type = other RNA
tcggtaccgtctaacaccta

SEQ ID NO: 278 - RNA, 20 nt, synthetic construct
  source 1..20: mol_type = other RNA
gtaccgtctaacacctaaac

SEQ ID NO: 279 - RNA, 20 nt, synthetic construct
  source 1..20: mol_type = other RNA
tcggtaccgtctaacaccta

SEQ ID NO: 280 - RNA, 20 nt, synthetic construct
  source 1..20: mol_type = other RNA
tgaaccgtttaggtgttaga

SEQ ID NO: 281 - RNA, 20 nt, synthetic construct
  source 1..20: mol_type = other RNA
ccttgaaccgtttaggtgtt

SEQ ID NO: 282 - RNA, 20 nt, synthetic construct
  source 1..20: mol_type = other RNA
gtcggtaccgtctaacacct

SEQ ID NO: 283 - RNA, 20 nt, synthetic construct
  source 1..20: mol_type = other RNA
gtctaacacctaaacggttc

SEQ ID NO: 284 - RNA, 20 nt, synthetic construct
  source 1..20: mol_type = other RNA
tctaacacctaaacggttca

SEQ ID NO: 285 - RNA, 20 nt, synthetic construct
  source 1..20: mol_type = other RNA
ctaacacctaaacggttcaa

SEQ ID NO: 286 - RNA, 20 nt, synthetic construct
  source 1..20: mol_type = other RNA
taacacctaaacggttcaag

SEQ ID NO: 287 - RNA, 20 nt, synthetic construct
  source 1..20: mol_type = other RNA
aacacctaaacggttcaagg

SEQ ID NO: 288 - RNA, 20 nt, synthetic construct
  source 1..20: mol_type = other RNA
acacctaaacggttcaaggg

SEQ ID NO: 289 - RNA, 20 nt, synthetic construct
  source 1..20: mol_type = other RNA
cacctaaacggttcaagggg

SEQ ID NO: 290 - RNA, 20 nt, synthetic construct
  source 1..20: mol_type = other RNA
ctaaacggttcaaggggggc

SEQ ID NO: 291 - RNA, 20 nt, synthetic construct
  source 1..20: mol_type = other RNA
tcggtaccgtctaacaccta

SEQ ID NO: 292 - RNA, 20 nt, synthetic construct
  source 1..20: mol_type = other RNA
cggtaccgtctaacacctaa

SEQ ID NO: 293 - RNA, 20 nt, synthetic construct
  source 1..20: mol_type = other RNA
ggtaccgtctaacacctaaa

SEQ ID NO: 294 - skipped

SEQ ID NO: 295 - RNA, 20 nt, synthetic construct
  source 1..20: mol_type = other RNA
gtaccgtctaacacctaaac

SEQ ID NO: 296 - RNA, 20 nt, synthetic construct
  source 1..20: mol_type = other RNA
taccgtctaacacctaaacg

SEQ ID NO: 297 - RNA, 20 nt, synthetic construct
  source 1..20: mol_type = other RNA
accgtctaacacctaaacgg

SEQ ID NO: 298 - RNA, 20 nt, synthetic construct
  source 1..20: mol_type = other RNA
ccgtctaacacctaaacggt

SEQ ID NO: 299 - RNA, 20 nt, synthetic construct
  source 1..20: mol_type = other RNA
cgtctaacacctaaacggtt

SEQ ID NO: 300 - RNA, 20 nt, synthetic construct
  source 1..20: mol_type = other RNA
acctaaacggttcaaggggg

SEQ ID NO: 301 - RNA, 20 nt, synthetic construct
  source 1..20: mol_type = other RNA
cctaaacggttcaagggggg

SEQ ID NO: 302 - RNA, 20 nt, synthetic construct
  source 1..20: mol_type = other RNA
aggtgttagacggtaccgac

SEQ ID NO: 303 - RNA, 20 nt, synthetic construct
  source 1..20: mol_type = other RNA
taggtgttagacggtaccga

SEQ ID NO: 304 - RNA, 20 nt, synthetic construct
  source 1..20: mol_type = other RNA
ttaggtgttagacggtaccg

SEQ ID NO: 305 - RNA, 20 nt, synthetic construct
  source 1..20: mol_type = other RNA
tttaggtgttagacggtacc

SEQ ID NO: 306 - RNA, 20 nt, synthetic construct
  source 1..20: mol_type = other RNA
gtttaggtgttagacggtac

SEQ ID NO: 307 - RNA, 20 nt, synthetic construct
  source 1..20: mol_type = other RNA
cgtttaggtgttagacggta

SEQ ID NO: 308 - RNA, 20 nt, synthetic construct
  source 1..20: mol_type = other RNA
ccgtttaggtgttagacggt

SEQ ID NO: 309 - RNA, 20 nt, synthetic construct
  source 1..20: mol_type = other RNA
aaccgtttaggtgttagacg

SEQ ID NO: 310 - RNA, 20 nt, synthetic construct
  source 1..20: mol_type = other RNA
accgtttaggtgttagacgg

SEQ ID NO: 311 - RNA, 20 nt, synthetic construct
  source 1..20: mol_type = other RNA
gaaccgtttaggtgttagac

SEQ ID NO: 312 - RNA, 20 nt, synthetic construct
  source 1..20: mol_type = other RNA
tgaaccgtttaggtgttaga

SEQ ID NO: 313 - RNA, 20 nt, synthetic construct
  source 1..20: mol_type = other RNA
ttgaaccgtttaggtgttag

SEQ ID NO: 314 - RNA, 20 nt, synthetic construct
  source 1..20: mol_type = other RNA
cttgaaccgtttaggtgtta

SEQ ID NO: 315 - RNA, 20 nt, synthetic construct
  source 1..20: mol_type = other RNA
ccttgaaccgtttaggtgtt

SEQ ID NO: 316 - RNA, 20 nt, synthetic construct
  source 1..20: mol_type = other RNA
cccttgaaccgtttaggtgt

SEQ ID NO: 317 - RNA, 20 nt, synthetic construct
  source 1..20: mol_type = other RNA
ccccttgaaccgtttaggtg

SEQ ID NO: 318 - RNA, 20 nt, synthetic construct
  source 1..20: mol_type = other RNA
cccccttgaaccgtttaggt

SEQ ID NO: 319 - RNA, 20 nt, synthetic construct
  source 1..20: mol_type = other RNA
ccccccttgaaccgtttagg

SEQ ID NO: 320 - RNA, 20 nt, synthetic construct
  source 1..20: mol_type = other RNA
gccccccttgaaccgtttag

SEQ ID NO: 321 - RNA, 20 nt, synthetic construct
  source 1..20: mol_type = other RNA
catggatttctccggtgaat

SEQ ID NO: 322 - DNA, 22 bp, synthetic construct
  source 1..22: mol_type = other DNA
gttgtttctgaccactgcttgc

SEQ ID NO: 323 - DNA, 22 bp, synthetic construct
  source 1..22: mol_type = other DNA
aaaaagatgtcaggcccctctg

SEQ ID NO: 324 - DNA, 20 bp, synthetic construct
  source 1..20: mol_type = other DNA
ttgaacttcggtttttgcag

SEQ ID NO: 325 - DNA, 20 bp, synthetic construct
  source 1..20: mol_type = other DNA
cctttcaagaaaacaaaaag

SEQ ID NO: 326 - DNA, 20 bp, synthetic construct
  source 1..20: mol_type = other DNA
atacctgtggcttcctcatc

SEQ ID NO: 327 - DNA, 24 bp, synthetic construct
  source 1..24: mol_type = other DNA
gtgatgtggaatcatgccccaagc

SEQ ID NO: 328 - DNA, 22 bp, synthetic construct
  source 1..22: mol_type = other DNA
ccactttgtagcgcatcacctg

SEQ ID NO: 329 - DNA, 22 bp, synthetic construct
  source 1..22: mol_type = other DNA
cttagcagtttccatgcgtgca

SEQ ID NO: 330 - DNA, 22 bp, synthetic construct
  source 1..22: mol_type = other DNA
gtgacttgacaagctctgccac

SEQ ID NO: 331 - DNA, 22 bp, synthetic construct
  source 1..22: mol_type = other DNA
atgtcacctttgatgcggcaag

SEQ ID NO: 332 - DNA, 20 bp, synthetic construct
  source 1..20: mol_type = other DNA
gacagtcagccgcatcttct

SEQ ID NO: 333 - DNA, 20 bp, synthetic construct
  source 1..20: mol_type = other DNA
atccgttgactccgaccttc

SEQ ID NO: 334 - DNA, 22 bp, synthetic construct
  source 1..22: mol_type = other DNA
aaagaatggtgcccctcggagt

SEQ ID NO: 335 - DNA, 22 bp, synthetic construct
  source 1..22: mol_type = other DNA
ttgcttgttcgagtcaggcacg

SEQ ID NO: 336 - DNA, 55 bp, synthetic construct
  source 1..55: mol_type = other DNA
tcgtcggcagcgtcagatgtgtataagagacagggagggcttgtctcttggtgat

SEQ ID NO: 337 - DNA, 56 bp, synthetic construct
  source 1..56: mol_type = other DNA
gtctcgtgggctcggagatgtgtataagagacagccactttgtagcgcatcacctg

SEQ ID NO: 338 - DNA, 57 bp, synthetic construct
  source 1..57: mol_type = other DNA
tcgtcggcagcgtcagatgtgtataagagacagatctctcaacacaaatttatcaac

SEQ ID NO: 339 - DNA, 50 bp, synthetic construct
  source 1..50: mol_type = other DNA
gtctcgtgggctcggagatgtgtataagagacagcactccagggtcagca

SEQ ID NO: 340 - DNA, 20 bp, synthetic construct
  source 1..20: mol_type = other DNA
gccttctagttgccagccat

SEQ ID NO: 341 - DNA, 19 bp, synthetic construct
  source 1..19: mol_type = other DNA
ggcaccttccagggtcaag

SEQ ID NO: 342 - DNA, 55 bp, synthetic construct
  source 1..55: mol_type = other DNA
tcgtcggcagcgtcagatgtgtataagagacagaagtgtggggttcaggccctga

SEQ ID NO: 343 - DNA, 56 bp, synthetic construct
  source 1..56: mol_type = other DNA
gtctcgtgggctcggagatgtgtataagagacagggagtagcatcaggcccgtgga

SEQ ID NO: 344 - DNA, 58 bp, synthetic construct
  source 1..58: mol_type = other DNA
tcgtcggcagcgtcagatgtgtataagagacagccctctcacacacagtagcagacca

SEQ ID NO: 345 - DNA, 56 bp, synthetic construct
  source 1..56: mol_type = other DNA
gtctcgtgggctcggagatgtgtataagagacaggcccaccttcctctcagaggca

SEQ ID NO: 346 - DNA, 55 bp, synthetic construct
  source 1..55: mol_type = other DNA
tcgtcggcagcgtcagatgtgtataagagacagggcaactgctggaaccaaagca

SEQ ID NO: 347 - DNA, 56 bp, synthetic construct
  source 1..56: mol_type = other DNA
gtctcgtgggctcggagatgtgtataagagacagttcaggtgcttggccgggattt

SEQ ID NO: 348 - DNA, 55 bp, synthetic construct
  source 1..55: mol_type = other DNA
tcgtcggcagcgtcagatgtgtataagagacagatgagtgcacaggcctcccctt

SEQ ID NO: 349 - DNA, 56 bp, synthetic construct
  source 1..56: mol_type = other DNA
gtctcgtgggctcggagatgtgtataagagacagagggtgaacagggccagaagct

SEQ ID NO: 350 - DNA, 55 bp, synthetic construct
  source 1..55: mol_type = other DNA
tcgtcggcagcgtcagatgtgtataagagacagcccggattgctgtgatgctgca

SEQ ID NO: 351 - DNA, 56 bp, synthetic construct
  source 1..56: mol_type = other DNA
gtctcgtgggctcggagatgtgtataagagacagtcaggctcccctacctcccact

SEQ ID NO: 352 - DNA, 55 bp, synthetic construct
  source 1..55: mol_type = other DNA
tcgtcggcagcgtcagatgtgtataagagacagtcacactgtcactgtgctccca

SEQ ID NO: 353 - DNA, 56 bp, synthetic construct
  source 1..56: mol_type = other DNA
gtctcgtgggctcggagatgtgtataagagacagggtgtccacgatggccttcagc

SEQ ID NO: 354 - DNA, 56 bp, synthetic construct
  source 1..56: mol_type = other DNA
tcgtcggcagcgtcagatgtgtataagagacagtgtgctgctccattgttaatgca

SEQ ID NO: 355 - DNA, 60 bp, synthetic construct
  source 1..60: mol_type = other DNA
gtctcgtgggctcggagatgtgtataagagacaggcatcatacaactagagtgaggggca

SEQ ID NO: 356 - DNA, 55 bp, synthetic construct
  source 1..55: mol_type = other DNA
tcgtcggcagcgtcagatgtgtataagagacagacaaactgctccaggcacaccg

SEQ ID NO: 357 - DNA, 56 bp, synthetic construct
  source 1..56: mol_type = other DNA
gtctcgtgggctcggagatgtgtataagagacagacttggcaccaagctggttgca

SEQ ID NO: 358 - DNA, 55 bp, synthetic construct
  source 1..55: mol_type = other DNA
tcgtcggcagcgtcagatgtgtataagagacagggacataagccagcccagataa

SEQ ID NO: 359 - DNA, 58 bp, synthetic construct
  source 1..58: mol_type = other DNA
gtctcgtgggctcggagatgtgtataagagacagtttcatcaccttcttcctcttcct

SEQ ID NO: 360 - DNA, 55 bp, synthetic construct
  source 1..55: mol_type = other DNA
tcgtcggcagcgtcagatgtgtataagagacagtgctgcagtgaacataggcatg

SEQ ID NO: 361 - DNA, 57 bp, synthetic construct
  source 1..57: mol_type = other DNA
gtctcgtgggctcggagatgtgtataagagacagaaagctcaacgtcactgatcatt

SEQ ID NO: 362 - DNA, 55 bp, synthetic construct
  source 1..55: mol_type = other DNA
tcgtcggcagcgtcagatgtgtataagagacagtggccagatatgttcatgccag

SEQ ID NO: 363 - DNA, 56 bp, synthetic construct
  source 1..56: mol_type = other DNA
gtctcgtgggctcggagatgtgtataagagacaggaagtccactgattagcctgca

SEQ ID NO: 364 - DNA, 55 bp, synthetic construct
  source 1..55: mol_type = other DNA
tcgtcggcagcgtcagatgtgtataagagacagaacagggcaccagataaccagg

SEQ ID NO: 365 - DNA, 56 bp, synthetic construct
  source 1..56: mol_type = other DNA
gtctcgtgggctcggagatgtgtataagagacagaattcacaaccaagaaccttaa

SEQ ID NO: 366 - DNA, 55 bp, synthetic construct
  source 1..55: mol_type = other DNA
tcgtcggcagcgtcagatgtgtataagagacagatggcttcctcattgtggtcct

SEQ ID NO: 367 - DNA, 54 bp, synthetic construct
  source 1..54: mol_type = other DNA
gtctcgtgggctcggagatgtgtataagagacaggtcttgatcccctgacctcg

SEQ ID NO: 368 - protein, 7 aa, synthetic construct
PKKKRKV

SEQ ID NO: 369 - protein, 16 aa, synthetic construct
KRPAATKKAGQAKKKK

SEQ ID NO: 370 - protein, 34 aa, synthetic construct
SGGSSGGSSGSETPGTSESATPESSGGSSGGSST

SEQ ID NO: 371 - protein, 95 aa, synthetic construct
SGGSSGGSSGSETPGTSESATPESSGGSSGGSSTLEPGEKPYKCPECGKSFSQSGALTRHQRTHTRDKKYSIGLDIGTNSVGWAVITDEYKVPLE

SEQ ID NO: 372 - protein, 29 aa, synthetic construct
ASGGGGSGGGGSGGGGSGGGGSGGGGSLE

SEQ ID NO: 373 - protein, 34 aa, synthetic construct; identical to SEQ ID NO: 370

SEQ ID NO: 374 - protein, 65 aa, synthetic construct
ASASSGGSSGGSSGSETPGTSESATPESSGGSSGGSGGGGSGGGGSGGGGSGGGGSGGGGSGTLE

SEQ ID NO: 375 - DNA, 169 bp, synthetic construct
  source 1..169: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttc

SEQ ID NO: 376 - DNA, 106 bp, synthetic construct
  source 1..106: mol_type = other DNA
ctgcgcgctcgctcgctcactgaggccgcccgggcaaagcccgggcgtcgggcgacctttggtcgcccggcctcagtgagcgagcgagcgcgcagagagggagtgg

SEQ ID NO: 377 - protein, 102 aa, synthetic construct
CLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 378 - protein, 35 aa, synthetic construct
IKIATRKYLGKQNVYDIGVERDHNFALKNGFIASN

SEQ ID NO: 379 - protein, 1451 aa, synthetic construct
MGPKKKRKVGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKVSGGSSGGSSGSETPGTSESATPESSGGSSGGSSTLEPGEKPYKCPECGKSFSQSGALTRHQRTHTRDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 380 - protein, 975 aa, synthetic construct
MKRTADGSEFESPKKKRKVIKIATRKYLGKQNVYDIGVERDHNFALKNGFIASNCFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSTNLSDIIEKETGKQLVIQESILMLPEEVEEVIGNKPESDILVHTAYDESTDENVMLLTSDAPEYKPWALVIQDSNGENKIKMLSYPYDVPDYAYPYDVPDYAYPYDVPDYASGGSPKKKRKV

SEQ ID NO: 381 - protein, 833 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVCLAGDTLITLADGRRVPIRELVSQQNFSVWALNPQTYRLERARVSRAFCTGIKPVYRLTTRLGRSIRATANHRFLTPQGWKRVDELQPGDYLALPRRIPTAS

SEQ ID NO: 382 - protein, 1438 aa, synthetic construct
MAAACPELRQLAQSDVYWDPIVSIEPDGVEEVFDLTVPGPHNFVANDIIAHNSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 383 - protein, 1060 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNCLAGDTLITLADGRRVPIRELVSQQNFSVWALNPQTYRLERARVSRAFCTGIKPVYRLTTRLGRSIRATANHRFLTPQGWKRVDELQPGDYLALPRRIPTAS

SEQ ID NO: 384 - protein, 1224 aa, synthetic construct
MAAACPELRQLAQSDVYWDPIVSIEPDGVEEVFDLTVPGPHNFVANDIIAHNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPIAGSKASPKKKRKVGRAGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 385 - protein, 1192 aa, synthetic construct
MDYKDHDGDYKDHDIDYKDDDDKMAPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGCLAGDTLITLADGRRVPIRELVSQQNFSVWALNPQTYRLERARVSRAFCTGIKPVYRLTTRLGRSIRATANHRFLTPQGWKRVDELQPGDYLALPRRIPTAS

SEQ ID NO: 386 - protein, 1226 aa, synthetic construct
MKRTADGSEFESPRKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGSGGSSGGSSGSETPGTSESATPESSGGSSGGSSCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNSGGSPKKKRKVPKKKRKV

SEQ ID NO: 387 - protein, 1251 aa, synthetic construct
MKRTADGSEFESPRKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSGGGGSGGGGSGGGGSCLAGDTLITLADGRRVPIRELVSQQNFSVWALNPQTYRLERARVSRAFCTGIKPVYRLTTRLGRSIRATANHRFLTPQGWKRVDELQPGDYLALPRRIPTAS

SEQ ID NO: 388 - protein, 679 aa, synthetic construct
MAAACPELRQLAQSDVYWDPIVSIEPDGVEEVFDLTVPGPHNFVANDIIAHNGGGGSGGGGSGGGGSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPL

SEQ ID NO: 389 - protein, 66 aa, synthetic construct
IKIATRKYLGKQNVYDIGVERDHNFALKNGFIASNGRAGGSSGSETPGTSESATPESSGGSSGGSS

SEQ ID NO: 390 - protein, 4 aa, synthetic construct
DSVD

SEQ ID NO: 391 - protein, 4 aa, synthetic construct
DEED

SEQ ID NO: 392 - protein, 4 aa, synthetic construct
DLND

SEQ ID NO: 393 - protein, 4 aa, synthetic construct
VLDG

SEQ ID NO: 394 - protein, 10 aa, synthetic construct
LDLFSPSVTV

SEQ ID NO: 395 - skipped

SEQ ID NO: 396 - protein, 8 aa, synthetic construct
LDPLGDRA

SEQ ID NO: 397 - DNA, 27 bp, synthetic construct
  source 1..27: mol_type = other DNA
caccaccccctcggtgtggaccgttga

SEQ ID NO: 398 - protein, 7 aa, synthetic construct
DPGLEDF

SEQ ID NO: 399 - DNA, 27 bp, synthetic construct
  source 1..27: mol_type = other DNA
gacccaggcctcgaagatttcggtgtc

SEQ ID NO: 400 - protein, 10 aa, synthetic construct
DPDFGVKGVQ

SEQ ID NO: 401 - DNA, 30 bp, synthetic construct
  source 1..30: mol_type = other DNA
cacccagatttcggtgtcaagggggtgcaa

SEQ ID NO: 402 - protein, 6 aa, synthetic construct
VEDFGV

SEQ ID NO: 403 - DNA, 27 bp, synthetic construct
  source 1..27: mol_type = other DNA
gtggaagatttcgataaggcaggtgtc

SEQ ID NO: 404 - protein, 9 aa, synthetic construct
VEDFARAGV

SEQ ID NO: 405 - DNA, 27 bp, synthetic construct
  source 1..27: mol_type = other DNA
gtgcaagatttcgctaaggcaggtgtc

SEQ ID NO: 406 - DNA, 71 bp, synthetic construct
  source 1..71: mol_type = other DNA
ttctttttcttaatttgttttcaaggtgttctttgcagatgtgttcatcatggtgtggtggaggtaggtaa

SEQ ID NO: 407 - DNA, 69 bp, synthetic construct
  source 1..69: mol_type = other DNA
aagaaaaagattaaacaaaagttccacaagaacgtctacacaagtagtaccacaccacctccatccatt

SEQ ID NO: 408 - DNA, 15 bp, synthetic construct
  source 1..15: mol_type = other DNA
gtggaggtaggtaaa

SEQ ID NO: 409 - protein, 8 aa, synthetic construct
VFFAEDVI

SEQ ID NO: 410 - DNA, 23 bp, synthetic construct
  source 1..23: mol_type = other DNA
aggtgttctttgcagaagatata

SEQ ID NO: 411 - protein, 4 aa, synthetic construct
KMWV

SEQ ID NO: 412 - DNA, 17 bp, synthetic construct
  source 1..17: mol_type = other DNA
agaagatgtgggtttag

SEQ ID NO: 413 - DNA, 10 bp, synthetic construct
  source 1..10: mol_type = other DNA
aggtaggtaa

SEQ ID NO: 414 - protein, 5 aa, synthetic construct
VEVDA

SEQ ID NO: 415 - DNA, 23 bp, synthetic construct
  source 1..23: mol_type = other DNA
gtggaggtaggtaggttgacgcc

SEQ ID NO: 416 - protein, 4 aa, synthetic construct
VEVG

SEQ ID NO: 417 - DNA, 15 bp, synthetic construct
  source 1..15: mol_type = other DNA
gtggaggtaggttga

SEQ ID NO: 418 - protein, 10 aa, synthetic construct
SLASIQELLS

SEQ ID NO: 419 - DNA, 30 bp, synthetic construct
  source 1..30: mol_type = other DNA
agcctggccagtatccaagagctcctgtct

SEQ ID NO: 420 - protein, 10 aa, synthetic construct
SLAYPRAPVS

SEQ ID NO: 421 - DNA, 30 bp, synthetic construct
  source 1..30: mol_type = other DNA
agcctggcgtatccaagagctcctgtctcc

SEQ ID NO: 422 - DNA, 14 bp, synthetic construct
  source 1..14: mol_type = other DNA
aggtgttagacggt

SEQ ID NO: 423 - DNA, 20 bp, synthetic construct
  source 1..20: mol_type = other DNA
cccttgaaccgtttaggtgt

SEQ ID NO: 424 - DNA, 12 bp, synthetic construct
  source 1..12: mol_type = other DNA
gtgttagacggt

SEQ ID NO: 425 - DNA, 17 bp, synthetic construct
  source 1..17: mol_type = other DNA
tttaggtgttagacggt

SEQ ID NO: 426 - DNA, 17 bp, synthetic construct
  source 1..17: mol_type = other DNA
tttaaatgttagacggt

SEQ ID NO: 427 - protein, 19 aa, synthetic construct
QVSEIVLDGQITGISMALQ

SEQ ID NO: 428 - DNA, 57 bp, synthetic construct
  source 1..57: mol_type = other DNA
caggtctctgaaattgtgttagacggtcagattacaggtatttccatggcccttcaa

SEQ ID NO: 429 - protein, 19 aa, synthetic construct
QVSEIVLDGQITGISMALQ

SEQ ID NO: 430 - DNA, 57 bp, synthetic construct; identical to SEQ ID NO: 428
  source 1..57: mol_type = other DNA

SEQ ID NO: 431 - protein, 14 aa, synthetic construct
QVSEIITGISMALQ

SEQ ID NO: 432 - DNA, 42 bp, synthetic construct
  source 1..42: mol_type = other DNA
caggtctctgaaattattacaggtatttccatggcccttcaa

SEQ ID NO: 433 - protein, 7 aa, synthetic construct
QVSEIPF

SEQ ID NO: 434 - DNA, 24 bp, synthetic construct
  source 1..24: mol_type = other DNA
caggtctctgaaattcccttctga

SEQ ID NO: 435 - DNA, 66 bp, synthetic construct
  source 1..66: mol_type = other DNA
caggtctctgaaattaacagccaggtgttagacggtcagattacaggtatttccatggcccttcaa

SEQ ID NO: 436 - protein, 15 aa, synthetic construct
QVSEIPPLNRLSVRR

SEQ ID NO: 437 - DNA, 45 bp, synthetic construct
  source 1..45: mol_type = other DNA
caggtctctgaaattcccttgaaccgtttagtggttagacggtga

SEQ ID NO: 438 - DNA, 20 bp, synthetic construct
  source 1..20: mol_type = other DNA
cccttgaaccgtttaggtgt

SEQ ID NO: 439 - DNA, 50 bp, synthetic construct
  source 1..50: mol_type = other DNA
cacagcccccttgaaccgtttaggtgttagacggtaccgacaaccagtat

SEQ ID NO: 440 - DNA, 50 bp, synthetic construct
  source 1..50: mol_type = other DNA
gtgtagggggaacttggcaaatccacaatctgccatggctgttggtcata

SEQ ID NO: 441 - protein, 9 aa, synthetic construct
VLDGTDNQY

SEQ ID NO: 442 - skipped

SEQ ID NO: 443 - RNA, 14 nt, synthetic construct
  source 1..14: mol_type = other RNA
ttcagcctcagtga

SEQ ID NO: 444 - RNA, 12 nt, synthetic construct
  source 1..12: mol_type = other RNA
cggtcacagcac

SEQ ID NO: 445 - RNA, 12 nt, synthetic construct
  source 1..12: mol_type = other RNA
gactcagtggat

SEQ ID NO: 446 - RNA, 12 nt, synthetic construct
  source 1..12: mol_type = other RNA
gatgaggaggat

SEQ ID NO: 447 - RNA, 12 nt, synthetic construct
  source 1..12: mol_type = other RNA
gacctgaatgat

SEQ ID NO: 448 - RNA, 12 nt, synthetic construct
  source 1..12: mol_type = other RNA
tcagctgttacc

SEQ ID NO: 449 - DNA, 16 bp, synthetic construct
  source 1..16: mol_type = other DNA
ttaggtgttagacggt

SEQ ID NO: 450 - protein, 4 aa, synthetic construct
RSQH

SEQ ID NO: 451 - protein, 4 aa, synthetic construct
DVSD

SEQ ID NO: 452 - DNA, 91 bp, synthetic construct
  source 1..91: mol_type = other DNA
tcacagcccccttgaaccgtttaggtcttacacggtaccgacaaccagtatttgggcctgcagattggacagccccaggatgaagatgagg

SEQ ID NO: 453 - skipped

SEQ ID NO: 454 - DNA, 20 bp, synthetic construct
  source 1..20: mol_type = other DNA
acagctattgggttcaaggg

SEQ ID NO: 455 - DNA, 20 bp, synthetic construct
  source 1..20: mol_type = other DNA
acagtcaaagggttcaaggg

SEQ ID NO: 456 - DNA, 20 bp, synthetic construct
  source 1..20: mol_type = other DNA
acatttaaatggttcaaggg

SEQ ID NO: 457 - DNA, 20 bp, synthetic construct
  source 1..20: mol_type = other DNA
acatttgaaccgttcaaggg

SEQ ID NO: 458 - DNA, 20 bp, synthetic construct
  source 1..20: mol_type = other DNA
agacacaaactgttcaaggg

SEQ ID NO: 459 - DNA, 20 bp, synthetic construct
  source 1..20: mol_type = other DNA
acaggttaacgtttcaaggg

SEQ ID NO: 460 - DNA, 20 bp, synthetic construct
  source 1..20: mol_type = other DNA
accctgaaacgtttcaaggg

SEQ ID NO: 461 - DNA, 20 bp, synthetic construct
  source 1..20: mol_type = other DNA
aatcctaaaaggttgaaggg

SEQ ID NO: 462 - skipped

SEQ ID NO: 463 - DNA, 20 bp, synthetic construct
  source 1..20: mol_type = other DNA
ttacaggtgttagacggtag

SEQ ID NO: 464 - DNA, 20 bp, synthetic construct
  source 1..20: mol_type = other DNA
ttactggtgttagatggtac

SEQ ID NO: 465 - DNA, 20 bp, synthetic construct
  source 1..20: mol_type = other DNA
ttaggtgttacacgctaccc

SEQ ID NO: 466 - DNA, 19 bp, synthetic construct
  source 1..19: mol_type = other DNA
ttagggttagacggtcacc

SEQ ID NO: 467 - DNA, 20 bp, synthetic construct
  source 1..20: mol_type = other DNA
ttaggtgtaagaggtccacc

SEQ ID NO: 468 - DNA, 22 bp, synthetic construct
  source 1..22: mol_type = other DNA
caccactctcttgtccgtttgc

SEQ ID NO: 469 - DNA, 23 bp, synthetic construct
  source 1..23: mol_type = other DNA
ggctgagcatatctgtaagctgc

SEQ ID NO: 470 - DNA, 23 bp, synthetic construct
  source 1..23: mol_type = other DNA
cacctacaggaaattgctggagg

SEQ ID NO: 471-479 - skipped (9 entries)

SEQ ID NO: 480 - DNA, 21 bp, synthetic construct
  source 1..21: mol_type = other DNA
ccacgatgttcctcttgaggt

SEQ ID NO: 481 - DNA, 22 bp, synthetic construct
  source 1..22: mol_type = other DNA
tctgccgtccaaacttgaagcc

SEQ ID NO: 482 - DNA, 20 bp, synthetic construct
  source 1..20: mol_type = other DNA
ctgtggctgaggagccgctg

SEQ ID NO: 483 - DNA, 24 bp, synthetic construct
  source 1..24: mol_type = other DNA
cctgcggtcgagctcctcctctat

SEQ ID NO: 484 - DNA, 22 bp, synthetic construct
  source 1..22: mol_type = other DNA
tggctgaggagccgctgcaccg

SEQ ID NO: 485 - DNA, 22 bp, synthetic construct
  source 1..22: mol_type = other DNA
tggctgaggagccgctgcaccg

SEQ ID NO: 486 - DNA, 20 bp, synthetic construct
  source 1..20: mol_type = other DNA
acatttaaatggttcaaggt

SEQ ID NO: 487 - DNA, 22 bp, synthetic construct
  source 1..22: mol_type = other DNA
ttacaggtgttagacggtagca

SEQ ID NO: 488 - DNA, 22 bp, synthetic construct
  source 1..22: mol_type = other DNA
ttactggtgttagatggtacct

SEQ ID NO: 489 - DNA, 21 bp, synthetic construct
  source 1..21: mol_type = other DNA
ttaggggttagacggtcacct

SEQ ID NO: 490 - DNA, 22 bp, synthetic construct
  source 1..22: mol_type = other DNA
ttaggtgtaagaaggtccaccg

SEQ ID NO: 491 - DNA, 21 bp, synthetic construct
  source 1..21: mol_type = other DNA
tgtaggtgtgagatggtaccg

SEQ ID NO: 492 - DNA, 22 bp, synthetic construct
  source 1..22: mol_type = other DNA
ttaggtgttagtgataccggat

SEQ ID NO: 493 - DNA, 22 bp, synthetic construct
  source 1..22: mol_type = other DNA
ttaggtgttgactttaccgagt

SEQ ID NO: 494 - DNA, 22 bp, synthetic construct
  source 1..22: mol_type = other DNA
ttaggtgttagaggtacaagag

SEQ ID NO: 495 - DNA, 22 bp, synthetic construct
  source 1..22: mol_type = other DNA
ttagatgttagacggtccaatg

SEQ ID NO: 496 - DNA, 22 bp, synthetic construct
  source 1..22: mol_type = other DNA
ttgatgttagacggcaccgaac

SEQ ID NO: 497 - DNA, 22 bp, synthetic construct
  source 1..22: mol_type = other DNA
ttaggtgttaacggtaaggtaa

SEQ ID NO: 498 - DNA, 22 bp, synthetic construct
  source 1..22: mol_type = other DNA
ttaggtgttgagggtacagcaa

SEQ ID NO: 499 - DNA, 22 bp, synthetic construct
  source 1..22: mol_type = other DNA
ttaggtgttagactgtaagtaa

SEQ ID NO: 500 - DNA, 22 bp, synthetic construct
  source 1..22: mol_type = other DNA
taggtgttagacagtactgttg

SEQ ID NO: 501 - DNA, 19 bp, synthetic construct
  source 1..19: mol_type = other DNA
ttaggtgttagaggtagag

SEQ ID NO: 502 - DNA, 19 bp, synthetic construct
  source 1..19: mol_type = other DNA
ttaggtgttgacgtcaccg

SEQ ID NO: 503 - DNA, 19 bp, synthetic construct
  source 1..19: mol_type = other DNA
ttaggtgttaagggtactg

SEQ ID NO: 504 - DNA, 19 bp, synthetic construct
  source 1..19: mol_type = other DNA
ttaggtggagtcggtaccg

SEQ ID NO: 505 - DNA, 20 bp, synthetic construct
  source 1..20: mol_type = other DNA
ttaggtgttagaaggaacag

SEQ ID NO: 506 - DNA, 20 bp, synthetic construct
  source 1..20: mol_type = other DNA
ataggtgttagacagtactg

SEQ ID NO: 507 - DNA, 20 bp, synthetic construct
  source 1..20: mol_type = other DNA
taaggtgttagacggtccct

SEQ ID NO: 508 - protein, 9 aa, synthetic construct
HHGQVGGPI

SEQ ID NO: 509 - DNA, 27 bp, synthetic construct
  source 1..27: mol_type = other DNA
caccacggccaggtgggcggacccatc

SEQ ID NO: 510 - protein, 7 aa, synthetic construct
HHGQSPQ

SEQ ID NO: 511 - DNA, 21 bp, synthetic construct
  source 1..21: mol_type = other DNA
caccacggccagagcccccag

SEQ ID NO: 512 - DNA, 30 bp, synthetic construct
  source 1..30: mol_type = other DNA
ctggacctgttcagcccctcggtgaccgtg

SEQ ID NO: 513 - protein, 4 aa, synthetic construct
SAVT

SEQ ID NO: 514 - protein, 7 aa, synthetic construct
QVSEIEC

SEQ ID NO: 515 - DNA, 24 bp, synthetic construct
  source 1..24: mol_type = other DNA
caggtctctgaaattgaatgttga

SEQ ID NO: 516 - protein, 8 aa, synthetic construct
QVSEISSQ

SEQ ID NO: 517 - DNA, 24 bp, synthetic construct
  source 1..24: mol_type = other DNA
caggtctctgaaattagcagccag

SEQ ID NO: 518 - protein, 17 aa, synthetic construct
NSVIKADSKKGAPPQKC

SEQ ID NO: 519 - DNA, 51 bp, synthetic construct
  source 1..51: mol_type = other DNA
aattctttaatcaaagctgattctaaaaaaggtgcccctcctcagaaatgc

SEQ ID NO: 520 - protein, 10 aa, synthetic construct
NSVIKCKSQQ

SEQ ID NO: 521 - DNA, 30 bp, synthetic construct
  source 1..30: mol_type = other DNA
aattctttaatcaaatgtaaatcccaacaa

SEQ ID NO: 522 - protein, 9 aa, synthetic construct
NSVIKPCSP

SEQ ID NO: 523 - DNA, 30 bp, synthetic construct
  source 1..30: mol_type = other DNA
aattctttaatcaaaccatgcagcccatag

SEQ ID NO: 524 - DNA, 26 bp, synthetic construct
  source 1..26: mol_type = other DNA
aggtgttctttgcagaagatgtgata

SEQ ID NO: 525 - protein, 9 aa, synthetic construct
DPGLEDFGV

SEQ ID NO: 526 - DNA, 71 bp, synthetic construct
  source 1..71: mol_type = other DNA
aagaaaaagaattaaacaaaagttccacaagaaacgtctacacaagtagtaccacaccacctccatccatt

SEQ ID NO: 527 - DNA, 51 bp, synthetic construct
  source 1..51: mol_type = other DNA
cacagccccccttgaaccgtttaggtgttagacggtaccgacaaccagtat

SEQ ID NO: 528 - DNA, 52 bp, synthetic construct
  source 1..52: mol_type = other DNA
gtgtagggggggaacttggcaaatccacaatctgccatggctgttggtcata

SEQ ID NO: 529 - DNA, 13 bp, synthetic construct
  source 1..13: mol_type = other DNA
ttcagcctcagtg

SEQ ID NO: 530 - DNA, 92 bp, synthetic construct
  source 1..92: mol_type = other DNA
tcacagccccccttgaaccgtttaggtgttagacggtaccgacaaccagtatttgggcctgcagattggacagccccaggatgaagatgagg